(19) United States
(12) Patent Application Publication
(10) Pub. No.: US 2024/0117440 A1
(43) Pub. Date: Apr. 11, 2024

(54) PANELS AND METHODS FOR TREATMENT OF DIFFUSE LARGE B-CELL LYMPHOMA

(71) Applicants: The Broad Institute, Inc., Cambridge, MA (US); Dana-Farber Cancer Institute, Inc., Boston, MA (US); The General Hospital Corporation, Boston, MA (US)

(72) Inventors: Bjoern CHAPUY, Boston, MA (US); Donald STEWART, Cambridge, MA (US); Tim WOOD, Cambridge, MA (US); Margaret SHIPP, Boston, MA (US); Gad GETZ, Boston, MA (US); Mark MURAKAMI, Boston, MA (US); Lee LAWTON, Boston, MA (US); Andrew DUNFORD, Cambridge, MA (US); Kirsty WIENAND, Boston, MA (US)

(73) Assignees: The Broad Institute, Inc., Cambridge, MA (US); Dana-Farber Cancer Institute, Inc., Boston, MA (US); The General Hospital Corporation, Boston, MA (US)

(21) Appl. No.: 18/468,384

(22) Filed: Sep. 15, 2023

Related U.S. Application Data
(63) Continuation of application No. PCT/US2022/020762, filed on Mar. 17, 2022.
(60) Provisional application No. 63/163,006, filed on Mar. 18, 2021.

(51) Int. Cl. C12Q 1/6886; C12Q 1/6869; G16B 20/20; G16B 40/20

(52) U.S. Cl. CPC C12Q 1/6886; C12Q 1/6869; G16B 20/20; G16B 40/20; C12Q 2600/106; C12Q 2600/112; C12Q 2600/156

(57) ABSTRACT

The invention provides a molecular classifier and a targeted sequencing assay for use in characterization and treatment of diffuse large B-cell lymphoma.

### CROSS-REFERENCE TO RELATED APPLICATIONS

This application is a continuation under 35 U.S.C. § 111(a) of PCT International Patent Application No. PCT/US2022/020762, filed Mar. 17, 2022, designating the United States and published in English, which claims priority to and the benefit of U.S. Provisional Application No. 63/163,006, filed Mar. 18, 2021, the entire contents of each of which are incorporated by reference herein.

### SEQUENCE LISTING

The present application contains a Sequence Listing which has been submitted electronically in XML format following conversion from the originally filed TXT format.

The content of the electronic XML Sequence Listing, (Date of creation: Sep. 14, 2023; Size: 13,992,026 bytes; Name: 167741-035002US-Sequence_Listing.xml), is herein incorporated by reference in its entirety.

### BACKGROUND OF THE INVENTION

Diffuse large B-cell lymphoma (DLBCL) is the most common lymphoid malignancy in adults. Over 35% of patients with DLBCL cannot be effectively treated using standard induction therapy. There remains a need for better approaches to treatment. Currently, novel targeted agents are tested in unselected patients or those defined solely by clinical prognostic categories, such as the International Prognostic Index (IPI), or tumor transcriptional subgroups, GCB vs ABC. This approach has led to negative phase III trials of multiple targeted agents and uncertain prospects for others. Outcomes for patients with DLBCL have not substantially improved in the last 20 years. Further, current diffuse large B-cell lymphoma (DLBCL) treatment strategies do not reflect the genomic complexity of the disease.

Thus, there remains a need for better methods for treatment of diffuse large B-cell lymphoma.

### SUMMARY OF THE INVENTION

As described below, the present invention features a molecular classifier and a targeted sequencing assay for use in characterization and treatment of diffuse large B-cell lymphoma.

In one aspect, the invention of the disclosure features a method for characterizing a diffuse large B-cell lymphoma (DLBCL) in a subject. The method involves (a) characterizing variants in a biological sample from the subject. The variants are selected from one or more of 10q23.31, 11p, 11q, 11q23.3, 12p, 12p13.2, 12q, 13q, 13q14.2, 13q31.3, 13q34, 14q32.31, 15q15.3, 16q12.1, 17p, 17q24.3, 17q25.1, 18p, 18q, 18q21.32, 18q22.2, 18q23, 19p13.2, 19p13.3, 19q, 19q13.32.1, 19q13.42, 1p13.1, 1p31.1, 1p36.11, 1p36.32, 1q, 1932.1, 1942.12, 21q, 2p16.1, 2q22.2, 3p, 3p21.31, 3q28, 4q21.22, 5p, 6p, 6p21.1, 6p21.33, 6q14.1, 6q21, 7p, 7q, 7q22.1, 8q12.1, 8q24.22, 9p21.3, 9p24.1, ACTB, ARID1A, ATP2A2, B2M, BCL10, BCL2, BCL6, BCL7A, BRAF, BTG1, BTG2, CARD11, CCDC27, CD274, CD58, CD70, CD79B^{mut}, CD83, CREBBP, CRIP1, CXCR4, DTX1, DUSP2, EBF1, EEF1A1, EP300, ETV6, EZH2, FADD, FAS, GNA13, GNAI2, GRHPR, HIST1H1B, HIST1H1C, HIST1H1D, HIST1H1E, HIST1H2AC, HIST1H2AM, HIST1H2BC, HIST1H2BD, HLA-A, HLA-B, HLA-C, HVCN1, IGLL5, IKZF3, IRF2BP2, IRF8, KLHL6, KMT2D, KRAS, LTB, LYN, MAP2K1, MEF2B, MEF2C, MYC, MYD88, MYD88L^{265P}, MYD88^{OTHER}, NFKBIA, NFKBIE, NOTCH2, OSBPL10, PABPC1, PDE4DIP, PIM1, POU2AF1, POU2F2, PRDM1, PTEN, PTPN6, RAC2, RHOA, SESN3, SF3B1, SGK1, SMG7, SOCS1, SPEN, STAT3, TBL1XR1, TET2, TMEM30A, TMSB4X, TNFAIP3, TNFRSF14, TNIP1, TOX, TP53, TUBGCP5, UBE2A, VMP1, YY1, ZC3H12A, ZEB2, ZFP36L1, ZNF423, and ZNF608. The variants are characterized by using a targeted sequencing panel to characterize classes of the variants in the sample based upon the characterization of alterations in the variants. The alterations are selected from the group one or more of a mutation, a structural variant (SV), and a somatic copy number alteration (SCNA). The method involves (b) assigning a classification-specific weighted value to each class of variant characterized. Each classification-specific weighted value reflects the magnitude of the characterized alteration in each class of variant. The method also involves (c) condensing the variant classification-specific weighted values into two or more metafeatures. The method further involves (d) using the metafeatures as input variables for a computational analysis to assign the DLBCL to one of DLBCL subclasses C1 to C5, thereby characterizing the DLBCL.

In another aspect, the invention of the disclosure features a method for selecting a treatment for a subject having a diffuse large B-cell lymphoma (DLBCL). The method involves (a) characterizing variants in a biological sample from the subject. The variants are selected from one or more of 10q23.31, 11p, 11q, 11q23.3, 12p, 12p13.2, 12q, 13q, 13q14.2, 13q31.3, 13q34, 14q32.31, 15q15.3, 16q12.1, 17p, 17q24.3, 17q25.1, 18p, 18q, 18q21.32, 18q22.2, 18q23, 19p13.2, 19p13.3, 19q, 19q13.32.1, 19q13.42, 1p13.1, 1p31.1, 1p36.11, 1p36.32, 1q, 1q32.1, 1q42.12, 21q, 2p16.1, 2q22.2, 3p, 3p21.31, 3q28, 4q21.22, 5p, 6p, 6p21.1, 6p21.33, 6q14.1, 6q21, 7p, 7q, 7q22.1, 8q12.1, 8q24.22, 9p21.3, 9p24.1, ACTB, ARID1A, ATP2A2, B2M, BCL10, BCL2, BCL6, BCL7A, BRAF, BTG1, BTG2, CARD11, CCDC27, CD274, CD58, CD70, CD79B^{mut}, CD83, CREBBP, CRIP1, CXCR4, DTX1, DUSP2, EBF1, EEF1A1, EP300, ETV6, EZH2, FADD, FAS, GNA13, GNAI2, GRHPR, HIST1H1B, HIST1H1C, HIST1H1D, HIST1H1E, HIST1H2AC, HIST1H2AM, HIST1H2BC, HIST1H2BD, HLA-A, HLA-B, HLA-C, HVCN1, IGLL5, IKZF3, IRF2BP2, IRF8, KLHL6, KMT2D, KRAS, LTB, LYN, MAP2K1, MEF2B, MEF2C, MYC, MYD88, MYD88^{L265P}, MYD88^{OTHER}, NFKBIA, NFKBIE, NOTCH2, OSBPL10, PABPC1, PDE4DIP, PIM1, POU2AF1, POU2F2, PRDM1, PTEN, PTPN6, RAC2, RHOA, SESN3, SF3B1, SGK1, SMG7, SOCS1, SPEN, STAT3, TBL1XR1, TET2, TMEM30A, TMSB4X, TNFAIP3, TNFRSF14, TNIP1, TOX, TP53, TUBGCP5, UBE2A, VMP1, YY1, ZC3H12A, ZEB2, ZFP36L1, ZNF423, and ZNF608. The variants are characterized by characterizing classes of the variants in the sample based upon the characterization of alterations in the variants. The alterations are selected from one or more of a mutation, a structural variant (SV), and a somatic copy number alteration (SCNA). The method involves (b) assigning a classification-specific weighted value to each class of variant characterized, where each classification-specific weighted value reflects the magnitude of the characterized alteration in each class of variant. The method involves (c) condensing the variant classification-specific weighted values into two or more metafeatures. The method also involves (d) assigning the DLBCL as belonging to one of DLBCL subclasses C1 to C5 using a computational analysis. The metafeatures are used as input variables for the computational analysis. The method further involves (e), which involves (i), (ii), or (iii), thereby selecting a treatment for the subject having a DLBCL. For (i), if the DLBCL is assigned to class C1 or C5, administering to the subject a treatment containing an agent selected from one or more of a NOTCH inhibitor, a BCL6 inhibitor and an activator of immune evasion, optionally an oligonucleotide inhibitor of NOTCH and/or BCL6, for administration to the subject, or an agent selected from one or more of a BCR/TLR signaling inhibitor and a BCL2 inhibitor, optionally oblimersen, ABT-263, Venetoclax (ABT-199), an antibody or oligonucleotide inhibitor of BCR/TLR signaling and/or an oligonucleotide inhibitor of BCL2. For (ii), if the DLBCL is assigned to DLBCL subclass C3 or C4 class, administering to the subject either a treatment containing an agent selected from one or more of a BCL2 inhibitor, a PI3K inhibitor and an epigenetic modifier, optionally oblimersen, ABT-263, Venetoclax (ABT-199), wortmannin, LY294002, an E2H2 inhibitor (optionally 3-deazaneplanocin A (DZNep), EPZ005687, EI1, GSK126, and/or UNC1999), a CREBBP inhibitor, an oligonucleotide inhibitor of BCL2, an oligonucleotide inhibitor of PI3K and/or an oligonucleotide inhibitor of an epigenetic modifier; or an agent selected from one or more of a JAK/STAT inhibitor and a BRAF/MEK1 inhibitor, optionally ruxolitinib, Vemurafenib, Cobimetinib, an oligonucleotide inhibitor of JAK/STAT and/or an oligonucleotide inhibitor of BRAF/MEK1. For (iii), if the DLBCL is assigned to DLBCL subclass C2, selecting a treatment containing a CDK inhibitor.

In another aspect, the invention of the disclosure features a targeted sequencing panel. The targeted sequencing panel contains oligonucleotides suitable for use in targeted sequencing to characterize two or more classes of variants in a biological sample based upon the characterization of alterations in the variants. The alterations are selected from one or more of a mutation, a structural variant (SV), and a somatic copy number alteration (SCNA). The variants are selected from one or more of 10q23.31, 11p, 11q, 11q23.3, 12p, 12p13.2, 12q, 13q, 13q14.2, 13q31.3, 13q34, 14q32.31, 15q15.3, 16q12.1, 17p, 17q24.3, 17q25.1, 18p, 18q, 18q21.32, 18q22.2, 18q23, 19p13.2, 19p13.3, 19q, 19q13.32.1, 19q13.42, 1p13.1, 1p31.1, 1p36.11, 1p36.32, 1q, 1q32.1, 1q42.12, 21q, 2p16.1, 2q22.2, 3p, 3p21.31, 3q28, 4q21.22, 5p, 6p, 6p21.1, 6p21.33, 6q14.1, 6q21, 7p, 7q, 7q22.1, 8q12.1, 8q24.22, 9p21.3, 9p24.1, ACTB, ARID1A, ATP2A2, B2M, BCL10, BCL2, BCL6, BCL7A, BRAF, BTG1, BTG2, CARD11, CCDC27, CD274, CD58, CD70, CD79B1^{mut}, CD83, CREBBP, CRIP1, CXCR4, DTX1, DUSP2, EBF1, EEF1A1, EP300, ETV6, EZH2, FADD, FAS, GNA13, GNAI2, GRHPR, HIST1H1B, HIST1H1C, HIST1H1D, HIST1H1E, HIST1H2AC, HIST1H2AM, HIST1H2BC, HIST1H2BD, HLA-A, HLA-B, HLA-C, HVCN1, IGLL5, IKZF3, IRF2BP2, IRF8, KLHL6, KMT2D, KRAS, LTB, LYN, MAP2K1, MEF2B, MEF2C, MYC, MYD88, MYD88^{L265P}, MYD88^{OTHER}, NFKBIA, NFKBIE, NOTCH2, OSBPL10, PABPC1, PDE4DIP, PIM1, POU2AF1, POU2F2, PRDM1, PTEN, PTPN6, RAC2, RHOA, SESN3, SF3B1, SGK1, SMG7, SOCS1, SPEN, STAT3, TBL1XR1, TET2, TMEM30A, TMSB4X, TNFAIP3, TNFRSF14, TNIP1, TOX, TP53, TUBGCP5, UBE2A, VMP1, YY1, ZC3H12A, ZEB2, ZFP36L1, ZNF423, and ZNF608.

In another aspect, the invention of the disclosure features a method involving instructing, by at least one processor, at least one computing device to render at least one diffuse large B-cell lymphoma (DLBCL) classification interface. The at least one DLBCL classification interface contains at least one gene sample matrix (GSM) array input element configured to accept at least one GSM array input file storing at least one GSM array associated with at least one patient. The method also involves receiving, by the at least one processor, via the at least one GSM array input element the at least one GSM array input file. The at least one GSM array input file represents at least one GSM array that characterizes classes of variants in the at least one sample of oligonucleotides. The classes of variants are characterized using a targeted sequencing panel containing the oligonucleotides suitable for use in targeted sequencing of the variant classes. The method also involves generating, by the at least one processor, at least one metafeature based at least in part on a weighted sum of the classes of the variants in the at least one GSM array. The method further involves utilizing, by the at least one processor, at least one DLBCL classification machine learning model to generate at least one cluster identification categorizing the at least one patient based at least in part on the at least one metafeature and at least one trained classification layer. The at least one cluster identification characterizes DLBCL burden on a subject associated with the at least one patient. The method also involves instructing, by the at least one processor, the at least one computing device to render at least one DLBCL classification results element in the at least one DLBCL classification interface. The at least one DLBCL classification results element depicts a representation of the at least one cluster identification categorizing the at least one patient.

In another aspect, the invention of the disclosure features a system. The system contains at least one processor configured to execute software instructions which, upon execution, cause the at least one processor to perform steps to: instruct at least one computing device to render at least one diffuse large B-cell lymphoma (DLBCL) classification interface, the at least one DLBCL classification interface containing at least one gene sample matrix (GSM) array input element configured to accept at least one GSM array input file storing at least one GSM array associated with at least one patient; receive via the at least one GSM array input element the at least one GSM array input file, where the at least one GSM array input file represents at least one GSM array that characterizes classes of variants in the at least one sample of oligonucleotides, where the classes of variants are characterized using a targeted sequencing panel containing the oligonucleotides suitable for use in targeted sequencing of the variant classes; generate at least one metafeature based at least in part on a weighted sum of the classes of the variants in the at least one GSM array; utilize at least one DLBCL classification machine learning model to generate at least one cluster identification categorizing the at least one patient based at least in part on the at least one metafeature and at least one trained classification layer, where the at least one cluster identification characterizes DLBCL burden on a subject associated with the at least one patient; and instruct the at least one computing device to render at least one DLBCL classification results element in the at least one DLBCL classification interface, where the at least one DLBCL classification results element depicts a representation of the at least one cluster identification categorizing the at least one patient.

In any of the above aspects, or embodiments thereof, the method further involves characterizing the DLBCL as high-risk if the DLBCL is assigned to subclass C5 or C3. In any of the above aspects, or embodiments thereof, the agent contains one or more of rituximab, cyclophosphamide adriamycin, vincristine, prednisone, doxorubicin hydrochloride, and vincristine sulfate. In any of the above aspects, or embodiments thereof, the agent contains one or more of rituximab, cyclophosphamide, doxorubicin hydrochloride, vincristine sulfate, and prednisone (R-CHOP).

In any of the above aspects, or embodiments thereof, less than 25 metafeatures are used as the input variables. In any of the above aspects, or embodiments thereof, 21 metafeatures or less are used as the input variables. In any of the above aspects, or embodiments thereof, 21 metafeatures are used as the input variables. In any of the above aspects, or embodiments thereof, the metafeatures are selected from one or more of M1, M2, M3, M4, M5, M6, M7, M8, M9, M10, M11, M12, M13, M14, M15, M16, M17, M18, M19, M20, and M21. In any of the above aspects, or embodiments thereof, at least one class of variant corresponding to each metafeature is characterized. In any of the above aspects, or embodiments thereof, all the classes of variants listed in Table 3 are characterized.

In any of the above aspects, or embodiments thereof, condensing the variant classification-specific weighted values involves summing the values corresponding to each metafeature. In embodiments, the weighted values are proportional to degree of variation from a reference sequence. In any of the above aspects, or embodiments thereof, in step (a), characterization involves (i) determining for the mutation classes of variants whether there is a mutation and, if there is a mutation, whether the mutation is a silent mutation or a non-synonymous mutation; (ii) determining for the SCNA mutation class of variants whether there is an SCNA and, if there is an SCNA, whether the SCNA is a low level copy number alteration or a high level copy number alteration; and/or (iii) determining for the SV mutation class of variants whether or not an SV is present.

In any of the above aspects, or embodiments thereof, the variant-specific weighted values are condensed into metafeatures as indicated in Table 3.

In any of the above aspects, or embodiments thereof, the computational method is an artificial neural network classification method, a random forest classification method, or a naïve Bayes classification method. In any of the above aspects, or embodiments thereof, the computational method is an artificial neural network classification method.

In any of the above aspects, or embodiments thereof, the biological sample contains cell free DNA. In any of the above aspects, or embodiments thereof, the method involves characterizing the variants in the cell free DNA. In any of the above aspects, or embodiments thereof, the biological sample contains a bodily fluid and/or a tissue sample. In embodiments, the bodily fluid contains ascites, blood, plasma, pleural fluid, serum, cerebrospinal fluid, phlegm, saliva, stool, urine, semen, prostate fluid, breast milk, and tears. In embodiments, the tissue sample is a biopsy. In embodiments, the biopsy contains a primary tumor sample.

In any of the above aspects, or embodiments thereof, subject is a human.

In any of the above aspects, or embodiments thereof, the classes of variants belong to two or more of metafeatures selected from one or more of M1, M2, M3, M4, M5, M6, M7, M8, M9, M10, M11, M12, M13, M14, M15, M16, M17, M18, M19, M20, and M21. In any of the above aspects, or embodiments thereof, the oligonucleotides are suitable for use in targeted sequencing to characterize at least one variant class corresponding to each of M1, M2, M3, M4, M5, M6, M7, M8, M9, M10, M11, M12, M13, M14, M15, M16, M17, M18, M19, M20, and M21.

In any of the above aspects, or embodiments thereof, the oligonucleotides are suitable for use in targeted sequencing to characterize all of the variant classes listed in Table 3.

In any of the above aspects, or embodiments thereof, the sequencing panel contains oligonucleotide sequences suitable for use in targeted sequencing to measure microsatellite instability, tumor mutational burden, and/or to detect Epstein Barr virus.

In any of the above aspects, or embodiments thereof, the targeted sequencing panel contains polynucleotides sharing at least 85% sequence identity over a span of at least 80 nucleotides to at least one sequence listed in SEQ ID NOs: 1-9244 targeting the classes of variants. In any of the above aspects, or embodiments thereof, the targeted sequencing panel collectively contains polynucleotides sharing at least 85% sequence identity over a span of at least 80 nucleotides each sequence of SEQ ID NOs: 1-9244 targeting the classes of variants. In any of the above aspects, or embodiments thereof, the targeted sequencing panel contains polynucleotides sharing at least 85% sequence identity over a span of at least 80 nucleotides to at least one sequence listed in Table 2. In any of the above aspects, or embodiments thereof, the targeted sequencing panel contains polynucleotides sharing at least 85% sequence identity over a span of at least 80 nucleotides to all of the sequences listed in Table 2. In any of the above aspects, or embodiments thereof, the targeted sequencing panel contains polynucleotides sharing at least 85% sequence identity over a span of at least 80 nucleotides to at least one sequence of SEQ ID NOs: 1-9244 targeting microsatellite instability. In any of the above aspects, or embodiments thereof, the targeted sequencing panel contains polynucleotides sharing at least 85% sequence identity over a span of at least 80 nucleotides to all sequences of SEQ ID NOs: 1-9244 targeting microsatellite instability. In any of the above aspects, or embodiments thereof, the targeted sequencing panel contains polynucleotides complementary to at least about 80% of chromosomal regions collectively targeted by sequences of SEQ ID NOs: 1-9244 corresponding to the classes of variants.

In any of the above aspects, or embodiments thereof, the method further involves receiving, by the at least one processor, at least one DLBCL classification request from the at least one computing device associated with the at least one patient, where the at least one DLBCL classification request involves at least one electronic request over a network; and generating, by the at least one processor, at least one rendering instruction in response to the at least one DLBCL classification request, where the at least one rendering instruction is configured to instruct the at least one computing device to render the at least one DLBCL classification interface.

In any of the above aspects, or embodiments thereof, the at least one trained classification layer contains an artificial neural network having learned weights for each of a plurality of neural network nodes.

In any of the above aspects, or embodiments thereof, the method further involves: utilizing, by the at least one processor, at least one dimensionality reduction model to create a two-dimensional representation of the at least one metafeature; and generating, by the at least one processor, the at least one DLBCL classification results element containing a two-dimensional visualization of the two-dimensional representation of the at least one metafeature, where the two-dimensional visualization contains at least one labelled data point representing the at least one metafeature and the at least one cluster identification associated with the at least one metafeature.

In any of the above aspects, or embodiments thereof, the at least one dimensionality reduction model involves uniform manifold approximation and projection (UMAP). In any of the above aspects, or embodiments thereof, the at least one DLBCL classification results element contains a listing of the at least one metafeature and the at least one cluster identification associated with the at least one metafeature.

In any of the above aspects, or embodiments thereof, the at least one trained classification layer is configured to produce at least one confidence score associated with the at least one cluster identification for the at least one metafeature; where the listing contains the at least one confidence score associated with the at least one cluster identification for the at least one metafeature.

In any of the above aspects, or embodiments thereof, the at least one DLBCL classification results element contains a heatmap depicting: i) at least one gene mutation associated with the at least one metafeature, and ii) the at least one cluster identification associated with the at least one metafeature.

In any of the above aspects, or embodiments thereof, the method further involves: generating, by the at least one processor, at least one treatment selection based at least in part on the at least one cluster identification; where the at least one DLBCL classification results element represents that at least one treatment selection.

In any of the above aspects, or embodiments thereof, the at least one processor is remote from the at least one computing device. In any of the above aspects, or embodiments thereof, the at least one processor is further configured to execute software instructions which, upon execution, further cause the at least one processor to perform steps to: receive at least one DLBCL classification request from the at least one computing device associated with the at least one patient, where the at least one DLBCL classification request involves at least one electronic request over a network; and generate at least one rendering instruction in response to the at least one DLBCL classification request, where the at least one rendering instruction is configured to instruct the at least one computing device to render the at least one DLBCL classification interface.

In any of the above aspects, or embodiments thereof, the at least one trained classification layer contains an artificial neural network having learned weights for each of a plurality of neural network nodes.

In any of the above aspects, or embodiments thereof, the at least one processor is further configured to execute software instructions which, upon execution, further cause the at least one processor to perform steps to: utilize at least one dimensionality reduction model to create a two-dimensional representation of the at least one metafeature; and generate the at least one DLBCL classification results element containing a two-dimensional visualization of the two-dimensional representation of the at least one metafeature, where the two-dimensional visualization contains at least one labelled data point representing the at least one metafeature and the at least one cluster identification associated with the at least one metafeature.

In any of the above aspects, or embodiments thereof, the at least one dimensionality reduction model contains uniform manifold approximation and projection (UMAP).

In any of the above aspects, or embodiments thereof, the at least one DLBCL classification results element contains a listing of the at least one metafeature and the at least one cluster identification associated with the at least one metafeature.

In any of the above aspects, or embodiments thereof, the at least one trained classification layer is configured to produce at least one confidence score associated with the at least one cluster identification for the at least one metafeature; where the listing contains the at least one confidence score associated with the at least one cluster identification for the at least one metafeature.

In any of the above aspects, or embodiments thereof, the at least one DLBCL classification results element contains a heatmap depicting: i) at least one gene mutation associated with the at least one metafeature, and ii) the at least one cluster identification associated with the at least one metafeature.

In any of the above aspects, or embodiments thereof, the at least one processor is further configured to execute software instructions which, upon execution, further cause the at least one processor to perform steps to: generate at least one treatment selection based at least in part on the at least one cluster identification; where the at least one DLBCL classification results element represents that at least one treatment selection.

In any of the above aspects, or embodiments thereof, the at least one processor is remote from the at least one computing device.

The invention provides a molecular classifier and a targeted sequencing assay for use in characterization and treatment of diffuse large B-cell lymphoma. Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “variant” is meant a genomic locus associated with an alteration associated with diffuse large B-cell lymphoma. The alteration can be a mutation, a somatic copy number alteration (SCNA), or a somatic variant (SV; including translocations). The alteration can be a focal alteration or an arm variation. The somatic copy number alteration (SCNA) can be a copy number gain or a copy number loss. A focal alteration is an alteration affecting a small region of a chromosome; for example, the small region can be about or less than about 100 bp, 200 bp, 300 bp, 400 bp, 500 bp, 1 kbp, 5 kbp, 10 kbp, 25 kbp, 50 kbp, 100 kbp, 250 kbp, 500 kbp, or 1 Mbp in size. An arm alteration is an alteration affecting a large region of a chromosome (e.g., an arm of a chromosome). An arm alteration can affect a large region containing about or at least about 500 bp, 1 kbp, 5 kbp, 10 kbp, 25 kbp, 50 kbp, 100 kbp, 250 kbp, 500 kbp, 1 Mbp, 2 Mbp, 3 Mbp, 4 Mbp, or 5 Mbp. The alteration can be an alteration affecting a gene (i.e., a “gene” alteration). A variant can be described as a “target” for sequencing and the variant can be assigned to a “type” where the type identifies the type of genomic locus associated with the variant; for example, a gene, a large chromosomal region (“arm”), a small chromosomal region (“focal”), or microsatellite instability (“MSI” or a microsatellite).

By “variant classification” or “class of variant” is meant a particular alteration associated with a variant.

By “metafeature” is meant one variable of a reduced set of variables determined using dimensionality reduction. In embodiments, a metafeature is a weighted sum (i.e., “metafeature value”) of variant classes observed in a sample (see, e.g., Tables 1 and 3). Table 3 below provides representative definitions of metafeatures. A variant class can be assigned a higher weight proportional to the magnitude of an associated alteration; for example, a higher alteration in copy number can be assigned a higher weight than a lower alteration in copy number (see, e.g., the weighting scheme provided in Table 1).

TABLE 1
Weighing scale used to calculate metafeature
values based on variant measurements.

| Type | Value | Meaning |
|---|---|---|
| Mutations | 0 | no mutation |
| | 1 | silent mutation |
| | 2 | non-synonymous mutation |
| SCNA | 0 | no SCNA |
| | 1 | low level copy number alteration |
| | 2 | high level copy number alteration |
| SV | 0 | no SV |
| | 3 | SV |

By “agent” is meant any small molecule chemical compound, antibody, nucleic acid molecule, or polypeptide, or fragments thereof.

As used herein, the term “algorithm” refers to any formula, model, mathematical equation, algorithmic, analytical, or programmed process, or statistical technique or classification analysis that takes one or more inputs or parameters, whether continuous or categorical, and calculates an output value, index, index value or score. Examples of algorithms include but are not limited to ratios, sums, regression operators such as exponents or coefficients, biomarker value transformations and normalizations (including, without limitation, normalization schemes that are based on clinical parameters such as age, gender, ethnicity, etc.), rules and guidelines, statistical classification models, statistical weights, and neural networks trained on populations or datasets.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

“Biological sample” as used herein refers to a sample obtained from a subject. Biological samples include samples of biological tissue or fluid origin, obtained, reached, or collected in vivo or in situ, that contains or is suspected of containing a polynucleotide. A biological sample also includes samples from a region of a biological subject containing precancerous or cancer cells or tissues. Such samples can be, but are not limited to, organs, tissues, fractions and cells isolated from mammals including, humans such as a patient, mice, and rats. Biological samples also may include sections of the biological sample including tissues, for example, frozen sections taken for histologic purposes. In embodiments, a sample is a blood, plasma, or serum sample comprising circulating tumor DNA.

By “circulating tumor DNA (ctDNA)” is meant cell-free DNA found in the bloodstream of a subject that is derived from neoplasm cells. In embodiments, the neoplasm is a cancer.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “control” or “reference” is meant a standard of comparison. In one aspect, as used herein, “changed as compared to a control” sample or subject is understood as having a level that is statistically different than a sample from a normal, untreated, or control sample. Control samples include, for example, cells in culture, one or more laboratory test animals, one or more human subjects, or biological samples from the same (e.g., cfDNA). Methods to select and test control samples are within the ability of those in the art. Determination of statistical significance is within the ability of those skilled in the art, e.g., the number of standard deviations from the mean that constitute a positive result. In embodiments, a reference is a subject or a sample from a subject that does not have a cancer or a subject prior to a change in a treatment or administration of a drug or treatment. In embodiments, the reference is a matched normal sample or a panel of normals (PoN), where in some instances the matched normal sample is a sample from a healthy subject and/or a subject that does not have a cancer (e.g., a subject prior to being diagnosed with a DLBCL).

By “consist essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight.

By “copy number variation (CNV),” “copy number alteration (CNA),” or “somatic copy number alteration (SCNA)” is meant an alteration that results in a gain or loss in copies of a section(s) of a genome. Non-limiting examples of SCNAs include duplications and deletions.

As used herein, the term “coverage” refers to the number of sequence reads that align to a specific locus in a reference sequence. In embodiments, the reference sequence is a reference genome. For example, with regard to the terminal base of the following reference sequence, because there is only one sample base aligned at this locus (the bold cytosine in Read 2), there is 1× coverage of the reference sequence at this locus. At the 5′ end, there is 3× coverage of the reference sequence at the 5′ terminus guanine.

Reference Sequence:
5′ GGGAAGGGCGATC 3′
Read 1
GGGAAGGGCGAT
Read 2
GGGAAGGGCGATC
Read 3
GGGAAGGGCG

When a genome is sequenced, there will be a large number of nucleotides sequenced. If an individual genome is sequenced only once, there will be a significant number of sequencing errors. To increase the sequencing accuracy, an individual genome will need to be sequenced a large number of times. The average coverage for a whole genome can be calculated from the length of the original genome (G), the number of reads (N), and the average read length (L) as N×L/G. In another example, a hypothetical genome with 2,000 base pairs reconstructed from 8 reads with an average length of 500 nucleotides will have 2× redundancy. This parameter also enables one to estimate other quantities, such as the percentage of the genome covered by reads (sometimes also called breadth of coverage). At a coverage of 0.1×, only 10% of a reference sequence is covered by sequence reads. In embodiments, a sample polynucleotide is sequenced to a coverage of about, at least about, and/or no more than about 1e-8×, 1e-7×, 1e-6×, 1e-5×, 1e-4×, 1e-3×, 1e-2×, 0.05×, 0.1×, 0.2×, 0.3×, 0.4×, 0.5×, 1×, 2×, 3×, 4×, 5×, 7×, 8×, 9×, 10×, 20×, 30×, 40×, 50×, 60×, 70×, 90×, 100×, 200×, 300×, 400×, 500×, 600×, 700×, 800×, 900×, 1000×, 5000×, 10000×, 15000×, 20000×, 25000×, 30000×, 50000×, 100000×, or more.

By “ultra-low coverage” is meant a coverage of less than at least 5×. In some instances, ultra-low coverage is a coverage of less than 0.5×, 0.2×, or 0.1×.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include cancer (e.g., Hodgkin's lymphoma, primary mediastinal B-cell lymphoma), and related diseases or disorders.

By “effective amount” is meant the amount of an agent required to provide a therapeutic benefit in the treatment of a condition or to delay or minimize one or more symptoms associated with the condition. In some instances, the effective amount ameliorates the symptoms of a disease relative to an untreated patient. A therapeutically effective amount of an agent can mean an amount of therapeutic agent, alone or in combination with other therapies, which provides a therapeutic benefit in the treatment of the condition. The term “effective amount” can encompass an amount that improves overall therapy, reduces or avoids symptoms, signs, or causes of the condition, and/or enhances the therapeutic efficacy of another therapeutic agent. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “immunotherapy” is meant a treatment that involves supplementing or stimulating the immune system. Non-limiting examples of immunotherapies include treatments involving administration of biologics, such as immune checkpoint blockades, and/or CAR T cells.

By “immune checkpoint blockade” is meant an agent that functions as an inhibitor of a polynucleotide and/or pathway that functions in inhibiting or stimulating an immune response. In embodiments, the agent is an antibody.

By “increases” is meant a positive alteration of at least 10%, 25%, 50%, 75%, or 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant a protein, polynucleotide, or other analyte having an alteration in sequence, copy number, structure, expression level or activity that is associated with a disease or disorder.

By “mutation” is meant an alteration to a polynucleotide sequence. Non-limiting examples of non-synonymous mutations include single-nucleotide polymorphisms (SNPs), single-nucleotide variations (SNVs), and insertions or deletions (indel mutations). In embodiments, a non-synonymous mutation corresponds to a genomic region about or less than about 1 bp, 2 bp, 3 bp, 4 bp, 5 bp, 10 bp, 50 bp, or 100 bp in size.

As used herein, the term “next-generation sequencing (NGS)” refers to a variety of high-throughput sequencing technologies that parallelize the sequencing process, producing thousands or millions of sequence reads at once. NGS parallelization of sequencing reactions can generate hundreds of megabases to gigabases of nucleotide sequence reads in a single instrument run. Unlike conventional sequencing techniques, such as Sanger sequencing, which typically report the average genotype of an aggregate collection of molecules, NGS technologies typically digitally tabulate the sequence of numerous individual DNA fragments (sequence reads discussed in detail below), such that low frequency variants (e.g., variants present at less than about 10%, 5% or 1% frequency in a heterogeneous population of nucleic acid molecules) can be detected. The term “massively parallel” can also be used to refer to the simultaneous generation of sequence information from many different template molecules by NGS. NGS sequencing platforms include, but are not limited to, the following: Massively Parallel Signature Sequencing (Lynx Therapeutics); 454 pyro-sequencing (454 Life Sciences/Roche Diagnostics); solid-phase, reversible dye-terminator sequencing (Solexa/Illumina); SOLiD technology (Applied Biosystems); Ion semiconductor sequencing (ion Torrent); and DNA nanoball sequencing (Complete Genomics). Descriptions of certain NGS platforms can be found in the following: Shendure, et al., “Next-generation DNA sequencing,” Nature, 2008, vol. 26, No. 10, 135-1 145; Mardis, “The impact of next-generation sequencing technology on genetics,” Trends in Genetics, 2007, vol. 24, No. 3, pp. 133-141; Su, et al., “Next-generation sequencing and its applications in molecular diagnostics” Expert Rev Mol Diagn, 2011, 11 (3):333-43; and Zhang et al., “The impact of next-generation sequencing on genomics,” J Genet Genomics, 201, 38(3): 95-109.

As used herein, “obtaining” as in “obtaining an agent” includes synthesizing, purchasing, or otherwise acquiring the agent.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects the invention embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

By “probe set” or “bait set” is meant a set of probes that hybridize to and characterize a target polynucleotide.

By “reduces” is meant a negative alteration of at least 10%, 25%, 50%, 75%, or 100%.

By “reference” is meant a standard or control condition. In embodiments, the reference is a reference sequence. A non-limiting example of a reference sequence is a genomic DNA sequence corresponding to a subject not having a DLBCL. As used herein, “changed as compared to a reference” sample or subject is understood as having a level that is statistically different than a sample from a normal, untreated, or reference sample. Reference samples include, for example, cells in culture, one or more laboratory test animals, or one or more human subjects. Methods to select and test reference samples are within the ability of those in the art. Determination of statistical significance is within the ability of those skilled in the art, e.g., the number of standard deviations from the mean that constitute a positive result.

A “reference genome” is a defined genome used as a basis for genome comparison or for alignment of sequencing reads thereto. A reference genome may be a subset of or the entirety of a specified genome; for example, a subset of a genome sequence, such as exome sequence, or the complete genome sequence.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

The phrase “pharmaceutically acceptable carrier” is recognized in the art and includes a pharmaceutically acceptable material, composition or vehicle, suitable for administering compounds of the present disclosure to a subject. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some non-limiting examples of materials which can serve as pharmaceutically acceptable carriers include the following: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

The term “salts” refers to the relatively non-toxic, inorganic and organic acid addition salts of compounds of the present disclosure. These salts can be prepared in situ during the final isolation and purification of compounds or by separately reacting a purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate and laurylsulphonate salts, and the like. Representative salts may further include cations based on the alkali and alkaline earth metals, such as sodium, lithium, potassium, calcium, magnesium, and the like, as well as non-toxic ammonium, tetramethylammonium, tetramethyl ammonium, methlyamine, dimethlyamine, trimethlyamine, triethlyamine, ethylamine, and the like. (See, for example, S. M. Barge et al., “Pharmaceutical Salts,” J. Pharm. Sci., 1977, 66:1-19 which is incorporated herein by reference).

By “structural variation (SV)” is meant a large alteration in the sequence of a genome. Non-limiting examples of structural variants include gene fusions, translocations, deletions, duplications, inversions, and translocations. In embodiments, a structural variation corresponds to a genomic region that is about or at least about 100 bp, 500 bp, 1 kb, 10 kb, 100 kb, 1 Mb, 2 Mb, 3 Mb 4 Mb, 5 Mb or 10 Mb in size.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant an animal. The animal can be a mammal. The mammal can be a human or non-human mammal, such as a bovine, equine, canine, ovine, rodent, or feline.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “targeted sequencing” is meant a sequencing method where polynucleotide sequences of interest from a biological sample are selectively sequenced. In embodiments, targeted contacting polynucleotides present in a biological sample with an oligonucleotide probe or panel of oligonucleotide probes. In embodiments, targeted sequencing involves enriching for polynucleotide sequences from a sample that hybridize to an oligonucleotide probe or panel of oligonucleotide probes. In various instances, targeted sequencing has the advantage of allowing for sequencing polynucleotide sequences of interest in a biological sample to a high sequencing coverage.

As used herein, the terms “treat,” treating,” “treatment,” and the like refer obtaining a desired pharmacologic and/or physiologic effect. The effect can involve reducing or ameliorating a disorder and/or symptoms associated therewith. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease or condition in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease. It will be appreciated that, although not precluded, treating a disorder or condition does not require that the disorder, condition or symptoms associated therewith be completely eliminated.

“Tumor-derived DNA” means DNA that is derived from a cancer cell rather than a healthy control cell. Tumor derived DNA often includes structural changes that are indicative of cancer.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A and 1B are bar graphs, a matrix, and mirror GISTIC plots relating to recurrently mutated candidate cancer genes (CCGs) (FIG. 1A) and SCNAs (FIG. 1B) in primary diffuse large B cell lymphomas (DLBCLs). FIG. 1A provides a matrix and two bar graphs showing number and frequency of recurrent mutations (left), a gene sample matrix of recurrently mutated genes (shaded by type, center), and a ranking of mutated genes by their significance (MutSig2CV q value, right). FIG. 1B provides mirror GISTIC plots showing GISTIC2.0-defined recurrent copy number gains (left panel) and losses (right panel), with arm-level events (left of each plot) and focal events (right). CCGs were more likely to reside within focal SCNAs* (p=1e-44).

FIGS. 2A and 2B present a matrix and plots showing DLBCL subsets with discrete genetic signatures (FIG. 2A) and outcomes (FIG. 2B). FIG. 2A presents a matrix showing types of genetic alterations—mutations (black), copy number gains or losses (grey), structural variants (green). Transcriptionally defined cell-of-origin (COO) classification is provided at the top of FIG. 2A (ABC, dark grey; GCB, medium grey; unclassified, light grey). FIG. 2B presents plots showing PFS for the genetically distinct GCB-DLBCL clusters (C3 and C4; left), the ABC-DLBCL clusters (C1 and C5; middle) and COO independent C2 DLBCLs.

FIG. 3 presents a matrix showing a visualization of the combined gene sample matrix (samples, columns; genetic alterations, rows), which included cases from the Boston (Chapuy) and NIH (Staudt) series. Assigned clusters by non-negative matrix factorization (NMF) are indicated and associated marker genes are boxed. Each genetic alteration is ranking from top to bottom in each cluster by significance (q value as histogram to the right). A reanalysis of the combined dataset confirmed identify, associated marker genes, and biology of C1-C5 DLBCL clusters in a combined cohort. Notably, the C1-C5 labels in this cohort were used as “gold-standard” to train a molecular classifier of the disclosure.

FIGS. 4A-4F present schematics, a formula, a matrix, plots, and a list providing an overview of workflow and evaluation criteria for the molecular DLBCL classifier of the disclosure. FIGS. 4A and 4B provide schematics showing overall design. The combined dataset of DLBCL tumors from FIG. 3 was used as gold-standard to define the C1-C5 DLBCL classes. The cohort was divided into a training (n=551) and test set (n=149). Different feature reduction technologies were evaluated and different computer-based classification methods were explored and optimized in the training set. The final model was evaluated in an independent test set and is suitable for application to different new datasets. The overall goals of the classifier were that it needed to be easy, robust and probabilistic, allowing an assignment into a given C1-C5 class and also defining specific probability (“certainty”) of the classification. FIG. 4C provides and describes a formula for calculating performance score. The evaluation of different classifier models leveraged a newly constructed “performance matrix”. This performance matrix was defined as a weighted harmonic mean between two core metrics (accuracy and kappa), with accuracy weighted twice as much as the calibration factor, kappa. FIG. 4D provides a matrix describing accuracy. To calculate the accuracy, the correctly assigned cases were divided though all classified cases. FIG. 4D provides plots corresponding to an example of an high accuracy model. FIG. 4E provides a schematic describing calibration/kappa. Kappa is a calibration factor that captures the correlation between confidence and accuracy. To calculate kappa, first correctly and incorrectly classified cases were plotted and grouped based on confidence bins. By analyzing the worst case residual, best case residual, and observed residual, mean calibration relative to these bounds was computed. FIG. 4F provides a list giving an overview of model evaluation steps. The steps during the model evaluation and classifier construction are listed.

FIG. 5 provides a schematic and plots showing an overview of step 1 of the classifier construction, namely, selecting optimal classification method and data reduction technology. Different classification methods, including artificial neural networks, random forest, and naïve bayes classifying methods, were explored. The input features were either used as full features (n=163) or reduced in their complexity (e.g., through the use of metafeatures) by different dimensionality reduction technologies (e.g., PCA, biological reduction, such as summing of biologically relevant groups). The Accuracy, kappa (k) and the computed performance was calculated for each combination and ranked by performance. A model with good performance was an artificial neural network that learned the classification from a biologically reduced input feature set (metafeatures, see Table 3).

FIGS. 6A-6E provide plot showing further optimization and testing of robustness of the molecular classifier. FIG. 6A provides a plot showing optimization of metafeatures by significance and size. A model performing well in a prior design step (FIG. 5) was further optimized. It was explored whether a more stringent selection of marker genes with increased q-value thresholds and/or the reduction of features increased the performance. Metafeatures were dropped by size of the feature in the genome space (larger features were removed first). Notably, with this step the final q-value at 0.05 was able to be defined and 5 features were dropped. This was a final version of the model. The final feature list is reported in Table 3. FIG. 6B provides plots showing an evaluation of alteration classes. Next, experiments were undertaken to determine if all datatypes (mutations, copy number alterations and structural variants) were needed. Notably, all variants were needed, as the removal of features/classes deteriorated the performance of the model. A further reduction of features reduces the performance of the molecular classifier. FIG. 6C provides plots showing an evaluation of orthogonal assays. The addition of orthogonal assays to capture molecular complexity in DLBCL, such as the cell of origin defined transcriptional subtypes and/or ploidy, did not improve the performance of the molecular classifier significantly. For this reason, these features were not included in some embodiments of the molecular classifier of the disclosure. FIGS. 6D and 6E provide plots showing robustness and evaluation of sensitivity and false positive rate. By simulating loss of input data (FIG. 6D) or adding of random noise/artifacts (FIG. 6E), the robustness of the model was evaluated and the sensitivity and false positive rate of the model were determined.

FIG. 7 provides a confusion matrix showing classification results. FIG. 7 shows the classification output of the molecular classifier as confusion matrix for the training and independent test sets. As this classifier is probabilistic, it allows for defining post-hoc thresholds on the confidence. In FIG. 7 accuracy is exemplified in training and test cohorts (top and bottom row, respectively) for no confidence threshold (left column, all cases are classified), the top 70% confident cases (middle panel) and above 90% confident cases, right column). The different confidence thresholds can be used to apply the classifier in a scenario in which all cases need to be classified with no confidence limit threshold. In a scenario in which the classifier is used to assign patients to a clinical trial, a higher confidence threshold might be used with the caveat of not classifying all patients.

FIG. 8 provides images of a web portal. As one example of a tool that allows the commutation of the classification results for new cases, a web-portal was designed. The web portal was password protected, had a landing page, allowed file/data upload, classification with the latest version of the molecular classifier and the download of a classification result (class and probability). In addition, the portal allowed visualization of the data in context to published data, including the generation of heatmaps and uniform manifold approximation and projections (UMAPs). Notably, the web-portal is only one tool that allows the communication of classification results. Other tools might be apps for smartphones, etc.

FIG. 9 provides a chart describing metafeatures used in a molecular classifier of the disclosure. The indicated 21 metafeatures, M1-M21, were composed of the listed genetic features. The specific features are noted and the alteration types are coded using shading (mutations, black; copy number gains, dark grey; copy number losses, medium-grey; structural variants such as translocations, light grey).

### DETAILED DESCRIPTION OF THE INVENTION

The invention features a molecular classifier and a targeted sequencing assay for use in characterization and treatment of diffuse large B-cell lymphoma.

The invention is based in part on the discovery outlined in the examples provided herein that there are distinct classes of Diffuse large B-cell lymphoma (DLBCL) characterized by genetic signatures. The classes were discovered by identifying recurrent mutations, somatic copy number alterations (SCNAs) and structural variants (SVs, including translocations) in 304 newly diagnosed DLBCLs with clinical annotation and outcome data. By integrating these recurrent alterations, the 5 distinct DLBCL classes were identified, which included 2 particularly high-risk groups. Characterization of a DLBCL as belonging to one of these classes using the methods and panels provided herein can assist with identifying specific targeted therapies (e.g., combination therapies) to administer to a patient. This approach can accelerate the development of promising targeted therapies in patients with the greatest need and the highest likelihood of clinical benefit. More broadly, the examples provided below establish a clinically applicable molecular framework for the analysis of targeted combination therapies in patients with DLBCL and other lymphoid malignancies. The DLBCL genetic signatures can guide development of rational combination therapies in patients with the greatest need. Thus, among other things, herein is provided a molecular classifier for identifying high-risk DLBCL classes.

### DLBCL

Diffuse large B-cell lymphoma is the most common type of non-Hodgkin lymphoma (NHL) accounting for about 22 percent of newly diagnosed cases of B-cell NHL in the United States. DLBCL is an aggressive (fast-growing) NHL that affects B-lymphocytes. The occurrence of DLBCL generally increases with age, and patients tend to be over the age of 60 at diagnosis. DLBCL can develop in the lymph nodes or in “extranodal sites” (areas outside the lymph nodes) such as the gastrointestinal tract, testes, thyroid, skin, breast, bone, brain, or essentially any organ of the body. It may be localized (in one spot) or generalized (spread throughout the body).

### DLBCL Classification

The methods and compositions described herein relate to identification of a new and clinically useful classifier for DLBCL, the development of which is based upon an assessment of a significantly powered cohort of DLBCL samples for variants across whole exome sequences, where such variant assessment included characterization and evaluation of each of the following types of variation: somatic single nucleotide variants (SNVs), small insertions and deletions (Indels), somatic copy number alterations (SCNAs) and structural variants (SVs), including identification of variation across all cancer causing genes (CCGs). The classifier can predict outcome for a subject having a DLBCL independent of current clinical prognostic classification systems (e.g., the International Prognostic Index (IPI)). Specific components of the instant DLBCL classifier include the following.

### Types of Samples

This invention provides methods to extract and sequence a polynucleotide present in a sample. In one embodiment, the samples are biological samples generally derived from a subject (e.g., mammal, such as a human), preferably as a bodily fluid (such as ascites, blood, plasma, pleural fluid, serum, cerebrospinal fluid, phlegm, saliva, stool, urine, semen, prostate fluid, breast milk, or tears), or tissue sample (e.g. biopsy (e.g., needle biopsy), primary tumor sample, tissue section). In still another embodiment, the samples are biological samples from in vitro sources (e.g., cell culture medium). In an embodiment, the biological sample is plasma containing cell free (cfDNA) or circulating tumor DNA (ctDNA)

In embodiments, a liquid sample (e.g., blood, plasma, serum) comprises at least about and/or less than about 1 μl, 10 μl, 100 μl, 200 μl, 300 μl, 400 μl, 500 μl, 600 μl, 700 μl, 800 μl, 900 μl, 1 ml, 2 ml, 3 ml, 4 ml, 5 ml, 6 ml, 7 ml, 8 ml, 9 ml, 10 ml, or 15 ml. In embodiments, a sample comprises at least about and/or less than about 1 mg, 10 mg, 100 mg, 200 mg, 300 mg, 400 mg, 500 mg, 600 mg, 700 mg, 800 mg, 900 mg, 1 g, 2 g, 3 g, 4 g, 5 g, 6 g, 7 g, 8 g, 9 g, 10 g, or 15 g. In various cases, the methods provided herein can be completed successfully using any of the above-listed sample volumes and/or masses.

### Reference Sequences

In certain aspects, the instant disclosure provides methods and kits that involve and/or allow for assessment of the presence or absence of one or more sequence variants and/or mutations (e.g., structural variants including translocations (SVs), somatic copy number alterations (SCNAs) and recurrent mutations) in a test subject, tissue, cell or sample, as compared to a corresponding reference sequence. In particular embodiments, a subject, tissue, cell and/or sample is assessed for one or more variants and/or sites of copy number variation.

Up to five alteration types (alternatively “classes”) were measured and can be used for the classifier (i.e., a prognostic classifier as exemplified herein):
1.) Mutations (single nucleotide variants and/or InDels)
2.) Copy number alterations (CN gain, amplifications, CN losses, Deletions)
3.) Structural variants (chromosomal translocations, inversions, tandem duplications, etc.)
4.) Genome doublings
5.) Mutational Signatures

In some instances, the alteration types used for the classifier include structural variants including translocations (SVs), somatic copy number alterations (SCNAs) and mutations.

For classifier performance consistent with the exemplar described below as “Neural Network classifier” the somatic variants (1-5) should be detected with at least 90% sensitivity and less than 5% false detection rate.

Mutations in Candidate Cancer Genes (CCGs), hereafter referred to as driver mutations, were identified with MutSig2CV and CLUMPS.

Recurrent copy number alterations were identified using GISTIC2.0, as described in U.S. Patent Application Publication No. 2019/0292602.

C1 and C5 DLBCLs had significantly different cAID signature activity.

It is expressly contemplated that either all or a subset of these five classes of alterations may be measured as part of the methods provided herein, where any combination of the individual members of each class, or even other genes, can be used within a classifier of the instant disclosure.

### Detection of Alterations

In some aspects, exome sequencing or probe-hybridization is performed upon a test sample (e.g., a biological sample containing circulating tumor DNA (ctDNA), cell free DNA (cfDNA), RNA, polypeptides, mixtures thereof, and the like) for purpose of detecting variants and/or copy number variation as described herein and identifying DLBCL classification and selecting a therapy. In certain embodiments, assessment of candidate and/or test DLBCL samples can be performed using one or more amplification and/or sequencing oligonucleotides flanking the above-referenced variant sequence and/or copy number variation regions. Assessment of candidate and/or test DLBCL samples can be performed using one or more baits (described further below) for use in targeted sequencing of variants. The assessment can involve using baits to target particular sequences from a sample for subsequent sequencing.

In some embodiments, assessment of candidate and/or test DLBCL samples can be performed by sequencing a library of polynucleotides prepared from a sample (e.g., circulating tumor DNA from a subject). The library of polynucleotides can be sequenced in some embodiments as described further below and in the Examples of the present disclosure using targeted sequencing using a targeted sequencing panel comprising baits (i.e., probes or polynucleotides targeting variants). The assessment can also be performed based upon binding of a labeled bait(s) (e.g., an oligonucleotide(s)) to a target sequence in the sample. Design and use of such amplification and sequencing oligonucleotides, and/or copy number detection probes/oligonucleotides (e.g., baits), can be performed by one of ordinary skill in the art.

The methods provided herein can be used for enriching for target polynucleotides. The polynucleotides are associated with a genetic alteration of interest (e.g., SVs, SCNAs, or mutations). The polynucleotides can be enriched from a sample by about or at least about 2, 3, 4, 5, 6, 7, 8, 9, 10, 15, 20, 25, 30, 35, 40, 45, 50, 55, 60, 65, 70, 75, 80, 85, 90, 95, or 100-fold.

In some instances the library is prepared using about, less than about, and/or at least about, 0.1 ng, 1 ng, 2 ng, 3 ng, 4 ng, 5 ng, 10 ng, 15 ng, 20 ng, 25 ng, 30 ng, 35 ng, 40 ng, 45 ng, 50 ng, 75 ng, 100 ng, 250 ng, 300 ng, 350 ng, 400 ng, 450 ng, 500 ng, 1,000 ng, or more of DNA. In some cases, the library is prepared using DNA fragments with an average size of about, at least about, and/or of no more than about 10 bp, 20 bp, 30 bp, 40 bp, 50 bp, 100 bp, 150 bp, 200 bp, 300 bp, 400 bp, 500 bp, or 1,000 bp.

Methods for preparing libraries of polynucleotides for sequencing are known to one of skill in the art. Library preparation can include the addition of nucleotide bar codes to the library polynucleotides according to methods known in the art.

As will be appreciated by one of ordinary skill in the art, any amplification, sequencing (e.g., targeted sequencing), and/or copy number detection oligonucleotides can be modified by any of a number of art-recognized moieties and/or exogenous sequences, e.g., to enhance the processes of amplification, hybridization, sequencing reactions and/or detection. Exemplary oligonucleotide modifications that are expressly contemplated for use with the oligonucleotides of the instant disclosure include, e.g., fluorescent and/or radioactive label modifications; labeling one or more oligonucleotides with a universal amplification sequence (optionally of exogenous origin) and/or labeling one or more oligonucleotides of the instant disclosure with a unique identification sequence (e.g., a “bar-code” sequence, optionally of exogenous origin), as well as other modifications known in the art and suitable for use with oligonucleotides.

In embodiments, the polynucleotides (e.g., baits, probes, or oligonucleotides) provided herein (e.g., baits, probes, or oligonucleotides) contain one or more modifications or analogs.

For example, in some embodiments a polynucleotide contains one or more analogs (e.g., altered backbone, sugar, or nucleobase). Some non-limiting examples of analogs include 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine.

In embodiments, the polynucleotide contains a modified backbone and/or linkages (e.g., between adjacent nucleosides). Non-limiting examples of modified backbones include those that contain a phosphorus atom in the backbone and those that do not contain a phosphorus atom in the backbone. Non-limiting examples of modified backbones include phosphorothioates, chiral phosphorothioates, phosphorodithioates, phosphotriesters, aminoalkyl phosphotriesters, methyl and other alkyl phosphonate such as 3′-alkylene phosphonates, 5′-alkylene phosphonates, chiral phosphonates, phosphinates, phosphoramidates including 3′-amino phosphoramidate and aminoalkyl phosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage.

In embodiments, a polynucleotide contains short chain alkyl or cycloalkyl linkages (e.g., between adjacent nucleosides), mixed heteroatom and alkyl or cycloalkyl internucleoside linkages, or one or more short chain heteroatomic or heterocyclic internucleoside linkages. In embodiments, a polynucleotide includes one or more of the following: morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In embodiments, a polynucleotide contains a nucleic acid mimetic. The term “mimetic” can be intended to include polynucleotides wherein only the furanose ring or both the furanose ring and the internucleotide linkage are replaced with non-furanose groups, replacement of only the furanose ring can also be referred as being a sugar surrogate. The heterocyclic base moiety or a modified heterocyclic base moiety can be maintained for hybridization with an appropriate target nucleic acid. One such nucleic acid can be a peptide nucleic acid (PNA). In a PNA, the sugar-backbone of a polynucleotide can be replaced with an amide containing backbone, in particular an aminoethylglycine backbone. The nucleotides can be retained and are bound directly or indirectly to aza nitrogen atoms of the amide portion of the backbone. In embodiments, the backbone in PNA compounds contains two or more linked aminoethylglycine units that give PNA an amide containing backbone. Heterocyclic base moieties can be bound directly or indirectly to aza nitrogen atoms of the amide portion of the backbone.

In embodiments, a polynucleotide contains a morpholino backbone structure. For example, a nucleic acid can contain a 6-membered morpholino ring in place of a ribose ring. In some of these embodiments, a phosphorodiamidate or other non-phosphodiester internucleoside linkage can replace a phosphodiester linkage.

A polynucleotide can contain linked morpholino units having heterocyclic bases attached to the morpholino ring. Linking groups can link morpholino monomeric units. Non-ionic morpholino-based oligomeric compounds can have less undesired interactions with cellular proteins. Morpholino-based polynucleotides can be nonionic mimics of nucleic acids. A variety of compounds within the morpholino class can be joined using different linking groups. A further class of polynucleotide mimetic can be referred to as cyclohexenyl nucleic acids (CeNA). In some instances, the furanose ring normally present in a nucleic acid molecule is replaced with a cyclohexenyl ring. CeNA DMT protected phosphoramidite monomers can be prepared and used for oligomeric compound synthesis using phosphoramidite chemistry. In some cases, incorporation of CeNA monomers into a nucleic acid chain increases the stability of a DNA/RNA hybrid. CeNA oligoadenylates can form complexes with nucleic acid complements with similar stability to the native complexes. In embodiments, a polynucleotide contains Locked Nucleic Acids (LNAs) in which the 2′-hydroxyl group is linked to the 4′ carbon atom of the sugar ring thereby forming a 2′-C, 4′-C-oxymethylene linkage, thereby forming a bicyclic sugar moiety. The linkage can be a methylene (—CH₂), group bridging the 2′ oxygen atom and the 4′ carbon atom wherein n is 1 or 2. LNA and LNA analogs can display very high duplex thermal stabilities with complementary nucleic acid (Tₘ=+3 to +10° C.), stability towards 3′-exonucleolytic degradation and good solubility properties.

In embodiments, a polynucleotide contains nucleobase modifications (often referred to simply as “base modifications”) or substitutions. In embodiments, unmodified nucleobases include one or more of the purine bases, (e.g., adenine (A) and guanine (G)), and/or the pyrimidine bases, (e.g., thymine (T), cytosine (C) and uracil (U)). Non-limiting examples of modified nucleobases include nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Further non-limiting examples of modified nucleobases include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g., 9-(2-aminoethoxy)-H-pyrimido(5,4-(b) (1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g., 9-(2-aminoethoxy)-H-pyrimido(5,4-(b) (1,4)benzoxazin-2(3H)-one), carbazole cytidine (2H-pyrimido(4,-b)indol-2-one), pyridoindole cytidine (H-pyrido(3′,2′:4,5)pyrrolo[2,3-d]pyrimidin-2-one).

Variants can be characterized by sequencing polynucleotides. Characterization of a variant can involve sequencing all or a portion of a variant targeted by an oligonucleotide probe with an oligonucleotide sequence corresponding to any of SEQ ID NOs: 1-9244 or to one or more of the baits described further below. The polynucleotides can be DNA fragments.

Sequencing may be performed on any high-throughput platform. Methods of sequencing oligonucleotides and nucleic acids are well known in the art (see, e.g., WO93/23564, WO98/28440 and WO98/13523; U.S. Pat. Nos. 5,525,464; 5,202,231; 5,695,940; 4,971,903; 5,902,723; 5,795,782; 5,547,839 and 5,403,708; Sanger et al., Proc. Natl. Acad. Sci. USA 74:5463 (1977); Drmanac et al., Genomics 4:114 (1989); Koster et al., Nature Biotechnology 14:1123 (1996); Hyman, Anal. Biochem. 174:423 (1988); Rosenthal, International Patent Application Publication 761107 (1989); Metzker et al., Nucl. Acids Res. 22:4259 (1994); Jones, Biotechniques 22:938 (1997); Ronaghi et al., Anal. Biochem. 242:84 (1996); Ronaghi et al., Science 281:363 (1998); Nyren et al., Anal. Biochem. 151:504 (1985); Canard and Arzumanov, Gene 11:1 (1994); Dyatkina and Arzumanov, Nucleic Acids Symp Ser 18:117 (1987); Johnson et al., Anal. Biochem. 136:192 (1984); and Elgen and Rigler, Proc. Natl. Acad. Sci. USA 91(13):5740 (1994), all of which are expressly incorporated by reference). In one embodiment, the sequencing of a DNA fragment is carried out using commercially available sequencing technology SBS (sequencing by synthesis) by Illumina. In another embodiment, the sequencing of the DNA fragment is carried out using chain termination method of DNA sequencing. In yet another embodiment, the sequencing of the DNA fragment is carried out using one of the commercially available next-generation sequencing technologies, including SMRT (single-molecule real-time) sequencing from Pacific Biosciences, Ion Torrent™ sequencing from ThermoFisher Scientific, Pyrosequencing (454) from Roche, and SOLiD© technology from Applied Biosystems. Any appropriate sequencing technology may be chosen for sequencing. Further examples of sequencing methods suitable for use in the methods of the present disclosure include those described in U.S. Patent Application Publication No. 2019/0078232, which is incorporated herein by reference in its entirety for all purposes. A DNA sample can be pre-screened using ultra low pass sequencing to confirm that the sample contains sufficient levels of tumor-derived DNA prior to further characterization, as described in U.S. Patent Application Publication No. 2019/0078232.

In various aspects, the methods provided herein involve sequencing of a sample. In some embodiments, the sequencing is whole-genome sequencing (WGS) or whole-exome sequencing (WES). The sequencing is performed upon a test sample for purpose of detecting alterations, such as somatic copy number alterations, mutations (e.g., single nucleotide polymorphisms), and/or structural variations. In certain embodiments, the sequencing can be performed with or without amplification of a sample to be sequenced. In embodiments, a sample is sequenced to a coverage of about, at least about, and/or no more than about 0.01×, 0.05×, 0.1×, 0.2×, 0.3×, 0.4×, 0.5×, 1×, 2×, 3×, 4×, 5×, 7×, 8×, 9×, 10×, 20×, 30×, 40×, 50×, 60×, 70×, 90×, 100×, 200×, 300×, 400×, 500×, 600×, 700×, 800×, 900×, 1000×, 5000×, 10000×, 15000×, 20000×, 25000×, 30000×, 50000×, 100000×, or more.

Whole genome sequencing (also known as “WGS”, full genome sequencing, complete genome sequencing, or entire genome sequencing) is a process that involves sequencing a complete DNA sequence of an organism's genome. A common strategy used for WGS is shotgun sequencing, in which DNA is broken up randomly into numerous small segments, which are sequenced. Sequence data obtained from one sequencing reaction is termed a “read.” The reads can be assembled together based on sequence overlap. The genome sequence is obtained by assembling the reads into a reconstructed sequence.

Whole exome sequencing (“WES”) is a technique used to sequence all the expressed genes in a cell or subject. WES includes first selecting only that portion of a polynucleotide sample that encodes proteins (e.g., cDNA, or a subset of a cfDNA sample), and then sequencing using any DNA sequencing technology well known in the art or as described herein. In a human being, there are about 180,000 exons, which constitute about 1% of the human genome, or approximately 30 million base pairs. In some embodiments, to sequence the exons of a genome, fragments of double-stranded genomic DNA are obtained (e.g., by methods such as sonication, nuclease digestion, or any other appropriate methods). Linkers or adapters are then attached to the DNA fragments, which are then hybridized to a library of polynucleotides designed to capture only the exons. The hybridized DNA fragments are then selectively isolated and subjected to sequencing using any sequencing method known in the art or described herein.

In aspects of the invention, a sample is analyzed by means of a biochip (also known as a microarray) containing targeted baits (oligonucleotides specific for a target alteration). Targeted baits specific for target alterations (e.g., select SV, SCNAs, and mutations) are useful as hybridizable array elements in a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28: e3. i-e3. vii, 2000), MacBeath et al., (Science 289:1760-1763, 2000), Zhu et al. (Nature Genet. 26:283-289), and in U.S. Pat. No. 6,436,665, hereby incorporated by reference.

In aspects of the invention, a sample is analyzed by means of a nucleic acid biochip (also known as a nucleic acid microarray). To produce a nucleic acid biochip, oligonucleotides may be synthesized or bound to the surface of a substrate using a chemical coupling procedure and an ink jet application apparatus, as described in PCT application WO95/251116 (Baldeschweiler et al.). Alternatively, a gridded array may be used to arrange and link cDNA fragments or oligonucleotides to the surface of a substrate using a vacuum system, thermal, UV, mechanical or chemical bonding procedure.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In embodiments, hybridization will occur at 37° C. in 500 mM NaCl, 50 mM trisodium citrate, 1% SDS, 35% formamide, and 100 μg/ml denatured salmon sperm DNA (ssDNA). In other embodiments, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., of at least about 42° C., or of at least about 68° C. In embodiments, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In other embodiments, wash steps will occur at 68° C. in 15 mM NaCl, 1.5 mM trisodium citrate, and 0.1% SDS. Additional variations on these conditions will be readily apparent to those skilled in the art.

Detection system for measuring the absence, presence, and amount of hybridization for all of the distinct nucleic acid sequences are well known in the art. For example, simultaneous detection is described in Heller et al., Proc. Natl. Acad. Sci. 94:2150-2155, 1997. In embodiments, a scanner is used to determine the levels and patterns of fluorescence.

For purpose of this disclosure, the term “amplification” means any method employing a primer and a polymerase for replicating a target sequence linearly or exponentially with reasonable fidelity. Amplification may be carried out by natural or recombinant DNA polymerases such as TaqGold™, T7 DNA polymerase, Klenow fragment of E. coli DNA polymerase, and reverse transcriptase. A preferred amplification method is PCR. Typically, the amplification of a sample results in an exponential increase in copy number of the amplified sequences. Amplification may involve thermocycling or isothermal amplification (such as through the methods RPA or LAMP).

Design and use of oligonucleotides for amplification and/or sequencing is within the knowledge of one of ordinary skill in the art. Oligonucleotides can be modified by any of a number of art-recognized moieties and/or exogenous sequences, e.g., to enhance the processes of amplification, sequencing reactions, and/or detection. Exemplary oligonucleotide modifications that are expressly contemplated for use with the oligonucleotides of the instant disclosure include, e.g., fluorescent and/or radioactive label modifications; labeling one or more oligonucleotides with a universal amplification sequence (optionally of exogenous origin) and/or labeling one or more oligonucleotides of the instant disclosure with a unique identification sequence (e.g., a “bar-code” sequence, optionally of exogenous origin), as well as other modifications known in the art and suitable for use with oligonucleotides.

### Classification

In the methods of the invention, a neural network classifier (i.e., a classification model) can be employed to define DLBCL classification groups (C1-C5). As would be appreciated by one of ordinary skill in the art, other forms of classifier (e.g., nearest-neighbor, decision trees, boosting, support-vector machines, Naive Bayes, bagging, random forests, and various others) can be applied to variant and/or copy number data, to perform such test sample classification. Methods for the design of classifiers are described, for example, in Baldi Pierre, and Brunak, Soren, Bioinformatics: The Machine Learning Approach. 2^{nd} Ed. Cambridge, MA, “A Bradford Book” 2001; Zhang, Y. and Rajapakse, J., Machine Learning in Bioinformatics, Wiley, 2009; and Srinivasa K., Siddesh G., Manisekhar S. (eds) Statistical Modelling and Machine Learning Principles for Bioinformatics Techniques, Tools, and Applications. Algorithms for Intelligent Systems. Springer, Singapore (2020), the disclosures of each of which are incorporated herein in their entirety by reference for all purposes.

In embodiments, the methods of the disclosure involve dimensionality reduction (e.g., through the use of metafeatures), where dimensionality reduction is the transformation of data from a high-dimensional space into a low-dimensional space so that the low-dimensional representation retains meaningful properties of the original data. Dimensionality reduction can involve a linear or a nonlinear approach, and/or feature selection or feature extraction. In feature selection a subset of input variables can be selected using any of a variety of methods, such as a filter strategy, a wrapper strategy, or an embedded strategy. Non-limiting examples of techniques that can be used for dimensionality reduction include feedforward neural networks, principal component analysis (PCA), non-negative matrix factorization (NMF) (see, Chapuy B, Stewart C, Dunford A J, et al. Molecular subtypes of diffuse large B cell lymphoma are associated with distinct pathogenic mechanisms and outcomes. Nat Med. 2018; 24(5):679-690, the disclosure of which is incorporated herein by reference in its entirety for all purposes), kernel PCA, graph-based kernel PCA, linear discriminant analysis (LDA), generalized discriminant analysis (GDA), autoencoder, t-distributed stochastic neighbor embedding (t-SNE), and uniform manifold approximation projection. In embodiments, dimensionality reduction is sued to avoid overtraining a classification algorithm.

A neural network consists of units (neurons), arranged in layers, which convert an input vector (e.g., a vector comprising 21 metafeature values) into some output In embodiments, the input vector contains about, at least about, and/or no more than about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 125, 150, 175, 200, 225, 250, 275, 300, 400, or 500 metafeature values. Each unit takes an input, applies a (often nonlinear) function to it and then passes the output on to the next layer. Generally the networks are defined to be feed-forward: a unit feeds its output to all the units on the next layer, but there is no feedback to the previous layer.

In embodiments, the molecular classifier is probabilistic and includes a post-hoc threshold on confidence. In some cases, the confidence threshold is about, at least about, and/or no more than about 0%, 5%, 10%, 20%, 30%, 40%, 50%, 60%, 70%, 75%, 80%, 81%, 82%, 83%, 84%, 85%, 86%, 87%, 88%, 89%, 90%, 91%, 92%9, 93%, 94%, 95%. 96%. 97%, 98%, 99%, or 100%. It is advantageous in some contexts to select a lower confidence threshold (e.g., below 5%, 10%, 20%, 30%, 40%, 50%, 60%, 70%, 80%, or 90%) to allow for classification of a larger number of samples, and it is advantageous in some contexts (e.g., in the clinical setting) to select a higher confidence threshold (e.g., above 70%, 80%, 90%, 95%, 96%, 97%, 98%, or 99%).

Weightings are applied to the signals passing from one unit to another, and it is these weightings which are tuned in the training phase to adapt a neural network to the particular problem at hand. This is the learning phase.

Neural networks have found application in a wide variety of problems. These range from function representation to pattern recognition, with pattern recognition being the focus of use of neural net classifiers of the instant disclosure.

In some embodiments, data derived from assays (e.g., genomic analyses) that are generated using samples such as “known samples” can then be used to “train” a classification model. A “known sample” is a sample that has been pre-classified. The data that are used to form the classification model can be referred to as a “training data set.” Once trained, the classification model can recognize patterns in data derived from unknown samples. The classification model can then be used to classify the unknown samples into classes (C1-C5). This can be useful, for example, in predicting whether or not a particular biological sample is associated with a certain biological condition (e.g., diseased versus non-diseased). The training data set that is used to form the classification model may comprise raw data or pre-processed data.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression), binary decision trees (e.g., recursive partitioning processes such as CART—classification and regression trees), artificial neural networks such as back propagation networks, discriminant analyses (e.g., Bayesian classifier or Fischer analysis), logistic classifiers, and support vector classifiers (support vector machines).

In embodiments, a supervised classification method is a recursive partitioning process. Recursive partitioning processes use recursive partitioning trees to classify data derived from unknown samples. Further details about recursive partitioning processes are provided in U.S. Patent Application No. 2002 0138208 A1 to Paulse et al., “Method for analyzing mass spectra.”

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in International Publication No. WO 01/31580 (Barnhill et al., “Methods and devices for identifying patterns in biological systems and methods of use thereof”), U.S. Patent Application No. 2002 0193950 A1 (Gavin et al., “Method or analyzing mass spectra”), U.S. Patent Application No. 2003 0004402 A1 (Hitt et al., “Process for discriminating between biological states based on hidden patterns from biological data”), and U.S. Patent Application No. 2003 0055615 A1 (Zhang and Zhang, “Systems and methods for processing biological expression data”).

The classification models can be formed on and used on any suitable digital computer. Suitable digital computers include micro, mini, or large computers using any standard or specialized operating system, such as a Unix, Windows™ or Linux™ based operating system. The digital computer that is used may be physically separate from an instrument used to generate data of interest, or it may be coupled to the instrument.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

### Clinical Classifier Scoring Algorithm

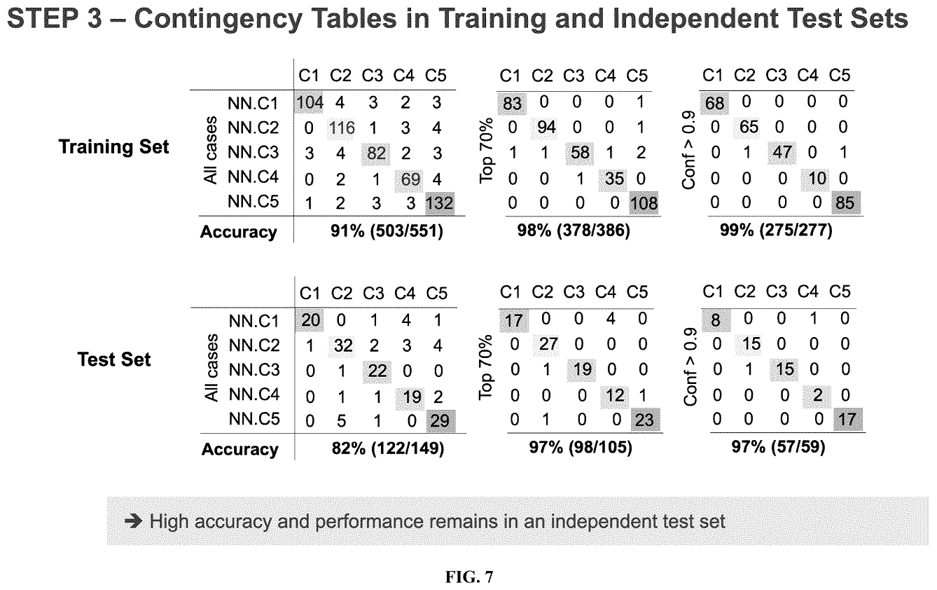

As an example, a neural net classifier was developed to prospectively identify DLBCL patients with the respective genetic signatures. The exemplified classifier utilizes 21 metafeatures (i.e., input parameters/variables), which are listed in Table 3 of the Examples below, that were selected based on biological significance for each DLBCL genetic cluster. Each metafeature contains one or more of the variants listed in Table 3. A neural network using these 21 metafeatures (each containing structural variants (SVs, which include translocations), somatic copy number alterations (SCNAs), and/or mutations) provided over 95% accuracy in assignment to classes at high confidence (e.g., >0.7 or >0.9) (FIG. 7). The output of the classifier can include a probability associated with a DLBCL falling within one of the five classes described herein (i.e., C1-C5).

Each metafeature can be calculated as a sum of variant class-specific weighted values (see Table 1) determined based upon the measurement of each variant included in the metafeature. For example, the metafeature BCL6.C1 listed in Table 3 could range from 0 (no mutation and no SV) to 5 (non-synonymous mutation and SV). The particular values assigned to each mutation, SCNA, or SV measured need not be limiting. For example, in some instances the relative magnitude of the various types of alterations listed in Table 1 (e.g., small, medium, and large) can be preserved while altering the absolute value assigned. For example, in the case of mutations, “no mutation” may be assigned a value of 100, “silent mutation” may be assigned a value of “1000” and “non-synonymous mutation” may be assigned a value of 2000. An intention of the weights/values listed in Table 1 can be to provide a means for quantifying the relative magnitude or severity of a particular observed alteration relative to a reference sequence (e.g., a wild type genome). For example, a non-synonymous mutation may be considered as being more severe than a silent mutation, which may be considered more severe than no mutation; therefore, a non-synonymous mutation may be assigned a greater weighting value than a silent mutation and both of these mutations may be assigned greater weighing values than no mutation. Similarly, an SV is considered as more severe than no SV and greater alterations in copy number are more severe than lower copy number alterations. Relative magnitudes of values assigned to mutations can be preserved across types of mutations; for example, a high level copy number alteration may be assigned a value equivalent to a non-synonymous mutation, a silent mutation can be assigned a value lower than (e.g., 3-fold lower than) than that assigned to an SV, etc. The particular values assigned to each alteration observed for each variant are not intended to be limiting.

A somatic copy number alteration can be a low level copy number alteration or a high level copy number alteration. Non-limiting examples of low-level copy number alterations include an increase or decrease in copy number of not more than about 2×, 3×, 4×, 5×, 6×, 7×, 8×, 9×, or 10×. Non-limiting examples of high-level copy number alterations include an increase or decrease in copy number of about or at least about 3×, 4×, 5×, 6×, 7×, 8×, 9×, 10×, 11×, 12×, 13×, 14×, 15×, 20×, 25×, 50×, 75×, 100×, 200×, 250×, 500×, 750×, 1,000×, 10,000×, or 100,000×.

It is expressly contemplated that a classifier of the instant disclosure can be used to link discrete genetic signatures, clinical outcome and specific targeted therapy in clinical trials and in practice. Specifically, it is contemplated that tumors of patients with DLBCL can be analyzed prospectively with an exemplified classifier or other classifier within the scope of the instant disclosure. The resulting cluster identifications are predictive of the likelihood of response to standard combination chemotherapy and suggest rational targeted therapies based on cluster-specific biology. It is further expressly contemplated that a classifier of the instant disclosure can also be applied retrospectively to archival tissue from patients on specific clinical trials or therapies.

### Clinical Classifier Computer Interface

In some embodiments, one or more computing devices may be configured to present a DCBCL classification interface that represents the cluster identifications of the metafeatures of a gene sample matrix (GSM) array representing classes of variants of a sample. The computing device(s) each at least include a computer-readable medium, such as a random-access memory (RAM) coupled to a processor or FLASH memory. In some embodiments, the processor may execute computer-executable program instructions stored in memory. In some embodiments, the processor may include a microprocessor, an ASIC, and/or a state machine. In some embodiments, the processor may include, or may be in communication with, media, for example computer-readable media, which stores instructions that, when executed by the processor, may cause the processor to perform one or more steps described herein. In some embodiments, examples of computer-readable media may include, but are not limited to, an electronic, optical, magnetic, or other storage or transmission device capable of providing a processor, such as the processor of a computing device, with computer-readable instructions. In some embodiments, other examples of suitable media may include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. In some embodiments, the instructions may comprise code from any computer-programming language, including, for example, C, C++, Visual Basic, Java, Python, Perl, JavaScript, and etc.

In some embodiments, computing devices may also comprise a number of external or internal devices such as a mouse, a CD-ROM, DVD, a physical or virtual keyboard, a display, or other input or output devices. In some embodiments, examples of computing devices may be any type of processor-based platforms that are connected to a network such as, without limitation, personal computers, digital assistants, personal digital assistants, smart phones, pagers, digital tablets, laptop computers, Internet appliances, and other processor-based devices. In some embodiments, computing devices may be specifically programmed with one or more application programs in accordance with one or more principles/methodologies detailed herein. In some embodiments, computing devices may operate on any operating system capable of supporting a browser or browser-enabled application, such as Microsoft™, Windows™, and/or Linux. In some embodiments, computing devices shown may include, for example, personal computers executing a browser application program such as Microsoft Corporation's Internet Explorer™, Apple Computer, Inc.'s Safari™, Mozilla Firefox, and/or Opera.

In some embodiments, through the computing devices, users may communicate over the exemplary network with each other and/or with other systems and/or devices coupled to the network. Server devices may include a processor as well as memory. In some embodiments, the server devices may be also coupled to the network. In some embodiments, one or more computing devices may be mobile clients.

In some embodiments, at least one database may be in communication with the server devices. The database may be any type of database, including a database managed by a database management system (DBMS). In some embodiments, an exemplary DBMS-managed database may be specifically programmed as an engine that controls organization, storage, management, and/or retrieval of data in the respective database. In some embodiments, the exemplary DBMS-managed database may be specifically programmed to provide the ability to query, backup and replicate, enforce rules, provide security, compute, perform change and access logging, and/or automate optimization. In some embodiments, the exemplary DBMS-managed database may be chosen from Oracle database, IBM DB2, Adaptive Server Enterprise, FileMaker, Microsoft Access, Microsoft SQL Server, MySQL, PostgreSQL, and a NoSQL implementation. In some embodiments, the exemplary DBMS-managed database may be specifically programmed to define each respective schema of each database in the exemplary DBMS, according to a particular database model of the present disclosure which may include a hierarchical model, network model, relational model, object model, or some other suitable organization that may result in one or more applicable data structures that may include fields, records, files, and/or objects. In some embodiments, the exemplary DBMS-managed database may be specifically programmed to include metadata about the data that is stored.

In some embodiments, the server(s) may be configured use the DCBCL classification model to produce the cluster identifications of the metafeatures. The server may provide instructions across the network to cause the computing device(s) to present the DCBCL classification interface that represents the cluster identifications of the metafeatures of a gene sample matrix (GSM) array representing classes of variants of a sample. In some embodiments, to provide the DLBCL classification, the server may be specifically configured to operate in a cloud computing/architecture such as, but not limiting to: infrastructure a service (IaaS), platform as a service (PaaS), and/or software as a service (SaaS) using a web browser, mobile app, thin client, terminal emulator or other endpoint including the computing device.

In some embodiments, the cluster identifications may be presented to the user via a DLBCL classification interface rendered on a display of the computing device, e.g., via direct instruction of the processor of the computing device or by instruction over the network by the server, or a combination thereof. The DLBCL classification interface may present, e.g., a heatmap including a visualization of variants and associated cluster identifications, confidence levels of each cluster identification, and gene mutations, a list/table of each sample/metafeature and the associated confidence levels for each possible cluster identification and/or a predicted cluster with the associated confidence level, a two-dimensional or three-dimensional representation of the metafeatures, among other visualizations. In some embodiments, the two-dimensional or three-dimensional representation of the metafeatures may include a label (e.g., via color coding, shape coding, text-based labelling, etc.) of each metafeature, where the label represents that associated cluster identification. Moreover, the two-dimensional or three-dimensional representation of the metafeatures may be produced via dimensionality reduction, such as by using a suitable dimensionality reduction model including uniform manifold approximation and projection (UMAP), principle component analysis (PCA), or other dimensionality reduction model or any combination thereof.

Accordingly, the DCBCL classification model may be implemented remotely from the computing device(s), e.g., as portal to a website or cloud-based service, or may be implemented locally to each computing device(s).

### Bait Sets

Provided herein are bait sets (e.g., sets of oligonucleotide probes) for characterization of variants in a biological sample (e.g., a biological sample containing circulating tumor DNA). The bait sets can be used in combination with the method for DLBCL classification described herein to provide a targeted sequencing assay for use in the clinic. The bait sets can comprise oligonucleotide sequences targeting structural variants including translocations (SVs), somatic copy number alterations (SCNAs), and mutations. The bait sets can contain primer sequences allowing for targeted sequencing of a sample or for preparation of an amplicon(s) from a sample. In embodiments, the bait sets make up part of a targeted sequencing panel. Methods for design and manufacture of a targeted sequencing panel are known in the art (see, e.g., Moorthie, et al. “Review of massively parallel DNA sequencing technologies”, The HUGO Journal, 5:1-12 (2011)). The targeted sequencing panel can be hybridization capture-based, circularization-based, or amplicon sequencing-based. The bait sets can be used to prepare one of the biochips described above.

The below “List of Sequence Descriptions” provides information relating to baits suitable for use in targeted sequencing according to methods of the present invention. The list provides SEQ ID NOs (i.e., SEQ ID NOs: 1-9244) for bait sequences that can be used to target the indicated variant. For each bait sequence, the List of Sequence Descriptions also lists the identity of the variant targeted (Variant) by each respective bait as well as the type (e.g., FOCAL, Gene, ARM, MSI (microsatellite instability), FP (fingerprinting), and SV).

List of Sequence Descriptions: The following list provides descriptions of the sequences provided in the Sequence Listing, where in the list each SEQ ID NO is followed by the name of the targeted genetic locus, which is followed by a description of the target type in parentheses: SEQ ID NO: 1, 1p36.32_DLBCL (FOCAL); SEQ ID NO: 2, 1p36.32_DLBCL (FOCAL); SEQ ID NO: 3, 1p36.32_DLBCL (FOCAL); SEQ ID NO: 4, 1p36.32_DLBCL (FOCAL); SEQ ID NO: 5, 1p36.32_DLBCL (FOCAL); SEQ ID NO: 6, GNB1 (Gene); SEQ ID NO: 7, GNB1 (Gene); SEQ ID NO: 8, GNB1 (Gene); SEQ ID NO: 9, GNB1 (Gene); SEQ ID NO: 10, GNB1 (Gene); SEQ ID NO: 11, GNB1 (Gene); SEQ ID NO: 12, GNB1 (Gene); SEQ ID NO: 13, GNB1 (Gene); SEQ ID NO: 14, GNB1 (Gene); SEQ ID NO: 15, 1p36.32_DLBCL (FOCAL); SEQ ID NO: 16, 1p36.32_DLBCL (FOCAL); SEQ ID NO: 17, 1p36.32_DLBCL (FOCAL); SEQ ID NO: 18, TNFRSF14 (Gene); SEQ ID NO: 19, TNFRSF14 (Gene); SEQ ID NO: 20, TNFRSF14 (Gene); SEQ ID NO: 21, TNFRSF14 (Gene); SEQ ID NO: 22, TNFRSF14 (Gene); SEQ ID NO: 23, TNFRSF14 (Gene); SEQ ID NO: 24, TNFRSF14 (Gene); SEQ ID NO: 25, TNFRSF14 (Gene); SEQ ID NO: 26, 1p36.32_DLBCL (FOCAL); SEQ ID NO: 27, 1p36.32_DLBCL (FOCAL); SEQ ID NO: 28, 1p36.32_DLBCL (FOCAL); SEQ ID NO: 29, 1p36.32_DLBCL (FOCAL); SEQ ID NO: 30, 1p36.32_DLBCL (FOCAL); SEQ ID NO: 31, 1p36.32_DLBCL (FOCAL); SEQ ID NO: 32, FP (FP); SEQ ID NO: 33, 1P (ARM); SEQ ID NO: 34, 1P (ARM); SEQ ID NO: 35, MSI (MSI); SEQ ID NO: 36, MSI (MSI); SEQ ID NO: 37, 1P (ARM); SEQ ID NO: 38, 1P (ARM); SEQ ID NO: 39, PIK3CD (Gene); SEQ ID NO: 40, PIK3CD (Gene); SEQ ID NO: 41, PIK3CD (Gene); SEQ ID NO: 42, PIK3CD (Gene); SEQ ID NO: 43, PIK3CD (Gene); SEQ ID NO: 44, PIK3CD (Gene); SEQ ID NO: 45, PIK3CD (Gene); SEQ ID NO: 46, PIK3CD (Gene); SEQ ID NO: 47, PIK3CD (Gene); SEQ ID NO: 48, PIK3CD (Gene); SEQ ID NO: 49, PIK3CD (Gene); SEQ ID NO: 50, PIK3CD (Gene); SEQ ID NO: 51, PIK3CD (Gene); SEQ ID NO: 52, PIK3CD (Gene); SEQ ID NO: 53, PIK3CD (Gene); SEQ ID NO: 54, PIK3CD (Gene); SEQ ID NO: 55, PIK3CD (Gene); SEQ ID NO: 56, PIK3CD (Gene); SEQ ID NO: 57, PIK3CD (Gene); SEQ ID NO: 58, PIK3CD (Gene); SEQ ID NO: 59, PIK3CD (Gene); SEQ ID NO: 60, PIK3CD (Gene); SEQ ID NO: 61, 1P (ARM); SEQ ID NO: 62, MTOR (Gene); SEQ ID NO: 63, MTOR (Gene); SEQ ID NO: 64, MTOR (Gene); SEQ ID NO: 65, MTOR (Gene); SEQ ID NO: 66, MTOR (Gene); SEQ ID NO: 67, MTOR (Gene); SEQ ID NO: 68, MTOR (Gene); SEQ ID NO: 69, MTOR (Gene); SEQ ID NO: 70, MTOR (Gene); SEQ ID NO: 71, MTOR (Gene); SEQ ID NO: 72, MTOR (Gene); SEQ ID NO: 73, MTOR (Gene); SEQ ID NO: 74, MTOR (Gene); SEQ ID NO: 75, MTOR (Gene); SEQ ID NO: 76, MTOR (Gene); SEQ ID NO: 77, MTOR (Gene); SEQ ID NO: 78, MTOR (Gene); SEQ ID NO: 79, MTOR (Gene); SEQ ID NO: 80, MTOR (Gene); SEQ ID NO: 81, MTOR (Gene); SEQ ID NO: 82, MTOR (Gene); SEQ ID NO: 83, MTOR (Gene); SEQ ID NO: 84, MTOR (Gene); SEQ ID NO: 85, MTOR (Gene); SEQ ID NO: 86, MTOR (Gene); SEQ ID NO: 87, MTOR (Gene); SEQ ID NO: 88, MTOR (Gene); SEQ ID NO: 89, MTOR (Gene); SEQ ID NO: 90, MTOR (Gene); SEQ ID NO: 91, MTOR (Gene); SEQ ID NO: 92, MTOR (Gene); SEQ ID NO: 93, MTOR (Gene); SEQ ID NO: 94, MTOR (Gene); SEQ ID NO: 95, MTOR (Gene); SEQ ID NO: 96, MTOR (Gene); SEQ ID NO: 97, MTOR (Gene); SEQ ID NO: 98, MTOR (Gene); SEQ ID NO: 99, MTOR (Gene); SEQ ID NO: 100, MTOR (Gene); SEQ ID NO: 101, MTOR (Gene); SEQ ID NO: 102, MTOR (Gene); SEQ ID NO: 103, MTOR (Gene); SEQ ID NO: 104, MTOR (Gene); SEQ ID NO: 105, MTOR (Gene); SEQ ID NO: 106, MTOR (Gene); SEQ ID NO: 107, MTOR (Gene); SEQ ID NO: 108, MTOR (Gene); SEQ ID NO: 109, MTOR (Gene); SEQ ID NO: 110, MTOR (Gene); SEQ ID NO: 111, MTOR (Gene); SEQ ID NO: 112, MTOR (Gene); SEQ ID NO: 113, MTOR (Gene); SEQ ID NO: 114, MTOR (Gene); SEQ ID NO: 115, MTOR (Gene); SEQ ID NO: 116, MTOR (Gene); SEQ ID NO: 117, MTOR (Gene); SEQ ID NO: 118, MTOR (Gene); SEQ ID NO: 119, MTOR (Gene); SEQ ID NO: 120, MTOR (Gene); SEQ ID NO: 121, MTOR (Gene); SEQ ID NO: 122, 1P (ARM); SEQ ID NO: 123, TNFRSF8 (Gene); SEQ ID NO: 124, TNFRSF8 (Gene); SEQ ID NO: 125, TNFRSF8 (Gene); SEQ ID NO: 126, TNFRSF8 (Gene); SEQ ID NO: 127, TNFRSF8 (Gene); SEQ ID NO: 128, TNFRSF8 (Gene); SEQ ID NO: 129, TNFRSF8 (Gene); SEQ ID NO: 130, TNFRSF8 (Gene); SEQ ID NO: 131, TNFRSF8 (Gene); SEQ ID NO: 132, TNFRSF8 (Gene); SEQ ID NO: 133, TNFRSF8 (Gene); SEQ ID NO: 134, TNFRSF8 (Gene); SEQ ID NO: 135, TNFRSF8 (Gene); SEQ ID NO: 136, TNFRSF8 (Gene); SEQ ID NO: 137, TNFRSF8 (Gene); SEQ ID NO: 138, TNFRSF8 (Gene); SEQ ID NO: 139, FP (FP); SEQ ID NO: 140, 1P (ARM); SEQ ID NO: 141, 1P (ARM); SEQ ID NO: 142, FP (FP); SEQ ID NO: 143, 1P (ARM); SEQ ID NO: 144, SPEN (Gene); SEQ ID NO: 145, SPEN (Gene); SEQ ID NO: 146, SPEN (Gene); SEQ ID NO: 147, SPEN (Gene); SEQ ID NO: 148, SPEN (Gene); SEQ ID NO: 149, SPEN (Gene); SEQ ID NO: 150, SPEN (Gene); SEQ ID NO: 151, SPEN (Gene); SEQ ID NO: 152, SPEN (Gene); SEQ ID NO: 153, SPEN (Gene); SEQ ID NO: 154, SPEN (Gene); SEQ ID NO: 155, SPEN (Gene); SEQ ID NO: 156, SPEN (Gene); SEQ ID NO: 157, SPEN (Gene); SEQ ID NO: 158, SPEN (Gene); SEQ ID NO: 159, SPEN (Gene); SEQ ID NO: 160, SPEN (Gene); SEQ ID NO: 161, SPEN (Gene); SEQ ID NO: 162, SPEN (Gene); SEQ ID NO: 163, SPEN (Gene); SEQ ID NO: 164, SPEN (Gene); SEQ ID NO: 165, SPEN (Gene); SEQ ID NO: 166, SPEN (Gene); SEQ ID NO: 167, SPEN (Gene); SEQ ID NO: 168, SPEN (Gene); SEQ ID NO: 169, SPEN (Gene); SEQ ID NO: 170, SPEN (Gene); SEQ ID NO: 171, SPEN (Gene); SEQ ID NO: 172, SPEN (Gene); SEQ ID NO: 173, SPEN (Gene); SEQ ID NO: 174, SPEN (Gene); SEQ ID NO: 175, SPEN (Gene); SEQ ID NO: 176, SPEN (Gene); SEQ ID NO: 177, SPEN (Gene); SEQ ID NO: 178, 1P (ARM); SEQ ID NO: 179, 1P (ARM); SEQ ID NO: 180, 1P (ARM); SEQ ID NO: 181, 1P (ARM); SEQ ID NO: 182, MSI (MSI); SEQ ID NO: 183, MSI (MSI); SEQ ID NO: 184, 1P (ARM); SEQ ID NO: 185, 1p36.11_DLBCL (FOCAL); SEQ ID NO: 186, 1p36.11_DLBCL (FOCAL); SEQ ID NO: 187, 1p36.11_DLBCL (FOCAL); SEQ ID NO: 188, 1p36.11_DLBCL (FOCAL); SEQ ID NO: 189, 1p36.11_DLBCL (FOCAL); SEQ ID NO: 190, ARID1A (Gene); SEQ ID NO: 191, ARID1A (Gene); SEQ ID NO: 192, ARID1A (Gene); SEQ ID NO: 193, ARID1A (Gene); SEQ ID NO: 194, ARID1A (Gene); SEQ ID NO: 195, ARID1A (Gene); SEQ ID NO: 196, ARID1A (Gene); SEQ ID NO: 197, ARID1A (Gene); SEQ ID NO: 198, ARID1A (Gene); SEQ ID NO: 199, ARID1A (Gene); SEQ ID NO: 200, ARID1A (Gene); SEQ ID NO: 201, ARID1A (Gene); SEQ ID NO: 202, ARID1A (Gene); SEQ ID NO: 203, ARID1A (Gene); SEQ ID NO: 204, ARID1A (Gene); SEQ ID NO: 205, ARID1A (Gene); SEQ ID NO: 206, ARID1A (Gene); SEQ ID NO: 207, ARID1A (Gene); SEQ ID NO: 208, ARID1A (Gene); SEQ ID NO: 209, ARID1A (Gene); SEQ ID NO: 210, ARID1A (Gene); SEQ ID NO: 211, ARID1A (Gene); SEQ ID NO: 212, ARID1A (Gene); SEQ ID NO: 213, ARID1A (Gene); SEQ ID NO: 214, ARID1A (Gene); SEQ ID NO: 215, ARID1A (Gene); SEQ ID NO: 216, ARID1A (Gene); SEQ ID NO: 217, 1p36.11_DLBCL (FOCAL); SEQ ID NO: 218, 1p36.11_DLBCL (FOCAL); SEQ ID NO: 219, MSI (MSI); SEQ ID NO: 220, MSI (MSI); SEQ ID NO: 221, 1p36.11_DLBCL (FOCAL); SEQ ID NO: 222, 1p36.11_DLBCL (FOCAL); SEQ ID NO: 223, 1p36.11_DLBCL (FOCAL); SEQ ID NO: 224, 1P (ARM); SEQ ID NO: 225, 1P (ARM); SEQ ID NO: 226, 1P (ARM); SEQ ID NO: 227, 1P (ARM); SEQ ID NO: 228, 1P (ARM); SEQ ID NO: 229, 1P (ARM); SEQ ID NO: 230, 1P (ARM); SEQ ID NO: 231, 1P (ARM); SEQ ID NO: 232, 1P (ARM); SEQ ID NO: 233, MSI (MSI); SEQ ID NO: 234, MSI (MSI); SEQ ID NO: 235, 1P (ARM); SEQ ID NO: 236, ZC3H12A (Gene); SEQ ID NO: 237, ZC3H12A (Gene); SEQ ID NO: 238, ZC3H12A (Gene); SEQ ID NO: 239, ZC3H12A (Gene); SEQ ID NO: 240, ZC3H12A (Gene); SEQ ID NO: 241, ZC3H12A (Gene); SEQ ID NO: 242, 1P (ARM); SEQ ID NO: 243, 1P (ARM); SEQ ID NO: 244, 1P (ARM); SEQ ID NO: 245, 1P (ARM); SEQ ID NO: 246, 1P (ARM); SEQ ID NO: 247, 1P (ARM); SEQ ID NO: 248, 1P (ARM); SEQ ID NO: 249, 1P (ARM); SEQ ID NO: 250, 1P (ARM); SEQ ID NO: 251, 1P (ARM); SEQ ID NO: 252, 1P (ARM); SEQ ID NO: 253, 1P (ARM); SEQ ID NO: 254, 1P (ARM); SEQ ID NO: 255, 1P (ARM); SEQ ID NO: 256, 1P (ARM); SEQ ID NO: 257, 1P (ARM); SEQ ID NO: 258, 1P (ARM); SEQ ID NO: 259, 1P (ARM); SEQ ID NO: 260, 1P (ARM); SEQ ID NO: 261, 1P (ARM); SEQ ID NO: 262, 1P (ARM); SEQ ID NO: 263, JAK1 (Gene); SEQ ID NO: 264, JAK1 (Gene); SEQ ID NO: 265, JAK1 (Gene); SEQ ID NO: 266, JAK1 (Gene); SEQ ID NO: 267, JAK1 (Gene); SEQ ID NO: 268, JAK1 (Gene); SEQ ID NO: 269, JAK1 (Gene); SEQ ID NO: 270, JAK1 (Gene); SEQ ID NO: 271, JAK1 (Gene); SEQ ID NO: 272, JAK1 (Gene); SEQ ID NO: 273, JAK1 (Gene); SEQ ID NO: 274, JAK1 (Gene); SEQ ID NO: 275, JAK1 (Gene); SEQ ID NO: 276, JAK1 (Gene); SEQ ID NO: 277, JAK1 (Gene); SEQ ID NO: 278, JAK1 (Gene); SEQ ID NO: 279, JAK1 (Gene); SEQ ID NO: 280, JAK1 (Gene); SEQ ID NO: 281, JAK1 (Gene); SEQ ID NO: 282, JAK1 (Gene); SEQ ID NO: 283, JAK1 (Gene); SEQ ID NO: 284, JAK1 (Gene); SEQ ID NO: 285, JAK1 (Gene); SEQ ID NO: 286, JAK1 (Gene); SEQ ID NO: 287, 1P (ARM); SEQ ID NO: 288, 1P (ARM); SEQ ID NO: 289, 1P (ARM); SEQ ID NO: 290, 1P (ARM); SEQ ID NO: 291, 1P (ARM); SEQ ID NO: 292, 1P (ARM); SEQ ID NO: 293, 1P (ARM); SEQ ID NO: 294, 1P (ARM); SEQ ID NO: 295, 1p31.1_DLBCL (FOCAL); SEQ ID NO: 296, 1p31.1_DLBCL (FOCAL); SEQ ID NO: 297, 1p31.1_DLBCL (FOCAL); SEQ ID NO: 298, 1p31.1_DLBCL (FOCAL); SEQ ID NO: 299, 1p31.1_DLBCL (FOCAL); SEQ ID NO: 300, 1p31.1_DLBCL (FOCAL); SEQ ID NO: 301, 1p31.1_DLBCL (FOCAL); SEQ ID NO: 302, 1p31.1_DLBCL (FOCAL); SEQ ID NO: 303, 1p31.1_DLBCL (FOCAL); SEQ ID NO: 304, 1p31.1_DLBCL (FOCAL); SEQ ID NO: 305, 1p31.1_DLBCL (FOCAL); SEQ ID NO: 306, 1p31.1_DLBCL (FOCAL); SEQ ID NO: 307, 1p31.1_DLBCL (FOCAL); SEQ ID NO: 308, 1p31.1_DLBCL (FOCAL); SEQ ID NO: 309, 1p31.1_DLBCL (FOCAL); SEQ ID NO: 310, 1p31.1_DLBCL (FOCAL); SEQ ID NO: 311, 1p31.1_DLBCL (FOCAL); SEQ ID NO: 312, 1p31.1_DLBCL (FOCAL); SEQ ID NO: 313, 1p31.1_DLBCL (FOCAL); SEQ ID NO: 314, 1p31.1_DLBCL (FOCAL); SEQ ID NO: 315, 1p31.1_DLBCL (FOCAL); SEQ ID NO: 316, 1p31.1_DLBCL (FOCAL); SEQ ID NO: 317, 1p31.1_DLBCL (FOCAL); SEQ ID NO: 318, 1p31.1_DLBCL (FOCAL); SEQ ID NO: 319, 1p31.1_DLBCL (FOCAL); SEQ ID NO: 320, 1p31.1_DLBCL (FOCAL); SEQ ID NO: 321, 1p31.1_DLBCL (FOCAL); SEQ ID NO: 322, 1p31.1_DLBCL (FOCAL); SEQ ID NO: 323, 1p21-p22_MCL (FOCAL); SEQ ID NO: 324, 1p21-p22_MCL (FOCAL); SEQ ID NO: 325, 1p21-p22_MCL (FOCAL); SEQ ID NO: 326, BCL10 (Gene); SEQ ID NO: 327, BCL10 (Gene); SEQ ID NO: 328, BCL10 (Gene); SEQ ID NO: 329, 1p21-p22_MCL (FOCAL); SEQ ID NO: 330, 1p21-p22_MCL (FOCAL); SEQ ID NO: 331, 1p21-p22_MCL (FOCAL); SEQ ID NO: 332, 1p21-p22_MCL (FOCAL); SEQ ID NO: 333, 1p21-p22_MCL (FOCAL); SEQ ID NO: 334, 1p21-p22_MCL (FOCAL); SEQ ID NO: 335, 1p21-p22_MCL (FOCAL); SEQ ID NO: 336, 1p21-p22_MCL (FOCAL); SEQ ID NO: 337, 1p21-p22_MCL (FOCAL); SEQ ID NO: 338, 1p21-p22_MCL (FOCAL); SEQ ID NO: 339, 1p21-p22_MCL (FOCAL); SEQ ID NO: 340, 1p21-p22_MCL (FOCAL); SEQ ID NO: 341, 1p21-p22_MCL (FOCAL); SEQ ID NO: 342, 1p21-p22_MCL (FOCAL); SEQ ID NO: 343, 1p21-p22_MCL (FOCAL); SEQ ID NO: 344, 1p21-p22_MCL (FOCAL); SEQ ID NO: 345, 1p21-p22_MCL (FOCAL); SEQ ID NO: 346, 1p21-p22_MCL (FOCAL); SEQ ID NO: 347, 1p21-p22_MCL (FOCAL); SEQ ID NO: 348, 1p21-p22_MCL (FOCAL); SEQ ID NO: 349, 1p21-p22_MCL (FOCAL); SEQ ID NO: 350, 1p21-p22_MCL (FOCAL); SEQ ID NO: 351, 1p21-p22_MCL (FOCAL); SEQ ID NO: 352, 1p21-p22_MCL (FOCAL); SEQ ID NO: 353, 1p21-p22_MCL (FOCAL); SEQ ID NO: 354, 1p21-p22_MCL (FOCAL); SEQ ID NO: 355, 1p21-p22_MCL (FOCAL); SEQ ID NO: 356, 1p21-p22_MCL (FOCAL); SEQ ID NO: 357, 1p21-p22_MCL (FOCAL); SEQ ID NO: 358, 1p21-p22_MCL (FOCAL); SEQ ID NO: 359, 1p21-p22_MCL (FOCAL); SEQ ID NO: 360, 1p21-p22_MCL (FOCAL); SEQ ID NO: 361, 1p21-p22_MCL (FOCAL); SEQ ID NO: 362, 1p21-p22_MCL (FOCAL); SEQ ID NO: 363, 1p21-p22_MCL (FOCAL); SEQ ID NO: 364, 1p21-p22_MCL (FOCAL); SEQ ID NO: 365, 1p21-p22_MCL (FOCAL); SEQ ID NO: 366, 1p21-p22_MCL (FOCAL); SEQ ID NO: 367, MSI (MSI); SEQ ID NO: 368, MSI (MSI); SEQ ID NO: 369, 1p21-p22_MCL (FOCAL); SEQ ID NO: 370, 1p21-p22_MCL (FOCAL); SEQ ID NO: 371, 1p21-p22_MCL (FOCAL); SEQ ID NO: 372, 1p21-p22_MCL (FOCAL); SEQ ID NO: 373, 1p21-p22_MCL (FOCAL); SEQ ID NO: 374, 1p21-p22_MCL (FOCAL); SEQ ID NO: 375, 1p21-p22_MCL (FOCAL); SEQ ID NO: 376, 1p21-p22_MCL (FOCAL); SEQ ID NO: 377, 1p21-p22_MCL (FOCAL); SEQ ID NO: 378, 1p21-p22_MCL (FOCAL); SEQ ID NO: 379, 1p21-p22_MCL (FOCAL); SEQ ID NO: 380, 1p21-p22_MCL (FOCAL); SEQ ID NO: 381, 1p21-p22_MCL (FOCAL); SEQ ID NO: 382, 1p21-p22_MCL (FOCAL); SEQ ID NO: 383, 1p21-p22_MCL (FOCAL); SEQ ID NO: 384, 1p21-p22_MCL (FOCAL); SEQ ID NO: 385, 1p21-p22_MCL (FOCAL); SEQ ID NO: 386, 1p21-p22_MCL (FOCAL); SEQ ID NO: 387, 1p21-p22_MCL (FOCAL); SEQ ID NO: 388, 1p21-p22_MCL (FOCAL); SEQ ID NO: 389, 1p21-p22_MCL (FOCAL); SEQ ID NO: 390, 1p21-p22_MCL (FOCAL); SEQ ID NO: 391, 1p21-p22_MCL (FOCAL); SEQ ID NO: 392, 1p21-p22_MCL (FOCAL); SEQ ID NO: 393, 1p21-p22_MCL (FOCAL); SEQ ID NO: 394, 1p21-p22_MCL (FOCAL); SEQ ID NO: 395, 1p21-p22_MCL (FOCAL); SEQ ID NO: 396, 1p21-p22_MCL (FOCAL); SEQ ID NO: 397, 1p21-p22_MCL (FOCAL); SEQ ID NO: 398, 1p21-p22_MCL (FOCAL); SEQ ID NO: 399, 1p21-p22_MCL (FOCAL); SEQ ID NO: 400, 1p21-p22_MCL (FOCAL); SEQ ID NO: 401, 1p21-p22_MCL (FOCAL); SEQ ID NO: 402, 1p21-p22_MCL (FOCAL); SEQ ID NO: 403, 1p21-p22_MCL (FOCAL); SEQ ID NO: 404, 1p21-p22_MCL (FOCAL); SEQ ID NO: 405, 1p21-p22_MCL (FOCAL); SEQ ID NO: 406, 1p21-p22_MCL (FOCAL); SEQ ID NO: 407, 1p21-p22_MCL (FOCAL); SEQ ID NO: 408, 1p21-p22_MCL (FOCAL); SEQ ID NO: 409, 1p21-p22_MCL (FOCAL); SEQ ID NO: 410, 1p21-p22_MCL (FOCAL); SEQ ID NO: 411, 1p21-p22_MCL (FOCAL); SEQ ID NO: 412, 1p21-p22_MCL (FOCAL); SEQ ID NO: 413, 1p21-p22_MCL (FOCAL); SEQ ID NO: 414, 1p21-p22_MCL (FOCAL); SEQ ID NO: 415, 1p21-p22_MCL (FOCAL); SEQ ID NO: 416, 1p21-p22_MCL (FOCAL); SEQ ID NO: 417, 1p21-p22_MCL (FOCAL); SEQ ID NO: 418, 1p21-p22_MCL (FOCAL); SEQ ID NO: 419, 1p21-p22_MCL (FOCAL); SEQ ID NO: 420, 1p21-p22_MCL (FOCAL); SEQ ID NO: 421, 1P (ARM); SEQ ID NO: 422, 1P (ARM); SEQ ID NO: 423, 1P (ARM); SEQ ID NO: 424, 1P (ARM); SEQ ID NO: 425, 1P (ARM); SEQ ID NO: 426, 1P (ARM); SEQ ID NO: 427, 1p13.1_DLBCL (FOCAL); SEQ ID NO: 428, 1p13.1_DLBCL (FOCAL); SEQ ID NO: 429, 1p13.1_DLBCL (FOCAL); SEQ ID NO: 430, 1p13.1_DLBCL (FOCAL); SEQ ID NO: 431, 1p13.1_DLBCL (FOCAL); SEQ ID NO: 432, NRAS (Gene); SEQ ID NO: 433, NRAS (Gene); SEQ ID NO: 434, NRAS (Gene); SEQ ID NO: 435, NRAS (Gene); SEQ ID NO: 436, 1p13.1_DLBCL (FOCAL); SEQ ID NO: 437, 1p13.1_DLBCL (FOCAL); SEQ ID NO: 438, 1p13.1_DLBCL (FOCAL); SEQ ID NO: 439, 1p13.1_DLBCL (FOCAL); SEQ ID NO: 440, 1p13.1_DLBCL (FOCAL); SEQ ID NO: 441, 1p13.1_DLBCL (FOCAL); SEQ ID NO: 442, 1p13.1_DLBCL (FOCAL); SEQ ID NO: 443, 1p13.1_DLBCL (FOCAL); SEQ ID NO: 444, CD58 (Gene); SEQ ID NO: 445, CD58 (Gene); SEQ ID NO: 446, CD58 (Gene); SEQ ID NO: 447, CD58 (Gene); SEQ ID NO: 448, CD58 (Gene); SEQ ID NO: 449, CD58 (Gene); SEQ ID NO: 450, 1p13.1_DLBCL (FOCAL); SEQ ID NO: 451, 1p13.1_DLBCL (FOCAL); SEQ ID NO: 452, 1p13.1_DLBCL (FOCAL); SEQ ID NO: 453, 1p13.1_DLBCL (FOCAL); SEQ ID NO: 454, 1p13.1_DLBCL (FOCAL); SEQ ID NO: 455, 1p13.1_DLBCL (FOCAL); SEQ ID NO: 456, 1P (ARM); SEQ ID NO: 457, NOTCH2 (Gene); SEQ ID NO: 458, NOTCH2 (Gene); SEQ ID NO: 459, NOTCH2 (Gene); SEQ ID NO: 460, NOTCH2 (Gene); SEQ ID NO: 461, NOTCH2 (Gene); SEQ ID NO: 462, NOTCH2 (Gene); SEQ ID NO: 463, NOTCH2 (Gene); SEQ ID NO: 464, NOTCH2 (Gene); SEQ ID NO: 465, NOTCH2 (Gene); SEQ ID NO: 466, NOTCH2 (Gene); SEQ ID NO: 467, NOTCH2 (Gene); SEQ ID NO: 468, NOTCH2 (Gene); SEQ ID NO: 469, NOTCH2 (Gene); SEQ ID NO: 470, NOTCH2 (Gene); SEQ ID NO: 471, NOTCH2 (Gene); SEQ ID NO: 472, NOTCH2 (Gene); SEQ ID NO: 473, NOTCH2 (Gene); SEQ ID NO: 474, NOTCH2 (Gene); SEQ ID NO: 475, NOTCH2 (Gene); SEQ ID NO: 476, NOTCH2 (Gene); SEQ ID NO: 477, NOTCH2 (Gene); SEQ ID NO: 478, NOTCH2 (Gene); SEQ ID NO: 479, NOTCH2 (Gene); SEQ ID NO: 480, NOTCH2 (Gene); SEQ ID NO: 481, NOTCH2 (Gene); SEQ ID NO: 482, NOTCH2 (Gene); SEQ ID NO: 483, NOTCH2 (Gene); SEQ ID NO: 484, NOTCH2 (Gene); SEQ ID NO: 485, NOTCH2 (Gene); SEQ ID NO: 486, NOTCH2 (Gene); SEQ ID NO: 487, NOTCH2 (Gene); SEQ ID NO: 488, NOTCH2 (Gene); SEQ ID NO: 489, NOTCH2 (Gene); SEQ ID NO: 490, NOTCH2 (Gene); SEQ ID NO: 491, NOTCH2 (Gene); SEQ ID NO: 492, NOTCH2 (Gene); SEQ ID NO: 493, NOTCH2 (Gene); SEQ ID NO: 494, 1q23.3_DLBCL (FOCAL); SEQ ID NO: 495, PDE4DIP (Gene); SEQ ID NO: 496, PDE4DIP (Gene); SEQ ID NO: 497, PDE4DIP (Gene); SEQ ID NO: 498, PDE4DIP (Gene); SEQ ID NO: 499, PDE4DIP (Gene); SEQ ID NO: 500, PDE4DIP (Gene); SEQ ID NO: 501, PDE4DIP (Gene); SEQ ID NO: 502, PDE4DIP (Gene); SEQ ID NO: 503, PDE4DIP (Gene); SEQ ID NO: 504, PDE4DIP (Gene); SEQ ID NO: 505, PDE4DIP (Gene); SEQ ID NO: 506, PDE4DIP (Gene); SEQ ID NO: 507, PDE4DIP (Gene); SEQ ID NO: 508, PDE4DIP (Gene); SEQ ID NO: 509, PDE4DIP (Gene); SEQ ID NO: 510, PDE4DIP (Gene); SEQ ID NO: 511, PDE4DIP (Gene); SEQ ID NO: 512, PDE4DIP (Gene); SEQ ID NO: 513, PDE4DIP (Gene); SEQ ID NO: 514, PDE4DIP (Gene); SEQ ID NO: 515, PDE4DIP (Gene); SEQ ID NO: 516, PDE4DIP (Gene); SEQ ID NO: 517, PDE4DIP (Gene); SEQ ID NO: 518, PDE4DIP (Gene); SEQ ID NO: 519, PDE4DIP (Gene); SEQ ID NO: 520, PDE4DIP (Gene); SEQ ID NO: 521, PDE4DIP (Gene); SEQ ID NO: 522, PDE4DIP (Gene); SEQ ID NO: 523, PDE4DIP (Gene); SEQ ID NO: 524, PDE4DIP (Gene); SEQ ID NO: 525, PDE4DIP (Gene); SEQ ID NO: 526, PDE4DIP (Gene); SEQ ID NO: 527, PDE4DIP (Gene); SEQ ID NO: 528, PDE4DIP (Gene); SEQ ID NO: 529, PDE4DIP (Gene); SEQ ID NO: 530, PDE4DIP (Gene); SEQ ID NO: 531, PDE4DIP (Gene); SEQ ID NO: 532, PDE4DIP (Gene); SEQ ID NO: 533, PDE4DIP (Gene); SEQ ID NO: 534, PDE4DIP (Gene); SEQ ID NO: 535, PDE4DIP (Gene); SEQ ID NO: 536, PDE4DIP (Gene); SEQ ID NO: 537, PDE4DIP (Gene); SEQ ID NO: 538, PDE4DIP (Gene); SEQ ID NO: 539, PDE4DIP (Gene); SEQ ID NO: 540, PDE4DIP (Gene); SEQ ID NO: 541, PDE4DIP (Gene); SEQ ID NO: 542, PDE4DIP (Gene); SEQ ID NO: 543, PDE4DIP (Gene); SEQ ID NO: 544, PDE4DIP (Gene); SEQ ID NO: 545, PDE4DIP (Gene); SEQ ID NO: 546, PDE4DIP (Gene); SEQ ID NO: 547, PDE4DIP (Gene); SEQ ID NO: 548, PDE4DIP (Gene); SEQ ID NO: 549, 1q23.3_DLBCL (FOCAL); SEQ ID NO: 550, PDE4DIP (Gene); SEQ ID NO: 551, 1q23.3_DLBCL (FOCAL); SEQ ID NO: 552, 1q23.3_DLBCL (FOCAL); SEQ ID NO: 553, 1q23.3_DLBCL (FOCAL); SEQ ID NO: 554, 1q23.3_DLBCL (FOCAL); SEQ ID NO: 555, 1q23.3_DLBCL (FOCAL); SEQ ID NO: 556, 1q23.3_DLBCL (FOCAL); SEQ ID NO: 557, 1q23.3_DLBCL (FOCAL); SEQ ID NO: 558, 1q23.3_DLBCL (FOCAL); SEQ ID NO: 559, 1q23.3_DLBCL (FOCAL); SEQ ID NO: 560, HIST2H2BE (Gene); SEQ ID NO: 561, 1q23.3_DLBCL (FOCAL); SEQ ID NO: 562, 1q23.3_DLBCL (FOCAL); SEQ ID NO: 563, 1q23.3_DLBCL (FOCAL); SEQ ID NO: 564, MCL1 (Gene); SEQ ID NO: 565, MCL1 (Gene); SEQ ID NO: 566, MCL1 (Gene); SEQ ID NO: 567, MCL1 (Gene); SEQ ID NO: 568, MCL1 (Gene); SEQ ID NO: 569, 1q23.3_DLBCL (FOCAL); SEQ ID NO: 570, SETDB1 (Gene); SEQ ID NO: 571, SETDB1 (Gene); SEQ ID NO: 572, SETDB1 (Gene); SEQ ID NO: 573, SETDB1 (Gene); SEQ ID NO: 574, SETDB1 (Gene); SEQ ID NO: 575, SETDB1 (Gene); SEQ ID NO: 576, SETDB1 (Gene); SEQ ID NO: 577, SETDB1 (Gene); SEQ ID NO: 578, SETDB1 (Gene); SEQ ID NO: 579, SETDB1 (Gene); SEQ ID NO: 580, SETDB1 (Gene); SEQ ID NO: 581, SETDB1 (Gene); SEQ ID NO: 582, SETDB1 (Gene); SEQ ID NO: 583, SETDB1 (Gene); SEQ ID NO: 584, SETDB1 (Gene); SEQ ID NO: 585, SETDB1 (Gene); SEQ ID NO: 586, SETDB1 (Gene); SEQ ID NO: 587, SETDB1 (Gene); SEQ ID NO: 588, SETDB1 (Gene); SEQ ID NO: 589, SETDB1 (Gene); SEQ ID NO: 590, SETDB1 (Gene); SEQ ID NO: 591, SETDB1 (Gene); SEQ ID NO: 592, SETDB1 (Gene); SEQ ID NO: 593, SETDB1 (Gene); SEQ ID NO: 594, 1q23.3_DLBCL (FOCAL); SEQ ID NO: 595, 1q23.3_DLBCL (FOCAL); SEQ ID NO: 596, 1q23.3_DLBCL (FOCAL); SEQ ID NO: 597, 1q23.3_DLBCL (FOCAL); SEQ ID NO: 598, 1q23.3_DLBCL (FOCAL); SEQ ID NO: 599, 1q23.3_DLBCL (FOCAL); SEQ ID NO: 600, 1q23.3_DLBCL (FOCAL); SEQ ID NO: 601, 1q23.3_DLBCL (FOCAL); SEQ ID NO: 602, 1q23.3_DLBCL (FOCAL); SEQ ID NO: 603, 1q23.3_DLBCL (FOCAL); SEQ ID NO: 604, 1q23.3_DLBCL (FOCAL); SEQ ID NO: 605, 1q23.3_DLBCL (FOCAL); SEQ ID NO: 606, 1q23.3_DLBCL (FOCAL); SEQ ID NO: 607, 1q23.3_DLBCL (FOCAL); SEQ ID NO: 608, 1q23.3_DLBCL (FOCAL); SEQ ID NO: 609, 1q23.3_DLBCL (FOCAL); SEQ ID NO: 610, 1q23.3_DLBCL (FOCAL); SEQ ID NO: 611, 1q23.3_DLBCL (FOCAL); SEQ ID NO: 612, 1q23.3_DLBCL (FOCAL); SEQ ID NO: 613, 1q23.3_DLBCL (FOCAL); SEQ ID NO: 614, 1q23.3_DLBCL (FOCAL); SEQ ID NO: 615, MSI (MSI); SEQ ID NO: 616, MSI (MSI); SEQ ID NO: 617, 1q23.3_DLBCL (FOCAL); SEQ ID NO: 618, 1q23.3_DLBCL (FOCAL); SEQ ID NO: 619, 1q23.3_DLBCL (FOCAL); SEQ ID NO: 620, 1q23.3_DLBCL (FOCAL); SEQ ID NO: 621, 1q23.3_DLBCL (FOCAL); SEQ ID NO: 622, 1q23.3_DLBCL (FOCAL); SEQ ID NO: 623, NTRK1 (Gene); SEQ ID NO: 624, NTRK1 (Gene); SEQ ID NO: 625, NTRK1 (Gene); SEQ ID NO: 626, NTRK1 (Gene); SEQ ID NO: 627, NTRK1 (Gene); SEQ ID NO: 628, NTRK1 (Gene); SEQ ID NO: 629, NTRK1 (Gene); SEQ ID NO: 630, NTRK1 (Gene); SEQ ID NO: 631, NTRK1 (Gene); SEQ ID NO: 632, NTRK1 (Gene); SEQ ID NO: 633, NTRK1 (Gene); SEQ ID NO: 634, NTRK1 (Gene); SEQ ID NO: 635, NTRK1 (Gene); SEQ ID NO: 636, NTRK1 (Gene); SEQ ID NO: 637, NTRK1 (Gene); SEQ ID NO: 638, NTRK1 (Gene); SEQ ID NO: 639, NTRK1 (Gene); SEQ ID NO: 640, NTRK1 (Gene); SEQ ID NO: 641, NTRK1 (Gene); SEQ ID NO: 642, NTRK1 (Gene); SEQ ID NO: 643, 1q23.3_DLBCL (FOCAL); SEQ ID NO: 644, 1q23.3_DLBCL (FOCAL); SEQ ID NO: 645, 1q23.3_DLBCL (FOCAL); SEQ ID NO: 646, 1q23.3_DLBCL (FOCAL); SEQ ID NO: 647, 1q23.3_DLBCL (FOCAL); SEQ ID NO: 648, 1q23.3_DLBCL (FOCAL); SEQ ID NO: 649, 1q23.3_DLBCL (FOCAL); SEQ ID NO: 650, FP (FP); SEQ ID NO: 651, 1q23.3_DLBCL (FOCAL); SEQ ID NO: 652, 1q23.3_DLBCL (FOCAL); SEQ ID NO: 653, 1q23.3_DLBCL (FOCAL); SEQ ID NO: 654, 1q23.3_DLBCL (FOCAL); SEQ ID NO: 655, 1q23.3_DLBCL (FOCAL); SEQ ID NO: 656, 1q23.3_DLBCL (FOCAL); SEQ ID NO: 657, 1q23.3_DLBCL (FOCAL); SEQ ID NO: 658, 1q23.3_DLBCL (FOCAL); SEQ ID NO: 659, 1q23.3_DLBCL (FOCAL); SEQ ID NO: 660, 1q23.3_DLBCL (FOCAL); SEQ ID NO: 661, 1q23.3_DLBCL (FOCAL); SEQ ID NO: 662, 1q23.3_DLBCL (FOCAL); SEQ ID NO: 663, 1q23.3_DLBCL (FOCAL); SEQ ID NO: 664, 1q23.3_DLBCL (FOCAL); SEQ ID NO: 665, 1q23.3_DLBCL (FOCAL); SEQ ID NO: 666, 1q23.3_DLBCL (FOCAL); SEQ ID NO: 667, 1q23.3_DLBCL (FOCAL); SEQ ID NO: 668, 1q23.3_DLBCL (FOCAL); SEQ ID NO: 669, 1q23.3_DLBCL (FOCAL); SEQ ID NO: 670, 1q23.3_DLBCL (FOCAL); SEQ ID NO: 671, 1q23.3_DLBCL (FOCAL); SEQ ID NO: 672, 1q23.3_DLBCL (FOCAL); SEQ ID NO: 673, 1q23.3_DLBCL (FOCAL); SEQ ID NO: 674, 1q23.3_DLBCL (FOCAL); SEQ ID NO: 675, 1q23.3_DLBCL (FOCAL); SEQ ID NO: 676, 1q23.3_DLBCL (FOCAL); SEQ ID NO: 677, 1q23.3_DLBCL (FOCAL); SEQ ID NO: 678, 1q23.3_DLBCL (FOCAL); SEQ ID NO: 679, 1q23.3_DLBCL (FOCAL); SEQ ID NO: 680, 1q23.3_DLBCL (FOCAL); SEQ ID NO: 681, 1q23.3_DLBCL (FOCAL); SEQ ID NO: 682, 1q23.3_DLBCL (FOCAL); SEQ ID NO: 683, 1q23.3_DLBCL (FOCAL); SEQ ID NO: 684, 1q23.3_DLBCL (FOCAL); SEQ ID NO: 685, 1q23.3_DLBCL (FOCAL); SEQ ID NO: 686, 1q23.3_DLBCL (FOCAL); SEQ ID NO: 687, 1q23.3_DLBCL (FOCAL); SEQ ID NO: 688, 1q23.3_DLBCL (FOCAL); SEQ ID NO: 689, 1q23.3_DLBCL (FOCAL); SEQ ID NO: 690, 1q23.3_DLBCL (FOCAL); SEQ ID NO: 691, 1q23.3_DLBCL (FOCAL); SEQ ID NO: 692, 1q23.3_DLBCL (FOCAL); SEQ ID NO: 693, 1q23.3_DLBCL (FOCAL); SEQ ID NO: 694, 1q23.3_DLBCL (FOCAL); SEQ ID NO: 695, 1q23.3_DLBCL (FOCAL); SEQ ID NO: 696, 1q23.3_DLBCL (FOCAL); SEQ ID NO: 697, 1q23.3_DLBCL (FOCAL); SEQ ID NO: 698, 1q23.3_DLBCL (FOCAL); SEQ ID NO: 699, 1q23.3_DLBCL (FOCAL); SEQ ID NO: 700, 1q23.3_DLBCL (FOCAL); SEQ ID NO: 701, 1q23.3_DLBCL (FOCAL); SEQ ID NO: 702, 1q23.3_DLBCL (FOCAL); SEQ ID NO: 703, 1q23.3_DLBCL (FOCAL); SEQ ID NO: 704, 1q23.3_DLBCL (FOCAL); SEQ ID NO: 705, 1q23.3_DLBCL (FOCAL); SEQ ID NO: 706, 1q23.3_DLBCL (FOCAL); SEQ ID NO: 707, 1q23.3_DLBCL (FOCAL); SEQ ID NO: 708, 1q23.3_DLBCL (FOCAL); SEQ ID NO: 709, 1q23.3_DLBCL (FOCAL); SEQ ID NO: 710, 1q23.3_DLBCL (FOCAL); SEQ ID NO: 711, 1q23.3_DLBCL (FOCAL); SEQ ID NO: 712, 1q23.3_DLBCL (FOCAL); SEQ ID NO: 713, 1q23.3_DLBCL (FOCAL); SEQ ID NO: 714, 1q23.3_DLBCL (FOCAL); SEQ ID NO: 715, 1q23.3_DLBCL (FOCAL); SEQ ID NO: 716, 1q23.3_DLBCL (FOCAL); SEQ ID NO: 717, 1q23.3_DLBCL (FOCAL); SEQ ID NO: 718, 1q23.3_DLBCL (FOCAL); SEQ ID NO: 719, 1q23.3_DLBCL (FOCAL); SEQ ID NO: 720, 1q23.3_DLBCL (FOCAL); SEQ ID NO: 721, 1q23.3_DLBCL (FOCAL); SEQ ID NO: 722, 1q23.3_DLBCL (FOCAL); SEQ ID NO: 723, 1q23.3_DLBCL (FOCAL); SEQ ID NO: 724, 1q23.3_DLBCL (FOCAL); SEQ ID NO: 725, 1q23.3_DLBCL (FOCAL); SEQ ID NO: 726, 1q23.3_DLBCL (FOCAL); SEQ ID NO: 727, 1q23.3_DLBCL (FOCAL); SEQ ID NO: 728, 1q23.3_DLBCL (FOCAL); SEQ ID NO: 729, 1Q_DLBCL (ARM); SEQ ID NO: 730, 1Q_DLBCL (ARM); SEQ ID NO: 731, 1Q_DLBCL (ARM); SEQ ID NO: 732, 1Q_DLBCL (ARM); SEQ ID NO: 733, 1Q_DLBCL (ARM); SEQ ID NO: 734, 1Q_DLBCL (ARM); SEQ ID NO: 735, 1Q_DLBCL (ARM); SEQ ID NO: 736, 1Q_DLBCL (ARM); SEQ ID NO: 737, 1Q_DLBCL (ARM); SEQ ID NO: 738, 1Q_DLBCL (ARM); SEQ ID NO: 739, 1Q_DLBCL (ARM); SEQ ID NO: 740, 1Q_DLBCL (ARM); SEQ ID NO: 741, 1Q_DLBCL (ARM); SEQ ID NO: 742, 1Q_DLBCL (ARM); SEQ ID NO: 743, 1Q_DLBCL (ARM); SEQ ID NO: 744, 1Q_DLBCL (ARM); SEQ ID NO: 745, 1Q_DLBCL (ARM); SEQ ID NO: 746, 1Q_DLBCL (ARM); SEQ ID NO: 747, 1Q_DLBCL (ARM); SEQ ID NO: 748, 1Q_DLBCL (ARM); SEQ ID NO: 749, 1Q_DLBCL (ARM); SEQ ID NO: 750, 1Q_DLBCL (ARM); SEQ ID NO: 751, 1Q_DLBCL (ARM); SEQ ID NO: 752, 1Q_DLBCL (ARM); SEQ ID NO: 753, 1Q_DLBCL (ARM); SEQ ID NO: 754, 1Q_DLBCL (ARM); SEQ ID NO: 755, 1Q_DLBCL (ARM); SEQ ID NO: 756, BTG2 (Gene); SEQ ID NO: 757, BTG2 (Gene); SEQ ID NO: 758, 1Q_DLBCL (ARM); SEQ ID NO: 759, 1Q_DLBCL (ARM); SEQ ID NO: 760, 1Q_DLBCL (ARM); SEQ ID NO: 761, 1Q_DLBCL (ARM); SEQ ID NO: 762, 1Q_DLBCL (ARM); SEQ ID NO: 763, 1Q_DLBCL (ARM); SEQ ID NO: 764, 1Q_DLBCL (ARM); SEQ ID NO: 765, 1Q_DLBCL (ARM); SEQ ID NO: 766, 1Q_DLBCL (ARM); SEQ ID NO: 767, 1Q_DLBCL (ARM); SEQ ID NO: 768, PTPN14 (Gene); SEQ ID NO: 769, PTPN14 (Gene); SEQ ID NO: 770, PTPN14 (Gene); SEQ ID NO: 771, PTPN14 (Gene); SEQ ID NO: 772, PTPN14 (Gene); SEQ ID NO: 773, PTPN14 (Gene); SEQ ID NO: 774, PTPN14 (Gene); SEQ ID NO: 775, PTPN14 (Gene); SEQ ID NO: 776, PTPN14 (Gene); SEQ ID NO: 777, PTPN14 (Gene); SEQ ID NO: 778, PTPN14 (Gene); SEQ ID NO: 779, PTPN14 (Gene); SEQ ID NO: 780, PTPN14 (Gene); SEQ ID NO: 781, PTPN14 (Gene); SEQ ID NO: 782, PTPN14 (Gene); SEQ ID NO: 783, PTPN14 (Gene); SEQ ID NO: 784, PTPN14 (Gene); SEQ ID NO: 785, PTPN14 (Gene); SEQ ID NO: 786, PTPN14 (Gene); SEQ ID NO: 787, PTPN14 (Gene); SEQ ID NO: 788, PTPN14 (Gene); SEQ ID NO: 789, 1Q_DLBCL (ARM); SEQ ID NO: 790, 1Q_DLBCL (ARM); SEQ ID NO: 791, 1Q_DLBCL (ARM); SEQ ID NO: 792, 1q42.12_DLBCL (FOCAL); SEQ ID NO: 793, 1q42.12_DLBCL (FOCAL); SEQ ID NO: 794, 1q42.12_DLBCL (FOCAL); SEQ ID NO: 795, 1q42.12_DLBCL (FOCAL); SEQ ID NO: 796, 1q42.12_DLBCL (FOCAL); SEQ ID NO: 797, 1q42.12_DLBCL (FOCAL); SEQ ID NO: 798, 1q42.12_DLBCL (FOCAL); SEQ ID NO: 799, 1q42.12_DLBCL (FOCAL); SEQ ID NO: 800, 1q42.12_DLBCL (FOCAL); SEQ ID NO: 801, 1q42.12_DLBCL (FOCAL); SEQ ID NO: 802, 1q42.12_DLBCL (FOCAL); SEQ ID NO: 803, 1q42.12_DLBCL (FOCAL); SEQ ID NO: 804, 1q42.12_DLBCL (FOCAL); SEQ ID NO: 805, 1q42.12_DLBCL (FOCAL); SEQ ID NO: 806, FP (FP); SEQ ID NO: 807, 1q42.12_DLBCL (FOCAL); SEQ ID NO: 808, 1q42.12_DLBCL (FOCAL); SEQ ID NO: 809, 1q42.12_DLBCL (FOCAL); SEQ ID NO: 810, 1q42.12_DLBCL (FOCAL); SEQ ID NO: 811, 1q42.12_DLBCL (FOCAL); SEQ ID NO: 812, 1q42.12_DLBCL (FOCAL); SEQ ID NO: 813, 1q42.12_DLBCL (FOCAL); SEQ ID NO: 814, 1q42.12_DLBCL (FOCAL); SEQ ID NO: 815, 1q42.12_DLBCL (FOCAL); SEQ ID NO: 816, 1q42.12_DLBCL (FOCAL); SEQ ID NO: 817, 1q42.12_DLBCL (FOCAL); SEQ ID NO: 818, 1q42.12_DLBCL (FOCAL); SEQ ID NO: 819, 1q42.12_DLBCL (FOCAL); SEQ ID NO: 820, 1q42.12_DLBCL (FOCAL); SEQ ID NO: 821, 1q42.12_DLBCL (FOCAL); SEQ ID NO: 822, 1q42.12_DLBCL (FOCAL); SEQ ID NO: 823, 1q42.12_DLBCL (FOCAL); SEQ ID NO: 824, 1q42.12_DLBCL (FOCAL); SEQ ID NO: 825, 1q42.12_DLBCL (FOCAL); SEQ ID NO: 826, 1q42.12_DLBCL (FOCAL); SEQ ID NO: 827, 1q42.12_DLBCL (FOCAL); SEQ ID NO: 828, 1q42.12_DLBCL (FOCAL); SEQ ID NO: 829, 1q42.12_DLBCL (FOCAL); SEQ ID NO: 830, 1q42.12_DLBCL (FOCAL); SEQ ID NO: 831, 1q42.12_DLBCL (FOCAL); SEQ ID NO: 832, 1q42.12_DLBCL (FOCAL); SEQ ID NO: 833, 1q42.12_DLBCL (FOCAL); SEQ ID NO: 834, 1q42.12_DLBCL (FOCAL); SEQ ID NO: 835, 1q42.12_DLBCL (FOCAL); SEQ ID NO: 836, 1q42.12_DLBCL (FOCAL); SEQ ID NO: 837, 1q42.12_DLBCL (FOCAL); SEQ ID NO: 838, 1q42.12_DLBCL (FOCAL); SEQ ID NO: 839, 1q42.12_DLBCL (FOCAL); SEQ ID NO: 840, 1q42.12_DLBCL (FOCAL); SEQ ID NO: 841, 1q42.12_DLBCL (FOCAL); SEQ ID NO: 842, 1q42.12_DLBCL (FOCAL); SEQ ID NO: 843, 1q42.12_DLBCL (FOCAL); SEQ ID NO: 844, 1q42.12_DLBCL (FOCAL); SEQ ID NO: 845, 1q42.12_DLBCL (FOCAL); SEQ ID NO: 846, 1q42.12_DLBCL (FOCAL); SEQ ID NO: 847, 1q42.12_DLBCL (FOCAL); SEQ ID NO: 848, 1q42.12_DLBCL (FOCAL); SEQ ID NO: 849, 1q42.12_DLBCL (FOCAL); SEQ ID NO: 850, 1q42.12_DLBCL (FOCAL); SEQ ID NO: 851, 1q42.12_DLBCL (FOCAL); SEQ ID NO: 852, 1q42.12_DLBCL (FOCAL); SEQ ID NO: 853, 1q42.12_DLBCL (FOCAL); SEQ ID NO: 854, 1q42.12_DLBCL (FOCAL); SEQ ID NO: 855, 1q42.12_DLBCL (FOCAL); SEQ ID NO: 856, 1q42.12_DLBCL (FOCAL); SEQ ID NO: 857, 1q42.12_DLBCL (FOCAL); SEQ ID NO: 858, ITPKB (Gene); SEQ ID NO: 859, ITPKB (Gene); SEQ ID NO: 860, ITPKB (Gene); SEQ ID NO: 861, ITPKB (Gene); SEQ ID NO: 862, ITPKB (Gene); SEQ ID NO: 863, ITPKB (Gene); SEQ ID NO: 864, 1q42.12_DLBCL (FOCAL); SEQ ID NO: 865, ITPKB (Gene); SEQ ID NO: 866, ITPKB (Gene); SEQ ID NO: 867, ITPKB (Gene); SEQ ID NO: 868, ITPKB (Gene); SEQ ID NO: 869, ITPKB (Gene); SEQ ID NO: 870, 1q42.12_DLBCL (FOCAL); SEQ ID NO: 871, 1q42.12_DLBCL (FOCAL); SEQ ID NO: 872, 1q42.12_DLBCL (FOCAL); SEQ ID NO: 873, 1q42.12_DLBCL (FOCAL); SEQ ID NO: 874, 1q42.12_DLBCL (FOCAL); SEQ ID NO: 875, 1q42.12_DLBCL (FOCAL); SEQ ID NO: 876, 1q42.12_DLBCL (FOCAL); SEQ ID NO: 877, 1q42.12_DLBCL (FOCAL); SEQ ID NO: 878, 1q42.12_DLBCL (FOCAL); SEQ ID NO: 879, 1q42.12_DLBCL (FOCAL); SEQ ID NO: 880, 1q42.12_DLBCL (FOCAL); SEQ ID NO: 881, 1q42.12_DLBCL (FOCAL); SEQ ID NO: 882, 1q42.12_DLBCL (FOCAL); SEQ ID NO: 883, 1q42.12_DLBCL (FOCAL); SEQ ID NO: 884, 1q42.12_DLBCL (FOCAL); SEQ ID NO: 885, 1q42.12_DLBCL (FOCAL); SEQ ID NO: 886, 1q42.12_DLBCL (FOCAL); SEQ ID NO: 887, 1q42.12_DLBCL (FOCAL); SEQ ID NO: 888, 1q42.12_DLBCL (FOCAL); SEQ ID NO: 889, 1q42.12_DLBCL (FOCAL); SEQ ID NO: 890, 1q42.12_DLBCL (FOCAL); SEQ ID NO: 891, 1q42.12_DLBCL (FOCAL); SEQ ID NO: 892, 1q42.12_DLBCL (FOCAL); SEQ ID NO: 893, 1q42.12_DLBCL (FOCAL); SEQ ID NO: 894, 1q42.12_DLBCL (FOCAL); SEQ ID NO: 895, 1q42.12_DLBCL (FOCAL); SEQ ID NO: 896, 1q42.12_DLBCL (FOCAL); SEQ ID NO: 897, 1q42.12_DLBCL (FOCAL); SEQ ID NO: 898, 1q42.12_DLBCL (FOCAL); SEQ ID NO: 899, 1q42.12_DLBCL (FOCAL); SEQ ID NO: 900, 1q42.12_DLBCL (FOCAL); SEQ ID NO: 901, 1q42.12_DLBCL (FOCAL); SEQ ID NO: 902, 1q42.12_DLBCL (FOCAL); SEQ ID NO: 903, 1q42.12_DLBCL (FOCAL); SEQ ID NO: 904, 1q42.12_DLBCL (FOCAL); SEQ ID NO: 905, 1q42.12_DLBCL (FOCAL); SEQ ID NO: 906, 1q42.12_DLBCL (FOCAL); SEQ ID NO: 907, 1q42.12_DLBCL (FOCAL); SEQ ID NO: 908, 1q42.12_DLBCL (FOCAL); SEQ ID NO: 909, 1q42.12_DLBCL (FOCAL); SEQ ID NO: 910, MSI (MSI); SEQ ID NO: 911, MSI (MSI); SEQ ID NO: 912, 1q42.12_DLBCL (FOCAL); SEQ ID NO: 913, 1q42.12_DLBCL (FOCAL); SEQ ID NO: 914, 1q42.12_DLBCL (FOCAL); SEQ ID NO: 915, 1q42.12_DLBCL (FOCAL); SEQ ID NO: 916, 1q42.12_DLBCL (FOCAL); SEQ ID NO: 917, 1q42.12_DLBCL (FOCAL); SEQ ID NO: 918, 1q42.12_DLBCL (FOCAL); SEQ ID NO: 919, 1q42.12_DLBCL (FOCAL); SEQ ID NO: 920, 1q42.12_DLBCL (FOCAL); SEQ ID NO: 921, 1q42.12_DLBCL (FOCAL); SEQ ID NO: 922, 1q42.12_DLBCL (FOCAL); SEQ ID NO: 923, 1q42.12_DLBCL (FOCAL); SEQ ID NO: 924, 1q42.12_DLBCL (FOCAL); SEQ ID NO: 925, 1q42.12_DLBCL (FOCAL); SEQ ID NO: 926, 1q42.12_DLBCL (FOCAL); SEQ ID NO: 927, 1q42.12_DLBCL (FOCAL); SEQ ID NO: 928, 1q42.12_DLBCL (FOCAL); SEQ ID NO: 929, 1q42.12_DLBCL (FOCAL); SEQ ID NO: 930, 1q42.12_DLBCL (FOCAL); SEQ ID NO: 931, 1q42.12_DLBCL (FOCAL); SEQ ID NO: 932, 1q42.12_DLBCL (FOCAL); SEQ ID NO: 933, 1q42.12_DLBCL (FOCAL); SEQ ID NO: 934, 1q42.12_DLBCL (FOCAL); SEQ ID NO: 935, 1q42.12_DLBCL (FOCAL); SEQ ID NO: 936, 1q42.12_DLBCL (FOCAL); SEQ ID NO: 937, 1q42.12_DLBCL (FOCAL); SEQ ID NO: 938, 1q42.12_DLBCL (FOCAL); SEQ ID NO: 939, 1q42.12_DLBCL (FOCAL); SEQ ID NO: 940, 1q42.12_DLBCL (FOCAL); SEQ ID NO: 941, 1q42.12_DLBCL (FOCAL); SEQ ID NO: 942, IRF2BP2 (Gene); SEQ ID NO: 943, IRF2BP2 (Gene); SEQ ID NO: 944, IRF2BP2 (Gene); SEQ ID NO: 945, IRF2BP2 (Gene); SEQ ID NO: 946, IRF2BP2 (Gene); SEQ ID NO: 947, 1q42.12_DLBCL (FOCAL); SEQ ID NO: 948, 1q42.12_DLBCL (FOCAL); SEQ ID NO: 949, 1q42.12_DLBCL (FOCAL); SEQ ID NO: 950, 1q42.12_DLBCL (FOCAL); SEQ ID NO: 951, 1q42.12_DLBCL (FOCAL); SEQ ID NO: 952, 1q42.12_DLBCL (FOCAL); SEQ ID NO: 953, 1q42.12_DLBCL (FOCAL); SEQ ID NO: 954, 1q42.12_DLBCL (FOCAL); SEQ ID NO: 955, 1q42.12_DLBCL (FOCAL); SEQ ID NO: 956, 1q42.12_DLBCL (FOCAL); SEQ ID NO: 957, 1q42.12_DLBCL (FOCAL); SEQ ID NO: 958, 1q42.12_DLBCL (FOCAL); SEQ ID NO: 959, 1q42.12_DLBCL (FOCAL); SEQ ID NO: 960, 1q42.12_DLBCL (FOCAL); SEQ ID NO: 961, 1q42.12_DLBCL (FOCAL); SEQ ID NO: 962, 1q42.12_DLBCL (FOCAL); SEQ ID NO: 963, 1q42.12_DLBCL (FOCAL); SEQ ID NO: 964, 1q42.12_DLBCL (FOCAL); SEQ ID NO: 965, 1q42.12_DLBCL (FOCAL); SEQ ID NO: 966, 1q42.12_DLBCL (FOCAL); SEQ ID NO: 967, 1q42.12_DLBCL (FOCAL); SEQ ID NO: 968, 1q42.12_DLBCL (FOCAL); SEQ ID NO: 969, 1q42.12_DLBCL (FOCAL); SEQ ID NO: 970, 1q42.12_DLBCL (FOCAL); SEQ ID NO: 971, 1q42.12_DLBCL (FOCAL); SEQ ID NO: 972, 1q42.12_DLBCL (FOCAL); SEQ ID NO: 973, 1q42.12_DLBCL (FOCAL); SEQ ID NO: 974, 1q42.12_DLBCL (FOCAL); SEQ ID NO: 975, 1q42.12_DLBCL (FOCAL); SEQ ID NO: 976, 1q42.12_DLBCL (FOCAL); SEQ ID NO: 977, 1q42.12_DLBCL (FOCAL); SEQ ID NO: 978, FP (FP); SEQ ID NO: 979, 1q42.12_DLBCL (FOCAL); SEQ ID NO: 980, 1q42.12_DLBCL (FOCAL); SEQ ID NO: 981, 1q42.12_DLBCL (FOCAL); SEQ ID NO: 982, 1q42.12_DLBCL (FOCAL); SEQ ID NO: 983, 1q42.12_DLBCL (FOCAL); SEQ ID NO: 984, 1q42.12_DLBCL (FOCAL); SEQ ID NO: 985, 1q42.12_DLBCL (FOCAL); SEQ ID NO: 986, 1q42.12_DLBCL (FOCAL); SEQ ID NO: 987, FP (FP); SEQ ID NO: 988, 1q42.12_DLBCL (FOCAL); SEQ ID NO: 989, 1q42.12_DLBCL (FOCAL); SEQ ID NO: 990, 1q42.12_DLBCL (FOCAL); SEQ ID NO: 991, 1q42.12_DLBCL (FOCAL); SEQ ID NO: 992, 1q42.12_DLBCL (FOCAL); SEQ ID NO: 993, 1q42.12_DLBCL (FOCAL); SEQ ID NO: 994, 1q42.12_DLBCL (FOCAL); SEQ ID NO: 995, 1q42.12_DLBCL (FOCAL); SEQ ID NO: 996, 1q42.12_DLBCL (FOCAL); SEQ ID NO: 997, 1q42.12_DLBCL (FOCAL); SEQ ID NO: 998, 1q42.12_DLBCL (FOCAL); SEQ ID NO: 999, 1q42.12_DLBCL (FOCAL); SEQ ID NO: 1000, 1q42.12_DLBCL (FOCAL); SEQ ID NO: 1001, 1q42.12_DLBCL (FOCAL); SEQ ID NO: 1002, 1q42.12_DLBCL (FOCAL); SEQ ID NO: 1003, 1q42.12_DLBCL (FOCAL); SEQ ID NO: 1004, 1q42.12_DLBCL (FOCAL); SEQ ID NO: 1005, 1q42.12_DLBCL (FOCAL); SEQ ID NO: 1006, 1q42.12_DLBCL (FOCAL); SEQ ID NO: 1007, 1q42.12_DLBCL (FOCAL); SEQ ID NO: 1008, 1q42.12_DLBCL (FOCAL); SEQ ID NO: 1009, FP (FP); SEQ ID NO: 1010, 1q42.12_DLBCL (FOCAL); SEQ ID NO: 1011, 1q42.12_DLBCL (FOCAL); SEQ ID NO: 1012, 1q42.12_DLBCL (FOCAL); SEQ ID NO: 1013, 1q42.12_DLBCL (FOCAL); SEQ ID NO: 1014, 1q42.12_DLBCL (FOCAL); SEQ ID NO: 1015, 1q42.12_DLBCL (FOCAL); SEQ ID NO: 1016, 1q42.12_DLBCL (FOCAL); SEQ ID NO: 1017, 1q42.12_DLBCL (FOCAL); SEQ ID NO: 1018, 1q42.12_DLBCL (FOCAL); SEQ ID NO: 1019, 1q42.12_DLBCL (FOCAL); SEQ ID NO: 1020, 1q42.12_DLBCL (FOCAL); SEQ ID NO: 1021, 1q42.12_DLBCL (FOCAL); SEQ ID NO: 1022, 1q42.12_DLBCL (FOCAL); SEQ ID NO: 1023, 1q42.12_DLBCL (FOCAL); SEQ ID NO: 1024, 1q42.12_DLBCL (FOCAL); SEQ ID NO: 1025, 1q42.12_DLBCL (FOCAL); SEQ ID NO: 1026, 1q42.12_DLBCL (FOCAL); SEQ ID NO: 1027, 1q42.12_DLBCL (FOCAL); SEQ ID NO: 1028, 1q42.12_DLBCL (FOCAL); SEQ ID NO: 1029, 1q42.12_DLBCL (FOCAL); SEQ ID NO: 1030, 1q42.12_DLBCL (FOCAL); SEQ ID NO: 1031, 1q42.12_DLBCL (FOCAL); SEQ ID NO: 1032, 1q42.12_DLBCL (FOCAL); SEQ ID NO: 1033, 1q42.12_DLBCL (FOCAL); SEQ ID NO: 1034, 1q42.12_DLBCL (FOCAL); SEQ ID NO: 1035, 1q42.12_DLBCL (FOCAL); SEQ ID NO: 1036, 1q42.12_DLBCL (FOCAL); SEQ ID NO: 1037, 1q42.12_DLBCL (FOCAL); SEQ ID NO: 1038, 1q42.12_DLBCL (FOCAL); SEQ ID NO: 1039, 1q42.12_DLBCL (FOCAL); SEQ ID NO: 1040, 1q42.12_DLBCL (FOCAL); SEQ ID NO: 1041, 1q42.12_DLBCL (FOCAL); SEQ ID NO: 1042, 1q42.12_DLBCL (FOCAL); SEQ ID NO: 1043, 1q42.12_DLBCL (FOCAL); SEQ ID NO: 1044, 1q42.12_DLBCL (FOCAL); SEQ ID NO: 1045, 1q42.12_DLBCL (FOCAL); SEQ ID NO: 1046, 1q42.12_DLBCL (FOCAL); SEQ ID NO: 1047, 1q42.12_DLBCL (FOCAL); SEQ ID NO: 1048, 1q42.12_DLBCL (FOCAL); SEQ ID NO: 1049, 1q42.12_DLBCL (FOCAL); SEQ ID NO: 1050, 1q42.12_DLBCL (FOCAL); SEQ ID NO: 1051, 1q42.12_DLBCL (FOCAL); SEQ ID NO: 1052, 1q42.12_DLBCL (FOCAL); SEQ ID NO: 1053, 1q42.12_DLBCL (FOCAL); SEQ ID NO: 1054, 10P (ARM); SEQ ID NO: 1055, 10P (ARM); SEQ ID NO: 1056, 10P (ARM); SEQ ID NO: 1057, 10P (ARM); SEQ ID NO: 1058, FP (FP); SEQ ID NO: 1059, 10P (ARM); SEQ ID NO: 1060, 10P (ARM); SEQ ID NO: 1061, 10P (ARM); SEQ ID NO: 1062, 10P (ARM); SEQ ID NO: 1063, 10P (ARM); SEQ ID NO: 1064, 10P (ARM); SEQ ID NO: 1065, 10P (ARM); SEQ ID NO: 1066, 10P (ARM); SEQ ID NO: 1067, 10P (ARM); SEQ ID NO: 1068, 10P (ARM); SEQ ID NO: 1069, 10P (ARM); SEQ ID NO: 1070, 10P (ARM); SEQ ID NO: 1071, 10P (ARM); SEQ ID NO: 1072, 10P (ARM); SEQ ID NO: 1073, 10P (ARM); SEQ ID NO: 1074, 10P (ARM); SEQ ID NO: 1075, 10P (ARM); SEQ ID NO: 1076, 10P (ARM); SEQ ID NO: 1077, 10P (ARM); SEQ ID NO: 1078, 10P (ARM); SEQ ID NO: 1079, 10P (ARM); SEQ ID NO: 1080, 10P (ARM); SEQ ID NO: 1081, 10P (ARM); SEQ ID NO: 1082, 10P (ARM); SEQ ID NO: 1083, WAC (Gene); SEQ ID NO: 1084, WAC (Gene); SEQ ID NO: 1085, WAC (Gene); SEQ ID NO: 1086, WAC (Gene); SEQ ID NO: 1087, WAC (Gene); SEQ ID NO: 1088, WAC (Gene); SEQ ID NO: 1089, WAC (Gene); SEQ ID NO: 1090, WAC (Gene); SEQ ID NO: 1091, WAC (Gene); SEQ ID NO: 1092, WAC (Gene); SEQ ID NO: 1093, WAC (Gene); SEQ ID NO: 1094, WAC (Gene); SEQ ID NO: 1095, WAC (Gene); SEQ ID NO: 1096, WAC (Gene); SEQ ID NO: 1097, WAC (Gene); SEQ ID NO: 1098, 10P (ARM); SEQ ID NO: 1099, 10P (ARM); SEQ ID NO: 1100, MSI (MSI); SEQ ID NO: 1101, MSI (MSI); SEQ ID NO: 1102, 10P (ARM); SEQ ID NO: 1103, 10P (ARM); SEQ ID NO: 1104, 10P (ARM); SEQ ID NO: 1105, 10P (ARM); SEQ ID NO: 1106, 10P (ARM); SEQ ID NO: 1107, RET (Gene); SEQ ID NO: 1108, RET (Gene); SEQ ID NO: 1109, RET (Gene); SEQ ID NO: 1110, RET (Gene); SEQ ID NO: 1111, RET (Gene); SEQ ID NO: 1112, RET (Gene); SEQ ID NO: 1113, RET (Gene); SEQ ID NO: 1114, RET (Gene); SEQ ID NO: 1115, RET (Gene); SEQ ID NO: 1116, RET (Gene); SEQ ID NO: 1117, RET (Gene); SEQ ID NO: 1118, RET (Gene); SEQ ID NO: 1119, RET (Gene); SEQ ID NO: 1120, RET (Gene); SEQ ID NO: 1121, RET (Gene); SEQ ID NO: 1122, RET (Gene); SEQ ID NO: 1123, RET (Gene); SEQ ID NO: 1124, RET (Gene); SEQ ID NO: 1125, RET (Gene); SEQ ID NO: 1126, RET (Gene); SEQ ID NO: 1127, RET (Gene); SEQ ID NO: 1128, 10P (ARM); SEQ ID NO: 1129, 10P (ARM); SEQ ID NO: 1130, 10P (ARM); SEQ ID NO: 1131, 10P (ARM); SEQ ID NO: 1132, 10P (ARM); SEQ ID NO: 1133, 10P (ARM); SEQ ID NO: 1134, 10P (ARM); SEQ ID NO: 1135, 10P (ARM); SEQ ID NO: 1136, 10P (ARM); SEQ ID NO: 1137, 10P (ARM); SEQ ID NO: 1138, 10P (ARM); SEQ ID NO: 1139, 10P (ARM); SEQ ID NO: 1140, FP (FP); SEQ ID NO: 1141, 10P (ARM); SEQ ID NO: 1142, 10P (ARM); SEQ ID NO: 1143, 10P (ARM); SEQ ID NO: 1144, 10P (ARM); SEQ ID NO: 1145, 10P (ARM); SEQ ID NO: 1146, ARID5B (Gene); SEQ ID NO: 1147, ARID5B (Gene); SEQ ID NO: 1148, ARID5B (Gene); SEQ ID NO: 1149, ARID5B (Gene); SEQ ID NO: 1150, ARID5B (Gene); SEQ ID NO: 1151, ARID5B (Gene); SEQ ID NO: 1152, ARID5B (Gene); SEQ ID NO: 1153, ARID5B (Gene); SEQ ID NO: 1154, ARID5B (Gene); SEQ ID NO: 1155, ARID5B (Gene); SEQ ID NO: 1156, ARID5B (Gene); SEQ ID NO: 1157, ARID5B (Gene); SEQ ID NO: 1158, ARID5B (Gene); SEQ ID NO: 1159, ARID5B (Gene); SEQ ID NO: 1160, ARID5B (Gene); SEQ ID NO: 1161, ARID5B (Gene); SEQ ID NO: 1162, ARID5B (Gene); SEQ ID NO: 1163, ARID5B (Gene); SEQ ID NO: 1164, ARID5B (Gene); SEQ ID NO: 1165, ARID5B (Gene); SEQ ID NO: 1166, 10P (ARM); SEQ ID NO: 1167, 10P (ARM); SEQ ID NO: 1168, 10P (ARM); SEQ ID NO: 1169, 10P (ARM); SEQ ID NO: 1170, 10P (ARM); SEQ ID NO: 1171, MSI (MSI); SEQ ID NO: 1172, MSI (MSI); SEQ ID NO: 1173, 10P (ARM); SEQ ID NO: 1174, 10P (ARM); SEQ ID NO: 1175, 10P (ARM); SEQ ID NO: 1176, 10P (ARM); SEQ ID NO: 1177, 10P (ARM); SEQ ID NO: 1178, 10P (ARM); SEQ ID NO: 1179, 10P (ARM); SEQ ID NO: 1180, 10P (ARM); SEQ ID NO: 1181, 10P (ARM); SEQ ID NO: 1182, 10P (ARM); SEQ ID NO: 1183, 10P (ARM); SEQ ID NO: 1184, 10P (ARM); SEQ ID NO: 1185, 10P (ARM); SEQ ID NO: 1186, 10P (ARM); SEQ ID NO: 1187, 10P (ARM); SEQ ID NO: 1188, 10P (ARM); SEQ ID NO: 1189, 10P (ARM); SEQ ID NO: 1190, 10P (ARM); SEQ ID NO: 1191, 10P (ARM); SEQ ID NO: 1192, 10P (ARM); SEQ ID NO: 1193, 10q23.31_DLBCL (FOCAL); SEQ ID NO: 1194, 10q23.31_DLBCL (FOCAL); SEQ ID NO: 1195, 10q23.31_DLBCL (FOCAL); SEQ ID NO: 1196, PTEN (Gene); SEQ ID NO: 1197, PTEN (Gene); SEQ ID NO: 1198, PTEN (Gene); SEQ ID NO: 1199, PTEN (Gene); SEQ ID NO: 1200, PTEN (Gene); SEQ ID NO: 1201, PTEN (Gene); SEQ ID NO: 1202, PTEN (Gene); SEQ ID NO: 1203, PTEN (Gene); SEQ ID NO: 1204, PTEN (Gene); SEQ ID NO: 1205, PTEN (Gene); SEQ ID NO: 1206, 10q23.31_DLBCL (FOCAL); SEQ ID NO: 1207, 10q23.31_DLBCL (FOCAL); SEQ ID NO: 1208, 10q23.31_DLBCL (FOCAL); SEQ ID NO: 1209, 10q23.31_DLBCL (FOCAL); SEQ ID NO: 1210, 10q23.31_DLBCL (FOCAL); SEQ ID NO: 1211, FAS (Gene); SEQ ID NO: 1212, FAS (Gene); SEQ ID NO: 1213, FAS (Gene); SEQ ID NO: 1214, FAS (Gene); SEQ ID NO: 1215, FAS (Gene); SEQ ID NO: 1216, FAS (Gene); SEQ ID NO: 1217, FAS (Gene); SEQ ID NO: 1218, FAS (Gene); SEQ ID NO: 1219, FAS (Gene); SEQ ID NO: 1220, 10q23.31_DLBCL (FOCAL); SEQ ID NO: 1221, 10q23.31_DLBCL (FOCAL); SEQ ID NO: 1222, 10P (ARM); SEQ ID NO: 1223, 10P (ARM); SEQ ID NO: 1224, 10P (ARM); SEQ ID NO: 1225, 10P (ARM); SEQ ID NO: 1226, 10P (ARM); SEQ ID NO: 1227, 10P (ARM); SEQ ID NO: 1228, MSI (MSI); SEQ ID NO: 1229, MSI (MSI); SEQ ID NO: 1230, 10P (ARM); SEQ ID NO: 1231, MSI (MSI); SEQ ID NO: 1232, MSI (MSI); SEQ ID NO: 1233, 10P (ARM); SEQ ID NO: 1234, 10Q (ARM); SEQ ID NO: 1235, 10Q (ARM); SEQ ID NO: 1236, 10Q (ARM); SEQ ID NO: 1237, 10Q (ARM); SEQ ID NO: 1238, 10Q (ARM); SEQ ID NO: 1239, 10Q (ARM); SEQ ID NO: 1240, 10Q (ARM); SEQ ID NO: 1241, 10Q (ARM); SEQ ID NO: 1242, MSI (MSI); SEQ ID NO: 1243, MSI (MSI); SEQ ID NO: 1244, 10Q (ARM); SEQ ID NO: 1245, 10Q (ARM); SEQ ID NO: 1246, 10Q (ARM); SEQ ID NO: 1247, 10Q (ARM); SEQ ID NO: 1248, 10Q (ARM); SEQ ID NO: 1249, 10Q (ARM); SEQ ID NO: 1250, 10Q (ARM); SEQ ID NO: 1251, 10Q (ARM); SEQ ID NO: 1252, 10Q (ARM); SEQ ID NO: 1253, 10Q (ARM); SEQ ID NO: 1254, 10Q (ARM); SEQ ID NO: 1255, 10Q (ARM); SEQ ID NO: 1256, 10Q (ARM); SEQ ID NO: 1257, 10Q (ARM); SEQ ID NO: 1258, 10Q (ARM); SEQ ID NO: 1259, 10Q (ARM); SEQ ID NO: 1260, 10Q (ARM); SEQ ID NO: 1261, 10Q (ARM); SEQ ID NO: 1262, 10Q (ARM); SEQ ID NO: 1263, 10Q (ARM); SEQ ID NO: 1264, FP (FP); SEQ ID NO: 1265, 10Q (ARM); SEQ ID NO: 1266, 10Q (ARM); SEQ ID NO: 1267, 10Q (ARM); SEQ ID NO: 1268, 10Q (ARM); SEQ ID NO: 1269, 10Q (ARM); SEQ ID NO: 1270, FP (FP); SEQ ID NO: 1271, 10Q (ARM); SEQ ID NO: 1272, 10Q (ARM); SEQ ID NO: 1273, 10Q (ARM); SEQ ID NO: 1274, 11P_DLBCL (ARM); SEQ ID NO: 1275, 11P_DLBCL (ARM); SEQ ID NO: 1276, 11P_DLBCL (ARM); SEQ ID NO: 1277, 11P_DLBCL (ARM); SEQ ID NO: 1278, 11P_DLBCL (ARM); SEQ ID NO: 1279, 11P_DLBCL (ARM); SEQ ID NO: 1280, 11P_DLBCL (ARM); SEQ ID NO: 1281, 11P_DLBCL (ARM); SEQ ID NO: 1282, 11P_DLBCL (ARM); SEQ ID NO: 1283, 11P_DLBCL (ARM); SEQ ID NO: 1284, FP (FP); SEQ ID NO: 1285, 11P_DLBCL (ARM); SEQ ID NO: 1286, 11P_DLBCL (ARM); SEQ ID NO: 1287, 11P_DLBCL (ARM); SEQ ID NO: 1288, 11P_DLBCL (ARM); SEQ ID NO: 1289, 11P_DLBCL (ARM); SEQ ID NO: 1290, 11P_DLBCL (ARM); SEQ ID NO: 1291, 11P_DLBCL (ARM); SEQ ID NO: 1292, 11P_DLBCL (ARM); SEQ ID NO: 1293, 11P_DLBCL (ARM); SEQ ID NO: 1294, 11P_DLBCL (ARM); SEQ ID NO: 1295, 11P_DLBCL (ARM); SEQ ID NO: 1296, 11P_DLBCL (ARM); SEQ ID NO: 1297, 11P_DLBCL (ARM); SEQ ID NO: 1298, 11P_DLBCL (ARM); SEQ ID NO: 1299, 11P_DLBCL (ARM); SEQ ID NO: 1300, 11P_DLBCL (ARM); SEQ ID NO: 1301, 11P_DLBCL (ARM); SEQ ID NO: 1302, 11P_DLBCL (ARM); SEQ ID NO: 1303, 11P_DLBCL (ARM); SEQ ID NO: 1304, 11P_DLBCL (ARM); SEQ ID NO: 1305, 11P_DLBCL (ARM); SEQ ID NO: 1306, 11P_DLBCL (ARM); SEQ ID NO: 1307, 11P_DLBCL (ARM); SEQ ID NO: 1308, 11P_DLBCL (ARM); SEQ ID NO: 1309, 11P_DLBCL (ARM); SEQ ID NO: 1310, 11P_DLBCL (ARM); SEQ ID NO: 1311, 11P_DLBCL (ARM); SEQ ID NO: 1312, 11P_DLBCL (ARM); SEQ ID NO: 1313, 11P_DLBCL (ARM); SEQ ID NO: 1314, 11P_DLBCL (ARM); SEQ ID NO: 1315, 11P_DLBCL (ARM); SEQ ID NO: 1316, 11P_DLBCL (ARM); SEQ ID NO: 1317, 11P_DLBCL (ARM); SEQ ID NO: 1318, 11P_DLBCL (ARM); SEQ ID NO: 1319, 11P_DLBCL (ARM); SEQ ID NO: 1320, 11P_DLBCL (ARM); SEQ ID NO: 1321, 11P_DLBCL (ARM); SEQ ID NO: 1322, 11Q_DLBCL (ARM); SEQ ID NO: 1323, MS4A1 (Gene); SEQ ID NO: 1324, MS4A1 (Gene); SEQ ID NO: 1325, MS4A1 (Gene); SEQ ID NO: 1326, 11Q_DLBCL (ARM); SEQ ID NO: 1327, MS4A1 (Gene); SEQ ID NO: 1328, MS4A1 (Gene); SEQ ID NO: 1329, MS4A1 (Gene); SEQ ID NO: 1330, 11Q_DLBCL (ARM); SEQ ID NO: 1331, 11Q_DLBCL (ARM); SEQ ID NO: 1332, MSI (MSI); SEQ ID NO: 1333, MSI (MSI); SEQ ID NO: 1334, 11Q_DLBCL (ARM); SEQ ID NO: 1335, 11Q_DLBCL (ARM); SEQ ID NO: 1336, CCND1_MTC (SV); SEQ ID NO: 1337, CCND1_MTC (SV); SEQ ID NO: 1338, CCND1_MTC (SV); SEQ ID NO: 1339, CCND1_MTC (SV); SEQ ID NO: 1340, CCND1_MTC (SV); SEQ ID NO: 1341, CCND1_MTC (SV); SEQ ID NO: 1342, CCND1_MTC (SV); SEQ ID NO: 1343, CCND1_MTC (SV); SEQ ID NO: 1344, CCND1_MTC (SV); SEQ ID NO: 1345, CCND1_MTC (SV); SEQ ID NO: 1346, CCND1_MTC (SV); SEQ ID NO: 1347, CCND1_MTC (SV); SEQ ID NO: 1348, CCND1_MTC (SV); SEQ ID NO: 1349, CCND1_MTC (SV); SEQ ID NO: 1350, CCND1_MTC (SV); SEQ ID NO: 1351, CCND1_MTC (SV); SEQ ID NO: 1352, CCND1_MTC (SV); SEQ ID NO: 1353, CCND1_MTC (SV); SEQ ID NO: 1354, CCND1_MTC (SV); SEQ ID NO: 1355, CCND1_MTC (SV); SEQ ID NO: 1356, CCND1_MTC (SV); SEQ ID NO: 1357, CCND1_MTC (SV); SEQ ID NO: 1358, CCND1_MTC (SV); SEQ ID NO: 1359, CCND1_MTC (SV); SEQ ID NO: 1360, CCND1_MTC (SV); SEQ ID NO: 1361, CCND1_MTC (SV); SEQ ID NO: 1362, CCND1_MTC (SV); SEQ ID NO: 1363, CCND1_MTC (SV); SEQ ID NO: 1364, CCND1_MTC (SV); SEQ ID NO: 1365, CCND1_MTC (SV); SEQ ID NO: 1366, CCND1_MTC (SV); SEQ ID NO: 1367, CCND1_MTC (SV); SEQ ID NO: 1368, CCND1_MTC (SV); SEQ ID NO: 1369, CCND1_MTC (SV); SEQ ID NO: 1370, CCND1_MTC (SV); SEQ ID NO: 1371, CCND1_MTC (SV); SEQ ID NO: 1372, CCND1_MTC (SV); SEQ ID NO: 1373, CCND1_MTC (SV); SEQ ID NO: 1374, CCND1_MTC (SV); SEQ ID NO: 1375, CCND1_MTC (SV); SEQ ID NO: 1376, CCND1_MTC (SV); SEQ ID NO: 1377, CCND1_MTC (SV); SEQ ID NO: 1378, CCND1_MTC (SV); SEQ ID NO: 1379, CCND1_MTC (SV); SEQ ID NO: 1380, CCND1_MTC (SV); SEQ ID NO: 1381, CCND1_MTC (SV); SEQ ID NO: 1382, CCND1_MTC (SV); SEQ ID NO: 1383, CCND1_MTC (SV); SEQ ID NO: 1384, CCND1_MTC (SV); SEQ ID NO: 1385, CCND1_MTC (SV); SEQ ID NO: 1386, CCND1_MTC (SV); SEQ ID NO: 1387, CCND1_MTC (SV); SEQ ID NO: 1388, CCND1_MTC (SV); SEQ ID NO: 1389, CCND1_MTC (SV); SEQ ID NO: 1390, CCND1_MTC (SV); SEQ ID NO: 1391, CCND1_MTC (SV); SEQ ID NO: 1392, CCND1_MTC (SV); SEQ ID NO: 1393, CCND1_promoter (SV); SEQ ID NO: 1394, CCND1_promoter (SV); SEQ ID NO: 1395, CCND1_promoter (SV); SEQ ID NO: 1396, CCND1_promoter (SV); SEQ ID NO: 1397, CCND1_promoter (SV); SEQ ID NO: 1398, CCND1_promoter (SV); SEQ ID NO: 1399, CCND1_promoter (SV); SEQ ID NO: 1400, CCND1_promoter (SV); SEQ ID NO: 1401, CCND1_promoter (SV); SEQ ID NO: 1402, CCND1_promoter (SV); SEQ ID NO: 1403, CCND1_promoter (SV); SEQ ID NO: 1404, CCND1_promoter (SV); SEQ ID NO: 1405, CCND1_promoter (SV); SEQ ID NO: 1406, CCND1_promoter (SV); SEQ ID NO: 1407, CCND1_promoter (SV); SEQ ID NO: 1408, CCND1_promoter (SV); SEQ ID NO: 1409, CCND1_promoter (SV); SEQ ID NO: 1410, CCND1_promoter (SV); SEQ ID NO: 1411, CCND1_promoter (SV); SEQ ID NO: 1412, CCND1_promoter (SV); SEQ ID NO: 1413, CCND1_promoter (SV); SEQ ID NO: 1414, CCND1_promoter (SV); SEQ ID NO: 1415, CCND1_promoter (SV); SEQ ID NO: 1416, CCND1_promoter (SV); SEQ ID NO: 1417, CCND1_promoter (SV); SEQ ID NO: 1418, CCND1_promoter (SV); SEQ ID NO: 1419, CCND1_promoter (SV); SEQ ID NO: 1420, CCND1_promoter (SV); SEQ ID NO: 1421, CCND1_promoter (SV); SEQ ID NO: 1422, CCND1_promoter (SV); SEQ ID NO: 1423, CCND1_promoter (SV); SEQ ID NO: 1424, CCND1_promoter (SV); SEQ ID NO: 1425, CCND1_promoter (SV); SEQ ID NO: 1426, CCND1_promoter (SV); SEQ ID NO: 1427, CCND1_promoter (SV); SEQ ID NO: 1428, CCND1_promoter (SV); SEQ ID NO: 1429, CCND1_promoter (SV); SEQ ID NO: 1430, CCND1_promoter (SV); SEQ ID NO: 1431, CCND1_promoter (SV); SEQ ID NO: 1432, CCND1_promoter (SV); SEQ ID NO: 1433, CCND1_promoter (SV); SEQ ID NO: 1434, CCND1_promoter (SV); SEQ ID NO: 1435, CCND1_promoter (SV); SEQ ID NO: 1436, CCND1_promoter (SV); SEQ ID NO: 1437, CCND1_promoter (SV); SEQ ID NO: 1438, CCND1_promoter (SV); SEQ ID NO: 1439, CCND1_promoter (SV); SEQ ID NO: 1440, CCND1_promoter (SV); SEQ ID NO: 1441, CCND1_promoter (SV); SEQ ID NO: 1442, CCND1_promoter (SV); SEQ ID NO: 1443, CCND1_promoter (SV); SEQ ID NO: 1444, CCND1_SV (SV); SEQ ID NO: 1445, CCND1_promoter (SV); SEQ ID NO: 1446, CCND1_promoter (SV); SEQ ID NO: 1447, CCND1_SV (SV); SEQ ID NO: 1448, CCND1_SV (SV); SEQ ID NO: 1449, CCND1 (Gene); SEQ ID NO: 1450, CCND1 (Gene); SEQ ID NO: 1451, CCND1 (Gene); SEQ ID NO: 1452, 11Q_DLBCL (ARM); SEQ ID NO: 1453, 11Q_DLBCL (ARM); SEQ ID NO: 1454, 11Q_DLBCL (ARM); SEQ ID NO: 1455, 11Q_DLBCL (ARM); SEQ ID NO: 1456, 11Q_DLBCL (ARM); SEQ ID NO: 1457, 11Q_DLBCL (ARM); SEQ ID NO: 1458, 11Q_DLBCL (ARM); SEQ ID NO: 1459, 11Q_DLBCL (ARM); SEQ ID NO: 1460, 11Q_DLBCL (ARM); SEQ ID NO: 1461, 11Q_DLBCL (ARM); SEQ ID NO: 1462, 11Q_DLBCL (ARM); SEQ ID NO: 1463, 11Q_DLBCL (ARM); SEQ ID NO: 1464, 11Q_DLBCL (ARM); SEQ ID NO: 1465, 11Q_DLBCL (ARM); SEQ ID NO: 1466, 11Q_DLBCL (ARM); SEQ ID NO: 1467, 11Q_DLBCL (ARM); SEQ ID NO: 1468, 11Q_DLBCL (ARM); SEQ ID NO: 1469, 11Q_DLBCL (ARM); SEQ ID NO: 1470, 11Q_DLBCL (ARM); SEQ ID NO: 1471, 11Q_DLBCL (ARM); SEQ ID NO: 1472, 11Q_DLBCL (ARM); SEQ ID NO: 1473, 11Q_DLBCL (ARM); SEQ ID NO: 1474, 11Q_DLBCL (ARM); SEQ ID NO: 1475, 11Q_DLBCL (ARM); SEQ ID NO: 1476, 11Q_DLBCL (ARM); SEQ ID NO: 1477, 11Q_DLBCL (ARM); SEQ ID NO: 1478, 11Q_DLBCL (ARM); SEQ ID NO: 1479, 11Q_DLBCL (ARM); SEQ ID NO: 1480, 11Q_DLBCL (ARM); SEQ ID NO: 1481, 11Q_DLBCL (ARM); SEQ ID NO: 1482, 11Q_DLBCL (ARM); SEQ ID NO: 1483, BIRC3 (Gene); SEQ ID NO: 1484, BIRC3 (Gene); SEQ ID NO: 1485, BIRC3 (Gene); SEQ ID NO: 1486, BIRC3 (Gene); SEQ ID NO: 1487, BIRC3 (Gene); SEQ ID NO: 1488, BIRC3 (Gene); SEQ ID NO: 1489, BIRC3 (Gene); SEQ ID NO: 1490, BIRC3 (Gene); SEQ ID NO: 1491, BIRC3 (Gene); SEQ ID NO: 1492, BIRC3 (Gene); SEQ ID NO: 1493, 11Q_DLBCL (ARM); SEQ ID NO: 1494, 11Q_DLBCL (ARM); SEQ ID NO: 1495, 11Q_DLBCL (ARM); SEQ ID NO: 1496, 11Q_DLBCL (ARM); SEQ ID NO: 1497, ATM (Gene); SEQ ID NO: 1498, ATM (Gene); SEQ ID NO: 1499, ATM (Gene); SEQ ID NO: 1500, ATM (Gene); SEQ ID NO: 1501, ATM (Gene); SEQ ID NO: 1502, ATM (Gene); SEQ ID NO: 1503, ATM (Gene); SEQ ID NO: 1504, ATM (Gene); SEQ ID NO: 1505, ATM (Gene); SEQ ID NO: 1506, ATM (Gene); SEQ ID NO: 1507, ATM (Gene); SEQ ID NO: 1508, ATM (Gene); SEQ ID NO: 1509, ATM (Gene); SEQ ID NO: 1510, ATM (Gene); SEQ ID NO: 1511, ATM (Gene); SEQ ID NO: 1512, ATM (Gene); SEQ ID NO: 1513, ATM (Gene); SEQ ID NO: 1514, ATM (Gene); SEQ ID NO: 1515, ATM (Gene); SEQ ID NO: 1516, ATM (Gene); SEQ ID NO: 1517, ATM (Gene); SEQ ID NO: 1518, ATM (Gene); SEQ ID NO: 1519, ATM (Gene); SEQ ID NO: 1520, ATM (Gene); SEQ ID NO: 1521, ATM (Gene); SEQ ID NO: 1522, ATM (Gene); SEQ ID NO: 1523, ATM (Gene); SEQ ID NO: 1524, ATM (Gene); SEQ ID NO: 1525, ATM (Gene); SEQ ID NO: 1526, ATM (Gene); SEQ ID NO: 1527, ATM (Gene); SEQ ID NO: 1528, ATM (Gene); SEQ ID NO: 1529, ATM (Gene); SEQ ID NO: 1530, ATM (Gene); SEQ ID NO: 1531, ATM (Gene); SEQ ID NO: 1532, ATM (Gene); SEQ ID NO: 1533, ATM (Gene); SEQ ID NO: 1534, ATM (Gene); SEQ ID NO: 1535, ATM (Gene); SEQ ID NO: 1536, ATM (Gene); SEQ ID NO: 1537, ATM (Gene); SEQ ID NO: 1538, ATM (Gene); SEQ ID NO: 1539, ATM (Gene); SEQ ID NO: 1540, ATM (Gene); SEQ ID NO: 1541, ATM (Gene); SEQ ID NO: 1542, ATM (Gene); SEQ ID NO: 1543, ATM (Gene); SEQ ID NO: 1544, ATM (Gene); SEQ ID NO: 1545, ATM (Gene); SEQ ID NO: 1546, ATM (Gene); SEQ ID NO: 1547, ATM (Gene); SEQ ID NO: 1548, ATM (Gene); SEQ ID NO: 1549, ATM (Gene); SEQ ID NO: 1550, ATM (Gene); SEQ ID NO: 1551, ATM (Gene); SEQ ID NO: 1552, ATM (Gene); SEQ ID NO: 1553, ATM (Gene); SEQ ID NO: 1554, ATM (Gene); SEQ ID NO: 1555, ATM (Gene); SEQ ID NO: 1556, ATM (Gene); SEQ ID NO: 1557, ATM (Gene); SEQ ID NO: 1558, ATM (Gene); SEQ ID NO: 1559, ATM (Gene); SEQ ID NO: 1560, 11Q_DLBCL (ARM); SEQ ID NO: 1561, FP (FP); SEQ ID NO: 1562, POU2AF1 (Gene); SEQ ID NO: 1563, POU2AF1 (Gene); SEQ ID NO: 1564, POU2AF1 (Gene); SEQ ID NO: 1565, POU2AF1 (Gene); SEQ ID NO: 1566, POU2AF1 (Gene); SEQ ID NO: 1567, 11Q_DLBCL (ARM); SEQ ID NO: 1568, 11Q_DLBCL (ARM); SEQ ID NO: 1569, 11Q_DLBCL (ARM); SEQ ID NO: 1570, 11Q_DLBCL (ARM); SEQ ID NO: 1571, 11Q_DLBCL (ARM); SEQ ID NO: 1572, 11Q_DLBCL (ARM); SEQ ID NO: 1573, 11Q_DLBCL (ARM); SEQ ID NO: 1574, 11Q_DLBCL (ARM); SEQ ID NO: 1575, 11Q_DLBCL (ARM); SEQ ID NO: 1576, 11q23.3_DLBCL (FOCAL); SEQ ID NO: 1577, 11q23.3_DLBCL (FOCAL); SEQ ID NO: 1578, 11q23.3_DLBCL (FOCAL); SEQ ID NO: 1579, 11q23.3_DLBCL (FOCAL); SEQ ID NO: 1580, 11q23.3_DLBCL (FOCAL); SEQ ID NO: 1581, IL10RA (Gene); SEQ ID NO: 1582, IL10RA (Gene); SEQ ID NO: 1583, IL10RA (Gene); SEQ ID NO: 1584, IL10RA (Gene); SEQ ID NO: 1585, IL10RA (Gene); SEQ ID NO: 1586, IL10RA (Gene); SEQ ID NO: 1587, IL10RA (Gene); SEQ ID NO: 1588, IL10RA (Gene); SEQ ID NO: 1589, 11q23.3_DLBCL (FOCAL); SEQ ID NO: 1590, 11q23.3_DLBCL (FOCAL); SEQ ID NO: 1591, MSI (MSI); SEQ ID NO: 1592, MSI (MSI); SEQ ID NO: 1593, 11Q_DLBCL (ARM); SEQ ID NO: 1594, 11Q_DLBCL (ARM); SEQ ID NO: 1595, 11Q_DLBCL (ARM); SEQ ID NO: 1596, 11Q_DLBCL (ARM); SEQ ID NO: 1597, 11Q_DLBCL (ARM); SEQ ID NO: 1598, 11Q_DLBCL (ARM); SEQ ID NO: 1599, 11Q_DLBCL (ARM); SEQ ID NO: 1600, 11Q_DLBCL (ARM); SEQ ID NO: 1601, 11Q_DLBCL (ARM); SEQ ID NO: 1602, 11Q_DLBCL (ARM); SEQ ID NO: 1603, 11Q_DLBCL (ARM); SEQ ID NO: 1604, MSI (MSI); SEQ ID NO: 1605, MSI (MSI); SEQ ID NO: 1606, 11Q_DLBCL (ARM); SEQ ID NO: 1607, 11Q_DLBCL (ARM); SEQ ID NO: 1608, 11Q_DLBCL (ARM); SEQ ID NO: 1609, 11Q_DLBCL (ARM); SEQ ID NO: 1610, ETS1 (Gene); SEQ ID NO: 1611, ETS1 (Gene); SEQ ID NO: 1612, ETS1 (Gene); SEQ ID NO: 1613, ETS1 (Gene); SEQ ID NO: 1614, ETS1 (Gene); SEQ ID NO: 1615, ETS1 (Gene); SEQ ID NO: 1616, ETS1 (Gene); SEQ ID NO: 1617, ETS1 (Gene); SEQ ID NO: 1618, ETS1 (Gene); SEQ ID NO: 1619, ETS1 (Gene); SEQ ID NO: 1620, ETS1 (Gene); SEQ ID NO: 1621, 11Q_DLBCL (ARM); SEQ ID NO: 1622, 11Q_DLBCL (ARM); SEQ ID NO: 1623, 11Q_DLBCL (ARM); SEQ ID NO: 1624, 11Q_DLBCL (ARM); SEQ ID NO: 1625, 11Q_DLBCL (ARM); SEQ ID NO: 1626, 11Q_DLBCL (ARM); SEQ ID NO: 1627, 11Q_DLBCL (ARM); SEQ ID NO: 1628, 11Q_DLBCL (ARM); SEQ ID NO: 1629, 11Q_DLBCL (ARM); SEQ ID NO: 1630, 11Q_DLBCL (ARM); SEQ ID NO: 1631, 11Q_DLBCL (ARM); SEQ ID NO: 1632, 11Q_DLBCL (ARM); SEQ ID NO: 1633, FP (FP); SEQ ID NO: 1634, 11Q_DLBCL (ARM); SEQ ID NO: 1635, MSI (MSI); SEQ ID NO: 1636, MSI (MSI); SEQ ID NO: 1637, FP (FP); SEQ ID NO: 1638, 12P_DLBCL (ARM); SEQ ID NO: 1639, 12P_DLBCL (ARM); SEQ ID NO: 1640, 12P_DLBCL (ARM); SEQ ID NO: 1641, 12P_DLBCL (ARM); SEQ ID NO: 1642, 12P_DLBCL (ARM); SEQ ID NO: 1643, 12P_DLBCL (ARM); SEQ ID NO: 1644, 12P_DLBCL (ARM); SEQ ID NO: 1645, 12P_DLBCL (ARM); SEQ ID NO: 1646, PTPN6 (Gene); SEQ ID NO: 1647, PTPN6 (Gene); SEQ ID NO: 1648, PTPN6 (Gene); SEQ ID NO: 1649, PTPN6 (Gene); SEQ ID NO: 1650, PTPN6 (Gene); SEQ ID NO: 1651, PTPN6 (Gene); SEQ ID NO: 1652, PTPN6 (Gene); SEQ ID NO: 1653, PTPN6 (Gene); SEQ ID NO: 1654, PTPN6 (Gene); SEQ ID NO: 1655, PTPN6 (Gene); SEQ ID NO: 1656, PTPN6 (Gene); SEQ ID NO: 1657, PTPN6 (Gene); SEQ ID NO: 1658, PTPN6 (Gene); SEQ ID NO: 1659, PTPN6 (Gene); SEQ ID NO: 1660, PTPN6 (Gene); SEQ ID NO: 1661, PTPN6 (Gene); SEQ ID NO: 1662, 12P_DLBCL (ARM); SEQ ID NO: 1663, 12p13.2_DLBCL (FOCAL); SEQ ID NO: 1664, 12p13.2_DLBCL (FOCAL); SEQ ID NO: 1665, 12p13.2_DLBCL (FOCAL); SEQ ID NO: 1666, 12p13.2_DLBCL (FOCAL); SEQ ID NO: 1667, 12p13.2_DLBCL (FOCAL); SEQ ID NO: 1668, ETV6 (Gene); SEQ ID NO: 1669, 12p13.2_DLBCL (FOCAL); SEQ ID NO: 1670, ETV6_SV (SV); SEQ ID NO: 1671, ETV6_SV (SV); SEQ ID NO: 1672, ETV6_SV (SV); SEQ ID NO: 1673, ETV6_SV (SV); SEQ ID NO: 1674, ETV6_SV (SV); SEQ ID NO: 1675, ETV6_SV (SV); SEQ ID NO: 1676, ETV6_SV (SV); SEQ ID NO: 1677, ETV6_SV (SV); SEQ ID NO: 1678, ETV6_SV (SV); SEQ ID NO: 1679, ETV6_SV (SV); SEQ ID NO: 1680, ETV6_SV (SV); SEQ ID NO: 1681, ETV6_SV (SV); SEQ ID NO: 1682, ETV6_SV (SV); SEQ ID NO: 1683, ETV6_SV (SV); SEQ ID NO: 1684, ETV6_SV (SV); SEQ ID NO: 1685, ETV6_SV (SV); SEQ ID NO: 1686, ETV6_SV (SV); SEQ ID NO: 1687, ETV6_SV (SV); SEQ ID NO: 1688, ETV6_SV (SV); SEQ ID NO: 1689, ETV6_SV (SV); SEQ ID NO: 1690, ETV6_SV (SV); SEQ ID NO: 1691, ETV6_SV (SV); SEQ ID NO: 1692, ETV6_SV (SV); SEQ ID NO: 1693, ETV6_SV (SV); SEQ ID NO: 1694, ETV6_SV (SV); SEQ ID NO: 1695, ETV6_SV (SV); SEQ ID NO: 1696, ETV6_SV (SV); SEQ ID NO: 1697, ETV6_SV (SV); SEQ ID NO: 1698, ETV6_SV (SV); SEQ ID NO: 1699, ETV6_SV (SV); SEQ ID NO: 1700, ETV6_SV (SV); SEQ ID NO: 1701, ETV6_SV (SV); SEQ ID NO: 1702, ETV6_SV (SV); SEQ ID NO: 1703, ETV6_SV (SV); SEQ ID NO: 1704, ETV6_SV (SV); SEQ ID NO: 1705, ETV6_SV (SV); SEQ ID NO: 1706, ETV6_SV (SV); SEQ ID NO: 1707, ETV6_SV (SV); SEQ ID NO: 1708, ETV6_SV (SV); SEQ ID NO: 1709, ETV6_SV (SV); SEQ ID NO: 1710, ETV6_SV (SV); SEQ ID NO: 1711, ETV6_SV (SV); SEQ ID NO: 1712, ETV6_SV (SV); SEQ ID NO: 1713, ETV6_SV (SV); SEQ ID NO: 1714, ETV6_SV (SV); SEQ ID NO: 1715, ETV6_SV (SV); SEQ ID NO: 1716, ETV6_SV (SV); SEQ ID NO: 1717, ETV6_SV (SV); SEQ ID NO: 1718, ETV6_SV (SV); SEQ ID NO: 1719, ETV6_SV (SV); SEQ ID NO: 1720, ETV6_SV (SV); SEQ ID NO: 1721, ETV6_SV (SV); SEQ ID NO: 1722, ETV6_SV (SV); SEQ ID NO: 1723, ETV6_SV (SV); SEQ ID NO: 1724, ETV6_SV (SV); SEQ ID NO: 1725, ETV6_SV (SV); SEQ ID NO: 1726, ETV6_SV (SV); SEQ ID NO: 1727, ETV6_SV (SV); SEQ ID NO: 1728, ETV6_SV (SV); SEQ ID NO: 1729, ETV6_SV (SV); SEQ ID NO: 1730, ETV6_SV (SV); SEQ ID NO: 1731, ETV6_SV (SV); SEQ ID NO: 1732, ETV6_SV (SV); SEQ ID NO: 1733, ETV6_SV (SV); SEQ ID NO: 1734, ETV6_SV (SV); SEQ ID NO: 1735, ETV6_SV (SV); SEQ ID NO: 1736, ETV6_SV (SV); SEQ ID NO: 1737, ETV6_SV (SV); SEQ ID NO: 1738, ETV6_SV (SV); SEQ ID NO: 1739, ETV6_SV (SV); SEQ ID NO: 1740, ETV6_SV (SV); SEQ ID NO: 1741, ETV6_SV (SV); SEQ ID NO: 1742, ETV6_SV (SV); SEQ ID NO: 1743, ETV6_SV (SV); SEQ ID NO: 1744, ETV6_SV (SV); SEQ ID NO: 1745, ETV6_SV (SV); SEQ ID NO: 1746, ETV6_SV (SV); SEQ ID NO: 1747, ETV6_SV (SV); SEQ ID NO: 1748, ETV6_SV (SV); SEQ ID NO: 1749, ETV6_SV (SV); SEQ ID NO: 1750, ETV6_SV (SV); SEQ ID NO: 1751, ETV6_SV (SV); SEQ ID NO: 1752, ETV6_SV (SV); SEQ ID NO: 1753, ETV6_SV (SV); SEQ ID NO: 1754, ETV6_SV (SV); SEQ ID NO: 1755, ETV6_SV (SV); SEQ ID NO: 1756, ETV6_SV (SV); SEQ ID NO: 1757, ETV6_SV (SV); SEQ ID NO: 1758, ETV6_SV (SV); SEQ ID NO: 1759, ETV6_SV (SV); SEQ ID NO: 1760, ETV6_SV (SV); SEQ ID NO: 1761, ETV6_SV (SV); SEQ ID NO: 1762, ETV6_SV (SV); SEQ ID NO: 1763, ETV6_SV (SV); SEQ ID NO: 1764, ETV6_SV (SV); SEQ ID NO: 1765, ETV6_SV (SV); SEQ ID NO: 1766, ETV6_SV (SV); SEQ ID NO: 1767, ETV6_SV (SV); SEQ ID NO: 1768, ETV6_SV (SV); SEQ ID NO: 1769, ETV6_SV (SV); SEQ ID NO: 1770, ETV6_SV (SV); SEQ ID NO: 1771, ETV6_SV (SV); SEQ ID NO: 1772, ETV6_SV (SV); SEQ ID NO: 1773, ETV6_SV (SV); SEQ ID NO: 1774, ETV6_SV (SV); SEQ ID NO: 1775, ETV6_SV (SV); SEQ ID NO: 1776, ETV6_SV (SV); SEQ ID NO: 1777, ETV6_SV (SV); SEQ ID NO: 1778, ETV6_SV (SV); SEQ ID NO: 1779, ETV6_SV (SV); SEQ ID NO: 1780, ETV6_SV (SV); SEQ ID NO: 1781, ETV6_SV (SV); SEQ ID NO: 1782, ETV6_SV (SV); SEQ ID NO: 1783, ETV6_SV (SV); SEQ ID NO: 1784, ETV6_SV (SV); SEQ ID NO: 1785, ETV6_SV (SV); SEQ ID NO: 1786, ETV6_SV (SV); SEQ ID NO: 1787, ETV6_SV (SV); SEQ ID NO: 1788, ETV6_SV (SV); SEQ ID NO: 1789, ETV6_SV (SV); SEQ ID NO: 1790, ETV6_SV (SV); SEQ ID NO: 1791, ETV6_SV (SV); SEQ ID NO: 1792, ETV6_SV (SV); SEQ ID NO: 1793, ETV6_SV (SV); SEQ ID NO: 1794, ETV6_SV (SV); SEQ ID NO: 1795, ETV6_SV (SV); SEQ ID NO: 1796, ETV6_SV (SV); SEQ ID NO: 1797, ETV6_SV (SV); SEQ ID NO: 1798, ETV6_SV (SV); SEQ ID NO: 1799, ETV6_SV (SV); SEQ ID NO: 1800, ETV6_SV (SV); SEQ ID NO: 1801, ETV6_SV (SV); SEQ ID NO: 1802, ETV6_SV (SV); SEQ ID NO: 1803, ETV6_SV (SV); SEQ ID NO: 1804, ETV6_SV (SV); SEQ ID NO: 1805, ETV6_SV (SV); SEQ ID NO: 1806, ETV6_SV (SV); SEQ ID NO: 1807, ETV6_SV (SV); SEQ ID NO: 1808, ETV6_SV (SV); SEQ ID NO: 1809, ETV6_SV (SV); SEQ ID NO: 1810, ETV6_SV (SV); SEQ ID NO: 1811, ETV6_SV (SV); SEQ ID NO: 1812, ETV6_SV (SV); SEQ ID NO: 1813, ETV6_SV (SV); SEQ ID NO: 1814, ETV6_SV (SV); SEQ ID NO: 1815, ETV6_SV (SV); SEQ ID NO: 1816, ETV6_SV (SV); SEQ ID NO: 1817, ETV6_SV (SV); SEQ ID NO: 1818, ETV6_SV (SV); SEQ ID NO: 1819, ETV6_SV (SV); SEQ ID NO: 1820, ETV6_SV (SV); SEQ ID NO: 1821, ETV6_SV (SV); SEQ ID NO: 1822, ETV6_SV (SV); SEQ ID NO: 1823, ETV6_SV (SV); SEQ ID NO: 1824, ETV6_SV (SV); SEQ ID NO: 1825, ETV6_SV (SV); SEQ ID NO: 1826, ETV6_SV (SV); SEQ ID NO: 1827, ETV6_SV (SV); SEQ ID NO: 1828, ETV6_SV (SV); SEQ ID NO: 1829, ETV6_SV (SV); SEQ ID NO: 1830, ETV6_SV (SV); SEQ ID NO: 1831, ETV6_SV (SV); SEQ ID NO: 1832, ETV6_SV (SV); SEQ ID NO: 1833, ETV6_SV (SV); SEQ ID NO: 1834, ETV6_SV (SV); SEQ ID NO: 1835, ETV6_SV (SV); SEQ ID NO: 1836, ETV6_SV (SV); SEQ ID NO: 1837, ETV6_SV (SV); SEQ ID NO: 1838, ETV6_SV (SV); SEQ ID NO: 1839, ETV6_SV (SV); SEQ ID NO: 1840, ETV6_SV (SV); SEQ ID NO: 1841, ETV6_SV (SV); SEQ ID NO: 1842, ETV6_SV (SV); SEQ ID NO: 1843, ETV6_SV (SV); SEQ ID NO: 1844, ETV6_SV (SV); SEQ ID NO: 1845, ETV6_SV (SV); SEQ ID NO: 1846, ETV6_SV (SV); SEQ ID NO: 1847, ETV6_SV (SV); SEQ ID NO: 1848, ETV6_SV (SV); SEQ ID NO: 1849, ETV6_SV (SV); SEQ ID NO: 1850, ETV6_SV (SV); SEQ ID NO: 1851, ETV6_SV (SV); SEQ ID NO: 1852, ETV6_SV (SV); SEQ ID NO: 1853, ETV6_SV (SV); SEQ ID NO: 1854, ETV6_SV (SV); SEQ ID NO: 1855, ETV6_SV (SV); SEQ ID NO: 1856, ETV6_SV (SV); SEQ ID NO: 1857, ETV6_SV (SV); SEQ ID NO: 1858, ETV6_SV (SV); SEQ ID NO: 1859, ETV6_SV (SV); SEQ ID NO: 1860, ETV6_SV (SV); SEQ ID NO: 1861, ETV6_SV (SV); SEQ ID NO: 1862, ETV6_SV (SV); SEQ ID NO: 1863, ETV6_SV (SV); SEQ ID NO: 1864, ETV6_SV (SV); SEQ ID NO: 1865, ETV6_SV (SV); SEQ ID NO: 1866, ETV6_SV (SV); SEQ ID NO: 1867, ETV6_SV (SV); SEQ ID NO: 1868, ETV6_SV (SV); SEQ ID NO: 1869, ETV6_SV (SV); SEQ ID NO: 1870, ETV6_SV (SV); SEQ ID NO: 1871, ETV6_SV (SV); SEQ ID NO: 1872, ETV6_SV (SV); SEQ ID NO: 1873, ETV6_SV (SV); SEQ ID NO: 1874, ETV6_SV (SV); SEQ ID NO: 1875, ETV6_SV (SV); SEQ ID NO: 1876, ETV6_SV (SV); SEQ ID NO: 1877, ETV6_SV (SV); SEQ ID NO: 1878, ETV6_SV (SV); SEQ ID NO: 1879, ETV6_SV (SV); SEQ ID NO: 1880, ETV6_SV (SV); SEQ ID NO: 1881, ETV6_SV (SV); SEQ ID NO: 1882, ETV6_SV (SV); SEQ ID NO: 1883, ETV6_SV (SV); SEQ ID NO: 1884, ETV6_SV (SV); SEQ ID NO: 1885, ETV6_SV (SV); SEQ ID NO: 1886, ETV6_SV (SV); SEQ ID NO: 1887, ETV6_SV (SV); SEQ ID NO: 1888, ETV6_SV (SV); SEQ ID NO: 1889, ETV6_SV (SV); SEQ ID NO: 1890, ETV6_SV (SV); SEQ ID NO: 1891, ETV6_SV (SV); SEQ ID NO: 1892, ETV6_SV (SV); SEQ ID NO: 1893, ETV6_SV (SV); SEQ ID NO: 1894, ETV6_SV (SV); SEQ ID NO: 1895, ETV6_SV (SV); SEQ ID NO: 1896, ETV6_SV (SV); SEQ ID NO: 1897, ETV6_SV (SV); SEQ ID NO: 1898, ETV6_SV (SV); SEQ ID NO: 1899, ETV6_SV (SV); SEQ ID NO: 1900, ETV6_SV (SV); SEQ ID NO: 1901, ETV6_SV (SV); SEQ ID NO: 1902, ETV6_SV (SV); SEQ ID NO: 1903, ETV6_SV (SV); SEQ ID NO: 1904, ETV6_SV (SV); SEQ ID NO: 1905, ETV6_SV (SV); SEQ ID NO: 1906, ETV6_SV (SV); SEQ ID NO: 1907, ETV6_SV (SV); SEQ ID NO: 1908, ETV6_SV (SV); SEQ ID NO: 1909, ETV6_SV (SV); SEQ ID NO: 1910, ETV6_SV (SV); SEQ ID NO: 1911, ETV6_SV (SV); SEQ ID NO: 1912, ETV6_SV (SV); SEQ ID NO: 1913, ETV6_SV (SV); SEQ ID NO: 1914, ETV6_SV (SV); SEQ ID NO: 1915, ETV6 (Gene); SEQ ID NO: 1916, ETV6 (Gene); SEQ ID NO: 1917, ETV6 (Gene); SEQ ID NO: 1918, ETV6 (Gene); SEQ ID NO: 1919, ETV6 (Gene); SEQ ID NO: 1920, ETV6 (Gene); SEQ ID NO: 1921, 12p13.2_DLBCL (FOCAL); SEQ ID NO: 1922, 12p13.2_DLBCL (FOCAL); SEQ ID NO: 1923, 12p13.2_DLBCL (FOCAL); SEQ ID NO: 1924, CDKN1B (Gene); SEQ ID NO: 1925, CDKN1B (Gene); SEQ ID NO: 1926, 12p13.2_DLBCL (FOCAL); SEQ ID NO: 1927, 12p13.2_DLBCL (FOCAL); SEQ ID NO: 1928, 12p13.2_DLBCL (FOCAL); SEQ ID NO: 1929, 12p13.2_DLBCL (FOCAL); SEQ ID NO: 1930, 12P_DLBCL (ARM); SEQ ID NO: 1931, 12P_DLBCL (ARM); SEQ ID NO: 1932, 12P_DLBCL (ARM); SEQ ID NO: 1933, 12P_DLBCL (ARM); SEQ ID NO: 1934, 12P_DLBCL (ARM); SEQ ID NO: 1935, 12P_DLBCL (ARM); SEQ ID NO: 1936, FP (FP); SEQ ID NO: 1937, 12P_DLBCL (ARM); SEQ ID NO: 1938, KRAS (Gene); SEQ ID NO: 1939, KRAS (Gene); SEQ ID NO: 1940, KRAS (Gene); SEQ ID NO: 1941, KRAS (Gene); SEQ ID NO: 1942, KRAS (Gene); SEQ ID NO: 1943, 12P_DLBCL (ARM); SEQ ID NO: 1944, FP (FP); SEQ ID NO: 1945, 12P_DLBCL (ARM); SEQ ID NO: 1946, 12P_DLBCL (ARM); SEQ ID NO: 1947, 12P_DLBCL (ARM); SEQ ID NO: 1948, 12P_DLBCL (ARM); SEQ ID NO: 1949, 12P_DLBCL (ARM); SEQ ID NO: 1950, 12P_DLBCL (ARM); SEQ ID NO: 1951, 12Q (ARM); SEQ ID NO: 1952, 12Q (ARM); SEQ ID NO: 1953, 12Q (ARM); SEQ ID NO: 1954, 12Q (ARM); SEQ ID NO: 1955, 12Q (ARM); SEQ ID NO: 1956, 12Q (ARM); SEQ ID NO: 1957, 12Q (ARM); SEQ ID NO: 1958, 12Q (ARM); SEQ ID NO: 1959, FP (FP); SEQ ID NO: 1960, 12Q (ARM); SEQ ID NO: 1961, 12Q (ARM); SEQ ID NO: 1962, KMT2D (Gene); SEQ ID NO: 1963, KMT2D (Gene); SEQ ID NO: 1964, KMT2D (Gene); SEQ ID NO: 1965, KMT2D (Gene); SEQ ID NO: 1966, KMT2D (Gene); SEQ ID NO: 1967, KMT2D (Gene); SEQ ID NO: 1968, KMT2D (Gene); SEQ ID NO: 1969, KMT2D (Gene); SEQ ID NO: 1970, KMT2D (Gene); SEQ ID NO: 1971, KMT2D (Gene); SEQ ID NO: 1972, KMT2D (Gene); SEQ ID NO: 1973, KMT2D (Gene); SEQ ID NO: 1974, KMT2D (Gene); SEQ ID NO: 1975, KMT2D (Gene); SEQ ID NO: 1976, KMT2D (Gene); SEQ ID NO: 1977, KMT2D (Gene); SEQ ID NO: 1978, KMT2D (Gene); SEQ ID NO: 1979, KMT2D (Gene); SEQ ID NO: 1980, KMT2D (Gene); SEQ ID NO: 1981, KMT2D (Gene); SEQ ID NO: 1982, KMT2D (Gene); SEQ ID NO: 1983, KMT2D (Gene); SEQ ID NO: 1984, KMT2D (Gene); SEQ ID NO: 1985, KMT2D (Gene); SEQ ID NO: 1986, KMT2D (Gene); SEQ ID NO: 1987, KMT2D (Gene); SEQ ID NO: 1988, KMT2D (Gene); SEQ ID NO: 1989, KMT2D (Gene); SEQ ID NO: 1990, KMT2D (Gene); SEQ ID NO: 1991, KMT2D (Gene); SEQ ID NO: 1992, KMT2D (Gene); SEQ ID NO: 1993, KMT2D (Gene); SEQ ID NO: 1994, KMT2D (Gene); SEQ ID NO: 1995, KMT2D (Gene); SEQ ID NO: 1996, KMT2D (Gene); SEQ ID NO: 1997, KMT2D (Gene); SEQ ID NO: 1998, KMT2D (Gene); SEQ ID NO: 1999, KMT2D (Gene); SEQ ID NO: 2000, KMT2D (Gene); SEQ ID NO: 2001, KMT2D (Gene); SEQ ID NO: 2002, KMT2D (Gene); SEQ ID NO: 2003, KMT2D (Gene); SEQ ID NO: 2004, KMT2D (Gene); SEQ ID NO: 2005, KMT2D (Gene); SEQ ID NO: 2006, KMT2D (Gene); SEQ ID NO: 2007, KMT2D (Gene); SEQ ID NO: 2008, KMT2D (Gene); SEQ ID NO: 2009, KMT2D (Gene); SEQ ID NO: 2010, KMT2D (Gene); SEQ ID NO: 2011, KMT2D (Gene); SEQ ID NO: 2012, KMT2D (Gene); SEQ ID NO: 2013, KMT2D (Gene); SEQ ID NO: 2014, KMT2D (Gene); SEQ ID NO: 2015, KMT2D (Gene); SEQ ID NO: 2016, KMT2D (Gene); SEQ ID NO: 2017, KMT2D (Gene); SEQ ID NO: 2018, KMT2D (Gene); SEQ ID NO: 2019, KMT2D (Gene); SEQ ID NO: 2020, KMT2D (Gene); SEQ ID NO: 2021, KMT2D (Gene); SEQ ID NO: 2022, KMT2D (Gene); SEQ ID NO: 2023, KMT2D (Gene); SEQ ID NO: 2024, KMT2D (Gene); SEQ ID NO: 2025, KMT2D (Gene); SEQ ID NO: 2026, KMT2D (Gene); SEQ ID NO: 2027, KMT2D (Gene); SEQ ID NO: 2028, KMT2D (Gene); SEQ ID NO: 2029, KMT2D (Gene); SEQ ID NO: 2030, KMT2D (Gene); SEQ ID NO: 2031, KMT2D (Gene); SEQ ID NO: 2032, KMT2D (Gene); SEQ ID NO: 2033, KMT2D (Gene); SEQ ID NO: 2034, KMT2D (Gene); SEQ ID NO: 2035, KMT2D (Gene); SEQ ID NO: 2036, KMT2D (Gene); SEQ ID NO: 2037, KMT2D (Gene); SEQ ID NO: 2038, KMT2D (Gene); SEQ ID NO: 2039, KMT2D (Gene); SEQ ID NO: 2040, KMT2D (Gene); SEQ ID NO: 2041, 12Q (ARM); SEQ ID NO: 2042, 12Q (ARM); SEQ ID NO: 2043, 12Q (ARM); SEQ ID NO: 2044, MSI (MSI); SEQ ID NO: 2045, MSI (MSI); SEQ ID NO: 2046, MSI (MSI); SEQ ID NO: 2047, MSI (MSI); SEQ ID NO: 2048, STAT6 (Gene); SEQ ID NO: 2049, STAT6 (Gene); SEQ ID NO: 2050, STAT6 (Gene); SEQ ID NO: 2051, STAT6 (Gene); SEQ ID NO: 2052, STAT6 (Gene); SEQ ID NO: 2053, STAT6 (Gene); SEQ ID NO: 2054, STAT6 (Gene); SEQ ID NO: 2055, STAT6 (Gene); SEQ ID NO: 2056, STAT6 (Gene); SEQ ID NO: 2057, STAT6 (Gene); SEQ ID NO: 2058, STAT6 (Gene); SEQ ID NO: 2059, STAT6 (Gene); SEQ ID NO: 2060, STAT6 (Gene); SEQ ID NO: 2061, STAT6 (Gene); SEQ ID NO: 2062, STAT6 (Gene); SEQ ID NO: 2063, STAT6 (Gene); SEQ ID NO: 2064, STAT6 (Gene); SEQ ID NO: 2065, STAT6 (Gene); SEQ ID NO: 2066, STAT6 (Gene); SEQ ID NO: 2067, STAT6 (Gene); SEQ ID NO: 2068, STAT6 (Gene); SEQ ID NO: 2069, 12Q (ARM); SEQ ID NO: 2070, 12Q (ARM); SEQ ID NO: 2071, 12Q (ARM); SEQ ID NO: 2072, 12Q (ARM); SEQ ID NO: 2073, 12Q (ARM); SEQ ID NO: 2074, 12Q (ARM); SEQ ID NO: 2075, 12Q (ARM); SEQ ID NO: 2076, 12Q (ARM); SEQ ID NO: 2077, 12Q (ARM); SEQ ID NO: 2078, 12Q (ARM); SEQ ID NO: 2079, 12Q (ARM); SEQ ID NO: 2080, 12Q (ARM); SEQ ID NO: 2081, 12Q (ARM); SEQ ID NO: 2082, 12Q (ARM); SEQ ID NO: 2083, 12Q (ARM); SEQ ID NO: 2084, 12Q (ARM); SEQ ID NO: 2085, 12Q (ARM); SEQ ID NO: 2086, 12Q (ARM); SEQ ID NO: 2087, 12Q (ARM); SEQ ID NO: 2088, 12Q (ARM); SEQ ID NO: 2089, 12Q (ARM); SEQ ID NO: 2090, 12Q (ARM); SEQ ID NO: 2091, 12Q (ARM); SEQ ID NO: 2092, BTG1 (Gene); SEQ ID NO: 2093, BTG1 (Gene); SEQ ID NO: 2094, BTG1 (Gene); SEQ ID NO: 2095, BTG1 (Gene); SEQ ID NO: 2096, 12Q (ARM); SEQ ID NO: 2097, 12Q (ARM); SEQ ID NO: 2098, 12Q (ARM); SEQ ID NO: 2099, 12Q (ARM); SEQ ID NO: 2100, 12Q (ARM); SEQ ID NO: 2101, 12Q (ARM); SEQ ID NO: 2102, 12Q (ARM); SEQ ID NO: 2103, 12Q (ARM); SEQ ID NO: 2104, 12Q (ARM); SEQ ID NO: 2105, 12Q (ARM); SEQ ID NO: 2106, FP (FP); SEQ ID NO: 2107, 12Q (ARM); SEQ ID NO: 2108, 12Q (ARM); SEQ ID NO: 2109, 12Q (ARM); SEQ ID NO: 2110, 12Q (ARM); SEQ ID NO: 2111, 12Q (ARM); SEQ ID NO: 2112, 12Q (ARM); SEQ ID NO: 2113, 12Q (ARM); SEQ ID NO: 2114, HVCN1 (Gene); SEQ ID NO: 2115, HVCN1 (Gene); SEQ ID NO: 2116, HVCN1 (Gene); SEQ ID NO: 2117, HVCN1 (Gene); SEQ ID NO: 2118, HVCN1 (Gene); SEQ ID NO: 2119, HVCN1 (Gene); SEQ ID NO: 2120, 12Q (ARM); SEQ ID NO: 2121, 12Q (ARM); SEQ ID NO: 2122, 12Q (ARM); SEQ ID NO: 2123, DTX1 (Gene); SEQ ID NO: 2124, DTX1 (Gene); SEQ ID NO: 2125, DTX1 (Gene); SEQ ID NO: 2126, DTX1 (Gene); SEQ ID NO: 2127, DTX1 (Gene); SEQ ID NO: 2128, DTX1 (Gene); SEQ ID NO: 2129, DTX1 (Gene); SEQ ID NO: 2130, DTX1 (Gene); SEQ ID NO: 2131, DTX1 (Gene); SEQ ID NO: 2132, DTX1 (Gene); SEQ ID NO: 2133, 12Q (ARM); SEQ ID NO: 2134, 12Q (ARM); SEQ ID NO: 2135, 12Q (ARM); SEQ ID NO: 2136, 12Q (ARM); SEQ ID NO: 2137, 12Q (ARM); SEQ ID NO: 2138, 12Q (ARM); SEQ ID NO: 2139, 12Q (ARM); SEQ ID NO: 2140, 12Q (ARM); SEQ ID NO: 2141, 12Q (ARM); SEQ ID NO: 2142, 12Q (ARM); SEQ ID NO: 2143, 12Q (ARM); SEQ ID NO: 2144, SETD1B (Gene); SEQ ID NO: 2145, SETD1B (Gene); SEQ ID NO: 2146, SETD1B (Gene); SEQ ID NO: 2147, SETD1B (Gene); SEQ ID NO: 2148, SETD1B (Gene); SEQ ID NO: 2149, SETD1B (Gene); SEQ ID NO: 2150, SETD1B (Gene); SEQ ID NO: 2151, SETD1B (Gene); SEQ ID NO: 2152, SETD1B (Gene); SEQ ID NO: 2153, SETD1B (Gene); SEQ ID NO: 2154, SETD1B (Gene); SEQ ID NO: 2155, SETD1B (Gene); SEQ ID NO: 2156, SETD1B (Gene); SEQ ID NO: 2157, SETD1B (Gene); SEQ ID NO: 2158, SETD1B (Gene); SEQ ID NO: 2159, SETD1B (Gene); SEQ ID NO: 2160, SETD1B (Gene); SEQ ID NO: 2161, SETD1B (Gene); SEQ ID NO: 2162, SETD1B (Gene); SEQ ID NO: 2163, SETD1B (Gene); SEQ ID NO: 2164, SETD1B (Gene); SEQ ID NO: 2165, SETD1B (Gene); SEQ ID NO: 2166, SETD1B (Gene); SEQ ID NO: 2167, SETD1B (Gene); SEQ ID NO: 2168, BCL7A (Gene); SEQ ID NO: 2169, BCL7A (Gene); SEQ ID NO: 2170, BCL7A (Gene); SEQ ID NO: 2171, BCL7A (Gene); SEQ ID NO: 2172, BCL7A (Gene); SEQ ID NO: 2173, BCL7A (Gene); SEQ ID NO: 2174, 12Q (ARM); SEQ ID NO: 2175, 12Q (ARM); SEQ ID NO: 2176, 12Q (ARM); SEQ ID NO: 2177, 12Q (ARM); SEQ ID NO: 2178, 12Q (ARM); SEQ ID NO: 2179, 12Q (ARM); SEQ ID NO: 2180, 12Q (ARM); SEQ ID NO: 2181, 12Q (ARM); SEQ ID NO: 2182, 12Q (ARM); SEQ ID NO: 2183, 12Q (ARM); SEQ ID NO: 2184, 12Q (ARM); SEQ ID NO: 2185, 12Q (ARM); SEQ ID NO: 2186, 13q-ARM_CLL (ARM); SEQ ID NO: 2187, 13q-ARM_CLL (ARM); SEQ ID NO: 2188, FP (FP); SEQ ID NO: 2189, 13q-ARM_CLL (ARM); SEQ ID NO: 2190, 13q-ARM_CLL (ARM); SEQ ID NO: 2191, 13q-ARM_CLL (ARM); SEQ ID NO: 2192, 13q-ARM_CLL (ARM); SEQ ID NO: 2193, 13q-ARM_CLL (ARM); SEQ ID NO: 2194, 13q-ARM_CLL (ARM); SEQ ID NO: 2195, PABPC3 (Gene); SEQ ID NO: 2196, PABPC3 (Gene); SEQ ID NO: 2197, PABPC3 (Gene); SEQ ID NO: 2198, PABPC3 (Gene); SEQ ID NO: 2199, 13q-ARM_CLL (ARM); SEQ ID NO: 2200, 13q-ARM_CLL (ARM); SEQ ID NO: 2201, 13q-ARM_CLL (ARM); SEQ ID NO: 2202, 13q-ARM_CLL (ARM); SEQ ID NO: 2203, 13q12.3-q13.1_MCL (FOCAL); SEQ ID NO: 2204, 13q12.3-q13.1_MCL (FOCAL); SEQ ID NO: 2205, 13q12.3-q13.1_MCL (FOCAL); SEQ ID NO: 2206, 13q12.3-q13.1_MCL (FOCAL); SEQ ID NO: 2207, 13q12.3-q13.1_MCL (FOCAL); SEQ ID NO: 2208, 13q12.3-q13.1_MCL (FOCAL); SEQ ID NO: 2209, 13q12.3-q13.1_MCL (FOCAL); SEQ ID NO: 2210, 13q12.3-q13.1_MCL (FOCAL); SEQ ID NO: 2211, 13q12.3-q13.1_MCL (FOCAL); SEQ ID NO: 2212, 13q12.3-q13.1_MCL (FOCAL); SEQ ID NO: 2213, 13q12.3-q13.1_MCL (FOCAL); SEQ ID NO: 2214, 13q12.3-q13.1_MCL (FOCAL); SEQ ID NO: 2215, 13q12.3-q13.1_MCL (FOCAL); SEQ ID NO: 2216, 13q12.3-q13.1_MCL (FOCAL); SEQ ID NO: 2217, 13q12.3-q13.1_MCL (FOCAL); SEQ ID NO: 2218, 13q12.3-q13.1_MCL (FOCAL); SEQ ID NO: 2219, 13q12.3-q13.1_MCL (FOCAL); SEQ ID NO: 2220, 13q12.3-q13.1_MCL (FOCAL); SEQ ID NO: 2221, 13q12.3-q13.1_MCL (FOCAL); SEQ ID NO: 2222, 13q12.3-q13.1_MCL (FOCAL); SEQ ID NO: 2223, 13q12.3-q13.1_MCL (FOCAL); SEQ ID NO: 2224, 13q12.3-q13.1_MCL (FOCAL); SEQ ID NO: 2225, 13q12.3-q13.1_MCL (FOCAL); SEQ ID NO: 2226, BRCA2 (Gene); SEQ ID NO: 2227, BRCA2 (Gene); SEQ ID NO: 2228, BRCA2 (Gene); SEQ ID NO: 2229, BRCA2 (Gene); SEQ ID NO: 2230, BRCA2 (Gene); SEQ ID NO: 2231, BRCA2 (Gene); SEQ ID NO: 2232, BRCA2 (Gene); SEQ ID NO: 2233, BRCA2 (Gene); SEQ ID NO: 2234, BRCA2 (Gene); SEQ ID NO: 2235, BRCA2 (Gene); SEQ ID NO: 2236, BRCA2 (Gene); SEQ ID NO: 2237, BRCA2 (Gene); SEQ ID NO: 2238, BRCA2 (Gene); SEQ ID NO: 2239, BRCA2 (Gene); SEQ ID NO: 2240, BRCA2 (Gene); SEQ ID NO: 2241, BRCA2 (Gene); SEQ ID NO: 2242, BRCA2 (Gene); SEQ ID NO: 2243, BRCA2 (Gene); SEQ ID NO: 2244, BRCA2 (Gene); SEQ ID NO: 2245, BRCA2 (Gene); SEQ ID NO: 2246, BRCA2 (Gene); SEQ ID NO: 2247, BRCA2 (Gene); SEQ ID NO: 2248, BRCA2 (Gene); SEQ ID NO: 2249, BRCA2 (Gene); SEQ ID NO: 2250, BRCA2 (Gene); SEQ ID NO: 2251, BRCA2 (Gene); SEQ ID NO: 2252, BRCA2 (Gene); SEQ ID NO: 2253, BRCA2 (Gene); SEQ ID NO: 2254, BRCA2 (Gene); SEQ ID NO: 2255, BRCA2 (Gene); SEQ ID NO: 2256, BRCA2 (Gene); SEQ ID NO: 2257, BRCA2 (Gene); SEQ ID NO: 2258, BRCA2 (Gene); SEQ ID NO: 2259, BRCA2 (Gene); SEQ ID NO: 2260, BRCA2 (Gene); SEQ ID NO: 2261, BRCA2 (Gene); SEQ ID NO: 2262, BRCA2 (Gene); SEQ ID NO: 2263, BRCA2 (Gene); SEQ ID NO: 2264, BRCA2 (Gene); SEQ ID NO: 2265, BRCA2 (Gene); SEQ ID NO: 2266, BRCA2 (Gene); SEQ ID NO: 2267, BRCA2 (Gene); SEQ ID NO: 2268, BRCA2 (Gene); SEQ ID NO: 2269, BRCA2 (Gene); SEQ ID NO: 2270, 13q12.3-q13.1_MCL (FOCAL); SEQ ID NO: 2271, 13q12.3-q13.1_MCL (FOCAL); SEQ ID NO: 2272, 13q12.3-q13.1_MCL (FOCAL); SEQ ID NO: 2273, 13q12.3-q13.1_MCL (FOCAL); SEQ ID NO: 2274, 13q12.3-q13.1_MCL (FOCAL); SEQ ID NO: 2275, 13q-ARM_CLL (ARM); SEQ ID NO: 2276, 13q-ARM_CLL (ARM); SEQ ID NO: 2277, FP (FP); SEQ ID NO: 2278, 13q-ARM_CLL (ARM); SEQ ID NO: 2279, 13q-ARM_CLL (ARM); SEQ ID NO: 2280, 13q-ARM_CLL (ARM); SEQ ID NO: 2281, 13q-ARM_CLL (ARM); SEQ ID NO: 2282, 13q-ARM_CLL (ARM); SEQ ID NO: 2283, FOXO1 (Gene); SEQ ID NO: 2284, FOXO1 (Gene); SEQ ID NO: 2285, FOXO1 (Gene); SEQ ID NO: 2286, FOXO1 (Gene); SEQ ID NO: 2287, FOXO1 (Gene); SEQ ID NO: 2288, 13q-ARM_CLL (ARM); SEQ ID NO: 2289, 13q-ARM_CLL (ARM); SEQ ID NO: 2290, 13q-ARM_CLL (ARM); SEQ ID NO: 2291, 13q-ARM_CLL (ARM); SEQ ID NO: 2292, 13q-ARM_CLL (ARM); SEQ ID NO: 2293, 13q-ARM_CLL (ARM); SEQ ID NO: 2294, 13q-ARM_CLL (ARM); SEQ ID NO: 2295, 13q14.2_DLBCL (FOCAL); SEQ ID NO: 2296, 13q14.2_DLBCL (FOCAL); SEQ ID NO: 2297, 13q14.2_DLBCL (FOCAL); SEQ ID NO: 2298, 13q14.2_DLBCL (FOCAL); SEQ ID NO: 2299, 13q14.2_DLBCL (FOCAL); SEQ ID NO: 2300, 13q14.2_DLBCL (FOCAL); SEQ ID NO: 2301, 13q14.2_DLBCL (FOCAL); SEQ ID NO: 2302, FP (FP); SEQ ID NO: 2303, 13q14.2_DLBCL (FOCAL); SEQ ID NO: 2304, RB1 (Gene); SEQ ID NO: 2305, RB1 (Gene); SEQ ID NO: 2306, RB1 (Gene); SEQ ID NO: 2307, RB1 (Gene); SEQ ID NO: 2308, RB1 (Gene); SEQ ID NO: 2309, RB1 (Gene); SEQ ID NO: 2310, RB1 (Gene); SEQ ID NO: 2311, RB1 (Gene); SEQ ID NO: 2312, RB1 (Gene); SEQ ID NO: 2313, RB1 (Gene); SEQ ID NO: 2314, RB1 (Gene); SEQ ID NO: 2315, RB1 (Gene); SEQ ID NO: 2316, RB1 (Gene); SEQ ID NO: 2317, RB1 (Gene); SEQ ID NO: 2318, RB1 (Gene); SEQ ID NO: 2319, RB1 (Gene); SEQ ID NO: 2320, RB1 (Gene); SEQ ID NO: 2321, RB1 (Gene); SEQ ID NO: 2322, RB1 (Gene); SEQ ID NO: 2323, RB1 (Gene); SEQ ID NO: 2324, RB1 (Gene); SEQ ID NO: 2325, RB1 (Gene); SEQ ID NO: 2326, RB1 (Gene); SEQ ID NO: 2327, RB1 (Gene); SEQ ID NO: 2328, RB1 (Gene); SEQ ID NO: 2329, RB1 (Gene); SEQ ID NO: 2330, RB1 (Gene); SEQ ID NO: 2331, 13q14.2_DLBCL (FOCAL); SEQ ID NO: 2332, 13q14.2_DLBCL (FOCAL); SEQ ID NO: 2333, 13q14.2_DLBCL (FOCAL); SEQ ID NO: 2334, SETDB2 (Gene); SEQ ID NO: 2335, SETDB2 (Gene); SEQ ID NO: 2336, SETDB2 (Gene); SEQ ID NO: 2337, SETDB2 (Gene); SEQ ID NO: 2338, SETDB2 (Gene); SEQ ID NO: 2339, SETDB2 (Gene); SEQ ID NO: 2340, SETDB2 (Gene); SEQ ID NO: 2341, SETDB2 (Gene); SEQ ID NO: 2342, SETDB2 (Gene); SEQ ID NO: 2343, SETDB2 (Gene); SEQ ID NO: 2344, SETDB2 (Gene); SEQ ID NO: 2345, SETDB2 (Gene); SEQ ID NO: 2346, SETDB2 (Gene); SEQ ID NO: 2347, SETDB2 (Gene); SEQ ID NO: 2348, SETDB2 (Gene); SEQ ID NO: 2349, 13q14.2_DLBCL (FOCAL); SEQ ID NO: 2350, 13q14.2_DLBCL (FOCAL); SEQ ID NO: 2351, 13q14.2_DLBCL (FOCAL); SEQ ID NO: 2352, 13q14.2_DLBCL (FOCAL); SEQ ID NO: 2353, 13q14.2_DLBCL (FOCAL); SEQ ID NO: 2354, 13q14.2_DLBCL (FOCAL); SEQ ID NO: 2355, 13q14.2_DLBCL (FOCAL); SEQ ID NO: 2356, 13q14.2_DLBCL (FOCAL); SEQ ID NO: 2357, 13q14.2_DLBCL (FOCAL); SEQ ID NO: 2358, 13q14.2_DLBCL (FOCAL); SEQ ID NO: 2359, 13q-ARM_CLL (ARM); SEQ ID NO: 2360, 13q-ARM_CLL (ARM); SEQ ID NO: 2361, 13q-ARM_CLL (ARM); SEQ ID NO: 2362, 13q-ARM_CLL (ARM); SEQ ID NO: 2363, 13q-ARM_CLL (ARM); SEQ ID NO: 2364, 13q-ARM_CLL (ARM); SEQ ID NO: 2365, 13q-ARM_CLL (ARM); SEQ ID NO: 2366, 13q-ARM_CLL (ARM); SEQ ID NO: 2367, 13q-ARM_CLL (ARM); SEQ ID NO: 2368, FP (FP); SEQ ID NO: 2369, 13q-ARM_CLL (ARM); SEQ ID NO: 2370, 13q-ARM_CLL (ARM); SEQ ID NO: 2371, 13q-ARM_CLL (ARM); SEQ ID NO: 2372, 13q-ARM_CLL (ARM); SEQ ID NO: 2373, 13q-ARM_CLL (ARM); SEQ ID NO: 2374, 13q-ARM_CLL (ARM); SEQ ID NO: 2375, 13q-ARM_CLL (ARM); SEQ ID NO: 2376, 13q-ARM_CLL (ARM); SEQ ID NO: 2377, 13q-ARM_CLL (ARM); SEQ ID NO: 2378, MYCBP2 (Gene); SEQ ID NO: 2379, MYCBP2 (Gene); SEQ ID NO: 2380, MYCBP2 (Gene); SEQ ID NO: 2381, MYCBP2 (Gene); SEQ ID NO: 2382, MYCBP2 (Gene); SEQ ID NO: 2383, MYCBP2 (Gene); SEQ ID NO: 2384, MYCBP2 (Gene); SEQ ID NO: 2385, MYCBP2 (Gene); SEQ ID NO: 2386, MYCBP2 (Gene); SEQ ID NO: 2387, MYCBP2 (Gene); SEQ ID NO: 2388, MYCBP2 (Gene); SEQ ID NO: 2389, MYCBP2 (Gene); SEQ ID NO: 2390, MYCBP2 (Gene); SEQ ID NO: 2391, MYCBP2 (Gene); SEQ ID NO: 2392, MYCBP2 (Gene); SEQ ID NO: 2393, MYCBP2 (Gene); SEQ ID NO: 2394, MYCBP2 (Gene); SEQ ID NO: 2395, MYCBP2 (Gene); SEQ ID NO: 2396, MYCBP2 (Gene); SEQ ID NO: 2397, MYCBP2 (Gene); SEQ ID NO: 2398, MYCBP2 (Gene); SEQ ID NO: 2399, MYCBP2 (Gene); SEQ ID NO: 2400, MYCBP2 (Gene); SEQ ID NO: 2401, MYCBP2 (Gene); SEQ ID NO: 2402, MYCBP2 (Gene); SEQ ID NO: 2403, MYCBP2 (Gene); SEQ ID NO: 2404, MYCBP2 (Gene); SEQ ID NO: 2405, MYCBP2 (Gene); SEQ ID NO: 2406, MYCBP2 (Gene); SEQ ID NO: 2407, MYCBP2 (Gene); SEQ ID NO: 2408, MYCBP2 (Gene); SEQ ID NO: 2409, MYCBP2 (Gene); SEQ ID NO: 2410, MYCBP2 (Gene); SEQ ID NO: 2411, MYCBP2 (Gene); SEQ ID NO: 2412, MYCBP2 (Gene); SEQ ID NO: 2413, MYCBP2 (Gene); SEQ ID NO: 2414, MYCBP2 (Gene); SEQ ID NO: 2415, MYCBP2 (Gene); SEQ ID NO: 2416, MYCBP2 (Gene); SEQ ID NO: 2417, MYCBP2 (Gene); SEQ ID NO: 2418, MYCBP2 (Gene); SEQ ID NO: 2419, MYCBP2 (Gene); SEQ ID NO: 2420, MYCBP2 (Gene); SEQ ID NO: 2421, MYCBP2 (Gene); SEQ ID NO: 2422, MYCBP2 (Gene); SEQ ID NO: 2423, MYCBP2 (Gene); SEQ ID NO: 2424, MYCBP2 (Gene); SEQ ID NO: 2425, MYCBP2 (Gene); SEQ ID NO: 2426, MYCBP2 (Gene); SEQ ID NO: 2427, MYCBP2 (Gene); SEQ ID NO: 2428, MYCBP2 (Gene); SEQ ID NO: 2429, MYCBP2 (Gene); SEQ ID NO: 2430, MYCBP2 (Gene); SEQ ID NO: 2431, MYCBP2 (Gene); SEQ ID NO: 2432, MYCBP2 (Gene); SEQ ID NO: 2433, MYCBP2 (Gene); SEQ ID NO: 2434, MYCBP2 (Gene); SEQ ID NO: 2435, MYCBP2 (Gene); SEQ ID NO: 2436, MYCBP2 (Gene); SEQ ID NO: 2437, MYCBP2 (Gene); SEQ ID NO: 2438, MYCBP2 (Gene); SEQ ID NO: 2439, MYCBP2 (Gene); SEQ ID NO: 2440, MYCBP2 (Gene); SEQ ID NO: 2441, MYCBP2 (Gene); SEQ ID NO: 2442, MYCBP2 (Gene); SEQ ID NO: 2443, MYCBP2 (Gene); SEQ ID NO: 2444, MYCBP2 (Gene); SEQ ID NO: 2445, MYCBP2 (Gene); SEQ ID NO: 2446, MYCBP2 (Gene); SEQ ID NO: 2447, MYCBP2 (Gene); SEQ ID NO: 2448, MYCBP2 (Gene); SEQ ID NO: 2449, MYCBP2 (Gene); SEQ ID NO: 2450, MYCBP2 (Gene); SEQ ID NO: 2451, MYCBP2 (Gene); SEQ ID NO: 2452, MYCBP2 (Gene); SEQ ID NO: 2453, MYCBP2 (Gene); SEQ ID NO: 2454, MYCBP2 (Gene); SEQ ID NO: 2455, MYCBP2 (Gene); SEQ ID NO: 2456, MYCBP2 (Gene); SEQ ID NO: 2457, MYCBP2 (Gene); SEQ ID NO: 2458, MYCBP2 (Gene); SEQ ID NO: 2459, MYCBP2 (Gene); SEQ ID NO: 2460, MYCBP2 (Gene); SEQ ID NO: 2461, MYCBP2 (Gene); SEQ ID NO: 2462, MYCBP2 (Gene); SEQ ID NO: 2463, MYCBP2 (Gene); SEQ ID NO: 2464, MYCBP2 (Gene); SEQ ID NO: 2465, MYCBP2 (Gene); SEQ ID NO: 2466, 13q-ARM_CLL (ARM); SEQ ID NO: 2467, 13q31.3_DLBCL (FOCAL); SEQ ID NO: 2468, 13q31.3_DLBCL (FOCAL); SEQ ID NO: 2469, 13q31.3_DLBCL (FOCAL); SEQ ID NO: 2470, 13q31.3_DLBCL (FOCAL); SEQ ID NO: 2471, 13q31.3_DLBCL (FOCAL); SEQ ID NO: 2472, 13q31.3_DLBCL (FOCAL); SEQ ID NO: 2473, 13q31.3_DLBCL (FOCAL); SEQ ID NO: 2474, 13q31.3_DLBCL (FOCAL); SEQ ID NO: 2475, 13q31.3_DLBCL (FOCAL); SEQ ID NO: 2476, 13q31.3_DLBCL (FOCAL); SEQ ID NO: 2477, 13q31.3_DLBCL (FOCAL); SEQ ID NO: 2478, 13q31.3_DLBCL (FOCAL); SEQ ID NO: 2479, 13q31.3_DLBCL (FOCAL); SEQ ID NO: 2480, 13q31.3_DLBCL (FOCAL); SEQ ID NO: 2481, 13q31.3_DLBCL (FOCAL); SEQ ID NO: 2482, 13q31.3_DLBCL (FOCAL); SEQ ID NO: 2483, 13q31.3_DLBCL (FOCAL); SEQ ID NO: 2484, 13q31.3_DLBCL (FOCAL); SEQ ID NO: 2485, 13q31.3_DLBCL (FOCAL); SEQ ID NO: 2486, 13q31.3_DLBCL (FOCAL); SEQ ID NO: 2487, 13q31.3_DLBCL (FOCAL); SEQ ID NO: 2488, 13q31.3_DLBCL (FOCAL); SEQ ID NO: 2489, 13q31.3_DLBCL (FOCAL); SEQ ID NO: 2490, 13q31.3_DLBCL (FOCAL); SEQ ID NO: 2491, 13q31.3_DLBCL (FOCAL); SEQ ID NO: 2492, 13q31.3_DLBCL (FOCAL); SEQ ID NO: 2493, 13q31.3_DLBCL (FOCAL); SEQ ID NO: 2494, 13q31.3_DLBCL (FOCAL); SEQ ID NO: 2495, 13q31.3_DLBCL (FOCAL); SEQ ID NO: 2496, 13q31.3_DLBCL (FOCAL); SEQ ID NO: 2497, 13q31.3_DLBCL (FOCAL); SEQ ID NO: 2498, 13q31.3_DLBCL (FOCAL); SEQ ID NO: 2499, 13q31.3_DLBCL (FOCAL); SEQ ID NO: 2500, 13q31.3_DLBCL (FOCAL); SEQ ID NO: 2501, 13q31.3_DLBCL (FOCAL); SEQ ID NO: 2502, 13q31.3_DLBCL (FOCAL); SEQ ID NO: 2503, 13q31.3_DLBCL (FOCAL); SEQ ID NO: 2504, 13q31.3_DLBCL (FOCAL); SEQ ID NO: 2505, 13q31.3_DLBCL (FOCAL); SEQ ID NO: 2506, 13q31.3_DLBCL (FOCAL); SEQ ID NO: 2507, 13q31.3_DLBCL (FOCAL); SEQ ID NO: 2508, 13q31.3_DLBCL (FOCAL); SEQ ID NO: 2509, 13q31.3_DLBCL (FOCAL); SEQ ID NO: 2510, 13q31.3_DLBCL (FOCAL); SEQ ID NO: 2511, 13q31.3_DLBCL (FOCAL); SEQ ID NO: 2512, 13q31.3_DLBCL (FOCAL); SEQ ID NO: 2513, 13q31.3_DLBCL (FOCAL); SEQ ID NO: 2514, 13q31.3_DLBCL (FOCAL); SEQ ID NO: 2515, 13q31.3_DLBCL (FOCAL); SEQ ID NO: 2516, 13q31.3_DLBCL (FOCAL); SEQ ID NO: 2517, 13q31.3_DLBCL (FOCAL); SEQ ID NO: 2518, 13q31.3_DLBCL (FOCAL); SEQ ID NO: 2519, 13q31.3_DLBCL (FOCAL); SEQ ID NO: 2520, 13q31.3_DLBCL (FOCAL); SEQ ID NO: 2521, 13q31.3_DLBCL (FOCAL); SEQ ID NO: 2522, 13q31.3_DLBCL (FOCAL); SEQ ID NO: 2523, 13q31.3_DLBCL (FOCAL); SEQ ID NO: 2524, 13q31.3_DLBCL (FOCAL); SEQ ID NO: 2525, 13q31.3_DLBCL (FOCAL); SEQ ID NO: 2526, 13q31.3_DLBCL (FOCAL); SEQ ID NO: 2527, 13q31.3_DLBCL (FOCAL); SEQ ID NO: 2528, 13q31.3_DLBCL (FOCAL); SEQ ID NO: 2529, 13q31.3_DLBCL (FOCAL); SEQ ID NO: 2530, 13q31.3_DLBCL (FOCAL); SEQ ID NO: 2531, 13q31.3_DLBCL (FOCAL); SEQ ID NO: 2532, 13q31.3_DLBCL (FOCAL); SEQ ID NO: 2533, 13q31.3_DLBCL (FOCAL); SEQ ID NO: 2534, 13q31.3_DLBCL (FOCAL); SEQ ID NO: 2535, 13q31.3_DLBCL (FOCAL); SEQ ID NO: 2536, 13q31.3_DLBCL (FOCAL); SEQ ID NO: 2537, 13q31.3_DLBCL (FOCAL); SEQ ID NO: 2538, 13q31.3_DLBCL (FOCAL); SEQ ID NO: 2539, 13q31.3_DLBCL (FOCAL); SEQ ID NO: 2540, 13q31.3_DLBCL (FOCAL); SEQ ID NO: 2541, 13q31.3_DLBCL (FOCAL); SEQ ID NO: 2542, 13q31.3_DLBCL (FOCAL); SEQ ID NO: 2543, 13q31.3_DLBCL (FOCAL); SEQ ID NO: 2544, 13q31.3_DLBCL (FOCAL); SEQ ID NO: 2545, 13q31.3_DLBCL (FOCAL); SEQ ID NO: 2546, 13q31.3_DLBCL (FOCAL); SEQ ID NO: 2547, 13q31.3_DLBCL (FOCAL); SEQ ID NO: 2548, 13q31.3_DLBCL (FOCAL); SEQ ID NO: 2549, 13q31.3_DLBCL (FOCAL); SEQ ID NO: 2550, 13q31.3_DLBCL (FOCAL); SEQ ID NO: 2551, 13q31.3_DLBCL (FOCAL); SEQ ID NO: 2552, 13q31.3_DLBCL (FOCAL); SEQ ID NO: 2553, 13q31.3_DLBCL (FOCAL); SEQ ID NO: 2554, 13q31.3_DLBCL (FOCAL); SEQ ID NO: 2555, 13q31.3_DLBCL (FOCAL); SEQ ID NO: 2556, 13q31.3_DLBCL (FOCAL); SEQ ID NO: 2557, 13q31.3_DLBCL (FOCAL); SEQ ID NO: 2558, 13q31.3_DLBCL (FOCAL); SEQ ID NO: 2559, 13q31.3_DLBCL (FOCAL); SEQ ID NO: 2560, 13q31.3_DLBCL (FOCAL); SEQ ID NO: 2561, 13q31.3_DLBCL (FOCAL); SEQ ID NO: 2562, 13q31.3_DLBCL (FOCAL); SEQ ID NO: 2563, 13q31.3_DLBCL (FOCAL); SEQ ID NO: 2564, 13q31.3_DLBCL (FOCAL); SEQ ID NO: 2565, 13q31.3_DLBCL (FOCAL); SEQ ID NO: 2566, 13q31.3_DLBCL (FOCAL); SEQ ID NO: 2567, 13q31.3_DLBCL (FOCAL); SEQ ID NO: 2568, 13q31.3_DLBCL (FOCAL); SEQ ID NO: 2569, 13q31.3_DLBCL (FOCAL); SEQ ID NO: 2570, 13q31.3_DLBCL (FOCAL); SEQ ID NO: 2571, 13q31.3_DLBCL (FOCAL); SEQ ID NO: 2572, 13q31.3_DLBCL (FOCAL); SEQ ID NO: 2573, 13q31.3_DLBCL (FOCAL); SEQ ID NO: 2574, 13q31.3_DLBCL (FOCAL); SEQ ID NO: 2575, 13q31.3_DLBCL (FOCAL); SEQ ID NO: 2576, 13q31.3_DLBCL (FOCAL); SEQ ID NO: 2577, 13q31.3_DLBCL (FOCAL); SEQ ID NO: 2578, 13q31.3_DLBCL (FOCAL); SEQ ID NO: 2579, 13q31.3_DLBCL (FOCAL); SEQ ID NO: 2580, 13q31.3_DLBCL (FOCAL); SEQ ID NO: 2581, 13q31.3_DLBCL (FOCAL); SEQ ID NO: 2582, 13q31.3_DLBCL (FOCAL); SEQ ID NO: 2583, 13q31.3_DLBCL (FOCAL); SEQ ID NO: 2584, 13q31.3_DLBCL (FOCAL); SEQ ID NO: 2585, 13q31.3_DLBCL (FOCAL); SEQ ID NO: 2586, 13q31.3_DLBCL (FOCAL); SEQ ID NO: 2587, 13q31.3_DLBCL (FOCAL); SEQ ID NO: 2588, 13q31.3_DLBCL (FOCAL); SEQ ID NO: 2589, 13q31.3_DLBCL (FOCAL); SEQ ID NO: 2590, 13q31.3_DLBCL (FOCAL); SEQ ID NO: 2591, 13q31.3_DLBCL (FOCAL); SEQ ID NO: 2592, 13q31.3_DLBCL (FOCAL); SEQ ID NO: 2593, 13q31.3_DLBCL (FOCAL); SEQ ID NO: 2594, 13q31.3_DLBCL (FOCAL); SEQ ID NO: 2595, 13q31.3_DLBCL (FOCAL); SEQ ID NO: 2596, 13q31.3_DLBCL (FOCAL); SEQ ID NO: 2597, 13q31.3_DLBCL (FOCAL); SEQ ID NO: 2598, 13q31.3_DLBCL (FOCAL); SEQ ID NO: 2599, 13q31.3_DLBCL (FOCAL); SEQ ID NO: 2600, 13q31.3_DLBCL (FOCAL); SEQ ID NO: 2601, FP (FP); SEQ ID NO: 2602, 13q31.3_DLBCL (FOCAL); SEQ ID NO: 2603, 13q31.3_DLBCL (FOCAL); SEQ ID NO: 2604, 13q31.3_DLBCL (FOCAL); SEQ ID NO: 2605, 13q31.3_DLBCL (FOCAL); SEQ ID NO: 2606, 13q31.3_DLBCL (FOCAL); SEQ ID NO: 2607, 13q31.3_DLBCL (FOCAL); SEQ ID NO: 2608, 13q31.3_DLBCL (FOCAL); SEQ ID NO: 2609, 13q31.3_DLBCL (FOCAL); SEQ ID NO: 2610, 13q31.3_DLBCL (FOCAL); SEQ ID NO: 2611, 13q34_DLBCL (FOCAL); SEQ ID NO: 2612, 13q34_DLBCL (FOCAL); SEQ ID NO: 2613, 13q34 DLBCL (FOCAL); SEQ ID NO: 2614, 13q34_DLBCL (FOCAL); SEQ ID NO: 2615, 13q34 DLBCL (FOCAL); SEQ ID NO: 2616, 13q34_DLBCL (FOCAL); SEQ ID NO: 2617, 13q34 DLBCL (FOCAL); SEQ ID NO: 2618, 13q34_DLBCL (FOCAL); SEQ ID NO: 2619, 13q34 DLBCL (FOCAL); SEQ ID NO: 2620, 13q34_DLBCL (FOCAL); SEQ ID NO: 2621, 13q34 DLBCL (FOCAL); SEQ ID NO: 2622, 13q34_DLBCL (FOCAL); SEQ ID NO: 2623, 13q34 DLBCL (FOCAL); SEQ ID NO: 2624, 13q34_DLBCL (FOCAL); SEQ ID NO: 2625, 13q34 DLBCL (FOCAL); SEQ ID NO: 2626, 13q34_DLBCL (FOCAL); SEQ ID NO: 2627, 13q34 DLBCL (FOCAL); SEQ ID NO: 2628, 13q34_DLBCL (FOCAL); SEQ ID NO: 2629, 13q34 DLBCL (FOCAL); SEQ ID NO: 2630, 13q34_DLBCL (FOCAL); SEQ ID NO: 2631, 13q34 DLBCL (FOCAL); SEQ ID NO: 2632, 13q34_DLBCL (FOCAL); SEQ ID NO: 2633, 13q34 DLBCL (FOCAL); SEQ ID NO: 2634, CHD8 (Gene); SEQ ID NO: 2635, CHD8 (Gene); SEQ ID NO: 2636, CHD8 (Gene); SEQ ID NO: 2637, CHD8 (Gene); SEQ ID NO: 2638, CHD8 (Gene); SEQ ID NO: 2639, CHD8 (Gene); SEQ ID NO: 2640, CHD8 (Gene); SEQ ID NO: 2641, CHD8 (Gene); SEQ ID NO: 2642, CHD8 (Gene); SEQ ID NO: 2643, CHD8 (Gene); SEQ ID NO: 2644, CHD8 (Gene); SEQ ID NO: 2645, CHD8 (Gene); SEQ ID NO: 2646, CHD8 (Gene); SEQ ID NO: 2647, CHD8 (Gene); SEQ ID NO: 2648, CHD8 (Gene); SEQ ID NO: 2649, CHD8 (Gene); SEQ ID NO: 2650, CHD8 (Gene); SEQ ID NO: 2651, CHD8 (Gene); SEQ ID NO: 2652, CHD8 (Gene); SEQ ID NO: 2653, CHD8 (Gene); SEQ ID NO: 2654, CHD8 (Gene); SEQ ID NO: 2655, CHD8 (Gene); SEQ ID NO: 2656, CHD8 (Gene); SEQ ID NO: 2657, CHD8 (Gene); SEQ ID NO: 2658, CHD8 (Gene); SEQ ID NO: 2659, CHD8 (Gene); SEQ ID NO: 2660, CHD8 (Gene); SEQ ID NO: 2661, CHD8 (Gene); SEQ ID NO: 2662, CHD8 (Gene); SEQ ID NO: 2663, CHD8 (Gene); SEQ ID NO: 2664, CHD8 (Gene); SEQ ID NO: 2665, CHD8 (Gene); SEQ ID NO: 2666, CHD8 (Gene); SEQ ID NO: 2667, CHD8 (Gene); SEQ ID NO: 2668, CHD8 (Gene); SEQ ID NO: 2669, CHD8 (Gene); SEQ ID NO: 2670, CHD8 (Gene); SEQ ID NO: 2671, CHD8 (Gene); SEQ ID NO: 2672, CHD8 (Gene); SEQ ID NO: 2673, CHD8 (Gene); SEQ ID NO: 2674, CHD8 (Gene); SEQ ID NO: 2675, CHD8 (Gene); SEQ ID NO: 2676, CHD8 (Gene); SEQ ID NO: 2677, CHD8 (Gene); SEQ ID NO: 2678, 14Q (ARM); SEQ ID NO: 2679, 14Q (ARM); SEQ ID NO: 2680, 14Q (ARM); SEQ ID NO: 2681, FP (FP); SEQ ID NO: 2682, 14Q (ARM); SEQ ID NO: 2683, 14Q (ARM); SEQ ID NO: 2684, FP (FP); SEQ ID NO: 2685, 14Q (ARM); SEQ ID NO: 2686, 14Q (ARM); SEQ ID NO: 2687, 14Q (ARM); SEQ ID NO: 2688, 14Q (ARM); SEQ ID NO: 2689, FP (FP); SEQ ID NO: 2690, 14Q (ARM); SEQ ID NO: 2691, 14Q (ARM); SEQ ID NO: 2692, NFKBIA (Gene); SEQ ID NO: 2693, NFKBIA (Gene); SEQ ID NO: 2694, NFKBIA (Gene); SEQ ID NO: 2695, NFKBIA (Gene); SEQ ID NO: 2696, NFKBIA (Gene); SEQ ID NO: 2697, NFKBIA (Gene); SEQ ID NO: 2698, 14Q (ARM); SEQ ID NO: 2699, 14Q (ARM); SEQ ID NO: 2700, 14Q (ARM); SEQ ID NO: 2701, 14Q (ARM); SEQ ID NO: 2702, 14Q (ARM); SEQ ID NO: 2703, 14Q (ARM); SEQ ID NO: 2704, 14Q (ARM); SEQ ID NO: 2705, 14Q (ARM); SEQ ID NO: 2706, 14Q (ARM); SEQ ID NO: 2707, 14Q (ARM); SEQ ID NO: 2708, 14Q (ARM); SEQ ID NO: 2709, 14Q (ARM); SEQ ID NO: 2710, 14Q (ARM); SEQ ID NO: 2711, 14Q (ARM); SEQ ID NO: 2712, 14Q (ARM); SEQ ID NO: 2713, 14Q (ARM); SEQ ID NO: 2714, 14Q (ARM); SEQ ID NO: 2715, 14Q (ARM); SEQ ID NO: 2716, 14Q (ARM); SEQ ID NO: 2717, 14Q (ARM); SEQ ID NO: 2718, 14Q (ARM); SEQ ID NO: 2719, 14Q (ARM); SEQ ID NO: 2720, 14Q (ARM); SEQ ID NO: 2721, 14Q (ARM); SEQ ID NO: 2722, 14Q (ARM); SEQ ID NO: 2723, 14Q (ARM); SEQ ID NO: 2724, FP (FP); SEQ ID NO: 2725, 14Q (ARM); SEQ ID NO: 2726, 14Q (ARM); SEQ ID NO: 2727, ZFP36L1 (Gene); SEQ ID NO: 2728, ZFP36L1 (Gene); SEQ ID NO: 2729, ZFP36L1 (Gene); SEQ ID NO: 2730, ZFP36L1 (Gene); SEQ ID NO: 2731, 14Q (ARM); SEQ ID NO: 2732, 14Q (ARM); SEQ ID NO: 2733, 14Q (ARM); SEQ ID NO: 2734, 14Q (ARM); SEQ ID NO: 2735, 14Q (ARM); SEQ ID NO: 2736, 14Q (ARM); SEQ ID NO: 2737, 14Q (ARM); SEQ ID NO: 2738, 14Q (ARM); SEQ ID NO: 2739, 14Q (ARM); SEQ ID NO: 2740, 14Q (ARM); SEQ ID NO: 2741, 14Q (ARM); SEQ ID NO: 2742, 14Q (ARM); SEQ ID NO: 2743, 14Q (ARM); SEQ ID NO: 2744, 14Q (ARM); SEQ ID NO: 2745, 14Q (ARM); SEQ ID NO: 2746, 14Q (ARM); SEQ ID NO: 2747, 14Q (ARM); SEQ ID NO: 2748, 14Q (ARM); SEQ ID NO: 2749, 14Q (ARM); SEQ ID NO: 2750, 14Q (ARM); SEQ ID NO: 2751, PPP4R3A (Gene); SEQ ID NO: 2752, PPP4R3A (Gene); SEQ ID NO: 2753, PPP4R3A (Gene); SEQ ID NO: 2754, PPP4R3A (Gene); SEQ ID NO: 2755, PPP4R3A (Gene); SEQ ID NO: 2756, PPP4R3A (Gene); SEQ ID NO: 2757, PPP4R3A (Gene); SEQ ID NO: 2758, PPP4R3A (Gene); SEQ ID NO: 2759, PPP4R3A (Gene); SEQ ID NO: 2760, PPP4R3A (Gene); SEQ ID NO: 2761, PPP4R3A (Gene); SEQ ID NO: 2762, PPP4R3A (Gene); SEQ ID NO: 2763, PPP4R3A (Gene); SEQ ID NO: 2764, PPP4R3A (Gene); SEQ ID NO: 2765, PPP4R3A (Gene); SEQ ID NO: 2766, PPP4R3A (Gene); SEQ ID NO: 2767, 14Q (ARM); SEQ ID NO: 2768, 14Q (ARM); SEQ ID NO: 2769, 14Q (ARM); SEQ ID NO: 2770, 14Q (ARM); SEQ ID NO: 2771, FP (FP); SEQ ID NO: 2772, TCL1A (Gene); SEQ ID NO: 2773, TCL1A (Gene); SEQ ID NO: 2774, TCL1A (Gene); SEQ ID NO: 2775, 14Q (ARM); SEQ ID NO: 2776, 14Q (ARM); SEQ ID NO: 2777, 14Q (ARM); SEQ ID NO: 2778, FP (FP); SEQ ID NO: 2779, 14Q (ARM); SEQ ID NO: 2780, 14Q (ARM); SEQ ID NO: 2781, 14Q (ARM); SEQ ID NO: 2782, YY1 (Gene); SEQ ID NO: 2783, YY1 (Gene); SEQ ID NO: 2784, YY1 (Gene); SEQ ID NO: 2785, YY1 (Gene); SEQ ID NO: 2786, YY1 (Gene); SEQ ID NO: 2787, YY1 (Gene); SEQ ID NO: 2788, 14Q (ARM); SEQ ID NO: 2789, 14q32.31_DLBCL (FOCAL); SEQ ID NO: 2790, 14q32.31_DLBCL (FOCAL); SEQ ID NO: 2791, 14q32.31_DLBCL (FOCAL); SEQ ID NO: 2792, RCOR1 (Gene); SEQ ID NO: 2793, RCOR1 (Gene); SEQ ID NO: 2794, RCOR1 (Gene); SEQ ID NO: 2795, RCOR1 (Gene); SEQ ID NO: 2796, RCOR1 (Gene); SEQ ID NO: 2797, RCOR1 (Gene); SEQ ID NO: 2798, RCOR1 (Gene); SEQ ID NO: 2799, RCOR1 (Gene); SEQ ID NO: 2800, RCOR1 (Gene); SEQ ID NO: 2801, RCOR1 (Gene); SEQ ID NO: 2802, RCOR1 (Gene); SEQ ID NO: 2803, RCOR1 (Gene); SEQ ID NO: 2804, 14q32.31_DLBCL (FOCAL); SEQ ID NO: 2805, TRAF3 (Gene); SEQ ID NO: 2806, TRAF3 (Gene); SEQ ID NO: 2807, TRAF3 (Gene); SEQ ID NO: 2808, TRAF3 (Gene); SEQ ID NO: 2809, TRAF3 (Gene); SEQ ID NO: 2810, TRAF3 (Gene); SEQ ID NO: 2811, TRAF3 (Gene); SEQ ID NO: 2812, TRAF3 (Gene); SEQ ID NO: 2813, TRAF3 (Gene); SEQ ID NO: 2814, TRAF3 (Gene); SEQ ID NO: 2815, TRAF3 (Gene); SEQ ID NO: 2816, TRAF3 (Gene); SEQ ID NO: 2817, 14q32.31_DLBCL (FOCAL); SEQ ID NO: 2818, 14q32.31_DLBCL (FOCAL); SEQ ID NO: 2819, 14q32.31_DLBCL (FOCAL); SEQ ID NO: 2820, 14q32.31_DLBCL (FOCAL); SEQ ID NO: 2821, 14q32.31_DLBCL (FOCAL); SEQ ID NO: 2822, 14Q (ARM); SEQ ID NO: 2823, CRIP1 (Gene); SEQ ID NO: 2824, CRIP1 (Gene); SEQ ID NO: 2825, CRIP1 (Gene); SEQ ID NO: 2826, CRIP1 (Gene); SEQ ID NO: 2827, IGH_SV (SV); SEQ ID NO: 2828, IGH_SV (SV); SEQ ID NO: 2829, IGH_SV (SV); SEQ ID NO: 2830, IGH_SV (SV); SEQ ID NO: 2831, IGH_SV (SV); SEQ ID NO: 2832, IGH_SV (SV); SEQ ID NO: 2833, IGH_SV (SV); SEQ ID NO: 2834, IGH_SV (SV); SEQ ID NO: 2835, IGH_SV (SV); SEQ ID NO: 2836, IGH_SV (SV); SEQ ID NO: 2837, IGH_SV (SV); SEQ ID NO: 2838, IGH_SV (SV); SEQ ID NO: 2839, IGH_SV (SV); SEQ ID NO: 2840, IGH_SV (SV); SEQ ID NO: 2841, IGH_SV (SV); SEQ ID NO: 2842, IGH_SV (SV); SEQ ID NO: 2843, IGH_SV (SV); SEQ ID NO: 2844, IGH_SV (SV); SEQ ID NO: 2845, IGH_SV (SV); SEQ ID NO: 2846, IGH_SV (SV); SEQ ID NO: 2847, IGH_SV (SV); SEQ ID NO: 2848, IGH_SV (SV); SEQ ID NO: 2849, IGH_SV (SV); SEQ ID NO: 2850, IGH_SV (SV); SEQ ID NO: 2851, IGH_SV (SV); SEQ ID NO: 2852, IGH_SV (SV); SEQ ID NO: 2853, IGH_SV (SV); SEQ ID NO: 2854, IGH_SV (SV); SEQ ID NO: 2855, IGH_SV (SV); SEQ ID NO: 2856, IGH_SV (SV); SEQ ID NO: 2857, IGH_SV (SV); SEQ ID NO: 2858, IGH_SV (SV); SEQ ID NO: 2859, IGH_SV (SV); SEQ ID NO: 2860, IGH_SV (SV); SEQ ID NO: 2861, IGH_SV (SV); SEQ ID NO: 2862, IGH_SV (SV); SEQ ID NO: 2863, IGH_SV (SV); SEQ ID NO: 2864, IGH_SV (SV); SEQ ID NO: 2865, IGH_SV (SV); SEQ ID NO: 2866, IGH_SV (SV); SEQ ID NO: 2867, IGH_SV (SV); SEQ ID NO: 2868, IGH_SV (SV); SEQ ID NO: 2869, IGH_SV (SV); SEQ ID NO: 2870, IGH_SV (SV); SEQ ID NO: 2871, IGH_SV (SV); SEQ ID NO: 2872, IGH_SV (SV); SEQ ID NO: 2873, IGH_SV (SV); SEQ ID NO: 2874, IGH_SV (SV); SEQ ID NO: 2875, IGH_SV (SV); SEQ ID NO: 2876, IGH_SV (SV); SEQ ID NO: 2877, IGH_SV (SV); SEQ ID NO: 2878, IGH_SV (SV); SEQ ID NO: 2879, IGH_SV (SV); SEQ ID NO: 2880, IGH_SV (SV); SEQ ID NO: 2881, IGH_SV (SV); SEQ ID NO: 2882, IGH_SV (SV); SEQ ID NO: 2883, IGH_SV (SV); SEQ ID NO: 2884, IGH_SV (SV); SEQ ID NO: 2885, IGH_SV (SV); SEQ ID NO: 2886, IGH_SV (SV); SEQ ID NO: 2887, IGH_SV (SV); SEQ ID NO: 2888, IGH_SV (SV); SEQ ID NO: 2889, IGH_SV (SV); SEQ ID NO: 2890, IGH_SV (SV); SEQ ID NO: 2891, IGH_SV (SV); SEQ ID NO: 2892, IGH_SV (SV); SEQ ID NO: 2893, IGH_SV (SV); SEQ ID NO: 2894, IGH_SV (SV); SEQ ID NO: 2895, IGH_SV (SV); SEQ ID NO: 2896, IGH_SV (SV); SEQ ID NO: 2897, IGH_SV (SV); SEQ ID NO: 2898, IGH_SV (SV); SEQ ID NO: 2899, IGH_SV (SV); SEQ ID NO: 2900, IGH_SV (SV); SEQ ID NO: 2901, IGH_SV (SV); SEQ ID NO: 2902, IGH_SV (SV); SEQ ID NO: 2903, IGH_SV (SV); SEQ ID NO: 2904, IGH_SV (SV); SEQ ID NO: 2905, IGH_SV (SV); SEQ ID NO: 2906, IGH_SV (SV); SEQ ID NO: 2907, IGH_SV (SV); SEQ ID NO: 2908, IGH_SV (SV); SEQ ID NO: 2909, IGH_SV (SV); SEQ ID NO: 2910, IGH_SV (SV); SEQ ID NO: 2911, IGH_SV (SV); SEQ ID NO: 2912, IGH_SV (SV); SEQ ID NO: 2913, IGH_SV (SV); SEQ ID NO: 2914, IGH_SV (SV); SEQ ID NO: 2915, IGH_SV (SV); SEQ ID NO: 2916, IGH_SV (SV); SEQ ID NO: 2917, IGH_SV (SV); SEQ ID NO: 2918, IGH_SV (SV); SEQ ID NO: 2919, IGH_SV (SV); SEQ ID NO: 2920, IGH_SV (SV); SEQ ID NO: 2921, IGH_SV (SV); SEQ ID NO: 2922, IGH_SV (SV); SEQ ID NO: 2923, IGH_SV (SV); SEQ ID NO: 2924, IGH_SV (SV); SEQ ID NO: 2925, IGH_SV (SV); SEQ ID NO: 2926, IGH_SV (SV); SEQ ID NO: 2927, IGH_SV (SV); SEQ ID NO: 2928, IGH_SV (SV); SEQ ID NO: 2929, IGH_SV (SV); SEQ ID NO: 2930, IGH_SV (SV); SEQ ID NO: 2931, IGH_SV (SV); SEQ ID NO: 2932, IGH_SV (SV); SEQ ID NO: 2933, IGH_SV (SV); SEQ ID NO: 2934, IGH_SV (SV); SEQ ID NO: 2935, IGH_SV (SV); SEQ ID NO: 2936, IGH_SV (SV); SEQ ID NO: 2937, IGH_SV (SV); SEQ ID NO: 2938, IGH_SV (SV); SEQ ID NO: 2939, IGH_SV (SV); SEQ ID NO: 2940, IGH_SV (SV); SEQ ID NO: 2941, IGH_SV (SV); SEQ ID NO: 2942, IGH_SV (SV); SEQ ID NO: 2943, IGH_SV (SV); SEQ ID NO: 2944, IGH_SV (SV); SEQ ID NO: 2945, IGH_SV (SV); SEQ ID NO: 2946, IGH_SV (SV); SEQ ID NO: 2947, IGH_SV (SV); SEQ ID NO: 2948, IGH_SV (SV); SEQ ID NO: 2949, IGH_SV (SV); SEQ ID NO: 2950, IGH_SV (SV); SEQ ID NO: 2951, IGH_SV (SV); SEQ ID NO: 2952, IGH_SV (SV); SEQ ID NO: 2953, IGH_SV (SV); SEQ ID NO: 2954, IGH_SV (SV); SEQ ID NO: 2955, IGH_SV (SV); SEQ ID NO: 2956, IGH_SV (SV); SEQ ID NO: 2957, IGH_SV (SV); SEQ ID NO: 2958, IGH_SV (SV); SEQ ID NO: 2959, IGH_SV (SV); SEQ ID NO: 2960, 15Q (ARM); SEQ ID NO: 2961, FP (FP); SEQ ID NO: 2962, 15Q (ARM); SEQ ID NO: 2963, 15Q (ARM); SEQ ID NO: 2964, 15Q (ARM); SEQ ID NO: 2965, 15Q (ARM); SEQ ID NO: 2966, 15Q (ARM); SEQ ID NO: 2967, 15Q (ARM); SEQ ID NO: 2968, 15Q (ARM); SEQ ID NO: 2969, 15Q (ARM); SEQ ID NO: 2970, 15Q (ARM); SEQ ID NO: 2971, 15Q (ARM); SEQ ID NO: 2972, 15Q (ARM); SEQ ID NO: 2973, 15Q (ARM); SEQ ID NO: 2974, 15Q (ARM); SEQ ID NO: 2975, 15Q (ARM); SEQ ID NO: 2976, 15Q (ARM); SEQ ID NO: 2977, 15Q (ARM); SEQ ID NO: 2978, 15q15.3_DLBCL (FOCAL); SEQ ID NO: 2979, 15q15.3_DLBCL (FOCAL); SEQ ID NO: 2980, 15q15.3_DLBCL (FOCAL); SEQ ID NO: 2981, 15q15.3_DLBCL (FOCAL); SEQ ID NO: 2982, 15q15.3_DLBCL (FOCAL); SEQ ID NO: 2983, MGA (Gene); SEQ ID NO: 2984, MGA (Gene); SEQ ID NO: 2985, MGA (Gene); SEQ ID NO: 2986, MGA (Gene); SEQ ID NO: 2987, MGA (Gene); SEQ ID NO: 2988, MGA (Gene); SEQ ID NO: 2989, MGA (Gene); SEQ ID NO: 2990, MGA (Gene); SEQ ID NO: 2991, MGA (Gene); SEQ ID NO: 2992, MGA (Gene); SEQ ID NO: 2993, MGA (Gene); SEQ ID NO: 2994, MGA (Gene); SEQ ID NO: 2995, MGA (Gene); SEQ ID NO: 2996, MGA (Gene); SEQ ID NO: 2997, MGA (Gene); SEQ ID NO: 2998, MGA (Gene); SEQ ID NO: 2999, MGA (Gene); SEQ ID NO: 3000, MGA (Gene); SEQ ID NO: 3001, MGA (Gene); SEQ ID NO: 3002, MGA (Gene); SEQ ID NO: 3003, MGA (Gene); SEQ ID NO: 3004, MGA (Gene); SEQ ID NO: 3005, MGA (Gene); SEQ ID NO: 3006, MGA (Gene); SEQ ID NO: 3007, MGA (Gene); SEQ ID NO: 3008, MGA (Gene); SEQ ID NO: 3009, MGA (Gene); SEQ ID NO: 3010, MGA (Gene); SEQ ID NO: 3011, MGA (Gene); SEQ ID NO: 3012, MGA (Gene); SEQ ID NO: 3013, MGA (Gene); SEQ ID NO: 3014, MGA (Gene); SEQ ID NO: 3015, MGA (Gene); SEQ ID NO: 3016, MGA (Gene); SEQ ID NO: 3017, 15q15.3_DLBCL (FOCAL); SEQ ID NO: 3018, 15q15.3_DLBCL (FOCAL); SEQ ID NO: 3019, 15q15.3_DLBCL (FOCAL); SEQ ID NO: 3020, 15q15.3_DLBCL (FOCAL); SEQ ID NO: 3021, 15q15.3_DLBCL (FOCAL); SEQ ID NO: 3022, 15q15.3_DLBCL (FOCAL); SEQ ID NO: 3023, 15q15.3_DLBCL (FOCAL); SEQ ID NO: 3024, 15q15.3_DLBCL (FOCAL); SEQ ID NO: 3025, 15q15.3_DLBCL (FOCAL); SEQ ID NO: 3026, 15q15.3_DLBCL (FOCAL); SEQ ID NO: 3027, 15q15.3_DLBCL (FOCAL); SEQ ID NO: 3028, 15q15.3_DLBCL (FOCAL); SEQ ID NO: 3029, B2M (Gene); SEQ ID NO: 3030, B2M (Gene); SEQ ID NO: 3031, B2M (Gene); SEQ ID NO: 3032, 15q15.3_DLBCL (FOCAL); SEQ ID NO: 3033, 15q15.3_DLBCL (FOCAL); SEQ ID NO: 3034, 15Q (ARM); SEQ ID NO: 3035, 15Q (ARM); SEQ ID NO: 3036, FP (FP); SEQ ID NO: 3037, 15Q (ARM); SEQ ID NO: 3038, 15Q (ARM); SEQ ID NO: 3039, 15Q (ARM); SEQ ID NO: 3040, 15Q (ARM); SEQ ID NO: 3041, SCG3 (Gene); SEQ ID NO: 3042, SCG3 (Gene); SEQ ID NO: 3043, SCG3 (Gene); SEQ ID NO: 3044, SCG3 (Gene); SEQ ID NO: 3045, SCG3 (Gene); SEQ ID NO: 3046, SCG3 (Gene); SEQ ID NO: 3047, SCG3 (Gene); SEQ ID NO: 3048, SCG3 (Gene); SEQ ID NO: 3049, SCG3 (Gene); SEQ ID NO: 3050, SCG3 (Gene); SEQ ID NO: 3051, SCG3 (Gene); SEQ ID NO: 3052, SCG3 (Gene); SEQ ID NO: 3053, 15Q (ARM); SEQ ID NO: 3054, 15Q (ARM); SEQ ID NO: 3055, FP (FP); SEQ ID NO: 3056, 15Q (ARM); SEQ ID NO: 3057, 15Q (ARM); SEQ ID NO: 3058, 15Q (ARM); SEQ ID NO: 3059, FP (FP); SEQ ID NO: 3060, 15Q (ARM); SEQ ID NO: 3061, 15Q (ARM); SEQ ID NO: 3062, 15Q (ARM); SEQ ID NO: 3063, 15Q (ARM); SEQ ID NO: 3064, 15Q (ARM); SEQ ID NO: 3065, 15Q (ARM); SEQ ID NO: 3066, 15Q (ARM); SEQ ID NO: 3067, 15Q (ARM); SEQ ID NO: 3068, 15Q (ARM); SEQ ID NO: 3069, 15Q (ARM); SEQ ID NO: 3070, 15Q (ARM); SEQ ID NO: 3071, MSI (MSI); SEQ ID NO: 3072, MSI (MSI); SEQ ID NO: 3073, MAP2K1 (Gene); SEQ ID NO: 3074, MAP2K1 (Gene); SEQ ID NO: 3075, MAP2K1 (Gene); SEQ ID NO: 3076, MAP2K1 (Gene); SEQ ID NO: 3077, MAP2K1 (Gene); SEQ ID NO: 3078, MAP2K1 (Gene); SEQ ID NO: 3079, MAP2K1 (Gene); SEQ ID NO: 3080, MAP2K1 (Gene); SEQ ID NO: 3081, MAP2K1 (Gene); SEQ ID NO: 3082, MAP2K1 (Gene); SEQ ID NO: 3083, MAP2K1 (Gene); SEQ ID NO: 3084, 15Q (ARM); SEQ ID NO: 3085, 15Q (ARM); SEQ ID NO: 3086, 15Q (ARM); SEQ ID NO: 3087, 15Q (ARM); SEQ ID NO: 3088, 15Q (ARM); SEQ ID NO: 3089, 15Q (ARM); SEQ ID NO: 3090, 15Q (ARM); SEQ ID NO: 3091, 15Q (ARM); SEQ ID NO: 3092, SIN3A (Gene); SEQ ID NO: 3093, SIN3A (Gene); SEQ ID NO: 3094, SIN3A (Gene); SEQ ID NO: 3095, SIN3A (Gene); SEQ ID NO: 3096, SIN3A (Gene); SEQ ID NO: 3097, SIN3A (Gene); SEQ ID NO: 3098, SIN3A (Gene); SEQ ID NO: 3099, SIN3A (Gene); SEQ ID NO: 3100, SIN3A (Gene); SEQ ID NO: 3101, SIN3A (Gene); SEQ ID NO: 3102, SIN3A (Gene); SEQ ID NO: 3103, SIN3A (Gene); SEQ ID NO: 3104, SIN3A (Gene); SEQ ID NO: 3105, SIN3A (Gene); SEQ ID NO: 3106, SIN3A (Gene); SEQ ID NO: 3107, SIN3A (Gene); SEQ ID NO: 3108, SIN3A (Gene); SEQ ID NO: 3109, SIN3A (Gene); SEQ ID NO: 3110, SIN3A (Gene); SEQ ID NO: 3111, SIN3A (Gene); SEQ ID NO: 3112, SIN3A (Gene); SEQ ID NO: 3113, 15Q (ARM); SEQ ID NO: 3114, 15Q (ARM); SEQ ID NO: 3115, MSI (MSI); SEQ ID NO: 3116, MSI (MSI); SEQ ID NO: 3117, 15Q (ARM); SEQ ID NO: 3118, 15Q (ARM); SEQ ID NO: 3119, 15Q (ARM); SEQ ID NO: 3120, 15Q (ARM); SEQ ID NO: 3121, 15Q (ARM); SEQ ID NO: 3122, 15Q (ARM); SEQ ID NO: 3123, 15Q (ARM); SEQ ID NO: 3124, 15Q (ARM); SEQ ID NO: 3125, 15Q (ARM); SEQ ID NO: 3126, 15Q (ARM); SEQ ID NO: 3127, IDH2 (Gene); SEQ ID NO: 3128, IDH2 (Gene); SEQ ID NO: 3129, IDH2 (Gene); SEQ ID NO: 3130, IDH2 (Gene); SEQ ID NO: 3131, IDH2 (Gene); SEQ ID NO: 3132, IDH2 (Gene); SEQ ID NO: 3133, IDH2 (Gene); SEQ ID NO: 3134, IDH2 (Gene); SEQ ID NO: 3135, IDH2 (Gene); SEQ ID NO: 3136, IDH2 (Gene); SEQ ID NO: 3137, IDH2 (Gene); SEQ ID NO: 3138, 15Q (ARM); SEQ ID NO: 3139, MSI (MSI); SEQ ID NO: 3140, MSI (MSI); SEQ ID NO: 3141, FP (FP); SEQ ID NO: 3142, 15Q (ARM); SEQ ID NO: 3143, 15Q (ARM); SEQ ID NO: 3144, 15Q (ARM); SEQ ID NO: 3145, 15Q (ARM); SEQ ID NO: 3146, 15Q (ARM); SEQ ID NO: 3147, 15Q (ARM); SEQ ID NO: 3148, 15Q (ARM); SEQ ID NO: 3149, 15Q (ARM); SEQ ID NO: 3150, 15Q (ARM); SEQ ID NO: 3151, 15Q (ARM); SEQ ID NO: 3152, 15Q (ARM); SEQ ID NO: 3153, 15Q (ARM); SEQ ID NO: 3154, 15Q (ARM); SEQ ID NO: 3155, TSC2 (Gene); SEQ ID NO: 3156, TSC2 (Gene); SEQ ID NO: 3157, TSC2 (Gene); SEQ ID NO: 3158, TSC2 (Gene); SEQ ID NO: 3159, TSC2 (Gene); SEQ ID NO: 3160, TSC2 (Gene); SEQ ID NO: 3161, TSC2 (Gene); SEQ ID NO: 3162, TSC2 (Gene); SEQ ID NO: 3163, TSC2 (Gene); SEQ ID NO: 3164, TSC2 (Gene); SEQ ID NO: 3165, TSC2 (Gene); SEQ ID NO: 3166, TSC2 (Gene); SEQ ID NO: 3167, TSC2 (Gene); SEQ ID NO: 3168, TSC2 (Gene); SEQ ID NO: 3169, TSC2 (Gene); SEQ ID NO: 3170, TSC2 (Gene); SEQ ID NO: 3171, TSC2 (Gene); SEQ ID NO: 3172, TSC2 (Gene); SEQ ID NO: 3173, TSC2 (Gene); SEQ ID NO: 3174, TSC2 (Gene); SEQ ID NO: 3175, TSC2 (Gene); SEQ ID NO: 3176, TSC2 (Gene); SEQ ID NO: 3177, TSC2 (Gene); SEQ ID NO: 3178, TSC2 (Gene); SEQ ID NO: 3179, TSC2 (Gene); SEQ ID NO: 3180, TSC2 (Gene); SEQ ID NO: 3181, TSC2 (Gene); SEQ ID NO: 3182, TSC2 (Gene); SEQ ID NO: 3183, TSC2 (Gene); SEQ ID NO: 3184, TSC2 (Gene); SEQ ID NO: 3185, TSC2 (Gene); SEQ ID NO: 3186, TSC2 (Gene); SEQ ID NO: 3187, TSC2 (Gene); SEQ ID NO: 3188, TSC2 (Gene); SEQ ID NO: 3189, TSC2 (Gene); SEQ ID NO: 3190, TSC2 (Gene); SEQ ID NO: 3191, TSC2 (Gene); SEQ ID NO: 3192, TSC2 (Gene); SEQ ID NO: 3193, TSC2 (Gene); SEQ ID NO: 3194, TSC2 (Gene); SEQ ID NO: 3195, TSC2 (Gene); SEQ ID NO: 3196, TSC2 (Gene); SEQ ID NO: 3197, TSC2 (Gene); SEQ ID NO: 3198, TSC2 (Gene); SEQ ID NO: 3199, TSC2 (Gene); SEQ ID NO: 3200, 16P (ARM); SEQ ID NO: 3201, CREBBP (Gene); SEQ ID NO: 3202, CREBBP (Gene); SEQ ID NO: 3203, CREBBP (Gene); SEQ ID NO: 3204, CREBBP (Gene); SEQ ID NO: 3205, CREBBP (Gene); SEQ ID NO: 3206, CREBBP (Gene); SEQ ID NO: 3207, CREBBP (Gene); SEQ ID NO: 3208, CREBBP (Gene); SEQ ID NO: 3209, CREBBP (Gene); SEQ ID NO: 3210, CREBBP (Gene); SEQ ID NO: 3211, CREBBP (Gene); SEQ ID NO: 3212, CREBBP (Gene); SEQ ID NO: 3213, CREBBP (Gene); SEQ ID NO: 3214, CREBBP (Gene); SEQ ID NO: 3215, CREBBP (Gene); SEQ ID NO: 3216, CREBBP (Gene); SEQ ID NO: 3217, CREBBP (Gene); SEQ ID NO: 3218, CREBBP (Gene); SEQ ID NO: 3219, CREBBP (Gene); SEQ ID NO: 3220, CREBBP (Gene); SEQ ID NO: 3221, CREBBP (Gene); SEQ ID NO: 3222, CREBBP (Gene); SEQ ID NO: 3223, CREBBP (Gene); SEQ ID NO: 3224, CREBBP (Gene); SEQ ID NO: 3225, CREBBP (Gene); SEQ ID NO: 3226, CREBBP (Gene); SEQ ID NO: 3227, CREBBP (Gene); SEQ ID NO: 3228, CREBBP (Gene); SEQ ID NO: 3229, CREBBP (Gene); SEQ ID NO: 3230, CREBBP (Gene); SEQ ID NO: 3231, CREBBP (Gene); SEQ ID NO: 3232, CREBBP (Gene); SEQ ID NO: 3233, CREBBP (Gene); SEQ ID NO: 3234, CREBBP (Gene); SEQ ID NO: 3235, CREBBP (Gene); SEQ ID NO: 3236, CREBBP (Gene); SEQ ID NO: 3237, CREBBP (Gene); SEQ ID NO: 3238, CREBBP (Gene); SEQ ID NO: 3239, CREBBP (Gene); SEQ ID NO: 3240, CREBBP (Gene); SEQ ID NO: 3241, FP (FP); SEQ ID NO: 3242, 16P (ARM); SEQ ID NO: 3243, 16P (ARM); SEQ ID NO: 3244, 16P (ARM); SEQ ID NO: 3245, 16P (ARM); SEQ ID NO: 3246, 16P (ARM); SEQ ID NO: 3247, 16P (ARM); SEQ ID NO: 3248, 16P (ARM); SEQ ID NO: 3249, 16P (ARM); SEQ ID NO: 3250, 16P (ARM); SEQ ID NO: 3251, MSI (MSI); SEQ ID NO: 3252, MSI (MSI); SEQ ID NO: 3253, CIITA_SV (SV); SEQ ID NO: 3254, CIITA_SV (SV); SEQ ID NO: 3255, CIITA_SV (SV); SEQ ID NO: 3256, CIITA_SV (SV); SEQ ID NO: 3257, CIITA_SV (SV); SEQ ID NO: 3258, CIITA_SV (SV); SEQ ID NO: 3259, CIITA_SV (SV); SEQ ID NO: 3260, CIITA_SV (SV); SEQ ID NO: 3261, CIITA_SV (SV); SEQ ID NO: 3262, CIITA_SV (SV); SEQ ID NO: 3263, CIITA_SV (SV); SEQ ID NO: 3264, CIITA_SV (SV); SEQ ID NO: 3265, CIITA_SV (SV); SEQ ID NO: 3266, CIITA_SV (SV); SEQ ID NO: 3267, CIITA_SV (SV); SEQ ID NO: 3268, CIITA_SV (SV); SEQ ID NO: 3269, CIITA_SV (SV); SEQ ID NO: 3270, CIITA_SV (SV); SEQ ID NO: 3271, CIITA_SV (SV); SEQ ID NO: 3272, CIITA_SV (SV); SEQ ID NO: 3273, CIITA_SV (SV); SEQ ID NO: 3274, CIITA_SV (SV); SEQ ID NO: 3275, CIITA_SV (SV); SEQ ID NO: 3276, CIITA_SV (SV); SEQ ID NO: 3277, CIITA_SV (SV); SEQ ID NO: 3278, CIITA_SV (SV); SEQ ID NO: 3279, CIITA_SV (SV); SEQ ID NO: 3280, CIITA_SV (SV); SEQ ID NO: 3281, CIITA_SV (SV); SEQ ID NO: 3282, CIITA_SV (SV); SEQ ID NO: 3283, CIITA_SV (SV); SEQ ID NO: 3284, CIITA_SV (SV); SEQ ID NO: 3285, CIITA_SV (SV); SEQ ID NO: 3286, CIITA_SV (SV); SEQ ID NO: 3287, CIITA_SV (SV); SEQ ID NO: 3288, CIITA_SV (SV); SEQ ID NO: 3289, CIITA_SV (SV); SEQ ID NO: 3290, CIITA (Gene); SEQ ID NO: 3291, CIITA (Gene); SEQ ID NO: 3292, CIITA (Gene); SEQ ID NO: 3293, CIITA (Gene); SEQ ID NO: 3294, CIITA (Gene); SEQ ID NO: 3295, CIITA (Gene); SEQ ID NO: 3296, CIITA (Gene); SEQ ID NO: 3297, CIITA (Gene); SEQ ID NO: 3298, CIITA (Gene); SEQ ID NO: 3299, CIITA (Gene); SEQ ID NO: 3300, CIITA (Gene); SEQ ID NO: 3301, CIITA (Gene); SEQ ID NO: 3302, CIITA (Gene); SEQ ID NO: 3303, CIITA (Gene); SEQ ID NO: 3304, CIITA (Gene); SEQ ID NO: 3305, CIITA (Gene); SEQ ID NO: 3306, CIITA (Gene); SEQ ID NO: 3307, CIITA (Gene); SEQ ID NO: 3308, CIITA (Gene); SEQ ID NO: 3309, CIITA (Gene); SEQ ID NO: 3310, CIITA (Gene); SEQ ID NO: 3311, CIITA (Gene); SEQ ID NO: 3312, CIITA (Gene); SEQ ID NO: 3313, SOCS1 (Gene); SEQ ID NO: 3314, SOCS1 (Gene); SEQ ID NO: 3315, 16P (ARM); SEQ ID NO: 3316, 16P (ARM); SEQ ID NO: 3317, 16P (ARM); SEQ ID NO: 3318, 16P (ARM); SEQ ID NO: 3319, 16P (ARM); SEQ ID NO: 3320, 16P (ARM); SEQ ID NO: 3321, 16P (ARM); SEQ ID NO: 3322, 16P (ARM); SEQ ID NO: 3323, PRKCB (Gene); SEQ ID NO: 3324, PRKCB (Gene); SEQ ID NO: 3325, PRKCB (Gene); SEQ ID NO: 3326, PRKCB (Gene); SEQ ID NO: 3327, PRKCB (Gene); SEQ ID NO: 3328, PRKCB (Gene); SEQ ID NO: 3329, PRKCB (Gene); SEQ ID NO: 3330, PRKCB (Gene); SEQ ID NO: 3331, PRKCB (Gene); SEQ ID NO: 3332, 16P (ARM); SEQ ID NO: 3333, PRKCB (Gene); SEQ ID NO: 3334, PRKCB (Gene); SEQ ID NO: 3335, PRKCB (Gene); SEQ ID NO: 3336, PRKCB (Gene); SEQ ID NO: 3337, PRKCB (Gene); SEQ ID NO: 3338, PRKCB (Gene); SEQ ID NO: 3339, PRKCB (Gene); SEQ ID NO: 3340, PRKCB (Gene); SEQ ID NO: 3341, PRKCB (Gene); SEQ ID NO: 3342, 16P (ARM); SEQ ID NO: 3343, 16P (ARM); SEQ ID NO: 3344, 16P (ARM); SEQ ID NO: 3345, 16P (ARM); SEQ ID NO: 3346, IL4R (Gene); SEQ ID NO: 3347, IL4R (Gene); SEQ ID NO: 3348, IL4R (Gene); SEQ ID NO: 3349, IL4R (Gene); SEQ ID NO: 3350, IL4R (Gene); SEQ ID NO: 3351, IL4R (Gene); SEQ ID NO: 3352, IL4R (Gene); SEQ ID NO: 3353, IL4R (Gene); SEQ ID NO: 3354, IL4R (Gene); SEQ ID NO: 3355, IL4R (Gene); SEQ ID NO: 3356, IL4R (Gene); SEQ ID NO: 3357, IL4R (Gene); SEQ ID NO: 3358, IL4R (Gene); SEQ ID NO: 3359, 16P (ARM); SEQ ID NO: 3360, CD19 (Gene); SEQ ID NO: 3361, CD19 (Gene); SEQ ID NO: 3362, CD19 (Gene); SEQ ID NO: 3363, CD19 (Gene); SEQ ID NO: 3364, CD19 (Gene); SEQ ID NO: 3365, CD19 (Gene); SEQ ID NO: 3366, CD19 (Gene); SEQ ID NO: 3367, CD19 (Gene); SEQ ID NO: 3368, CD19 (Gene); SEQ ID NO: 3369, CD19 (Gene); SEQ ID NO: 3370, CD19 (Gene); SEQ ID NO: 3371, CD19 (Gene); SEQ ID NO: 3372, CD19 (Gene); SEQ ID NO: 3373, CD19 (Gene); SEQ ID NO: 3374, 16q12.1_DLBCL_trim (FOCAL); SEQ ID NO: 3375, 16q12.1_DLBCL_trim (FOCAL); SEQ ID NO: 3376, 16q12.1_DLBCL_trim (FOCAL); SEQ ID NO: 3377, 16q12.1_DLBCL_trim (FOCAL); SEQ ID NO: 3378, 16q12.1_DLBCL_trim (FOCAL); SEQ ID NO: 3379, 16q12.1_DLBCL_trim (FOCAL); SEQ ID NO: 3380, 16q12.1_DLBCL_trim (FOCAL); SEQ ID NO: 3381, 16q12.1_DLBCL_trim (FOCAL); SEQ ID NO: 3382, 16q12.1_DLBCL_trim (FOCAL); SEQ ID NO: 3383, 16q12.1_DLBCL_trim (FOCAL); SEQ ID NO: 3384, 16q12.1_DLBCL_trim (FOCAL); SEQ ID NO: 3385, 16q12.1_DLBCL_trim (FOCAL); SEQ ID NO: 3386, 16q12.1_DLBCL_trim (FOCAL); SEQ ID NO: 3387, 16q12.1_DLBCL_trim (FOCAL); SEQ ID NO: 3388, 16q12.1_DLBCL_trim (FOCAL); SEQ ID NO: 3389, 16q12.1_DLBCL_trim (FOCAL); SEQ ID NO: 3390, 16q12.1_DLBCL_trim (FOCAL); SEQ ID NO: 3391, 16q12.1_DLBCL_trim (FOCAL); SEQ ID NO: 3392, 16q12.1_DLBCL_trim (FOCAL); SEQ ID NO: 3393, 16q12.1_DLBCL_trim (FOCAL); SEQ ID NO: 3394, 16q12.1_DLBCL_trim (FOCAL); SEQ ID NO: 3395, FP (FP); SEQ ID NO: 3396, 16q12.1_DLBCL_trim (FOCAL); SEQ ID NO: 3397, 16q12.1_DLBCL_trim (FOCAL); SEQ ID NO: 3398, 16q12.1_DLBCL_trim (FOCAL); SEQ ID NO: 3399, 16q12.1_DLBCL_trim (FOCAL); SEQ ID NO: 3400, 16q12.1_DLBCL_trim (FOCAL); SEQ ID NO: 3401, 16q12.1_DLBCL_trim (FOCAL); SEQ ID NO: 3402, 16q12.1_DLBCL_trim (FOCAL); SEQ ID NO: 3403, 16q12.1_DLBCL_trim (FOCAL); SEQ ID NO: 3404, 16q12.1_DLBCL_trim (FOCAL); SEQ ID NO: 3405, 16q12.1_DLBCL_trim (FOCAL); SEQ ID NO: 3406, 16q12.1_DLBCL_trim (FOCAL); SEQ ID NO: 3407, 16q12.1_DLBCL_trim (FOCAL); SEQ ID NO: 3408, 16q12.1_DLBCL_trim (FOCAL); SEQ ID NO: 3409, 16q12.1_DLBCL_trim (FOCAL); SEQ ID NO: 3410, 16q12.1_DLBCL_trim (FOCAL); SEQ ID NO: 3411, 16q12.1_DLBCL_trim (FOCAL); SEQ ID NO: 3412, 16q12.1_DLBCL_trim (FOCAL); SEQ ID NO: 3413, 16q12.1_DLBCL_trim (FOCAL); SEQ ID NO: 3414, 16q12.1_DLBCL_trim (FOCAL); SEQ ID NO: 3415, 16q12.1_DLBCL_trim (FOCAL); SEQ ID NO: 3416, 16q12.1_DLBCL_trim (FOCAL); SEQ ID NO: 3417, 16q12.1_DLBCL_trim (FOCAL); SEQ ID NO: 3418, 16q12.1_DLBCL_trim (FOCAL); SEQ ID NO: 3419, 16q12.1_DLBCL_trim (FOCAL); SEQ ID NO: 3420, 16q12.1_DLBCL_trim (FOCAL); SEQ ID NO: 3421, 16q12.1_DLBCL_trim (FOCAL); SEQ ID NO: 3422, 16q12.1_DLBCL_trim (FOCAL); SEQ ID NO: 3423, 16q12.1_DLBCL_trim (FOCAL); SEQ ID NO: 3424, 16q12.1_DLBCL_trim (FOCAL); SEQ ID NO: 3425, 16q12.1_DLBCL_trim (FOCAL); SEQ ID NO: 3426, 16q12.1_DLBCL_trim (FOCAL); SEQ ID NO: 3427, 16q12.1_DLBCL_trim (FOCAL); SEQ ID NO: 3428, 16q12.1_DLBCL_trim (FOCAL); SEQ ID NO: 3429, 16q12.1_DLBCL_trim (FOCAL); SEQ ID NO: 3430, 16q12.1_DLBCL_trim (FOCAL); SEQ ID NO: 3431, 16q12.1_DLBCL_trim (FOCAL); SEQ ID NO: 3432, 16q12.1_DLBCL_trim (FOCAL); SEQ ID NO: 3433, 16q12.1_DLBCL_trim (FOCAL); SEQ ID NO: 3434, 16q12.1_DLBCL_trim (FOCAL); SEQ ID NO: 3435, 16q12.1_DLBCL_trim (FOCAL); SEQ ID NO: 3436, 16q12.1_DLBCL_trim (FOCAL); SEQ ID NO: 3437, 16q12.1_DLBCL_trim (FOCAL); SEQ ID NO: 3438, 16q12.1_DLBCL_trim (FOCAL); SEQ ID NO: 3439, 16q12.1_DLBCL_trim (FOCAL); SEQ ID NO: 3440, 16q12.1_DLBCL_trim (FOCAL); SEQ ID NO: 3441, 16q12.1_DLBCL_trim (FOCAL); SEQ ID NO: 3442, 16q12.1_DLBCL_trim (FOCAL); SEQ ID NO: 3443, 16q12.1_DLBCL_trim (FOCAL); SEQ ID NO: 3444, 16q12.1_DLBCL_trim (FOCAL); SEQ ID NO: 3445, 16q12.1_DLBCL_trim (FOCAL); SEQ ID NO: 3446, 16q12.1_DLBCL_trim (FOCAL); SEQ ID NO: 3447, 16q12.1_DLBCL_trim (FOCAL); SEQ ID NO: 3448, 16q12.1_DLBCL_trim (FOCAL); SEQ ID NO: 3449, 16Q (ARM); SEQ ID NO: 3450, 16Q (ARM); SEQ ID NO: 3451, 16Q (ARM); SEQ ID NO: 3452, 16Q (ARM); SEQ ID NO: 3453, 16Q (ARM); SEQ ID NO: 3454, 16Q (ARM); SEQ ID NO: 3455, MSI (MSI); SEQ ID NO: 3456, MSI (MSI); SEQ ID NO: 3457, 16Q (ARM); SEQ ID NO: 3458, 16Q (ARM); SEQ ID NO: 3459, 16Q (ARM); SEQ ID NO: 3460, 16Q (ARM); SEQ ID NO: 3461, 16Q (ARM); SEQ ID NO: 3462, 16Q (ARM); SEQ ID NO: 3463, 16Q (ARM); SEQ ID NO: 3464, 16Q (ARM); SEQ ID NO: 3465, 16Q (ARM); SEQ ID NO: 3466, 16Q (ARM); SEQ ID NO: 3467, 16Q (ARM); SEQ ID NO: 3468, 16Q (ARM); SEQ ID NO: 3469, 16Q (ARM); SEQ ID NO: 3470, 16Q (ARM); SEQ ID NO: 3471, 16Q (ARM); SEQ ID NO: 3472, FP (FP); SEQ ID NO: 3473, 16Q (ARM); SEQ ID NO: 3474, 16Q (ARM); SEQ ID NO: 3475, PLCG2 (Gene); SEQ ID NO: 3476, PLCG2 (Gene); SEQ ID NO: 3477, PLCG2 (Gene); SEQ ID NO: 3478, PLCG2 (Gene); SEQ ID NO: 3479, PLCG2 (Gene); SEQ ID NO: 3480, PLCG2 (Gene); SEQ ID NO: 3481, PLCG2 (Gene); SEQ ID NO: 3482, PLCG2 (Gene); SEQ ID NO: 3483, PLCG2 (Gene); SEQ ID NO: 3484, PLCG2 (Gene); SEQ ID NO: 3485, PLCG2 (Gene); SEQ ID NO: 3486, PLCG2 (Gene); SEQ ID NO: 3487, PLCG2 (Gene); SEQ ID NO: 3488, PLCG2 (Gene); SEQ ID NO: 3489, PLCG2 (Gene); SEQ ID NO: 3490, PLCG2 (Gene); SEQ ID NO: 3491, PLCG2 (Gene); SEQ ID NO: 3492, PLCG2 (Gene); SEQ ID NO: 3493, PLCG2 (Gene); SEQ ID NO: 3494, PLCG2 (Gene); SEQ ID NO: 3495, PLCG2 (Gene); SEQ ID NO: 3496, 16Q (ARM); SEQ ID NO: 3497, PLCG2 (Gene); SEQ ID NO: 3498, PLCG2 (Gene); SEQ ID NO: 3499, PLCG2 (Gene); SEQ ID NO: 3500, PLCG2 (Gene); SEQ ID NO: 3501, PLCG2 (Gene); SEQ ID NO: 3502, PLCG2 (Gene); SEQ ID NO: 3503, PLCG2 (Gene); SEQ ID NO: 3504, PLCG2 (Gene); SEQ ID NO: 3505, PLCG2 (Gene); SEQ ID NO: 3506, PLCG2 (Gene); SEQ ID NO: 3507, PLCG2 (Gene); SEQ ID NO: 3508, 16Q (ARM); SEQ ID NO: 3509, 16Q (ARM); SEQ ID NO: 3510, 16Q (ARM); SEQ ID NO: 3511, 16Q (ARM); SEQ ID NO: 3512, 16Q (ARM); SEQ ID NO: 3513, 16Q (ARM); SEQ ID NO: 3514, MSI (MSI); SEQ ID NO: 3515, MSI (MSI); SEQ ID NO: 3516, IRF8 (Gene); SEQ ID NO: 3517, IRF8 (Gene); SEQ ID NO: 3518, IRF8 (Gene); SEQ ID NO: 3519, IRF8 (Gene); SEQ ID NO: 3520, IRF8 (Gene); SEQ ID NO: 3521, IRF8 (Gene); SEQ ID NO: 3522, IRF8 (Gene); SEQ ID NO: 3523, IRF8 (Gene); SEQ ID NO: 3524, IRF8 (Gene); SEQ ID NO: 3525, 16Q (ARM); SEQ ID NO: 3526, 16Q (ARM); SEQ ID NO: 3527, 16Q (ARM); SEQ ID NO: 3528, ANKRD11 (Gene); SEQ ID NO: 3529, ANKRD11 (Gene); SEQ ID NO: 3530, ANKRD11 (Gene); SEQ ID NO: 3531, ANKRD11 (Gene); SEQ ID NO: 3532, ANKRD11 (Gene); SEQ ID NO: 3533, ANKRD11 (Gene); SEQ ID NO: 3534, ANKRD11 (Gene); SEQ ID NO: 3535, ANKRD11 (Gene); SEQ ID NO: 3536, ANKRD11 (Gene); SEQ ID NO: 3537, ANKRD11 (Gene); SEQ ID NO: 3538, ANKRD11 (Gene); SEQ ID NO: 3539, ANKRD11 (Gene); SEQ ID NO: 3540, ANKRD11 (Gene); SEQ ID NO: 3541, ANKRD11 (Gene); SEQ ID NO: 3542, ANKRD11 (Gene); SEQ ID NO: 3543, ANKRD11 (Gene); SEQ ID NO: 3544, ANKRD11 (Gene); SEQ ID NO: 3545, ANKRD11 (Gene); SEQ ID NO: 3546, ANKRD11 (Gene); SEQ ID NO: 3547, ANKRD11 (Gene); SEQ ID NO: 3548, ANKRD11 (Gene); SEQ ID NO: 3549, ANKRD11 (Gene); SEQ ID NO: 3550, ANKRD11 (Gene); SEQ ID NO: 3551, ANKRD11 (Gene); SEQ ID NO: 3552, ANKRD11 (Gene); SEQ ID NO: 3553, 16Q (ARM); SEQ ID NO: 3554, 16Q (ARM); SEQ ID NO: 3555, 17p_CLL (TP53) (ARM); SEQ ID NO: 3556, 17p_CLL (TP53) (ARM); SEQ ID NO: 3557, 17p_CLL (TP53) (ARM); SEQ ID NO: 3558, 17p_CLL (TP53) (ARM); SEQ ID NO: 3559, 17p_CLL (TP53) (ARM); SEQ ID NO: 3560, FP (FP); SEQ ID NO: 3561, 17p_CLL (TP53) (ARM); SEQ ID NO: 3562, 17p_CLL (TP53) (ARM); SEQ ID NO: 3563, GPS2 (Gene); SEQ ID NO: 3564, GPS2 (Gene); SEQ ID NO: 3565, GPS2 (Gene); SEQ ID NO: 3566, GPS2 (Gene); SEQ ID NO: 3567, GPS2 (Gene); SEQ ID NO: 3568, GPS2 (Gene); SEQ ID NO: 3569, GPS2 (Gene); SEQ ID NO: 3570, GPS2 (Gene); SEQ ID NO: 3571, GPS2 (Gene); SEQ ID NO: 3572, GPS2 (Gene); SEQ ID NO: 3573, TP53 (Gene); SEQ ID NO: 3574, TP53 (Gene); SEQ ID NO: 3575, TP53 (Gene); SEQ ID NO: 3576, TP53 (Gene); SEQ ID NO: 3577, TP53 (Gene); SEQ ID NO: 3578, TP53 (Gene); SEQ ID NO: 3579, TP53 (Gene); SEQ ID NO: 3580, TP53 (Gene); SEQ ID NO: 3581, TP53 (Gene); SEQ ID NO: 3582, TP53 (Gene); SEQ ID NO: 3583, TP53 (Gene); SEQ ID NO: 3584, DNAH2 (Gene); SEQ ID NO: 3585, DNAH2 (Gene); SEQ ID NO: 3586, DNAH2 (Gene); SEQ ID NO: 3587, DNAH2 (Gene); SEQ ID NO: 3588, DNAH2 (Gene); SEQ ID NO: 3589, DNAH2 (Gene); SEQ ID NO: 3590, DNAH2 (Gene); SEQ ID NO: 3591, DNAH2 (Gene); SEQ ID NO: 3592, DNAH2 (Gene); SEQ ID NO: 3593, DNAH2 (Gene); SEQ ID NO: 3594, DNAH2 (Gene); SEQ ID NO: 3595, DNAH2 (Gene); SEQ ID NO: 3596, DNAH2 (Gene); SEQ ID NO: 3597, DNAH2 (Gene); SEQ ID NO: 3598, DNAH2 (Gene); SEQ ID NO: 3599, DNAH2 (Gene); SEQ ID NO: 3600, DNAH2 (Gene); SEQ ID NO: 3601, DNAH2 (Gene); SEQ ID NO: 3602, DNAH2 (Gene); SEQ ID NO: 3603, DNAH2 (Gene); SEQ ID NO: 3604, DNAH2 (Gene); SEQ ID NO: 3605, DNAH2 (Gene); SEQ ID NO: 3606, DNAH2 (Gene); SEQ ID NO: 3607, DNAH2 (Gene); SEQ ID NO: 3608, DNAH2 (Gene); SEQ ID NO: 3609, DNAH2 (Gene); SEQ ID NO: 3610, DNAH2 (Gene); SEQ ID NO: 3611, DNAH2 (Gene); SEQ ID NO: 3612, DNAH2 (Gene); SEQ ID NO: 3613, DNAH2 (Gene); SEQ ID NO: 3614, DNAH2 (Gene); SEQ ID NO: 3615, DNAH2 (Gene); SEQ ID NO: 3616, DNAH2 (Gene); SEQ ID NO: 3617, DNAH2 (Gene); SEQ ID NO: 3618, DNAH2 (Gene); SEQ ID NO: 3619, DNAH2 (Gene); SEQ ID NO: 3620, DNAH2 (Gene); SEQ ID NO: 3621, DNAH2 (Gene); SEQ ID NO: 3622, DNAH2 (Gene); SEQ ID NO: 3623, DNAH2 (Gene); SEQ ID NO: 3624, DNAH2 (Gene); SEQ ID NO: 3625, DNAH2 (Gene); SEQ ID NO: 3626, DNAH2 (Gene); SEQ ID NO: 3627, DNAH2 (Gene); SEQ ID NO: 3628, DNAH2 (Gene); SEQ ID NO: 3629, DNAH2 (Gene); SEQ ID NO: 3630, DNAH2 (Gene); SEQ ID NO: 3631, DNAH2 (Gene); SEQ ID NO: 3632, DNAH2 (Gene); SEQ ID NO: 3633, DNAH2 (Gene); SEQ ID NO: 3634, DNAH2 (Gene); SEQ ID NO: 3635, DNAH2 (Gene); SEQ ID NO: 3636, DNAH2 (Gene); SEQ ID NO: 3637, DNAH2 (Gene); SEQ ID NO: 3638, DNAH2 (Gene); SEQ ID NO: 3639, DNAH2 (Gene); SEQ ID NO: 3640, DNAH2 (Gene); SEQ ID NO: 3641, DNAH2 (Gene); SEQ ID NO: 3642, DNAH2 (Gene); SEQ ID NO: 3643, DNAH2 (Gene); SEQ ID NO: 3644, DNAH2 (Gene); SEQ ID NO: 3645, DNAH2 (Gene); SEQ ID NO: 3646, DNAH2 (Gene); SEQ ID NO: 3647, DNAH2 (Gene); SEQ ID NO: 3648, DNAH2 (Gene); SEQ ID NO: 3649, DNAH2 (Gene); SEQ ID NO: 3650, DNAH2 (Gene); SEQ ID NO: 3651, DNAH2 (Gene); SEQ ID NO: 3652, DNAH2 (Gene); SEQ ID NO: 3653, DNAH2 (Gene); SEQ ID NO: 3654, DNAH2 (Gene); SEQ ID NO: 3655, DNAH2 (Gene); SEQ ID NO: 3656, DNAH2 (Gene); SEQ ID NO: 3657, DNAH2 (Gene); SEQ ID NO: 3658, DNAH2 (Gene); SEQ ID NO: 3659, DNAH2 (Gene); SEQ ID NO: 3660, DNAH2 (Gene); SEQ ID NO: 3661, DNAH2 (Gene); SEQ ID NO: 3662, DNAH2 (Gene); SEQ ID NO: 3663, DNAH2 (Gene); SEQ ID NO: 3664, DNAH2 (Gene); SEQ ID NO: 3665, DNAH2 (Gene); SEQ ID NO: 3666, DNAH2 (Gene); SEQ ID NO: 3667, DNAH2 (Gene); SEQ ID NO: 3668, DNAH2 (Gene); SEQ ID NO: 3669, DNAH2 (Gene); SEQ ID NO: 3670, DNAH2 (Gene); SEQ ID NO: 3671, MSI (MSI); SEQ ID NO: 3672, MSI (MSI); SEQ ID NO: 3673, 17p_CLL (TP53) (ARM); SEQ ID NO: 3674, 17p_CLL (TP53) (ARM); SEQ ID NO: 3675, 17p_CLL (TP53) (ARM); SEQ ID NO: 3676, 17p_CLL (TP53) (ARM); SEQ ID NO: 3677, FP (FP); SEQ ID NO: 3678, 17p_CLL (TP53) (ARM); SEQ ID NO: 3679, 17p_CLL (TP53) (ARM); SEQ ID NO: 3680, 17p_CLL (TP53) (ARM); SEQ ID NO: 3681, 17p_CLL (TP53) (ARM); SEQ ID NO: 3682, 17p_CLL (TP53) (ARM); SEQ ID NO: 3683, NCOR1 (Gene); SEQ ID NO: 3684, NCOR1 (Gene); SEQ ID NO: 3685, NCOR1 (Gene); SEQ ID NO: 3686, NCOR1 (Gene); SEQ ID NO: 3687, NCOR1 (Gene); SEQ ID NO: 3688, NCOR1 (Gene); SEQ ID NO: 3689, NCOR1 (Gene); SEQ ID NO: 3690, NCOR1 (Gene); SEQ ID NO: 3691, NCOR1 (Gene); SEQ ID NO: 3692, NCOR1 (Gene); SEQ ID NO: 3693, NCOR1 (Gene); SEQ ID NO: 3694, NCOR1 (Gene); SEQ ID NO: 3695, NCOR1 (Gene); SEQ ID NO: 3696, NCOR1 (Gene); SEQ ID NO: 3697, NCOR1 (Gene); SEQ ID NO: 3698, NCOR1 (Gene); SEQ ID NO: 3699, NCOR1 (Gene); SEQ ID NO: 3700, NCOR1 (Gene); SEQ ID NO: 3701, NCOR1 (Gene); SEQ ID NO: 3702, NCOR1 (Gene); SEQ ID NO: 3703, NCOR1 (Gene); SEQ ID NO: 3704, NCOR1 (Gene); SEQ ID NO: 3705, NCOR1 (Gene); SEQ ID NO: 3706, NCOR1 (Gene); SEQ ID NO: 3707, NCOR1 (Gene); SEQ ID NO: 3708, NCOR1 (Gene); SEQ ID NO: 3709, NCOR1 (Gene); SEQ ID NO: 3710, NCOR1 (Gene); SEQ ID NO: 3711, NCOR1 (Gene); SEQ ID NO: 3712, NCOR1 (Gene); SEQ ID NO: 3713, NCOR1 (Gene); SEQ ID NO: 3714, NCOR1 (Gene); SEQ ID NO: 3715, NCOR1 (Gene); SEQ ID NO: 3716, NCOR1 (Gene); SEQ ID NO: 3717, NCOR1 (Gene); SEQ ID NO: 3718, NCOR1 (Gene); SEQ ID NO: 3719, NCOR1 (Gene); SEQ ID NO: 3720, NCOR1 (Gene); SEQ ID NO: 3721, NCOR1 (Gene); SEQ ID NO: 3722, NCOR1 (Gene); SEQ ID NO: 3723, NCOR1 (Gene); SEQ ID NO: 3724, NCOR1 (Gene); SEQ ID NO: 3725, NCOR1 (Gene); SEQ ID NO: 3726, NCOR1 (Gene); SEQ ID NO: 3727, NCOR1 (Gene); SEQ ID NO: 3728, NCOR1 (Gene); SEQ ID NO: 3729, NCOR1 (Gene); SEQ ID NO: 3730, NCOR1 (Gene); SEQ ID NO: 3731, 17p_CLL (TP53) (ARM); SEQ ID NO: 3732, 17p_CLL (TP53) (ARM); SEQ ID NO: 3733, 17p_CLL (TP53) (ARM); SEQ ID NO: 3734, FP (FP); SEQ ID NO: 3735, 17Q (ARM); SEQ ID NO: 3736, 17Q (ARM); SEQ ID NO: 3737, 17Q (ARM); SEQ ID NO: 3738, 17Q (ARM); SEQ ID NO: 3739, 17Q (ARM); SEQ ID NO: 3740, 17Q (ARM); SEQ ID NO: 3741, 17Q (ARM); SEQ ID NO: 3742, CCL4 (Gene); SEQ ID NO: 3743, CCL4 (Gene); SEQ ID NO: 3744, CCL4 (Gene); SEQ ID NO: 3745, 17Q (ARM); SEQ ID NO: 3746, 17Q (ARM); SEQ ID NO: 3747, IKZF3 (Gene); SEQ ID NO: 3748, IKZF3 (Gene); SEQ ID NO: 3749, IKZF3 (Gene); SEQ ID NO: 3750, IKZF3 (Gene); SEQ ID NO: 3751, IKZF3 (Gene); SEQ ID NO: 3752, IKZF3 (Gene); SEQ ID NO: 3753, IKZF3 (Gene); SEQ ID NO: 3754, IKZF3 (Gene); SEQ ID NO: 3755, IKZF3 (Gene); SEQ ID NO: 3756, 17Q (ARM); SEQ ID NO: 3757, MSI (MSI); SEQ ID NO: 3758, MSI (MSI); SEQ ID NO: 3759, FP (FP); SEQ ID NO: 3760, STAT5B (Gene); SEQ ID NO: 3761, STAT5B (Gene); SEQ ID NO: 3762, STAT5B (Gene); SEQ ID NO: 3763, STAT5B (Gene); SEQ ID NO: 3764, STAT5B (Gene); SEQ ID NO: 3765, STAT5B (Gene); SEQ ID NO: 3766, STAT5B (Gene); SEQ ID NO: 3767, STAT5B (Gene); SEQ ID NO: 3768, STAT5B (Gene); SEQ ID NO: 3769, STAT5B (Gene); SEQ ID NO: 3770, STAT5B (Gene); SEQ ID NO: 3771, STAT5B (Gene); SEQ ID NO: 3772, STAT5B (Gene); SEQ ID NO: 3773, STAT5B (Gene); SEQ ID NO: 3774, STAT5B (Gene); SEQ ID NO: 3775, STAT3 (Gene); SEQ ID NO: 3776, STAT3 (Gene); SEQ ID NO: 3777, STAT3 (Gene); SEQ ID NO: 3778, STAT3 (Gene); SEQ ID NO: 3779, STAT3 (Gene); SEQ ID NO: 3780, STAT3 (Gene); SEQ ID NO: 3781, STAT3 (Gene); SEQ ID NO: 3782, STAT3 (Gene); SEQ ID NO: 3783, STAT3 (Gene); SEQ ID NO: 3784, STAT3 (Gene); SEQ ID NO: 3785, STAT3 (Gene); SEQ ID NO: 3786, STAT3 (Gene); SEQ ID NO: 3787, STAT3 (Gene); SEQ ID NO: 3788, STAT3 (Gene); SEQ ID NO: 3789, STAT3 (Gene); SEQ ID NO: 3790, STAT3 (Gene); SEQ ID NO: 3791, STAT3 (Gene); SEQ ID NO: 3792, STAT3 (Gene); SEQ ID NO: 3793, STAT3 (Gene); SEQ ID NO: 3794, STAT3 (Gene); SEQ ID NO: 3795, STAT3 (Gene); SEQ ID NO: 3796, STAT3 (Gene); SEQ ID NO: 3797, STAT3 (Gene); SEQ ID NO: 3798, 17Q (ARM); SEQ ID NO: 3799, 17Q (ARM); SEQ ID NO: 3800, BRCA1 (Gene); SEQ ID NO: 3801, BRCA1 (Gene); SEQ ID NO: 3802, BRCA1 (Gene); SEQ ID NO: 3803, BRCA1 (Gene); SEQ ID NO: 3804, BRCA1 (Gene); SEQ ID NO: 3805, BRCA1 (Gene); SEQ ID NO: 3806, BRCA1 (Gene); SEQ ID NO: 3807, BRCA1 (Gene); SEQ ID NO: 3808, BRCA1 (Gene); SEQ ID NO: 3809, BRCA1 (Gene); SEQ ID NO: 3810, BRCA1 (Gene); SEQ ID NO: 3811, BRCA1 (Gene); SEQ ID NO: 3812, BRCA1 (Gene); SEQ ID NO: 3813, BRCA1 (Gene); SEQ ID NO: 3814, BRCA1 (Gene); SEQ ID NO: 3815, BRCA1 (Gene); SEQ ID NO: 3816, BRCA1 (Gene); SEQ ID NO: 3817, BRCA1 (Gene); SEQ ID NO: 3818, BRCA1 (Gene); SEQ ID NO: 3819, BRCA1 (Gene); SEQ ID NO: 3820, BRCA1 (Gene); SEQ ID NO: 3821, BRCA1 (Gene); SEQ ID NO: 3822, BRCA1 (Gene); SEQ ID NO: 3823, BRCA1 (Gene); SEQ ID NO: 3824, BRCA1 (Gene); SEQ ID NO: 3825, BRCA1 (Gene); SEQ ID NO: 3826, BRCA1 (Gene); SEQ ID NO: 3827, BRCA1 (Gene); SEQ ID NO: 3828, BRCA1 (Gene); SEQ ID NO: 3829, BRCA1 (Gene); SEQ ID NO: 3830, MSI (MSI); SEQ ID NO: 3831, MSI (MSI); SEQ ID NO: 3832, 17Q (ARM); SEQ ID NO: 3833, FP (FP); SEQ ID NO: 3834, 17Q (ARM); SEQ ID NO: 3835, 17Q (ARM); SEQ ID NO: 3836, MSI (MSI); SEQ ID NO: 3837, MSI (MSI); SEQ ID NO: 3838, 17Q (ARM); SEQ ID NO: 3839, 17Q (ARM); SEQ ID NO: 3840, FP (FP); SEQ ID NO: 3841, 17Q (ARM); SEQ ID NO: 3842, 17Q (ARM); SEQ ID NO: 3843, 17Q (ARM); SEQ ID NO: 3844, 17Q (ARM); SEQ ID NO: 3845, 17Q (ARM); SEQ ID NO: 3846, 17Q (ARM); SEQ ID NO: 3847, 17Q (ARM); SEQ ID NO: 3848, MSI (MSI); SEQ ID NO: 3849, MSI (MSI); SEQ ID NO: 3850, 17Q (ARM); SEQ ID NO: 3851, VMP1 (Gene); SEQ ID NO: 3852, VMP1 (Gene); SEQ ID NO: 3853, VMP1 (Gene); SEQ ID NO: 3854, VMP1 (Gene); SEQ ID NO: 3855, VMP1 (Gene); SEQ ID NO: 3856, VMP1 (Gene); SEQ ID NO: 3857, VMP1 (Gene); SEQ ID NO: 3858, VMP1 (Gene); SEQ ID NO: 3859, VMP1 (Gene); SEQ ID NO: 3860, VMP1 (Gene); SEQ ID NO: 3861, VMP1 (Gene); SEQ ID NO: 3862, PPM1D (Gene); SEQ ID NO: 3863, PPM1D (Gene); SEQ ID NO: 3864, PPM1D (Gene); SEQ ID NO: 3865, PPM1D (Gene); SEQ ID NO: 3866, PPM1D (Gene); SEQ ID NO: 3867, PPM1D (Gene); SEQ ID NO: 3868, PPM1D (Gene); SEQ ID NO: 3869, PPM1D (Gene); SEQ ID NO: 3870, 17Q (ARM); SEQ ID NO: 3871, 17Q (ARM); SEQ ID NO: 3872, CD79B (Gene); SEQ ID NO: 3873, CD79B (Gene); SEQ ID NO: 3874, CD79B (Gene); SEQ ID NO: 3875, CD79B (Gene); SEQ ID NO: 3876, CD79B (Gene); SEQ ID NO: 3877, CD79B (Gene); SEQ ID NO: 3878, 17Q (ARM); SEQ ID NO: 3879, GNA13 (Gene); SEQ ID NO: 3880, GNA13 (Gene); SEQ ID NO: 3881, GNA13 (Gene); SEQ ID NO: 3882, GNA13 (Gene); SEQ ID NO: 3883, GNA13 (Gene); SEQ ID NO: 3884, 17Q (ARM); SEQ ID NO: 3885, 17Q (ARM); SEQ ID NO: 3886, 17Q (ARM); SEQ ID NO: 3887, 17q24.3_DLBCL (FOCAL); SEQ ID NO: 3888, 17q24.3_DLBCL (FOCAL); SEQ ID NO: 3889, 17q24.3_DLBCL (FOCAL); SEQ ID NO: 3890, 17q24.3 DLBCL (FOCAL); SEQ ID NO: 3891, 17q24.3_DLBCL (FOCAL); SEQ ID NO: 3892, 17q24.3_DLBCL (FOCAL); SEQ ID NO: 3893, 17q24.3_DLBCL (FOCAL); SEQ ID NO: 3894, 17q24.3_DLBCL (FOCAL); SEQ ID NO: 3895, 17q24.3_DLBCL (FOCAL); SEQ ID NO: 3896, 17q24.3_DLBCL (FOCAL); SEQ ID NO: 3897, 17Q (ARM); SEQ ID NO: 3898, 17Q (ARM); SEQ ID NO: 3899, 17Q (ARM); SEQ ID NO: 3900, 17Q (ARM); SEQ ID NO: 3901, 17Q (ARM); SEQ ID NO: 3902, 17q25.1_DLBCL (FOCAL); SEQ ID NO: 3903, 17q25.1_DLBCL (FOCAL); SEQ ID NO: 3904, 17q25.1_DLBCL (FOCAL); SEQ ID NO: 3905, 17q25.1_DLBCL (FOCAL); SEQ ID NO: 3906, 17q25.1_DLBCL (FOCAL); SEQ ID NO: 3907, GRB2 (Gene); SEQ ID NO: 3908, GRB2 (Gene); SEQ ID NO: 3909, GRB2 (Gene); SEQ ID NO: 3910, GRB2 (Gene); SEQ ID NO: 3911, 17q25.1_DLBCL (FOCAL); SEQ ID NO: 3912, GRB2 (Gene); SEQ ID NO: 3913, 17q25.1_DLBCL (FOCAL); SEQ ID NO: 3914, 17q25.1_DLBCL (FOCAL); SEQ ID NO: 3915, 17q25.1_DLBCL (FOCAL); SEQ ID NO: 3916, 17Q (ARM); SEQ ID NO: 3917, 17Q (ARM); SEQ ID NO: 3918, 17Q (ARM); SEQ ID NO: 3919, 17Q (ARM); SEQ ID NO: 3920, 17Q (ARM); SEQ ID NO: 3921, 17Q (ARM); SEQ ID NO: 3922, 17Q (ARM); SEQ ID NO: 3923, 18P_DLBCL (ARM); SEQ ID NO: 3924, FP (FP); SEQ ID NO: 3925, 18P_DLBCL (ARM); SEQ ID NO: 3926, 18P_DLBCL (ARM); SEQ ID NO: 3927, 18P_DLBCL (ARM); SEQ ID NO: 3928, 18P_DLBCL (ARM); SEQ ID NO: 3929, FP (FP); SEQ ID NO: 3930, 18P_DLBCL (ARM); SEQ ID NO: 3931, 18P_DLBCL (ARM); SEQ ID NO: 3932, 18P_DLBCL (ARM); SEQ ID NO: 3933, 18P_DLBCL (ARM); SEQ ID NO: 3934, 18P_DLBCL (ARM); SEQ ID NO: 3935, 18P_DLBCL (ARM); SEQ ID NO: 3936, 18P_DLBCL (ARM); SEQ ID NO: 3937, 18P_DLBCL (ARM); SEQ ID NO: 3938, 18P_DLBCL (ARM); SEQ ID NO: 3939, 18P_DLBCL (ARM); SEQ ID NO: 3940, 18Q_DLBCL (ARM); SEQ ID NO: 3941, MSI (MSI); SEQ ID NO: 3942, MSI (MSI); SEQ ID NO: 3943, 18Q_DLBCL (ARM); SEQ ID NO: 3944, 18Q_DLBCL (ARM); SEQ ID NO: 3945, 18Q_DLBCL (ARM); SEQ ID NO: 3946, 18Q_DLBCL (ARM); SEQ ID NO: 3947, 18Q_DLBCL (ARM); SEQ ID NO: 3948, 18Q_DLBCL (ARM); SEQ ID NO: 3949, 18Q_DLBCL (ARM); SEQ ID NO: 3950, 18Q_DLBCL (ARM); SEQ ID NO: 3951, 18Q_DLBCL (ARM); SEQ ID NO: 3952, KLHL14 (Gene); SEQ ID NO: 3953, KLHL14 (Gene); SEQ ID NO: 3954, KLHL14 (Gene); SEQ ID NO: 3955, KLHL14 (Gene); SEQ ID NO: 3956, KLHL14 (Gene); SEQ ID NO: 3957, KLHL14 (Gene); SEQ ID NO: 3958, KLHL14 (Gene); SEQ ID NO: 3959, KLHL14 (Gene); SEQ ID NO: 3960, KLHL14 (Gene); SEQ ID NO: 3961, 18Q_DLBCL (ARM); SEQ ID NO: 3962, 18Q_DLBCL (ARM); SEQ ID NO: 3963, 18Q_DLBCL (ARM); SEQ ID NO: 3964, 18Q_DLBCL (ARM); SEQ ID NO: 3965, 18Q_DLBCL (ARM); SEQ ID NO: 3966, 18Q_DLBCL (ARM); SEQ ID NO: 3967, 18Q_DLBCL (ARM); SEQ ID NO: 3968, 18Q_DLBCL (ARM); SEQ ID NO: 3969, 18Q_DLBCL (ARM); SEQ ID NO: 3970, 18Q_DLBCL (ARM); SEQ ID NO: 3971, 18Q_DLBCL (ARM); SEQ ID NO: 3972, 18Q_DLBCL (ARM); SEQ ID NO: 3973, 18Q_DLBCL (ARM); SEQ ID NO: 3974, 18Q_DLBCL (ARM); SEQ ID NO: 3975, 18Q_DLBCL (ARM); SEQ ID NO: 3976, 18Q_DLBCL (ARM); SEQ ID NO: 3977, 18Q_DLBCL (ARM); SEQ ID NO: 3978, 18Q_DLBCL (ARM); SEQ ID NO: 3979, 18Q_DLBCL (ARM); SEQ ID NO: 3980, 18Q_DLBCL (ARM); SEQ ID NO: 3981, 18Q_DLBCL (ARM); SEQ ID NO: 3982, 18q22.2_DLBCL (FOCAL); SEQ ID NO: 3983, 18q22.2_DLBCL (FOCAL); SEQ ID NO: 3984, 18q22.2_DLBCL (FOCAL); SEQ ID NO: 3985, 18q22.2_DLBCL (FOCAL); SEQ ID NO: 3986, 18q22.2 DLBCL (FOCAL); SEQ ID NO: 3987, 18q22.2_DLBCL (FOCAL); SEQ ID NO: 3988, 18q22.2_DLBCL (FOCAL); SEQ ID NO: 3989, 18q22.2_DLBCL (FOCAL); SEQ ID NO: 3990, 18q22.2_DLBCL (FOCAL); SEQ ID NO: 3991, 18q22.2_DLBCL (FOCAL); SEQ ID NO: 3992, 18q22.2_DLBCL (FOCAL); SEQ ID NO: 3993, 18q22.2_DLBCL (FOCAL); SEQ ID NO: 3994, 18q22.2_DLBCL (FOCAL); SEQ ID NO: 3995, 18q22.2_DLBCL (FOCAL); SEQ ID NO: 3996, 18q22.2_DLBCL (FOCAL); SEQ ID NO: 3997, 18q22.2 DLBCL (FOCAL); SEQ ID NO: 3998, 18q22.2_DLBCL (FOCAL); SEQ ID NO: 3999, 18q22.2_DLBCL (FOCAL); SEQ ID NO: 4000, MALT1 (Gene); SEQ ID NO: 4001, MALT1 (Gene); SEQ ID NO: 4002, MALT1 (Gene); SEQ ID NO: 4003, MALT1 (Gene); SEQ ID NO: 4004, MALT1 (Gene); SEQ ID NO: 4005, MALT1 (Gene); SEQ ID NO: 4006, MALT1 (Gene); SEQ ID NO: 4007, MALT1 (Gene); SEQ ID NO: 4008, MALT1 (Gene); SEQ ID NO: 4009, MALT1 (Gene); SEQ ID NO: 4010, MALT1 (Gene); SEQ ID NO: 4011, MALT1 (Gene); SEQ ID NO: 4012, MALT1 (Gene); SEQ ID NO: 4013, MALT1 (Gene); SEQ ID NO: 4014, MALT1 (Gene); SEQ ID NO: 4015, MALT1 (Gene); SEQ ID NO: 4016, MALT1 (Gene); SEQ ID NO: 4017, 18q22.2_DLBCL (FOCAL); SEQ ID NO: 4018, MSI (MSI); SEQ ID NO: 4019, MSI (MSI); SEQ ID NO: 4020, 18q22.2_DLBCL (FOCAL); SEQ ID NO: 4021, 18q22.2_DLBCL (FOCAL); SEQ ID NO: 4022, 18q22.2_DLBCL (FOCAL); SEQ ID NO: 4023, 18q22.2 DLBCL (FOCAL); SEQ ID NO: 4024, 18q22.2_DLBCL (FOCAL); SEQ ID NO: 4025, 18q22.2_DLBCL (FOCAL); SEQ ID NO: 4026, 18q22.2_DLBCL (FOCAL); SEQ ID NO: 4027, 18q22.2_DLBCL (FOCAL); SEQ ID NO: 4028, 18q22.2_DLBCL (FOCAL); SEQ ID NO: 4029, 18q22.2_DLBCL (FOCAL); SEQ ID NO: 4030, 18q22.2_DLBCL (FOCAL); SEQ ID NO: 4031, 18q22.2_DLBCL (FOCAL); SEQ ID NO: 4032, 18q22.2_DLBCL (FOCAL); SEQ ID NO: 4033, 18q22.2_DLBCL (FOCAL); SEQ ID NO: 4034, 18q22.2 DLBCL (FOCAL); SEQ ID NO: 4035, 18q22.2_DLBCL (FOCAL); SEQ ID NO: 4036, 18q22.2_DLBCL (FOCAL); SEQ ID NO: 4037, BCL2_SV (SV); SEQ ID NO: 4038, BCL2_SV (SV); SEQ ID NO: 4039, BCL2_SV (SV); SEQ ID NO: 4040, BCL2_SV (SV); SEQ ID NO: 4041, BCL2_SV (SV); SEQ ID NO: 4042, BCL2_SV (SV); SEQ ID NO: 4043, BCL2_SV (SV); SEQ ID NO: 4044, BCL2_SV (SV); SEQ ID NO: 4045, BCL2_SV (SV); SEQ ID NO: 4046, BCL2_SV (SV); SEQ ID NO: 4047, BCL2_SV (SV); SEQ ID NO: 4048, BCL2_SV (SV); SEQ ID NO: 4049, BCL2_SV (SV); SEQ ID NO: 4050, BCL2_SV (SV); SEQ ID NO: 4051, BCL2_SV (SV); SEQ ID NO: 4052, BCL2_SV (SV); SEQ ID NO: 4053, BCL2_SV (SV); SEQ ID NO: 4054, BCL2_SV (SV); SEQ ID NO: 4055, BCL2_SV (SV); SEQ ID NO: 4056, BCL2_SV (SV); SEQ ID NO: 4057, BCL2_SV (SV); SEQ ID NO: 4058, BCL2_SV (SV); SEQ ID NO: 4059, BCL2_SV (SV); SEQ ID NO: 4060, BCL2_SV (SV); SEQ ID NO: 4061, BCL2_SV (SV); SEQ ID NO: 4062, BCL2_SV (SV); SEQ ID NO: 4063, BCL2_SV (SV); SEQ ID NO: 4064, BCL2_SV (SV); SEQ ID NO: 4065, BCL2_SV (SV); SEQ ID NO: 4066, BCL2_SV (SV); SEQ ID NO: 4067, BCL2_SV (SV); SEQ ID NO: 4068, BCL2_SV (SV); SEQ ID NO: 4069, BCL2_SV (SV); SEQ ID NO: 4070, BCL2_SV (SV); SEQ ID NO: 4071, BCL2_SV (SV); SEQ ID NO: 4072, BCL2_SV (SV); SEQ ID NO: 4073, BCL2_SV (SV); SEQ ID NO: 4074, BCL2_SV (SV); SEQ ID NO: 4075, BCL2_SV (SV); SEQ ID NO: 4076, BCL2_SV (SV); SEQ ID NO: 4077, BCL2_SV (SV); SEQ ID NO: 4078, BCL2_SV (SV); SEQ ID NO: 4079, BCL2_SV (SV); SEQ ID NO: 4080, BCL2_SV (SV); SEQ ID NO: 4081, BCL2_SV (SV); SEQ ID NO: 4082, BCL2_SV (SV); SEQ ID NO: 4083, BCL2_SV (SV); SEQ ID NO: 4084, BCL2_SV (SV); SEQ ID NO: 4085, BCL2_SV (SV); SEQ ID NO: 4086, BCL2_SV (SV); SEQ ID NO: 4087, BCL2_SV (SV); SEQ ID NO: 4088, BCL2_SV (SV); SEQ ID NO: 4089, BCL2_SV (SV); SEQ ID NO: 4090, BCL2_SV (SV); SEQ ID NO: 4091, BCL2_SV (SV); SEQ ID NO: 4092, BCL2_SV (SV); SEQ ID NO: 4093, BCL2_SV (SV); SEQ ID NO: 4094, BCL2_SV (SV); SEQ ID NO: 4095, BCL2_SV (SV); SEQ ID NO: 4096, BCL2_SV (SV); SEQ ID NO: 4097, BCL2_SV (SV); SEQ ID NO: 4098, BCL2_SV (SV); SEQ ID NO: 4099, BCL2_SV (SV); SEQ ID NO: 4100, BCL2_SV (SV); SEQ ID NO: 4101, BCL2_SV (SV); SEQ ID NO: 4102, BCL2_SV (SV); SEQ ID NO: 4103, BCL2_SV (SV); SEQ ID NO: 4104, BCL2_SV (SV); SEQ ID NO: 4105, BCL2_SV (SV); SEQ ID NO: 4106, BCL2_SV (SV); SEQ ID NO: 4107, BCL2_SV (SV); SEQ ID NO: 4108, BCL2_SV (SV); SEQ ID NO: 4109, BCL2_SV (SV); SEQ ID NO: 4110, BCL2_SV (SV); SEQ ID NO: 4111, BCL2_SV (SV); SEQ ID NO: 4112, BCL2_SV (SV); SEQ ID NO: 4113, BCL2_SV (SV); SEQ ID NO: 4114, BCL2_SV (SV); SEQ ID NO: 4115, BCL2_SV (SV); SEQ ID NO: 4116, BCL2_SV (SV); SEQ ID NO: 4117, BCL2_SV (SV); SEQ ID NO: 4118, BCL2_SV (SV); SEQ ID NO: 4119, BCL2_SV (SV); SEQ ID NO: 4120, BCL2_SV (SV); SEQ ID NO: 4121, BCL2_SV (SV); SEQ ID NO: 4122, BCL2_SV (SV); SEQ ID NO: 4123, BCL2_SV (SV); SEQ ID NO: 4124, BCL2_SV (SV); SEQ ID NO: 4125, BCL2_SV (SV); SEQ ID NO: 4126, BCL2_SV (SV); SEQ ID NO: 4127, BCL2_SV (SV); SEQ ID NO: 4128, BCL2_SV (SV); SEQ ID NO: 4129, BCL2_SV (SV); SEQ ID NO: 4130, BCL2_SV (SV); SEQ ID NO: 4131, BCL2_SV (SV); SEQ ID NO: 4132, BCL2_SV (SV); SEQ ID NO: 4133, BCL2_SV (SV); SEQ ID NO: 4134, BCL2_SV (SV); SEQ ID NO: 4135, BCL2_SV (SV); SEQ ID NO: 4136, BCL2_SV (SV); SEQ ID NO: 4137, 18q22.2_DLBCL (FOCAL); SEQ ID NO: 4138, 18q22.2_DLBCL (FOCAL); SEQ ID NO: 4139, 18q22.2_DLBCL (FOCAL); SEQ ID NO: 4140, 18q22.2_DLBCL (FOCAL); SEQ ID NO: 4141, 18q22.2_DLBCL (FOCAL); SEQ ID NO: 4142, 18q22.2_DLBCL (FOCAL); SEQ ID NO: 4143, 18q22.2_DLBCL (FOCAL); SEQ ID NO: 4144, 18q22.2_DLBCL (FOCAL); SEQ ID NO: 4145, 18q22.2 DLBCL (FOCAL); SEQ ID NO: 4146, 18q22.2_DLBCL (FOCAL); SEQ ID NO: 4147, 18q22.2_DLBCL (FOCAL); SEQ ID NO: 4148, 18q22.2_DLBCL (FOCAL); SEQ ID NO: 4149, 18q22.2_DLBCL (FOCAL); SEQ ID NO: 4150, 18q22.2_DLBCL (FOCAL); SEQ ID NO: 4151, 18q22.2_DLBCL (FOCAL); SEQ ID NO: 4152, 18q22.2_DLBCL (FOCAL); SEQ ID NO: 4153, 18q22.2_DLBCL (FOCAL); SEQ ID NO: 4154, 18q22.2_DLBCL (FOCAL); SEQ ID NO: 4155, 18q22.2_DLBCL (FOCAL); SEQ ID NO: 4156, 18q22.2 DLBCL (FOCAL); SEQ ID NO: 4157, 18q22.2_DLBCL (FOCAL); SEQ ID NO: 4158, 18q22.2_DLBCL (FOCAL); SEQ ID NO: 4159, 18q22.2_DLBCL (FOCAL); SEQ ID NO: 4160, 18q22.2_DLBCL (FOCAL); SEQ ID NO: 4161, 18q22.2_DLBCL (FOCAL); SEQ ID NO: 4162, 18q22.2_DLBCL (FOCAL); SEQ ID NO: 4163, 18q22.2_DLBCL (FOCAL); SEQ ID NO: 4164, 18q22.2_DLBCL (FOCAL); SEQ ID NO: 4165, 18q22.2_DLBCL (FOCAL); SEQ ID NO: 4166, 18q22.2_DLBCL (FOCAL); SEQ ID NO: 4167, 18q22.2 DLBCL (FOCAL); SEQ ID NO: 4168, 18q22.2_DLBCL (FOCAL); SEQ ID NO: 4169, 18q22.2_DLBCL (FOCAL); SEQ ID NO: 4170, 18q22.2_DLBCL (FOCAL); SEQ ID NO: 4171, SOCS6 (Gene); SEQ ID NO: 4172, SOCS6 (Gene); SEQ ID NO: 4173, SOCS6 (Gene); SEQ ID NO: 4174, SOCS6 (Gene); SEQ ID NO: 4175, 18q22.2_DLBCL (FOCAL); SEQ ID NO: 4176, 18q22.2_DLBCL (FOCAL); SEQ ID NO: 4177, 18q22.2_DLBCL (FOCAL); SEQ ID NO: 4178, 18q22.2_DLBCL (FOCAL); SEQ ID NO: 4179, 18q22.2_DLBCL (FOCAL); SEQ ID NO: 4180, 18q22.2_DLBCL (FOCAL); SEQ ID NO: 4181, 18q22.2 DLBCL (FOCAL); SEQ ID NO: 4182, 18q22.2_DLBCL (FOCAL); SEQ ID NO: 4183, 18q22.2_DLBCL (FOCAL); SEQ ID NO: 4184, 18q22.2_DLBCL (FOCAL); SEQ ID NO: 4185, 18q22.2_DLBCL (FOCAL); SEQ ID NO: 4186, 18q22.2_DLBCL (FOCAL); SEQ ID NO: 4187, 18q22.2_DLBCL (FOCAL); SEQ ID NO: 4188, 18q22.2_DLBCL (FOCAL); SEQ ID NO: 4189, 18q22.2_DLBCL (FOCAL); SEQ ID NO: 4190, 18q22.2_DLBCL (FOCAL); SEQ ID NO: 4191, 18q22.2_DLBCL (FOCAL); SEQ ID NO: 4192, 18q22.2 DLBCL (FOCAL); SEQ ID NO: 4193, 18q22.2_DLBCL (FOCAL); SEQ ID NO: 4194, 18q22.2_DLBCL (FOCAL); SEQ ID NO: 4195, 18q22.2_DLBCL (FOCAL); SEQ ID NO: 4196, 18q22.2_DLBCL (FOCAL); SEQ ID NO: 4197, 18q22.2_DLBCL (FOCAL); SEQ ID NO: 4198, 18q22.2_DLBCL (FOCAL); SEQ ID NO: 4199, 18q22.2_DLBCL (FOCAL); SEQ ID NO: 4200, 18q22.2_DLBCL (FOCAL); SEQ ID NO: 4201, 18q22.2_DLBCL (FOCAL); SEQ ID NO: 4202, 18q22.2_DLBCL (FOCAL); SEQ ID NO: 4203, 18q22.2 DLBCL (FOCAL); SEQ ID NO: 4204, 18q22.2_DLBCL (FOCAL); SEQ ID NO: 4205, 18q22.2_DLBCL (FOCAL); SEQ ID NO: 4206, 18q22.2_DLBCL (FOCAL); SEQ ID NO: 4207, 18q22.2_DLBCL (FOCAL); SEQ ID NO: 4208, 18q22.2_DLBCL (FOCAL); SEQ ID NO: 4209, FP (FP); SEQ ID NO: 4210, 18q22.2_DLBCL (FOCAL); SEQ ID NO: 4211, 18q22.2_DLBCL (FOCAL); SEQ ID NO: 4212, 18q22.2_DLBCL (FOCAL); SEQ ID NO: 4213, 18q22.2 DLBCL (FOCAL); SEQ ID NO: 4214, 18q22.2_DLBCL (FOCAL); SEQ ID NO: 4215, 18q22.2_DLBCL (FOCAL); SEQ ID NO: 4216, 18q22.2_DLBCL (FOCAL); SEQ ID NO: 4217, 18q22.2_DLBCL (FOCAL); SEQ ID NO: 4218, 18q22.2_DLBCL (FOCAL); SEQ ID NO: 4219, 18q22.2_DLBCL (FOCAL); SEQ ID NO: 4220, 18q22.2_DLBCL (FOCAL); SEQ ID NO: 4221, 18q22.2_DLBCL (FOCAL); SEQ ID NO: 4222, 18q22.2_DLBCL (FOCAL); SEQ ID NO: 4223, DAZAP1 (Gene); SEQ ID NO: 4224, DAZAP1 (Gene); SEQ ID NO: 4225, DAZAP1 (Gene); SEQ ID NO: 4226, DAZAP1 (Gene); SEQ ID NO: 4227, DAZAP1 (Gene); SEQ ID NO: 4228, DAZAP1 (Gene); SEQ ID NO: 4229, DAZAP1 (Gene); SEQ ID NO: 4230, DAZAP1 (Gene); SEQ ID NO: 4231, DAZAP1 (Gene); SEQ ID NO: 4232, DAZAP1 (Gene); SEQ ID NO: 4233, DAZAP1 (Gene); SEQ ID NO: 4234, DAZAP1 (Gene); SEQ ID NO: 4235, DAZAP1 (Gene); SEQ ID NO: 4236, RPS15 (Gene); SEQ ID NO: 4237, RPS15 (Gene); SEQ ID NO: 4238, RPS15 (Gene); SEQ ID NO: 4239, RPS15 (Gene); SEQ ID NO: 4240, RPS15 (Gene); SEQ ID NO: 4241, 19P (ARM); SEQ ID NO: 4242, 19P (ARM); SEQ ID NO: 4243, 19p13.3_DLBCL (FOCAL); SEQ ID NO: 4244, 19p13.3_DLBCL (FOCAL); SEQ ID NO: 4245, CD70 (Gene); SEQ ID NO: 4246, CD70 (Gene); SEQ ID NO: 4247, CD70 (Gene); SEQ ID NO: 4248, CD70 (Gene); SEQ ID NO: 4249, 19p13.3_DLBCL (FOCAL); SEQ ID NO: 4250, 19p13.3_DLBCL (FOCAL); SEQ ID NO: 4251, 19p13.3_DLBCL (FOCAL); SEQ ID NO: 4252, VAV1 (Gene); SEQ ID NO: 4253, VAV1 (Gene); SEQ ID NO: 4254, VAV1 (Gene); SEQ ID NO: 4255, VAV1 (Gene); SEQ ID NO: 4256, VAV1 (Gene); SEQ ID NO: 4257, VAV1 (Gene); SEQ ID NO: 4258, VAV1 (Gene); SEQ ID NO: 4259, VAV1 (Gene); SEQ ID NO: 4260, VAV1 (Gene); SEQ ID NO: 4261, VAV1 (Gene); SEQ ID NO: 4262, VAV1 (Gene); SEQ ID NO: 4263, VAV1 (Gene); SEQ ID NO: 4264, VAV1 (Gene); SEQ ID NO: 4265, VAV1 (Gene); SEQ ID NO: 4266, VAV1 (Gene); SEQ ID NO: 4267, VAV1 (Gene); SEQ ID NO: 4268, VAV1 (Gene); SEQ ID NO: 4269, VAV1 (Gene); SEQ ID NO: 4270, VAV1 (Gene); SEQ ID NO: 4271, VAV1 (Gene); SEQ ID NO: 4272, VAV1 (Gene); SEQ ID NO: 4273, VAV1 (Gene); SEQ ID NO: 4274, VAV1 (Gene); SEQ ID NO: 4275, VAV1 (Gene); SEQ ID NO: 4276, VAV1 (Gene); SEQ ID NO: 4277, VAV1 (Gene); SEQ ID NO: 4278, VAV1 (Gene); SEQ ID NO: 4279, 19p13.3_DLBCL (FOCAL); SEQ ID NO: 4280, 19p13.3_DLBCL (FOCAL); SEQ ID NO: 4281, 19p13.3_DLBCL (FOCAL); SEQ ID NO: 4282, 19P (ARM); SEQ ID NO: 4283, 19P (ARM); SEQ ID NO: 4284, 19p13.2_DLBCL (FOCAL); SEQ ID NO: 4285, 19p13.2_DLBCL (FOCAL); SEQ ID NO: 4286, 19p13.2_DLBCL (FOCAL); SEQ ID NO: 4287, 19p13.2_DLBCL (FOCAL); SEQ ID NO: 4288, 19p13.2_DLBCL (FOCAL); SEQ ID NO: 4289, 19p13.2_DLBCL (FOCAL); SEQ ID NO: 4290, 19p13.2_DLBCL (FOCAL); SEQ ID NO: 4291, 19p13.2_DLBCL (FOCAL); SEQ ID NO: 4292, 19p13.2_DLBCL (FOCAL); SEQ ID NO: 4293, SMARCA4 (Gene); SEQ ID NO: 4294, SMARCA4 (Gene); SEQ ID NO: 4295, SMARCA4 (Gene); SEQ ID NO: 4296, SMARCA4 (Gene); SEQ ID NO: 4297, SMARCA4 (Gene); SEQ ID NO: 4298, SMARCA4 (Gene); SEQ ID NO: 4299, SMARCA4 (Gene); SEQ ID NO: 4300, SMARCA4 (Gene); SEQ ID NO: 4301, SMARCA4 (Gene); SEQ ID NO: 4302, SMARCA4 (Gene); SEQ ID NO: 4303, SMARCA4 (Gene); SEQ ID NO: 4304, SMARCA4 (Gene); SEQ ID NO: 4305, SMARCA4 (Gene); SEQ ID NO: 4306, SMARCA4 (Gene); SEQ ID NO: 4307, SMARCA4 (Gene); SEQ ID NO: 4308, SMARCA4 (Gene); SEQ ID NO: 4309, SMARCA4 (Gene); SEQ ID NO: 4310, SMARCA4 (Gene); SEQ ID NO: 4311, SMARCA4 (Gene); SEQ ID NO: 4312, SMARCA4 (Gene); SEQ ID NO: 4313, SMARCA4 (Gene); SEQ ID NO: 4314, SMARCA4 (Gene); SEQ ID NO: 4315, SMARCA4 (Gene); SEQ ID NO: 4316, SMARCA4 (Gene); SEQ ID NO: 4317, SMARCA4 (Gene); SEQ ID NO: 4318, SMARCA4 (Gene); SEQ ID NO: 4319, SMARCA4 (Gene); SEQ ID NO: 4320, SMARCA4 (Gene); SEQ ID NO: 4321, SMARCA4 (Gene); SEQ ID NO: 4322, SMARCA4 (Gene); SEQ ID NO: 4323, SMARCA4 (Gene); SEQ ID NO: 4324, SMARCA4 (Gene); SEQ ID NO: 4325, SMARCA4 (Gene); SEQ ID NO: 4326, SMARCA4 (Gene); SEQ ID NO: 4327, SMARCA4 (Gene); SEQ ID NO: 4328, SMARCA4 (Gene); SEQ ID NO: 4329, 19p13.1_MCL (FOCAL); SEQ ID NO: 4330, 19p13.1_MCL (FOCAL); SEQ ID NO: 4331, 19p13.1_MCL (FOCAL); SEQ ID NO: 4332, 19p13.1_MCL (FOCAL); SEQ ID NO: 4333, 19p13.1_MCL (FOCAL); SEQ ID NO: 4334, 19p13.1_MCL (FOCAL); SEQ ID NO: 4335, 19p13.1_MCL (FOCAL); SEQ ID NO: 4336, 19p13.1_MCL (FOCAL); SEQ ID NO: 4337, 19p13.1_MCL (FOCAL); SEQ ID NO: 4338, 19p13.1_MCL (FOCAL); SEQ ID NO: 4339, 19p13.1_MCL (FOCAL); SEQ ID NO: 4340, 19p13.1_MCL (FOCAL); SEQ ID NO: 4341, KLF2 (Gene); SEQ ID NO: 4342, KLF2 (Gene); SEQ ID NO: 4343, KLF2 (Gene); SEQ ID NO: 4344, KLF2 (Gene); SEQ ID NO: 4345, 19p13.1_MCL (FOCAL); SEQ ID NO: 4346, 19p13.1_MCL (FOCAL); SEQ ID NO: 4347, 19p13.1_MCL (FOCAL); SEQ ID NO: 4348, 19p13.1_MCL (FOCAL); SEQ ID NO: 4349, 19p13.1_MCL (FOCAL); SEQ ID NO: 4350, 19p13.1_MCL (FOCAL); SEQ ID NO: 4351, 19p13.1_MCL (FOCAL); SEQ ID NO: 4352, JAK3 (Gene); SEQ ID NO: 4353, JAK3 (Gene); SEQ ID NO: 4354, JAK3 (Gene); SEQ ID NO: 4355, JAK3 (Gene); SEQ ID NO: 4356, JAK3 (Gene); SEQ ID NO: 4357, JAK3 (Gene); SEQ ID NO: 4358, JAK3 (Gene); SEQ ID NO: 4359, JAK3 (Gene); SEQ ID NO: 4360, JAK3 (Gene); SEQ ID NO: 4361, JAK3 (Gene); SEQ ID NO: 4362, JAK3 (Gene); SEQ ID NO: 4363, JAK3 (Gene); SEQ ID NO: 4364, JAK3 (Gene); SEQ ID NO: 4365, JAK3 (Gene); SEQ ID NO: 4366, JAK3 (Gene); SEQ ID NO: 4367, JAK3 (Gene); SEQ ID NO: 4368, JAK3 (Gene); SEQ ID NO: 4369, JAK3 (Gene); SEQ ID NO: 4370, JAK3 (Gene); SEQ ID NO: 4371, JAK3 (Gene); SEQ ID NO: 4372, JAK3 (Gene); SEQ ID NO: 4373, JAK3 (Gene); SEQ ID NO: 4374, JAK3 (Gene); SEQ ID NO: 4375, 19p13.1_MCL (FOCAL); SEQ ID NO: 4376, 19p13.1_MCL (FOCAL); SEQ ID NO: 4377, 19p13.1_MCL (FOCAL); SEQ ID NO: 4378, 19p13.1_MCL (FOCAL); SEQ ID NO: 4379, 19p13.1_MCL (FOCAL); SEQ ID NO: 4380, 19p13.1_MCL (FOCAL); SEQ ID NO: 4381, MEF2B (Gene); SEQ ID NO: 4382, MEF2B (Gene); SEQ ID NO: 4383, MEF2B (Gene); SEQ ID NO: 4384, MEF2B (Gene); SEQ ID NO: 4385, MEF2B (Gene); SEQ ID NO: 4386, MEF2B (Gene); SEQ ID NO: 4387, MEF2B (Gene); SEQ ID NO: 4388, MEF2B (Gene); SEQ ID NO: 4389, 19p13.1_MCL (FOCAL); SEQ ID NO: 4390, 19p13.1_MCL (FOCAL); SEQ ID NO: 4391, 19p13.1_MCL (FOCAL); SEQ ID NO: 4392, 19P (ARM); SEQ ID NO: 4393, 19P (ARM); SEQ ID NO: 4394, 19Q_DLBCL (ARM); SEQ ID NO: 4395, FP (FP); SEQ ID NO: 4396, 19Q_DLBCL (ARM); SEQ ID NO: 4397, 19Q_DLBCL (ARM); SEQ ID NO: 4398, 19Q_DLBCL (ARM); SEQ ID NO: 4399, 19Q_DLBCL (ARM); SEQ ID NO: 4400, 19Q_DLBCL (ARM); SEQ ID NO: 4401, 19Q_DLBCL (ARM); SEQ ID NO: 4402, 19Q_DLBCL (ARM); SEQ ID NO: 4403, 19Q_DLBCL (ARM); SEQ ID NO: 4404, 19Q_DLBCL (ARM); SEQ ID NO: 4405, 19Q_DLBCL (ARM); SEQ ID NO: 4406, CD79A (Gene); SEQ ID NO: 4407, CD79A (Gene); SEQ ID NO: 4408, CD79A (Gene); SEQ ID NO: 4409, CD79A (Gene); SEQ ID NO: 4410, CD79A (Gene); SEQ ID NO: 4411, ARHGEF1 (Gene); SEQ ID NO: 4412, ARHGEF1 (Gene); SEQ ID NO: 4413, ARHGEF1 (Gene); SEQ ID NO: 4414, ARHGEF1 (Gene); SEQ ID NO: 4415, ARHGEF1 (Gene); SEQ ID NO: 4416, ARHGEF1 (Gene); SEQ ID NO: 4417, ARHGEF1 (Gene); SEQ ID NO: 4418, ARHGEF1 (Gene); SEQ ID NO: 4419, ARHGEF1 (Gene); SEQ ID NO: 4420, ARHGEF1 (Gene); SEQ ID NO: 4421, ARHGEF1 (Gene); SEQ ID NO: 4422, ARHGEF1 (Gene); SEQ ID NO: 4423, ARHGEF1 (Gene); SEQ ID NO: 4424, ARHGEF1 (Gene); SEQ ID NO: 4425, ARHGEF1 (Gene); SEQ ID NO: 4426, ARHGEF1 (Gene); SEQ ID NO: 4427, ARHGEF1 (Gene); SEQ ID NO: 4428, ARHGEF1 (Gene); SEQ ID NO: 4429, ARHGEF1 (Gene); SEQ ID NO: 4430, ARHGEF1 (Gene); SEQ ID NO: 4431, ARHGEF1 (Gene); SEQ ID NO: 4432, ARHGEF1 (Gene); SEQ ID NO: 4433, ARHGEF1 (Gene); SEQ ID NO: 4434, ARHGEF1 (Gene); SEQ ID NO: 4435, ARHGEF1 (Gene); SEQ ID NO: 4436, ARHGEF1 (Gene); SEQ ID NO: 4437, ARHGEF1 (Gene); SEQ ID NO: 4438, ARHGEF1 (Gene); SEQ ID NO: 4439, ARHGEF1 (Gene); SEQ ID NO: 4440, POU2F2 (Gene); SEQ ID NO: 4441, POU2F2 (Gene); SEQ ID NO: 4442, POU2F2 (Gene); SEQ ID NO: 4443, POU2F2 (Gene); SEQ ID NO: 4444, POU2F2 (Gene); SEQ ID NO: 4445, POU2F2 (Gene); SEQ ID NO: 4446, POU2F2 (Gene); SEQ ID NO: 4447, POU2F2 (Gene); SEQ ID NO: 4448, POU2F2 (Gene); SEQ ID NO: 4449, POU2F2 (Gene); SEQ ID NO: 4450, POU2F2 (Gene); SEQ ID NO: 4451, POU2F2 (Gene); SEQ ID NO: 4452, POU2F2 (Gene); SEQ ID NO: 4453, POU2F2 (Gene); SEQ ID NO: 4454, 19Q_DLBCL (ARM); SEQ ID NO: 4455, 19Q_DLBCL (ARM); SEQ ID NO: 4456, ZNF296 (Gene); SEQ ID NO: 4457, ZNF296 (Gene); SEQ ID NO: 4458, ZNF296 (Gene); SEQ ID NO: 4459, ZNF296 (Gene); SEQ ID NO: 4460, ZNF296 (Gene); SEQ ID NO: 4461, 19Q_DLBCL (ARM); SEQ ID NO: 4462, MSI (MSI); SEQ ID NO: 4463, MSI (MSI); SEQ ID NO: 4464, 19Q_DLBCL (ARM); SEQ ID NO: 4465, MSI (MSI); SEQ ID NO: 4466, MSI (MSI); SEQ ID NO: 4467, SPIB (Gene); SEQ ID NO: 4468, SPIB (Gene); SEQ ID NO: 4469, SPIB (Gene); SEQ ID NO: 4470, SPIB (Gene); SEQ ID NO: 4471, SPIB (Gene); SEQ ID NO: 4472, SPIB (Gene); SEQ ID NO: 4473, 19Q_DLBCL (ARM); SEQ ID NO: 4474, 19Q_DLBCL (ARM); SEQ ID NO: 4475, 19q13.42_DLBCL (FOCAL); SEQ ID NO: 4476, 19q13.42_DLBCL (FOCAL); SEQ ID NO: 4477, 19q13.42_DLBCL (FOCAL); SEQ ID NO: 4478, 19q13.42_DLBCL (FOCAL); SEQ ID NO: 4479, 19q13.42_DLBCL (FOCAL); SEQ ID NO: 4480, 19q13.42_DLBCL (FOCAL); SEQ ID NO: 4481, 19q13.42_DLBCL (FOCAL); SEQ ID NO: 4482, 19q13.42_DLBCL (FOCAL); SEQ ID NO: 4483, 19q13.42_DLBCL (FOCAL); SEQ ID NO: 4484, 19q13.42_DLBCL (FOCAL); SEQ ID NO: 4485, 19q13.42_DLBCL (FOCAL); SEQ ID NO: 4486, 19q13.42_DLBCL (FOCAL); SEQ ID NO: 4487, 19q13.42_DLBCL (FOCAL); SEQ ID NO: 4488, 19q13.42_DLBCL (FOCAL); SEQ ID NO: 4489, NLRP8 (Gene); SEQ ID NO: 4490, NLRP8 (Gene); SEQ ID NO: 4491, NLRP8 (Gene); SEQ ID NO: 4492, NLRP8 (Gene); SEQ ID NO: 4493, NLRP8 (Gene); SEQ ID NO: 4494, NLRP8 (Gene); SEQ ID NO: 4495, NLRP8 (Gene); SEQ ID NO: 4496, NLRP8 (Gene); SEQ ID NO: 4497, NLRP8 (Gene); SEQ ID NO: 4498, NLRP8 (Gene); SEQ ID NO: 4499, NLRP8 (Gene); SEQ ID NO: 4500, NLRP8 (Gene); SEQ ID NO: 4501, NLRP8 (Gene); SEQ ID NO: 4502, NLRP8 (Gene); SEQ ID NO: 4503, NLRP8 (Gene); SEQ ID NO: 4504, 19q13.42_DLBCL (FOCAL); SEQ ID NO: 4505, 19q13.42_DLBCL (FOCAL); SEQ ID NO: 4506, 19q13.42_DLBCL (FOCAL); SEQ ID NO: 4507, 19q13.42_DLBCL (FOCAL); SEQ ID NO: 4508, 19q13.42_DLBCL (FOCAL); SEQ ID NO: 4509, 19q13.42_DLBCL (FOCAL); SEQ ID NO: 4510, 19q13.42_DLBCL (FOCAL); SEQ ID NO: 4511, 19q13.42_DLBCL (FOCAL); SEQ ID NO: 4512, FP (FP); SEQ ID NO: 4513, 2P (ARM); SEQ ID NO: 4514, 2P (ARM); SEQ ID NO: 4515, 2P (ARM); SEQ ID NO: 4516, 2P (ARM); SEQ ID NO: 4517, 2P (ARM); SEQ ID NO: 4518, 2P (ARM); SEQ ID NO: 4519, 2P (ARM); SEQ ID NO: 4520, 2P (ARM); SEQ ID NO: 4521, 2P (ARM); SEQ ID NO: 4522, FP (FP); SEQ ID NO: 4523, 2P (ARM); SEQ ID NO: 4524, 2P (ARM); SEQ ID NO: 4525, 2P (ARM); SEQ ID NO: 4526, 2P (ARM); SEQ ID NO: 4527, 2P (ARM); SEQ ID NO: 4528, 2P (ARM); SEQ ID NO: 4529, 2P (ARM); SEQ ID NO: 4530, FP (FP); SEQ ID NO: 4531, 2P (ARM); SEQ ID NO: 4532, 2P (ARM); SEQ ID NO: 4533, 2P (ARM); SEQ ID NO: 4534, 2P (ARM); SEQ ID NO: 4535, 2P (ARM); SEQ ID NO: 4536, 2P (ARM); SEQ ID NO: 4537, 2P (ARM); SEQ ID NO: 4538, 2P (ARM); SEQ ID NO: 4539, 2P (ARM); SEQ ID NO: 4540, 2P (ARM); SEQ ID NO: 4541, 2P (ARM); SEQ ID NO: 4542, 2P (ARM); SEQ ID NO: 4543, 2P (ARM); SEQ ID NO: 4544, DNMT3A (Gene); SEQ ID NO: 4545, DNMT3A (Gene); SEQ ID NO: 4546, DNMT3A (Gene); SEQ ID NO: 4547, DNMT3A (Gene); SEQ ID NO: 4548, DNMT3A (Gene); SEQ ID NO: 4549, DNMT3A (Gene); SEQ ID NO: 4550, DNMT3A (Gene); SEQ ID NO: 4551, DNMT3A (Gene); SEQ ID NO: 4552, DNMT3A (Gene); SEQ ID NO: 4553, DNMT3A (Gene); SEQ ID NO: 4554, DNMT3A (Gene); SEQ ID NO: 4555, DNMT3A (Gene); SEQ ID NO: 4556, DNMT3A (Gene); SEQ ID NO: 4557, DNMT3A (Gene); SEQ ID NO: 4558, DNMT3A (Gene); SEQ ID NO: 4559, DNMT3A (Gene); SEQ ID NO: 4560, DNMT3A (Gene); SEQ ID NO: 4561, DNMT3A (Gene); SEQ ID NO: 4562, DNMT3A (Gene); SEQ ID NO: 4563, DNMT3A (Gene); SEQ ID NO: 4564, DNMT3A (Gene); SEQ ID NO: 4565, DNMT3A (Gene); SEQ ID NO: 4566, DNMT3A (Gene); SEQ ID NO: 4567, DNMT3A (Gene); SEQ ID NO: 4568, DNMT3A (Gene); SEQ ID NO: 4569, 2P (ARM); SEQ ID NO: 4570, 2P (ARM); SEQ ID NO: 4571, ALK (Gene); SEQ ID NO: 4572, ALK (Gene); SEQ ID NO: 4573, ALK (Gene); SEQ ID NO: 4574, ALK (Gene); SEQ ID NO: 4575, ALK (Gene); SEQ ID NO: 4576, ALK (Gene); SEQ ID NO: 4577, ALK (Gene); SEQ ID NO: 4578, ALK (Gene); SEQ ID NO: 4579, ALK (Gene); SEQ ID NO: 4580, ALK (Gene); SEQ ID NO: 4581, ALK (Gene); SEQ ID NO: 4582, ALK (Gene); SEQ ID NO: 4583, ALK (Gene); SEQ ID NO: 4584, ALK (Gene); SEQ ID NO: 4585, ALK (Gene); SEQ ID NO: 4586, ALK (Gene); SEQ ID NO: 4587, ALK (Gene); SEQ ID NO: 4588, ALK (Gene); SEQ ID NO: 4589, ALK (Gene); SEQ ID NO: 4590, ALK (Gene); SEQ ID NO: 4591, ALK (Gene); SEQ ID NO: 4592, ALK (Gene); SEQ ID NO: 4593, ALK (Gene); SEQ ID NO: 4594, ALK (Gene); SEQ ID NO: 4595, ALK (Gene); SEQ ID NO: 4596, ALK (Gene); SEQ ID NO: 4597, ALK (Gene); SEQ ID NO: 4598, ALK (Gene); SEQ ID NO: 4599, ALK (Gene); SEQ ID NO: 4600, 2P (ARM); SEQ ID NO: 4601, 2P (ARM); SEQ ID NO: 4602, 2P (ARM); SEQ ID NO: 4603, 2P (ARM); SEQ ID NO: 4604, 2P (ARM); SEQ ID NO: 4605, 2P (ARM); SEQ ID NO: 4606, 2P (ARM); SEQ ID NO: 4607, 2P (ARM); SEQ ID NO: 4608, 2P (ARM); SEQ ID NO: 4609, 2P (ARM); SEQ ID NO: 4610, 2P (ARM); SEQ ID NO: 4611, 2P (ARM); SEQ ID NO: 4612, 2P (ARM); SEQ ID NO: 4613, 2P (ARM); SEQ ID NO: 4614, 2P (ARM); SEQ ID NO: 4615, 2P (ARM); SEQ ID NO: 4616, 2P (ARM); SEQ ID NO: 4617, 2P (ARM); SEQ ID NO: 4618, 2P (ARM); SEQ ID NO: 4619, 2P (ARM); SEQ ID NO: 4620, 2P (ARM); SEQ ID NO: 4621, 2P (ARM); SEQ ID NO: 4622, 2P (ARM); SEQ ID NO: 4623, 2P (ARM); SEQ ID NO: 4624, 2P (ARM); SEQ ID NO: 4625, 2P (ARM); SEQ ID NO: 4626, 2p15_HL_Region (FOCAL); SEQ ID NO: 4627, 2p15_HL_Region (FOCAL); SEQ ID NO: 4628, 2p15_HL_Region (FOCAL); SEQ ID NO: 4629, 2p15_HL_Region (FOCAL); SEQ ID NO: 4630, 2p15_HL_Region (FOCAL); SEQ ID NO: 4631, 2p15_HL_Region (FOCAL); SEQ ID NO: 4632, 2p15_HL_Region (FOCAL); SEQ ID NO: 4633, 2p15_HL_Region (FOCAL); SEQ ID NO: 4634, 2p15_HL_Region (FOCAL); SEQ ID NO: 4635, 2p15_HL_Region (FOCAL); SEQ ID NO: 4636, 2p15_HL_Region (FOCAL); SEQ ID NO: 4637, 2p15_HL_Region (FOCAL); SEQ ID NO: 4638, BCL11A (Gene); SEQ ID NO: 4639, BCL11A (Gene); SEQ ID NO: 4640, BCL11A (Gene); SEQ ID NO: 4641, BCL11A (Gene); SEQ ID NO: 4642, BCL11A (Gene); SEQ ID NO: 4643, BCL11A (Gene); SEQ ID NO: 4644, BCL11A (Gene); SEQ ID NO: 4645, BCL11A (Gene); SEQ ID NO: 4646, BCL11A (Gene); SEQ ID NO: 4647, 2p15_HL_Region (FOCAL); SEQ ID NO: 4648, REL (Gene); SEQ ID NO: 4649, REL (Gene); SEQ ID NO: 4650, REL (Gene); SEQ ID NO: 4651, REL (Gene); SEQ ID NO: 4652, REL (Gene); SEQ ID NO: 4653, REL (Gene); SEQ ID NO: 4654, REL (Gene); SEQ ID NO: 4655, REL (Gene); SEQ ID NO: 4656, REL (Gene); SEQ ID NO: 4657, REL (Gene); SEQ ID NO: 4658, REL (Gene); SEQ ID NO: 4659, 2p15_HL_Region (FOCAL); SEQ ID NO: 4660, 2p15_HL_Region (FOCAL); SEQ ID NO: 4661, 2p15_HL_Region (FOCAL); SEQ ID NO: 4662, 2p15_HL_Region (FOCAL); SEQ ID NO: 4663, XPO1 (Gene); SEQ ID NO: 4664, XPO1 (Gene); SEQ ID NO: 4665, XPO1 (Gene); SEQ ID NO: 4666, XPO1 (Gene); SEQ ID NO: 4667, XPO1 (Gene); SEQ ID NO: 4668, XPO1 (Gene); SEQ ID NO: 4669, XPO1 (Gene); SEQ ID NO: 4670, XPO1 (Gene); SEQ ID NO: 4671, XPO1 (Gene); SEQ ID NO: 4672, XPO1 (Gene); SEQ ID NO: 4673, XPO1 (Gene); SEQ ID NO: 4674, XPO1 (Gene); SEQ ID NO: 4675, XPO1 (Gene); SEQ ID NO: 4676, XPO1 (Gene); SEQ ID NO: 4677, XPO1 (Gene); SEQ ID NO: 4678, XPO1 (Gene); SEQ ID NO: 4679, XPO1 (Gene); SEQ ID NO: 4680, XPO1 (Gene); SEQ ID NO: 4681, XPO1 (Gene); SEQ ID NO: 4682, XPO1 (Gene); SEQ ID NO: 4683, XPO1 (Gene); SEQ ID NO: 4684, XPO1 (Gene); SEQ ID NO: 4685, XPO1 (Gene); SEQ ID NO: 4686, XPO1 (Gene); SEQ ID NO: 4687, 2p15_HL_Region (FOCAL); SEQ ID NO: 4688, 2p15_HL_Region (FOCAL); SEQ ID NO: 4689, 2p15_HL_Region (FOCAL); SEQ ID NO: 4690, 2p15_HL_Region (FOCAL); SEQ ID NO: 4691, 2p15_HL_Region (FOCAL); SEQ ID NO: 4692, 2p15_HL_Region (FOCAL); SEQ ID NO: 4693, 2P (ARM); SEQ ID NO: 4694, 2P (ARM); SEQ ID NO: 4695, 2P (ARM); SEQ ID NO: 4696, 2P (ARM); SEQ ID NO: 4697, 2P (ARM); SEQ ID NO: 4698, FP (FP); SEQ ID NO: 4699, 2P (ARM); SEQ ID NO: 4700, 2P (ARM); SEQ ID NO: 4701, ARHGAP25 (Gene); SEQ ID NO: 4702, ARHGAP25 (Gene); SEQ ID NO: 4703, ARHGAP25 (Gene); SEQ ID NO: 4704, ARHGAP25 (Gene); SEQ ID NO: 4705, ARHGAP25 (Gene); SEQ ID NO: 4706, ARHGAP25 (Gene); SEQ ID NO: 4707, ARHGAP25 (Gene); SEQ ID NO: 4708, ARHGAP25 (Gene); SEQ ID NO: 4709, ARHGAP25 (Gene); SEQ ID NO: 4710, ARHGAP25 (Gene); SEQ ID NO: 4711, ARHGAP25 (Gene); SEQ ID NO: 4712, ARHGAP25 (Gene); SEQ ID NO: 4713, 2P (ARM); SEQ ID NO: 4714, 2P (ARM); SEQ ID NO: 4715, 2P (ARM); SEQ ID NO: 4716, 2P (ARM); SEQ ID NO: 4717, MSI (MSI); SEQ ID NO: 4718, MSI (MSI); SEQ ID NO: 4719, 2P (ARM); SEQ ID NO: 4720, 2P (ARM); SEQ ID NO: 4721, 2P (ARM); SEQ ID NO: 4722, 2P (ARM); SEQ ID NO: 4723, FP (FP); SEQ ID NO: 4724, 2P (ARM); SEQ ID NO: 4725, 2P (ARM); SEQ ID NO: 4726, 2P (ARM); SEQ ID NO: 4727, 2P (ARM); SEQ ID NO: 4728, 2P (ARM); SEQ ID NO: 4729, 2P (ARM); SEQ ID NO: 4730, IGK_SV (SV); SEQ ID NO: 4731, IGK_SV (SV); SEQ ID NO: 4732, IGK_SV (SV); SEQ ID NO: 4733, IGK_SV (SV); SEQ ID NO: 4734, IGK_SV (SV); SEQ ID NO: 4735, IGK_SV (SV); SEQ ID NO: 4736, IGK_SV (SV); SEQ ID NO: 4737, IGK_SV (SV); SEQ ID NO: 4738, IGK_SV (SV); SEQ ID NO: 4739, IGK_SV (SV); SEQ ID NO: 4740, IGK_SV (SV); SEQ ID NO: 4741, IGK_SV (SV); SEQ ID NO: 4742, IGK_SV (SV); SEQ ID NO: 4743, IGK_SV (SV); SEQ ID NO: 4744, IGK_SV (SV); SEQ ID NO: 4745, IGK_SV (SV); SEQ ID NO: 4746, IGK_SV (SV); SEQ ID NO: 4747, IGK_SV (SV); SEQ ID NO: 4748, IGK_SV (SV); SEQ ID NO: 4749, IGK_SV (SV); SEQ ID NO: 4750, IGK_SV (SV); SEQ ID NO: 4751, IGK_SV (SV); SEQ ID NO: 4752, DUSP2 (Gene); SEQ ID NO: 4753, DUSP2 (Gene); SEQ ID NO: 4754, DUSP2 (Gene); SEQ ID NO: 4755, DUSP2 (Gene); SEQ ID NO: 4756, 2P (ARM); SEQ ID NO: 4757, 2P (ARM); SEQ ID NO: 4758, 2P (ARM); SEQ ID NO: 4759, 2P (ARM); SEQ ID NO: 4760, 2P (ARM); SEQ ID NO: 4761, 2P (ARM); SEQ ID NO: 4762, 2P (ARM); SEQ ID NO: 4763, 2P (ARM); SEQ ID NO: 4764, 2P (ARM); SEQ ID NO: 4765, 2q13_MCL (ARM); SEQ ID NO: 4766, 2q13 MCL (ARM); SEQ ID NO: 4767, 2q13 MCL (ARM); SEQ ID NO: 4768, 2q13 MCL (ARM); SEQ ID NO: 4769, 2q13 MCL (ARM); SEQ ID NO: 4770, 2q13_MCL (ARM); SEQ ID NO: 4771, 2q13 MCL (ARM); SEQ ID NO: 4772, 2q13_MCL (ARM); SEQ ID NO: 4773, 2q13 MCL (ARM); SEQ ID NO: 4774, 2q13 MCL (ARM); SEQ ID NO: 4775, 2q13 MCL (ARM); SEQ ID NO: 4776, 2q13 MCL (ARM); SEQ ID NO: 4777, 2q13_MCL (ARM); SEQ ID NO: 4778, 2q13 MCL (ARM); SEQ ID NO: 4779, 2q13_MCL (ARM); SEQ ID NO: 4780, 2q13 MCL (ARM); SEQ ID NO: 4781, 2q13 MCL (ARM); SEQ ID NO: 4782, 2q13 MCL (ARM); SEQ ID NO: 4783, 2q13 MCL (ARM); SEQ ID NO: 4784, 2q13_MCL (ARM); SEQ ID NO: 4785, 2P (ARM); SEQ ID NO: 4786, 2P (ARM); SEQ ID NO: 4787, 2P (ARM); SEQ ID NO: 4788, 2P (ARM); SEQ ID NO: 4789, 2P (ARM); SEQ ID NO: 4790, 2P (ARM); SEQ ID NO: 4791, 2P (ARM); SEQ ID NO: 4792, 2P (ARM); SEQ ID NO: 4793, 2P (ARM); SEQ ID NO: 4794, 2P (ARM); SEQ ID NO: 4795, 2P (ARM); SEQ ID NO: 4796, 2P (ARM); SEQ ID NO: 4797, 2P (ARM); SEQ ID NO: 4798, 2P (ARM); SEQ ID NO: 4799, 2P (ARM); SEQ ID NO: 4800, 2P (ARM); SEQ ID NO: 4801, 2P (ARM); SEQ ID NO: 4802, 2P (ARM); SEQ ID NO: 4803, 2P (ARM); SEQ ID NO: 4804, 2P (ARM); SEQ ID NO: 4805, 2P (ARM); SEQ ID NO: 4806, CXCR4 (Gene); SEQ ID NO: 4807, CXCR4 (Gene); SEQ ID NO: 4808, CXCR4 (Gene); SEQ ID NO: 4809, CXCR4 (Gene); SEQ ID NO: 4810, CXCR4 (Gene); SEQ ID NO: 4811, 2P (ARM); SEQ ID NO: 4812, 2P (ARM); SEQ ID NO: 4813, 2P (ARM); SEQ ID NO: 4814, 2P (ARM); SEQ ID NO: 4815, 2P (ARM); SEQ ID NO: 4816, 2q22.2_DLBCL (FOCAL); SEQ ID NO: 4817, 2q22.2_DLBCL (FOCAL); SEQ ID NO: 4818, 2q22.2_DLBCL (FOCAL); SEQ ID NO: 4819, 2q22.2_DLBCL (FOCAL); SEQ ID NO: 4820, 2q22.2_DLBCL (FOCAL); SEQ ID NO: 4821, 2q22.2_DLBCL (FOCAL); SEQ ID NO: 4822, ZEB2 (Gene); SEQ ID NO: 4823, ZEB2 (Gene); SEQ ID NO: 4824, ZEB2 (Gene); SEQ ID NO: 4825, ZEB2 (Gene); SEQ ID NO: 4826, ZEB2 (Gene); SEQ ID NO: 4827, ZEB2 (Gene); SEQ ID NO: 4828, ZEB2 (Gene); SEQ ID NO: 4829, ZEB2 (Gene); SEQ ID NO: 4830, ZEB2 (Gene); SEQ ID NO: 4831, ZEB2 (Gene); SEQ ID NO: 4832, ZEB2 (Gene); SEQ ID NO: 4833, ZEB2 (Gene); SEQ ID NO: 4834, ZEB2 (Gene); SEQ ID NO: 4835, ZEB2 (Gene); SEQ ID NO: 4836, 2q22.2_DLBCL (FOCAL); SEQ ID NO: 4837, 2q22.2_DLBCL (FOCAL); SEQ ID NO: 4838, 2q22.2_DLBCL (FOCAL); SEQ ID NO: 4839, 2q22.2_DLBCL (FOCAL); SEQ ID NO: 4840, 2q22.2_DLBCL (FOCAL); SEQ ID NO: 4841, 2q22.2_DLBCL (FOCAL); SEQ ID NO: 4842, 2q22.2_DLBCL (FOCAL); SEQ ID NO: 4843, 2q22.2_DLBCL (FOCAL); SEQ ID NO: 4844, 2q22.2_DLBCL (FOCAL); SEQ ID NO: 4845, 2q22.2_DLBCL (FOCAL); SEQ ID NO: 4846, 2q22.2_DLBCL (FOCAL); SEQ ID NO: 4847, 2q22.2_DLBCL (FOCAL); SEQ ID NO: 4848, 2q22.2_DLBCL (FOCAL); SEQ ID NO: 4849, 2q22.2_DLBCL (FOCAL); SEQ ID NO: 4850, 2q22.2_DLBCL (FOCAL); SEQ ID NO: 4851, MSI (MSI); SEQ ID NO: 4852, MSI (MSI); SEQ ID NO: 4853, 2P (ARM); SEQ ID NO: 4854, 2P (ARM); SEQ ID NO: 4855, 2P (ARM); SEQ ID NO: 4856, 2P (ARM); SEQ ID NO: 4857, 2P (ARM); SEQ ID NO: 4858, 2P (ARM); SEQ ID NO: 4859, 2P (ARM); SEQ ID NO: 4860, 2P (ARM); SEQ ID NO: 4861, 2P (ARM); SEQ ID NO: 4862, 2P (ARM); SEQ ID NO: 4863, MSI (MSI); SEQ ID NO: 4864, MSI (MSI); SEQ ID NO: 4865, 2P (ARM); SEQ ID NO: 4866, 2P (ARM); SEQ ID NO: 4867, 2P (ARM); SEQ ID NO: 4868, 2P (ARM); SEQ ID NO: 4869, 2P (ARM); SEQ ID NO: 4870, 2P (ARM); SEQ ID NO: 4871, 2P (ARM); SEQ ID NO: 4872, 2P (ARM); SEQ ID NO: 4873, 2P (ARM); SEQ ID NO: 4874, 2P (ARM); SEQ ID NO: 4875, 2P (ARM); SEQ ID NO: 4876, 2P (ARM); SEQ ID NO: 4877, 2P (ARM); SEQ ID NO: 4878, 2P (ARM); SEQ ID NO: 4879, 2P (ARM); SEQ ID NO: 4880, 2P (ARM); SEQ ID NO: 4881, 2P (ARM); SEQ ID NO: 4882, SF3B1 (Gene); SEQ ID NO: 4883, SF3B1 (Gene); SEQ ID NO: 4884, SF3B1 (Gene); SEQ ID NO: 4885, SF3B1 (Gene); SEQ ID NO: 4886, SF3B1 (Gene); SEQ ID NO: 4887, SF3B1 (Gene); SEQ ID NO: 4888, SF3B1 (Gene); SEQ ID NO: 4889, SF3B1 (Gene); SEQ ID NO: 4890, SF3B1 (Gene); SEQ ID NO: 4891, SF3B1 (Gene); SEQ ID NO: 4892, SF3B1 (Gene); SEQ ID NO: 4893, SF3B1 (Gene); SEQ ID NO: 4894, SF3B1 (Gene); SEQ ID NO: 4895, SF3B1 (Gene); SEQ ID NO: 4896, SF3B1 (Gene); SEQ ID NO: 4897, SF3B1 (Gene); SEQ ID NO: 4898, SF3B1 (Gene); SEQ ID NO: 4899, SF3B1 (Gene); SEQ ID NO: 4900, SF3B1 (Gene); SEQ ID NO: 4901, SF3B1 (Gene); SEQ ID NO: 4902, SF3B1 (Gene); SEQ ID NO: 4903, SF3B1 (Gene); SEQ ID NO: 4904, SF3B1 (Gene); SEQ ID NO: 4905, SF3B1 (Gene); SEQ ID NO: 4906, SF3B1 (Gene); SEQ ID NO: 4907, SF3B1 (Gene); SEQ ID NO: 4908, 2P (ARM); SEQ ID NO: 4909, 2P (ARM); SEQ ID NO: 4910, 2P (ARM); SEQ ID NO: 4911, 2P (ARM); SEQ ID NO: 4912, 2P (ARM); SEQ ID NO: 4913, MSI (MSI); SEQ ID NO: 4914, MSI (MSI); SEQ ID NO: 4915, 2P (ARM); SEQ ID NO: 4916, 2P (ARM); SEQ ID NO: 4917, MSI (MSI); SEQ ID NO: 4918, MSI (MSI); SEQ ID NO: 4919, 2P (ARM); SEQ ID NO: 4920, 2P (ARM); SEQ ID NO: 4921, IDH1 (Gene); SEQ ID NO: 4922, IDH1 (Gene); SEQ ID NO: 4923, IDH1 (Gene); SEQ ID NO: 4924, IDH1 (Gene); SEQ ID NO: 4925, IDH1 (Gene); SEQ ID NO: 4926, IDH1 (Gene); SEQ ID NO: 4927, IDH1 (Gene); SEQ ID NO: 4928, IDH1 (Gene); SEQ ID NO: 4929, 2P (ARM); SEQ ID NO: 4930, ERBB4 (Gene); SEQ ID NO: 4931, ERBB4 (Gene); SEQ ID NO: 4932, ERBB4 (Gene); SEQ ID NO: 4933, ERBB4 (Gene); SEQ ID NO: 4934, ERBB4 (Gene); SEQ ID NO: 4935, ERBB4 (Gene); SEQ ID NO: 4936, ERBB4 (Gene); SEQ ID NO: 4937, ERBB4 (Gene); SEQ ID NO: 4938, ERBB4 (Gene); SEQ ID NO: 4939, ERBB4 (Gene); SEQ ID NO: 4940, ERBB4 (Gene); SEQ ID NO: 4941, ERBB4 (Gene); SEQ ID NO: 4942, ERBB4 (Gene); SEQ ID NO: 4943, ERBB4 (Gene); SEQ ID NO: 4944, ERBB4 (Gene); SEQ ID NO: 4945, ERBB4 (Gene); SEQ ID NO: 4946, ERBB4 (Gene); SEQ ID NO: 4947, ERBB4 (Gene); SEQ ID NO: 4948, ERBB4 (Gene); SEQ ID NO: 4949, ERBB4 (Gene); SEQ ID NO: 4950, ERBB4 (Gene); SEQ ID NO: 4951, ERBB4 (Gene); SEQ ID NO: 4952, ERBB4 (Gene); SEQ ID NO: 4953, ERBB4 (Gene); SEQ ID NO: 4954, ERBB4 (Gene); SEQ ID NO: 4955, ERBB4 (Gene); SEQ ID NO: 4956, ERBB4 (Gene); SEQ ID NO: 4957, ERBB4 (Gene); SEQ ID NO: 4958, 2P (ARM); SEQ ID NO: 4959, 2P (ARM); SEQ ID NO: 4960, 2P (ARM); SEQ ID NO: 4961, 2P (ARM); SEQ ID NO: 4962, 2P (ARM); SEQ ID NO: 4963, 2P (ARM); SEQ ID NO: 4964, 2P (ARM); SEQ ID NO: 4965, 2P (ARM); SEQ ID NO: 4966, 2P (ARM); SEQ ID NO: 4967, FP (FP); SEQ ID NO: 4968, 2P (ARM); SEQ ID NO: 4969, 2P (ARM); SEQ ID NO: 4970, 2P (ARM); SEQ ID NO: 4971, 2P (ARM); SEQ ID NO: 4972, 2P (ARM); SEQ ID NO: 4973, 2P (ARM); SEQ ID NO: 4974, 2P (ARM); SEQ ID NO: 4975, 2P (ARM); SEQ ID NO: 4976, MSI (MSI); SEQ ID NO: 4977, MSI (MSI); SEQ ID NO: 4978, 2P (ARM); SEQ ID NO: 4979, 2P (ARM); SEQ ID NO: 4980, 2P (ARM); SEQ ID NO: 4981, 2P (ARM); SEQ ID NO: 4982, FP (FP); SEQ ID NO: 4983, 2P (ARM); SEQ ID NO: 4984, 2P (ARM); SEQ ID NO: 4985, 2P (ARM); SEQ ID NO: 4986, PDCD1 (Gene); SEQ ID NO: 4987, PDCD1 (Gene); SEQ ID NO: 4988, PDCD1 (Gene); SEQ ID NO: 4989, PDCD1 (Gene); SEQ ID NO: 4990, PDCD1 (Gene); SEQ ID NO: 4991, 20P (ARM); SEQ ID NO: 4992, 20P (ARM); SEQ ID NO: 4993, 20P (ARM); SEQ ID NO: 4994, 20P (ARM); SEQ ID NO: 4995, 20P (ARM); SEQ ID NO: 4996, 20P (ARM); SEQ ID NO: 4997, FP (FP); SEQ ID NO: 4998, 20P (ARM); SEQ ID NO: 4999, 20P (ARM); SEQ ID NO: 5000, 20P (ARM); SEQ ID NO: 5001, 20P (ARM); SEQ ID NO: 5002, 20P (ARM); SEQ ID NO: 5003, 20P (ARM); SEQ ID NO: 5004, 20P (ARM); SEQ ID NO: 5005, 20P (ARM); SEQ ID NO: 5006, 20P (ARM); SEQ ID NO: 5007, 20P (ARM); SEQ ID NO: 5008, 20P (ARM); SEQ ID NO: 5009, 20P (ARM); SEQ ID NO: 5010, 20P (ARM); SEQ ID NO: 5011, 20P (ARM); SEQ ID NO: 5012, 20P (ARM); SEQ ID NO: 5013, 20P (ARM); SEQ ID NO: 5014, 20P (ARM); SEQ ID NO: 5015, 20P (ARM); SEQ ID NO: 5016, 20P (ARM); SEQ ID NO: 5017, 20P (ARM); SEQ ID NO: 5018, 20P (ARM); SEQ ID NO: 5019, 20P (ARM); SEQ ID NO: 5020, 20P (ARM); SEQ ID NO: 5021, 20P (ARM); SEQ ID NO: 5022, 20P (ARM); SEQ ID NO: 5023, 20P (ARM); SEQ ID NO: 5024, ASXL1 (Gene); SEQ ID NO: 5025, ASXL1 (Gene); SEQ ID NO: 5026, ASXL1 (Gene); SEQ ID NO: 5027, ASXL1 (Gene); SEQ ID NO: 5028, ASXL1 (Gene); SEQ ID NO: 5029, ASXL1 (Gene); SEQ ID NO: 5030, ASXL1 (Gene); SEQ ID NO: 5031, ASXL1 (Gene); SEQ ID NO: 5032, ASXL1 (Gene); SEQ ID NO: 5033, ASXL1 (Gene); SEQ ID NO: 5034, ASXL1 (Gene); SEQ ID NO: 5035, ASXL1 (Gene); SEQ ID NO: 5036, ASXL1 (Gene); SEQ ID NO: 5037, ASXL1 (Gene); SEQ ID NO: 5038, ASXL1 (Gene); SEQ ID NO: 5039, ASXL1 (Gene); SEQ ID NO: 5040, ASXL1 (Gene); SEQ ID NO: 5041, ASXL1 (Gene); SEQ ID NO: 5042, ASXL1 (Gene); SEQ ID NO: 5043, ASXL1 (Gene); SEQ ID NO: 5044, ASXL1 (Gene); SEQ ID NO: 5045, 20Q (ARM); SEQ ID NO: 5046, 20Q (ARM); SEQ ID NO: 5047, 20Q (ARM); SEQ ID NO: 5048, 20Q (ARM); SEQ ID NO: 5049, 20Q (ARM); SEQ ID NO: 5050, 20Q (ARM); SEQ ID NO: 5051, 20Q (ARM); SEQ ID NO: 5052, FP (FP); SEQ ID NO: 5053, PLCG1 (Gene); SEQ ID NO: 5054, PLCG1 (Gene); SEQ ID NO: 5055, PLCG1 (Gene); SEQ ID NO: 5056, PLCG1 (Gene); SEQ ID NO: 5057, PLCG1 (Gene); SEQ ID NO: 5058, PLCG1 (Gene); SEQ ID NO: 5059, PLCG1 (Gene); SEQ ID NO: 5060, PLCG1 (Gene); SEQ ID NO: 5061, PLCG1 (Gene); SEQ ID NO: 5062, PLCG1 (Gene); SEQ ID NO: 5063, PLCG1 (Gene); SEQ ID NO: 5064, PLCG1 (Gene); SEQ ID NO: 5065, PLCG1 (Gene); SEQ ID NO: 5066, PLCG1 (Gene); SEQ ID NO: 5067, PLCG1 (Gene); SEQ ID NO: 5068, PLCG1 (Gene); SEQ ID NO: 5069, PLCG1 (Gene); SEQ ID NO: 5070, PLCG1 (Gene); SEQ ID NO: 5071, PLCG1 (Gene); SEQ ID NO: 5072, PLCG1 (Gene); SEQ ID NO: 5073, PLCG1 (Gene); SEQ ID NO: 5074, PLCG1 (Gene); SEQ ID NO: 5075, PLCG1 (Gene); SEQ ID NO: 5076, PLCG1 (Gene); SEQ ID NO: 5077, PLCG1 (Gene); SEQ ID NO: 5078, PLCG1 (Gene); SEQ ID NO: 5079, PLCG1 (Gene); SEQ ID NO: 5080, PLCG1 (Gene); SEQ ID NO: 5081, PLCG1 (Gene); SEQ ID NO: 5082, PLCG1 (Gene); SEQ ID NO: 5083, PLCG1 (Gene); SEQ ID NO: 5084, PLCG1 (Gene); SEQ ID NO: 5085, 20Q (ARM); SEQ ID NO: 5086, 20Q (ARM); SEQ ID NO: 5087, 20Q (ARM); SEQ ID NO: 5088, 20Q (ARM); SEQ ID NO: 5089, 20Q (ARM); SEQ ID NO: 5090, 20Q (ARM); SEQ ID NO: 5091, 20Q (ARM); SEQ ID NO: 5092, 20Q (ARM); SEQ ID NO: 5093, 20Q (ARM); SEQ ID NO: 5094, MSI (MSI); SEQ ID NO: 5095, MSI (MSI); SEQ ID NO: 5096, 20Q (ARM); SEQ ID NO: 5097, 20Q (ARM); SEQ ID NO: 5098, PTPN1 (Gene); SEQ ID NO: 5099, PTPN1 (Gene); SEQ ID NO: 5100, PTPN1 (Gene); SEQ ID NO: 5101, PTPN1 (Gene); SEQ ID NO: 5102, PTPN1 (Gene); SEQ ID NO: 5103, PTPN1 (Gene); SEQ ID NO: 5104, PTPN1 (Gene); SEQ ID NO: 5105, PTPN1 (Gene); SEQ ID NO: 5106, PTPN1 (Gene); SEQ ID NO: 5107, PTPN1 (Gene); SEQ ID NO: 5108, MSI (MSI); SEQ ID NO: 5109, MSI (MSI); SEQ ID NO: 5110, 20Q (ARM); SEQ ID NO: 5111, 20Q (ARM); SEQ ID NO: 5112, 20Q (ARM); SEQ ID NO: 5113, 20Q (ARM); SEQ ID NO: 5114, ZNF217 (Gene); SEQ ID NO: 5115, ZNF217 (Gene); SEQ ID NO: 5116, ZNF217 (Gene); SEQ ID NO: 5117, ZNF217 (Gene); SEQ ID NO: 5118, ZNF217 (Gene); SEQ ID NO: 5119, ZNF217 (Gene); SEQ ID NO: 5120, ZNF217 (Gene); SEQ ID NO: 5121, ZNF217 (Gene); SEQ ID NO: 5122, ZNF217 (Gene); SEQ ID NO: 5123, 20Q (ARM); SEQ ID NO: 5124, 20Q (ARM); SEQ ID NO: 5125, 20Q (ARM); SEQ ID NO: 5126, 20Q (ARM); SEQ ID NO: 5127, 20Q (ARM); SEQ ID NO: 5128, RBM38 (Gene); SEQ ID NO: 5129, RBM38 (Gene); SEQ ID NO: 5130, RBM38 (Gene); SEQ ID NO: 5131, RBM38 (Gene); SEQ ID NO: 5132, RBM38 (Gene); SEQ ID NO: 5133, 20Q (ARM); SEQ ID NO: 5134, 20Q (ARM); SEQ ID NO: 5135, 20Q (ARM); SEQ ID NO: 5136, 20Q (ARM); SEQ ID NO: 5137, MSI (MSI); SEQ ID NO: 5138, MSI (MSI); SEQ ID NO: 5139, 20Q (ARM); SEQ ID NO: 5140, 20Q (ARM); SEQ ID NO: 5141, 20Q (ARM); SEQ ID NO: 5142, 20Q (ARM); SEQ ID NO: 5143, 21q11.2_MCL (FOCAL); SEQ ID NO: 5144, 21q11.2_MCL (FOCAL); SEQ ID NO: 5145, 21q11.2_MCL (FOCAL); SEQ ID NO: 5146, 21q11.2_MCL (FOCAL); SEQ ID NO: 5147, 21q11.2_MCL (FOCAL); SEQ ID NO: 5148, 21q11.2_MCL (FOCAL); SEQ ID NO: 5149, 21q11.2_MCL (FOCAL); SEQ ID NO: 5150, 21q11.2_MCL (FOCAL); SEQ ID NO: 5151, 21q11.2_MCL (FOCAL); SEQ ID NO: 5152, 21q11.2_MCL (FOCAL); SEQ ID NO: 5153, FP (FP); SEQ ID NO: 5154, 21Q_DLBCL (ARM); SEQ ID NO: 5155, 21Q_DLBCL (ARM); SEQ ID NO: 5156, 21Q_DLBCL (ARM); SEQ ID NO: 5157, 21Q_DLBCL (ARM); SEQ ID NO: 5158, 21Q_DLBCL (ARM); SEQ ID NO: 5159, 21Q_DLBCL (ARM); SEQ ID NO: 5160, 21Q_DLBCL (ARM); SEQ ID NO: 5161, 21Q_DLBCL (ARM); SEQ ID NO: 5162, 21Q_DLBCL (ARM); SEQ ID NO: 5163, 21Q_DLBCL (ARM); SEQ ID NO: 5164, 21Q_DLBCL (ARM); SEQ ID NO: 5165, FP (FP); SEQ ID NO: 5166, 21Q_DLBCL (ARM); SEQ ID NO: 5167, FP (FP); SEQ ID NO: 5168, 21Q_DLBCL (ARM); SEQ ID NO: 5169, 21Q_DLBCL (ARM); SEQ ID NO: 5170, 21Q_DLBCL (ARM); SEQ ID NO: 5171, 21Q_DLBCL (ARM); SEQ ID NO: 5172, 21Q_DLBCL (ARM); SEQ ID NO: 5173, 21Q_DLBCL (ARM); SEQ ID NO: 5174, MSI (MSI); SEQ ID NO: 5175, MSI (MSI); SEQ ID NO: 5176, 21Q_DLBCL (ARM); SEQ ID NO: 5177, 21Q_DLBCL (ARM); SEQ ID NO: 5178, 21Q_DLBCL (ARM); SEQ ID NO: 5179, 21Q_DLBCL (ARM); SEQ ID NO: 5180, 21Q_DLBCL (ARM); SEQ ID NO: 5181, 21Q_DLBCL (ARM); SEQ ID NO: 5182, 21Q_DLBCL (ARM); SEQ ID NO: 5183, FP (FP); SEQ ID NO: 5184, 21Q_DLBCL (ARM); SEQ ID NO: 5185, 21Q_DLBCL (ARM); SEQ ID NO: 5186, 21Q_DLBCL (ARM); SEQ ID NO: 5187, 21Q_DLBCL (ARM); SEQ ID NO: 5188, FP (FP); SEQ ID NO: 5189, 22Q (ARM); SEQ ID NO: 5190, 22Q (ARM); SEQ ID NO: 5191, FP (FP); SEQ ID NO: 5192, MAPK1 (Gene); SEQ ID NO: 5193, MAPK1 (Gene); SEQ ID NO: 5194, MAPK1 (Gene); SEQ ID NO: 5195, MAPK1 (Gene); SEQ ID NO: 5196, MAPK1 (Gene); SEQ ID NO: 5197, MAPK1 (Gene); SEQ ID NO: 5198, MAPK1 (Gene); SEQ ID NO: 5199, IGLV2-14 (SV); SEQ ID NO: 5200, IGLV2-14 (SV); SEQ ID NO: 5201, IGLV2-14 (SV); SEQ ID NO: 5202, IGLL5 (Gene); SEQ ID NO: 5203, IGLL5_intron_SV (SV); SEQ ID NO: 5204, IGLL5 (Gene); SEQ ID NO: 5205, IgL_SV (SV); SEQ ID NO: 5206, IgL_SV (SV); SEQ ID NO: 5207, IgL_SV (SV); SEQ ID NO: 5208, IgL_SV (SV); SEQ ID NO: 5209, IgL_SV (SV); SEQ ID NO: 5210, IgL_SV (SV); SEQ ID NO: 5211, IgL_SV (SV); SEQ ID NO: 5212, IgL_SV (SV); SEQ ID NO: 5213, IgL_SV (SV); SEQ ID NO: 5214, IgL_SV (SV); SEQ ID NO: 5215, IgL_SV (SV); SEQ ID NO: 5216, IGLL1_SV (SV); SEQ ID NO: 5217, IGLL1_SV (SV); SEQ ID NO: 5218, IGLL1_SV (SV); SEQ ID NO: 5219, IGLL1_SV (SV); SEQ ID NO: 5220, IGLL1_SV (SV); SEQ ID NO: 5221, IGLL1_SV (SV); SEQ ID NO: 5222, IGLL1_SV (SV); SEQ ID NO: 5223, IGLL1_SV (SV); SEQ ID NO: 5224, IGLL1_SV (SV); SEQ ID NO: 5225, IGLL1_SV (SV); SEQ ID NO: 5226, IGLL1_SV (SV); SEQ ID NO: 5227, IGLL1_SV (SV); SEQ ID NO: 5228, IGLL1_SV (SV); SEQ ID NO: 5229, IGLL1_SV (SV); SEQ ID NO: 5230, IGLL1_SV (SV); SEQ ID NO: 5231, 22Q (ARM); SEQ ID NO: 5232, 22Q (ARM); SEQ ID NO: 5233, 22Q (ARM); SEQ ID NO: 5234, 22Q (ARM); SEQ ID NO: 5235, FP (FP); SEQ ID NO: 5236, 22Q (ARM); SEQ ID NO: 5237, CHEK2 (Gene); SEQ ID NO: 5238, CHEK2 (Gene); SEQ ID NO: 5239, CHEK2 (Gene); SEQ ID NO: 5240, CHEK2 (Gene); SEQ ID NO: 5241, CHEK2 (Gene); SEQ ID NO: 5242, CHEK2 (Gene); SEQ ID NO: 5243, CHEK2 (Gene); SEQ ID NO: 5244, CHEK2 (Gene); SEQ ID NO: 5245, CHEK2 (Gene); SEQ ID NO: 5246, CHEK2 (Gene); SEQ ID NO: 5247, CHEK2 (Gene); SEQ ID NO: 5248, CHEK2 (Gene); SEQ ID NO: 5249, CHEK2 (Gene); SEQ ID NO: 5250, CHEK2 (Gene); SEQ ID NO: 5251, CHEK2 (Gene); SEQ ID NO: 5252, FP (FP); SEQ ID NO: 5253, 22Q (ARM); SEQ ID NO: 5254, FP (FP); SEQ ID NO: 5255, 22Q (ARM); SEQ ID NO: 5256, FP (FP); SEQ ID NO: 5257, 22Q (ARM); SEQ ID NO: 5258, 22Q (ARM); SEQ ID NO: 5259, 22Q (ARM); SEQ ID NO: 5260, 22Q (ARM); SEQ ID NO: 5261, 22Q (ARM); SEQ ID NO: 5262, 22Q (ARM); SEQ ID NO: 5263, 22Q (ARM); SEQ ID NO: 5264, 22Q (ARM); SEQ ID NO: 5265, 22Q (ARM); SEQ ID NO: 5266, EP300 (Gene); SEQ ID NO: 5267, EP300 (Gene); SEQ ID NO: 5268, EP300 (Gene); SEQ ID NO: 5269, EP300 (Gene); SEQ ID NO: 5270, EP300 (Gene); SEQ ID NO: 5271, EP300 (Gene); SEQ ID NO: 5272, EP300 (Gene); SEQ ID NO: 5273, EP300 (Gene); SEQ ID NO: 5274, EP300 (Gene); SEQ ID NO: 5275, EP300 (Gene); SEQ ID NO: 5276, EP300 (Gene); SEQ ID NO: 5277, EP300 (Gene); SEQ ID NO: 5278, EP300 (Gene); SEQ ID NO: 5279, EP300 (Gene); SEQ ID NO: 5280, EP300 (Gene); SEQ ID NO: 5281, EP300 (Gene); SEQ ID NO: 5282, EP300 (Gene); SEQ ID NO: 5283, EP300 (Gene); SEQ ID NO: 5284, EP300 (Gene); SEQ ID NO: 5285, EP300 (Gene); SEQ ID NO: 5286, EP300 (Gene); SEQ ID NO: 5287, EP300 (Gene); SEQ ID NO: 5288, EP300 (Gene); SEQ ID NO: 5289, EP300 (Gene); SEQ ID NO: 5290, EP300 (Gene); SEQ ID NO: 5291, EP300 (Gene); SEQ ID NO: 5292, EP300 (Gene); SEQ ID NO: 5293, EP300 (Gene); SEQ ID NO: 5294, EP300 (Gene); SEQ ID NO: 5295, EP300 (Gene); SEQ ID NO: 5296, EP300 (Gene); SEQ ID NO: 5297, EP300 (Gene); SEQ ID NO: 5298, EP300 (Gene); SEQ ID NO: 5299, EP300 (Gene); SEQ ID NO: 5300, EP300 (Gene); SEQ ID NO: 5301, EP300 (Gene); SEQ ID NO: 5302, EP300 (Gene); SEQ ID NO: 5303, 22Q (ARM); SEQ ID NO: 5304, 22Q (ARM); SEQ ID NO: 5305, 22Q (ARM); SEQ ID NO: 5306, 22Q (ARM); SEQ ID NO: 5307, 22Q (ARM); SEQ ID NO: 5308, 22Q (ARM); SEQ ID NO: 5309, 22Q (ARM); SEQ ID NO: 5310, MSI (MSI); SEQ ID NO: 5311, MSI (MSI); SEQ ID NO: 5312, 22Q (ARM); SEQ ID NO: 5313, 3P_DLBCL (ARM); SEQ ID NO: 5314, 3P_DLBCL (ARM); SEQ ID NO: 5315, FP (FP); SEQ ID NO: 5316, 3P_DLBCL (ARM); SEQ ID NO: 5317, 3P_DLBCL (ARM); SEQ ID NO: 5318, 3P_DLBCL (ARM); SEQ ID NO: 5319, CRBN (Gene); SEQ ID NO: 5320, CRBN (Gene); SEQ ID NO: 5321, CRBN (Gene); SEQ ID NO: 5322, CRBN (Gene); SEQ ID NO: 5323, CRBN (Gene); SEQ ID NO: 5324, CRBN (Gene); SEQ ID NO: 5325, CRBN (Gene); SEQ ID NO: 5326, CRBN (Gene); SEQ ID NO: 5327, CRBN (Gene); SEQ ID NO: 5328, CRBN (Gene); SEQ ID NO: 5329, CRBN (Gene); SEQ ID NO: 5330, 3P_DLBCL (ARM); SEQ ID NO: 5331, 3P_DLBCL (ARM); SEQ ID NO: 5332, 3P_DLBCL (ARM); SEQ ID NO: 5333, 3P_DLBCL (ARM); SEQ ID NO: 5334, 3P_DLBCL (ARM); SEQ ID NO: 5335, 3P_DLBCL (ARM); SEQ ID NO: 5336, 3P_DLBCL (ARM); SEQ ID NO: 5337, 3P_DLBCL (ARM); SEQ ID NO: 5338, SETD5 (Gene); SEQ ID NO: 5339, SETD5 (Gene); SEQ ID NO: 5340, SETD5 (Gene); SEQ ID NO: 5341, SETD5 (Gene); SEQ ID NO: 5342, SETD5 (Gene); SEQ ID NO: 5343, SETD5 (Gene); SEQ ID NO: 5344, SETD5 (Gene); SEQ ID NO: 5345, SETD5 (Gene); SEQ ID NO: 5346, SETD5 (Gene); SEQ ID NO: 5347, SETD5 (Gene); SEQ ID NO: 5348, SETD5 (Gene); SEQ ID NO: 5349, SETD5 (Gene); SEQ ID NO: 5350, SETD5 (Gene); SEQ ID NO: 5351, SETD5 (Gene); SEQ ID NO: 5352, SETD5 (Gene); SEQ ID NO: 5353, SETD5 (Gene); SEQ ID NO: 5354, SETD5 (Gene); SEQ ID NO: 5355, SETD5 (Gene); SEQ ID NO: 5356, SETD5 (Gene); SEQ ID NO: 5357, SETD5 (Gene); SEQ ID NO: 5358, SETD5 (Gene); SEQ ID NO: 5359, SETD5 (Gene); SEQ ID NO: 5360, SETD5 (Gene); SEQ ID NO: 5361, SETD5 (Gene); SEQ ID NO: 5362, SETD5 (Gene); SEQ ID NO: 5363, SETD5 (Gene); SEQ ID NO: 5364, SETD5 (Gene); SEQ ID NO: 5365, SETD5 (Gene); SEQ ID NO: 5366, SETD5 (Gene); SEQ ID NO: 5367, 3P_DLBCL (ARM); SEQ ID NO: 5368, 3P_DLBCL (ARM); SEQ ID NO: 5369, 3P_DLBCL (ARM); SEQ ID NO: 5370, RAF1 (Gene); SEQ ID NO: 5371, RAF1 (Gene); SEQ ID NO: 5372, RAF1 (Gene); SEQ ID NO: 5373, RAF1 (Gene); SEQ ID NO: 5374, RAF1 (Gene); SEQ ID NO: 5375, RAF1 (Gene); SEQ ID NO: 5376, RAF1 (Gene); SEQ ID NO: 5377, RAF1 (Gene); SEQ ID NO: 5378, RAF1 (Gene); SEQ ID NO: 5379, RAF1 (Gene); SEQ ID NO: 5380, RAF1 (Gene); SEQ ID NO: 5381, RAF1 (Gene); SEQ ID NO: 5382, RAF1 (Gene); SEQ ID NO: 5383, RAF1 (Gene); SEQ ID NO: 5384, RAF1 (Gene); SEQ ID NO: 5385, RAF1 (Gene); SEQ ID NO: 5386, RAF1 (Gene); SEQ ID NO: 5387, 3P_DLBCL (ARM); SEQ ID NO: 5388, 3P_DLBCL (ARM); SEQ ID NO: 5389, 3P_DLBCL (ARM); SEQ ID NO: 5390, 3P_DLBCL (ARM); SEQ ID NO: 5391, 3P_DLBCL (ARM); SEQ ID NO: 5392, 3P_DLBCL (ARM); SEQ ID NO: 5393, 3P_DLBCL (ARM); SEQ ID NO: 5394, FP (FP); SEQ ID NO: 5395, 3P_DLBCL (ARM); SEQ ID NO: 5396, 3P_DLBCL (ARM); SEQ ID NO: 5397, 3P_DLBCL (ARM); SEQ ID NO: 5398, 3P_DLBCL (ARM); SEQ ID NO: 5399, 3P_DLBCL (ARM); SEQ ID NO: 5400, 3P_DLBCL (ARM); SEQ ID NO: 5401, 3P_DLBCL (ARM); SEQ ID NO: 5402, 3P_DLBCL (ARM); SEQ ID NO: 5403, 3P_DLBCL (ARM); SEQ ID NO: 5404, 3P_DLBCL (ARM); SEQ ID NO: 5405, 3P_DLBCL (ARM); SEQ ID NO: 5406, 3P_DLBCL (ARM); SEQ ID NO: 5407, 3P_DLBCL (ARM); SEQ ID NO: 5408, MSI (MSI); SEQ ID NO: 5409, MSI (MSI); SEQ ID NO: 5410, 3P_DLBCL (ARM); SEQ ID NO: 5411, CCR4 (Gene); SEQ ID NO: 5412, CCR4 (Gene); SEQ ID NO: 5413, CCR4 (Gene); SEQ ID NO: 5414, CCR4 (Gene); SEQ ID NO: 5415, CCR4 (Gene); SEQ ID NO: 5416, 3P_DLBCL (ARM); SEQ ID NO: 5417, 3P_DLBCL (ARM); SEQ ID NO: 5418, 3P_DLBCL (ARM); SEQ ID NO: 5419, 3P_DLBCL (ARM); SEQ ID NO: 5420, 3P_DLBCL (ARM); SEQ ID NO: 5421, FP (FP); SEQ ID NO: 5422, 3P_DLBCL (ARM); SEQ ID NO: 5423, MYD88 (Gene); SEQ ID NO: 5424, MYD88 (Gene); SEQ ID NO: 5425, MYD88 (Gene); SEQ ID NO: 5426, MYD88 (Gene); SEQ ID NO: 5427, MYD88 (Gene); SEQ ID NO: 5428, 3P_DLBCL (ARM); SEQ ID NO: 5429, 3P_DLBCL (ARM); SEQ ID NO: 5430, 3P_DLBCL (ARM); SEQ ID NO: 5431, 3P_DLBCL (ARM); SEQ ID NO: 5432, 3P_DLBCL (ARM); SEQ ID NO: 5433, 3P_DLBCL (ARM); SEQ ID NO: 5434, 3P_DLBCL (ARM); SEQ ID NO: 5435, 3P_DLBCL (ARM); SEQ ID NO: 5436, SETD2 (Gene); SEQ ID NO: 5437, SETD2 (Gene); SEQ ID NO: 5438, SETD2 (Gene); SEQ ID NO: 5439, SETD2 (Gene); SEQ ID NO: 5440, SETD2 (Gene); SEQ ID NO: 5441, SETD2 (Gene); SEQ ID NO: 5442, SETD2 (Gene); SEQ ID NO: 5443, SETD2 (Gene); SEQ ID NO: 5444, SETD2 (Gene); SEQ ID NO: 5445, SETD2 (Gene); SEQ ID NO: 5446, SETD2 (Gene); SEQ ID NO: 5447, SETD2 (Gene); SEQ ID NO: 5448, SETD2 (Gene); SEQ ID NO: 5449, SETD2 (Gene); SEQ ID NO: 5450, SETD2 (Gene); SEQ ID NO: 5451, SETD2 (Gene); SEQ ID NO: 5452, SETD2 (Gene); SEQ ID NO: 5453, SETD2 (Gene); SEQ ID NO: 5454, SETD2 (Gene); SEQ ID NO: 5455, SETD2 (Gene); SEQ ID NO: 5456, SETD2 (Gene); SEQ ID NO: 5457, SETD2 (Gene); SEQ ID NO: 5458, SETD2 (Gene); SEQ ID NO: 5459, SETD2 (Gene); SEQ ID NO: 5460, SETD2 (Gene); SEQ ID NO: 5461, SETD2 (Gene); SEQ ID NO: 5462, SETD2 (Gene); SEQ ID NO: 5463, SETD2 (Gene); SEQ ID NO: 5464, SETD2 (Gene); SEQ ID NO: 5465, SETD2 (Gene); SEQ ID NO: 5466, SETD2 (Gene); SEQ ID NO: 5467, SETD2 (Gene); SEQ ID NO: 5468, 3p21.31_DLBCL (FOCAL); SEQ ID NO: 5469, 3p21.31_DLBCL (FOCAL); SEQ ID NO: 5470, 3p21.31_DLBCL (FOCAL); SEQ ID NO: 5471, 3p21.31_DLBCL (FOCAL); SEQ ID NO: 5472, 3p21.31_DLBCL (FOCAL); SEQ ID NO: 5473, 3p21.31_DLBCL (FOCAL); SEQ ID NO: 5474, 3p21.31_DLBCL (FOCAL); SEQ ID NO: 5475, 3p21.31_DLBCL (FOCAL); SEQ ID NO: 5476, 3p21.31_DLBCL (FOCAL); SEQ ID NO: 5477, 3p21.31_DLBCL (FOCAL); SEQ ID NO: 5478, RHOA (Gene); SEQ ID NO: 5479, RHOA (Gene); SEQ ID NO: 5480, RHOA (Gene); SEQ ID NO: 5481, RHOA (Gene); SEQ ID NO: 5482, 3p21.31_DLBCL (FOCAL); SEQ ID NO: 5483, 3p21.31_DLBCL (FOCAL); SEQ ID NO: 5484, 3p21.31_DLBCL (FOCAL); SEQ ID NO: 5485, GNAI2 (Gene); SEQ ID NO: 5486, GNAI2 (Gene); SEQ ID NO: 5487, GNAI2 (Gene); SEQ ID NO: 5488, GNAI2 (Gene); SEQ ID NO: 5489, GNAI2 (Gene); SEQ ID NO: 5490, GNAI2 (Gene); SEQ ID NO: 5491, GNAI2 (Gene); SEQ ID NO: 5492, GNAI2 (Gene); SEQ ID NO: 5493, GNAI2 (Gene); SEQ ID NO: 5494, GNAI2 (Gene); SEQ ID NO: 5495, GNAI2 (Gene); SEQ ID NO: 5496, MSI (MSI); SEQ ID NO: 5497, MSI (MSI); SEQ ID NO: 5498, 3P_DLBCL (ARM); SEQ ID NO: 5499, 3P_DLBCL (ARM); SEQ ID NO: 5500, 3P_DLBCL (ARM); SEQ ID NO: 5501, 3P_DLBCL (ARM); SEQ ID NO: 5502, 3P_DLBCL (ARM); SEQ ID NO: 5503, 3P_DLBCL (ARM); SEQ ID NO: 5504, 3P_DLBCL (ARM); SEQ ID NO: 5505, 3P_DLBCL (ARM); SEQ ID NO: 5506, 3P_DLBCL (ARM); SEQ ID NO: 5507, 3P_DLBCL (ARM); SEQ ID NO: 5508, 3P_DLBCL (ARM); SEQ ID NO: 5509, 3P_DLBCL (ARM); SEQ ID NO: 5510, 3P_DLBCL (ARM); SEQ ID NO: 5511, 3P_DLBCL (ARM); SEQ ID NO: 5512, 3P_DLBCL (ARM); SEQ ID NO: 5513, 3P_DLBCL (ARM); SEQ ID NO: 5514, 3P_DLBCL (ARM); SEQ ID NO: 5515, 3P_DLBCL (ARM); SEQ ID NO: 5516, 3P_DLBCL (ARM); SEQ ID NO: 5517, 3P_DLBCL (ARM); SEQ ID NO: 5518, 3P_DLBCL (ARM); SEQ ID NO: 5519, 3P_DLBCL (ARM); SEQ ID NO: 5520, 3P_DLBCL (ARM); SEQ ID NO: 5521, 3P_DLBCL (ARM); SEQ ID NO: 5522, 3P_DLBCL (ARM); SEQ ID NO: 5523, 3P_DLBCL (ARM); SEQ ID NO: 5524, 3P_DLBCL (ARM); SEQ ID NO: 5525, 3P_DLBCL (ARM); SEQ ID NO: 5526, 3P_DLBCL (ARM); SEQ ID NO: 5527, 3P_DLBCL (ARM); SEQ ID NO: 5528, 3P_DLBCL (ARM); SEQ ID NO: 5529, 3Q_DLBCL (ARM); SEQ ID NO: 5530, 3Q_DLBCL (ARM); SEQ ID NO: 5531, 3Q_DLBCL (ARM); SEQ ID NO: 5532, 3Q_DLBCL (ARM); SEQ ID NO: 5533, 3Q_DLBCL (ARM); SEQ ID NO: 5534, 3Q_DLBCL (ARM); SEQ ID NO: 5535, MSI (MSI); SEQ ID NO: 5536, MSI (MSI); SEQ ID NO: 5537, 3Q_DLBCL (ARM); SEQ ID NO: 5538, 3Q_DLBCL (ARM); SEQ ID NO: 5539, NFKBIZ (Gene); SEQ ID NO: 5540, NFKBIZ (Gene); SEQ ID NO: 5541, NFKBIZ (Gene); SEQ ID NO: 5542, NFKBIZ (Gene); SEQ ID NO: 5543, NFKBIZ (Gene); SEQ ID NO: 5544, NFKBIZ (Gene); SEQ ID NO: 5545, NFKBIZ (Gene); SEQ ID NO: 5546, NFKBIZ (Gene); SEQ ID NO: 5547, NFKBIZ (Gene); SEQ ID NO: 5548, NFKBIZ (Gene); SEQ ID NO: 5549, NFKBIZ (Gene); SEQ ID NO: 5550, NFKBIZ (Gene); SEQ ID NO: 5551, NFKBIZ 3′UTR (SV); SEQ ID NO: 5552, NFKBIZ 3′UTR (SV); SEQ ID NO: 5553, NFKBIZ 3′UTR (SV); SEQ ID NO: 5554, NFKBIZ 3′UTR (SV); SEQ ID NO: 5555, 3Q_DLBCL (ARM); SEQ ID NO: 5556, 3Q_DLBCL (ARM); SEQ ID NO: 5557, 3Q_DLBCL (ARM); SEQ ID NO: 5558, 3Q_DLBCL (ARM); SEQ ID NO: 5559, 3Q_DLBCL (ARM); SEQ ID NO: 5560, 3Q_DLBCL (ARM); SEQ ID NO: 5561, 3Q_DLBCL (ARM); SEQ ID NO: 5562, 3Q_DLBCL (ARM); SEQ ID NO: 5563, MSI (MSI); SEQ ID NO: 5564, MSI (MSI); SEQ ID NO: 5565, 3Q_DLBCL (ARM); SEQ ID NO: 5566, 3Q_DLBCL (ARM); SEQ ID NO: 5567, 3Q_DLBCL (ARM); SEQ ID NO: 5568, 3Q_DLBCL (ARM); SEQ ID NO: 5569, 3Q_DLBCL (ARM); SEQ ID NO: 5570, 3Q_DLBCL (ARM); SEQ ID NO: 5571, 3Q_DLBCL (ARM); SEQ ID NO: 5572, 3Q_DLBCL (ARM); SEQ ID NO: 5573, 3Q_DLBCL (ARM); SEQ ID NO: 5574, 3Q_DLBCL (ARM); SEQ ID NO: 5575, 3Q_DLBCL (ARM); SEQ ID NO: 5576, 3Q_DLBCL (ARM); SEQ ID NO: 5577, 3Q_DLBCL (ARM); SEQ ID NO: 5578, 3Q_DLBCL (ARM); SEQ ID NO: 5579, 3Q_DLBCL (ARM); SEQ ID NO: 5580, 3Q_DLBCL (ARM); SEQ ID NO: 5581, 3Q_DLBCL (ARM); SEQ ID NO: 5582, FP (FP); SEQ ID NO: 5583, 3Q_DLBCL (ARM); SEQ ID NO: 5584, 3Q_DLBCL (ARM); SEQ ID NO: 5585, 3Q_DLBCL (ARM); SEQ ID NO: 5586, 3Q_DLBCL (ARM); SEQ ID NO: 5587, 3Q_DLBCL (ARM); SEQ ID NO: 5588, ATR (Gene); SEQ ID NO: 5589, ATR (Gene); SEQ ID NO: 5590, ATR (Gene); SEQ ID NO: 5591, ATR (Gene); SEQ ID NO: 5592, ATR (Gene); SEQ ID NO: 5593, ATR (Gene); SEQ ID NO: 5594, ATR (Gene); SEQ ID NO: 5595, ATR (Gene); SEQ ID NO: 5596, ATR (Gene); SEQ ID NO: 5597, ATR (Gene); SEQ ID NO: 5598, ATR (Gene); SEQ ID NO: 5599, ATR (Gene); SEQ ID NO: 5600, ATR (Gene); SEQ ID NO: 5601, ATR (Gene); SEQ ID NO: 5602, ATR (Gene); SEQ ID NO: 5603, ATR (Gene); SEQ ID NO: 5604, ATR (Gene); SEQ ID NO: 5605, ATR (Gene); SEQ ID NO: 5606, ATR (Gene); SEQ ID NO: 5607, ATR (Gene); SEQ ID NO: 5608, ATR (Gene); SEQ ID NO: 5609, ATR (Gene); SEQ ID NO: 5610, ATR (Gene); SEQ ID NO: 5611, ATR (Gene); SEQ ID NO: 5612, ATR (Gene); SEQ ID NO: 5613, ATR (Gene); SEQ ID NO: 5614, ATR (Gene); SEQ ID NO: 5615, ATR (Gene); SEQ ID NO: 5616, ATR (Gene); SEQ ID NO: 5617, ATR (Gene); SEQ ID NO: 5618, ATR (Gene); SEQ ID NO: 5619, ATR (Gene); SEQ ID NO: 5620, ATR (Gene); SEQ ID NO: 5621, ATR (Gene); SEQ ID NO: 5622, ATR (Gene); SEQ ID NO: 5623, ATR (Gene); SEQ ID NO: 5624, ATR (Gene); SEQ ID NO: 5625, ATR (Gene); SEQ ID NO: 5626, ATR (Gene); SEQ ID NO: 5627, ATR (Gene); SEQ ID NO: 5628, ATR (Gene); SEQ ID NO: 5629, ATR (Gene); SEQ ID NO: 5630, ATR (Gene); SEQ ID NO: 5631, ATR (Gene); SEQ ID NO: 5632, ATR (Gene); SEQ ID NO: 5633, ATR (Gene); SEQ ID NO: 5634, ATR (Gene); SEQ ID NO: 5635, ATR (Gene); SEQ ID NO: 5636, 3Q_DLBCL (ARM); SEQ ID NO: 5637, 3Q_DLBCL (ARM); SEQ ID NO: 5638, 3Q_DLBCL (ARM); SEQ ID NO: 5639, 3Q_DLBCL (ARM); SEQ ID NO: 5640, 3Q_DLBCL (ARM); SEQ ID NO: 5641, 3Q_DLBCL (ARM); SEQ ID NO: 5642, 3Q_DLBCL (ARM); SEQ ID NO: 5643, FP (FP); SEQ ID NO: 5644, 3Q_DLBCL (ARM); SEQ ID NO: 5645, 3Q_DLBCL (ARM); SEQ ID NO: 5646, 3Q_DLBCL (ARM); SEQ ID NO: 5647, MSI (MSI); SEQ ID NO: 5648, MSI (MSI); SEQ ID NO: 5649, 3Q_DLBCL (ARM); SEQ ID NO: 5650, 3Q_DLBCL (ARM); SEQ ID NO: 5651, 3Q_DLBCL (ARM); SEQ ID NO: 5652, 3Q_DLBCL (ARM); SEQ ID NO: 5653, 3Q_DLBCL (ARM); SEQ ID NO: 5654, 3Q_DLBCL (ARM); SEQ ID NO: 5655, 3Q_DLBCL (ARM); SEQ ID NO: 5656, 3Q_DLBCL (ARM); SEQ ID NO: 5657, 3Q_DLBCL (ARM); SEQ ID NO: 5658, 3Q_DLBCL (ARM); SEQ ID NO: 5659, TBL1XR1 (Gene); SEQ ID NO: 5660, TBL1XR1 (Gene); SEQ ID NO: 5661, TBL1XR1_SV (SV); SEQ ID NO: 5662, TBL1XR1_SV (SV); SEQ ID NO: 5663, TBL1XR1_SV (SV); SEQ ID NO: 5664, TBL1XR1_SV (SV); SEQ ID NO: 5665, TBL1XR1 (Gene); SEQ ID NO: 5666, TBL1XR1 (Gene); SEQ ID NO: 5667, TBL1XR1_SV (SV); SEQ ID NO: 5668, TBL1XR1_SV (SV); SEQ ID NO: 5669, TBL1XR1_SV (SV); SEQ ID NO: 5670, TBL1XR1_SV (SV); SEQ ID NO: 5671, TBL1XR1 (Gene); SEQ ID NO: 5672, TBL1XR1 (Gene); SEQ ID NO: 5673, TBL1XR1 (Gene); SEQ ID NO: 5674, TBL1XR1 (Gene); SEQ ID NO: 5675, TBL1XR1 (Gene); SEQ ID NO: 5676, TBL1XR1 (Gene); SEQ ID NO: 5677, TBL1XR1_SV (SV); SEQ ID NO: 5678, TBL1XR1_SV (SV); SEQ ID NO: 5679, TBL1XR1_SV (SV); SEQ ID NO: 5680, TBL1XR1 (Gene); SEQ ID NO: 5681, TBL1XR1 (Gene); SEQ ID NO: 5682, TBL1XR1_SV (SV); SEQ ID NO: 5683, TBL1XR1_SV (SV); SEQ ID NO: 5684, TBL1XR1_SV (SV); SEQ ID NO: 5685, TBL1XR1_SV (SV); SEQ ID NO: 5686, TBL1XR1_SV (SV); SEQ ID NO: 5687, TBL1XR1_SV (SV); SEQ ID NO: 5688, TBL1XR1_SV (SV); SEQ ID NO: 5689, TBL1XR1_SV (SV); SEQ ID NO: 5690, TBL1XR1_SV (SV); SEQ ID NO: 5691, TBL1XR1_SV (SV); SEQ ID NO: 5692, TBL1XR1_SV (SV); SEQ ID NO: 5693, TBL1XR1_SV (SV); SEQ ID NO: 5694, TBL1XR1_SV (SV); SEQ ID NO: 5695, TBL1XR1_SV (SV); SEQ ID NO: 5696, TBL1XR1_SV (SV); SEQ ID NO: 5697, TBL1XR1_SV (SV); SEQ ID NO: 5698, TBL1XR1_SV (SV); SEQ ID NO: 5699, TBL1XR1_SV (SV); SEQ ID NO: 5700, TBL1XR1_SV (SV); SEQ ID NO: 5701, TBL1XR1_SV (SV); SEQ ID NO: 5702, TBL1XR1_SV (SV); SEQ ID NO: 5703, TBL1XR1_SV (SV); SEQ ID NO: 5704, TBL1XR1_SV (SV); SEQ ID NO: 5705, TBL1XR1_SV (SV); SEQ ID NO: 5706, TBL1XR1_SV (SV); SEQ ID NO: 5707, TBL1XR1_SV (SV); SEQ ID NO: 5708, TBL1XR1_SV (SV); SEQ ID NO: 5709, TBL1XR1_SV (SV); SEQ ID NO: 5710, TBL1XR1_SV (SV); SEQ ID NO: 5711, TBL1XR1_SV (SV); SEQ ID NO: 5712, TBL1XR1_SV (SV); SEQ ID NO: 5713, TBL1XR1_SV (SV); SEQ ID NO: 5714, TBL1XR1_SV (SV); SEQ ID NO: 5715, TBL1XR1_SV (SV); SEQ ID NO: 5716, TBL1XR1_SV (SV); SEQ ID NO: 5717, TBL1XR1_SV (SV); SEQ ID NO: 5718, TBL1XR1_SV (SV); SEQ ID NO: 5719, TBL1XR1_SV (SV); SEQ ID NO: 5720, TBL1XR1_SV (SV); SEQ ID NO: 5721, TBL1XR1_SV (SV); SEQ ID NO: 5722, TBL1XR1_SV (SV); SEQ ID NO: 5723, TBL1XR1_SV (SV); SEQ ID NO: 5724, TBL1XR1_SV (SV); SEQ ID NO: 5725, TBL1XR1_SV (SV); SEQ ID NO: 5726, TBL1XR1_SV (SV); SEQ ID NO: 5727, TBL1XR1_SV (SV); SEQ ID NO: 5728, TBL1XR1_SV (SV); SEQ ID NO: 5729, TBL1XR1_SV (SV); SEQ ID NO: 5730, TBL1XR1_SV (SV); SEQ ID NO: 5731, TBL1XR1_SV (SV); SEQ ID NO: 5732, TBL1XR1_SV (SV); SEQ ID NO: 5733, TBL1XR1_SV (SV); SEQ ID NO: 5734, TBL1XR1_SV (SV); SEQ ID NO: 5735, TBL1XR1_SV (SV); SEQ ID NO: 5736, TBL1XR1_SV (SV); SEQ ID NO: 5737, TBL1XR1_SV (SV); SEQ ID NO: 5738, TBL1XR1_SV (SV); SEQ ID NO: 5739, TBL1XR1_SV (SV); SEQ ID NO: 5740, TBL1XR1_SV (SV); SEQ ID NO: 5741, TBL1XR1_SV (SV); SEQ ID NO: 5742, 3Q_DLBCL (ARM); SEQ ID NO: 5743, 3Q_DLBCL (ARM); SEQ ID NO: 5744, PIK3CA (Gene); SEQ ID NO: 5745, PIK3CA (Gene); SEQ ID NO: 5746, PIK3CA (Gene); SEQ ID NO: 5747, PIK3CA (Gene); SEQ ID NO: 5748, PIK3CA (Gene); SEQ ID NO: 5749, PIK3CA (Gene); SEQ ID NO: 5750, PIK3CA (Gene); SEQ ID NO: 5751, PIK3CA (Gene); SEQ ID NO: 5752, PIK3CA (Gene); SEQ ID NO: 5753, PIK3CA (Gene); SEQ ID NO: 5754, PIK3CA (Gene); SEQ ID NO: 5755, PIK3CA (Gene); SEQ ID NO: 5756, PIK3CA (Gene); SEQ ID NO: 5757, PIK3CA (Gene); SEQ ID NO: 5758, PIK3CA (Gene); SEQ ID NO: 5759, PIK3CA (Gene); SEQ ID NO: 5760, PIK3CA (Gene); SEQ ID NO: 5761, PIK3CA (Gene); SEQ ID NO: 5762, PIK3CA (Gene); SEQ ID NO: 5763, PIK3CA (Gene); SEQ ID NO: 5764, PIK3CA (Gene); SEQ ID NO: 5765, PIK3CA (Gene); SEQ ID NO: 5766, 3Q_DLBCL (ARM); SEQ ID NO: 5767, KLHL6 (Gene); SEQ ID NO: 5768, KLHL6 (Gene); SEQ ID NO: 5769, KLHL6 (Gene); SEQ ID NO: 5770, KLHL6 (Gene); SEQ ID NO: 5771, KLHL6 (Gene); SEQ ID NO: 5772, KLHL6 (Gene); SEQ ID NO: 5773, KLHL6 (Gene); SEQ ID NO: 5774, 3Q_DLBCL (ARM); SEQ ID NO: 5775, 3Q_DLBCL (ARM); SEQ ID NO: 5776, 3Q_DLBCL (ARM); SEQ ID NO: 5777, FP (FP); SEQ ID NO: 5778, 3Q_DLBCL (ARM); SEQ ID NO: 5779, 3q28_DLBCL (FOCAL); SEQ ID NO: 5780, BCL6 (Gene); SEQ ID NO: 5781, BCL6 (Gene); SEQ ID NO: 5782, BCL6 (Gene); SEQ ID NO: 5783, BCL6 (Gene); SEQ ID NO: 5784, BCL6 (Gene); SEQ ID NO: 5785, BCL6 (Gene); SEQ ID NO: 5786, BCL6 (Gene); SEQ ID NO: 5787, BCL6 (Gene); SEQ ID NO: 5788, BCL6 (Gene); SEQ ID NO: 5789, BCL6 (Gene); SEQ ID NO: 5790, BCL6 (Gene); SEQ ID NO: 5791, BCL6 (Gene); SEQ ID NO: 5792, BCL6 (Gene); SEQ ID NO: 5793, BCL6 (Gene); SEQ ID NO: 5794, BCL6 (Gene); SEQ ID NO: 5795, BCL6_SV (SV); SEQ ID NO: 5796, BCL6_SV (SV); SEQ ID NO: 5797, BCL6_SV (SV); SEQ ID NO: 5798, BCL6_SV (SV); SEQ ID NO: 5799, BCL6_SV (SV); SEQ ID NO: 5800, BCL6_SV (SV); SEQ ID NO: 5801, BCL6_SV (SV); SEQ ID NO: 5802, 3q28 DLBCL (FOCAL); SEQ ID NO: 5803, 3q28 DLBCL (FOCAL); SEQ ID NO: 5804, 3q28 DLBCL (FOCAL); SEQ ID NO: 5805, 3q28 DLBCL (FOCAL); SEQ ID NO: 5806, 3q28_DLBCL (FOCAL); SEQ ID NO: 5807, 3q28_DLBCL (FOCAL); SEQ ID NO: 5808, 3q28_DLBCL (FOCAL); SEQ ID NO: 5809, 3q28_DLBCL (FOCAL); SEQ ID NO: 5810, 3q28 DLBCL (FOCAL); SEQ ID NO: 5811, TP63 (Gene); SEQ ID NO: 5812, TP63 (Gene); SEQ ID NO: 5813, TP63 (Gene); SEQ ID NO: 5814, TP63_SV (SV); SEQ ID NO: 5815, TP63_SV (SV); SEQ ID NO: 5816, TP63_SV (SV); SEQ ID NO: 5817, TP63_SV (SV); SEQ ID NO: 5818, TP63_SV (SV); SEQ ID NO: 5819, TP63_SV (SV); SEQ ID NO: 5820, TP63_SV (SV); SEQ ID NO: 5821, TP63_SV (SV); SEQ ID NO: 5822, TP63_SV (SV); SEQ ID NO: 5823, TP63_SV (SV); SEQ ID NO: 5824, TP63_SV (SV); SEQ ID NO: 5825, TP63_SV (SV); SEQ ID NO: 5826, TP63_SV (SV); SEQ ID NO: 5827, TP63_SV (SV); SEQ ID NO: 5828, TP63_SV (SV); SEQ ID NO: 5829, TP63_SV (SV); SEQ ID NO: 5830, TP63_SV (SV); SEQ ID NO: 5831, TP63_SV (SV); SEQ ID NO: 5832, TP63_SV (SV); SEQ ID NO: 5833, TP63_SV (SV); SEQ ID NO: 5834, TP63_SV (SV); SEQ ID NO: 5835, TP63_SV (SV); SEQ ID NO: 5836, TP63_SV (SV); SEQ ID NO: 5837, TP63_SV (SV); SEQ ID NO: 5838, TP63_SV (SV); SEQ ID NO: 5839, TP63_SV (SV); SEQ ID NO: 5840, TP63_SV (SV); SEQ ID NO: 5841, TP63_SV (SV); SEQ ID NO: 5842, TP63_SV (SV); SEQ ID NO: 5843, TP63_SV (SV); SEQ ID NO: 5844, TP63_SV (SV); SEQ ID NO: 5845, TP63_SV (SV); SEQ ID NO: 5846, TP63_SV (SV); SEQ ID NO: 5847, TP63_SV (SV); SEQ ID NO: 5848, TP63_SV (SV); SEQ ID NO: 5849, TP63_SV (SV); SEQ ID NO: 5850, TP63_SV (SV); SEQ ID NO: 5851, TP63_SV (SV); SEQ ID NO: 5852, TP63_SV (SV); SEQ ID NO: 5853, TP63_SV (SV); SEQ ID NO: 5854, TP63_SV (SV); SEQ ID NO: 5855, TP63_SV (SV); SEQ ID NO: 5856, TP63_SV (SV); SEQ ID NO: 5857, TP63_SV (SV); SEQ ID NO: 5858, TP63_SV (SV); SEQ ID NO: 5859, TP63_SV (SV); SEQ ID NO: 5860, TP63_SV (SV); SEQ ID NO: 5861, TP63_SV (SV); SEQ ID NO: 5862, TP63_SV (SV); SEQ ID NO: 5863, TP63_SV (SV); SEQ ID NO: 5864, TP63_SV (SV); SEQ ID NO: 5865, TP63_SV (SV); SEQ ID NO: 5866, TP63_SV (SV); SEQ ID NO: 5867, TP63_SV (SV); SEQ ID NO: 5868, TP63_SV (SV); SEQ ID NO: 5869, TP63_SV (SV); SEQ ID NO: 5870, TP63_SV (SV); SEQ ID NO: 5871, TP63_SV (SV); SEQ ID NO: 5872, TP63_SV (SV); SEQ ID NO: 5873, TP63_SV (SV); SEQ ID NO: 5874, TP63_SV (SV); SEQ ID NO: 5875, TP63_SV (SV); SEQ ID NO: 5876, TP63_SV (SV); SEQ ID NO: 5877, TP63_SV (SV); SEQ ID NO: 5878, TP63_SV (SV); SEQ ID NO: 5879, TP63_SV (SV); SEQ ID NO: 5880, TP63_SV (SV); SEQ ID NO: 5881, TP63_SV (SV); SEQ ID NO: 5882, TP63_SV (SV); SEQ ID NO: 5883, TP63_SV (SV); SEQ ID NO: 5884, TP63_SV (SV); SEQ ID NO: 5885, TP63_SV (SV); SEQ ID NO: 5886, TP63_SV (SV); SEQ ID NO: 5887, TP63_SV (SV); SEQ ID NO: 5888, TP63_SV (SV); SEQ ID NO: 5889, TP63_SV (SV); SEQ ID NO: 5890, TP63_SV (SV); SEQ ID NO: 5891, TP63_SV (SV); SEQ ID NO: 5892, TP63_SV (SV); SEQ ID NO: 5893, TP63_SV (SV); SEQ ID NO: 5894, TP63_SV (SV); SEQ ID NO: 5895, TP63_SV (SV); SEQ ID NO: 5896, TP63_SV (SV); SEQ ID NO: 5897, TP63_SV (SV); SEQ ID NO: 5898, TP63_SV (SV); SEQ ID NO: 5899, TP63_SV (SV); SEQ ID NO: 5900, TP63_SV (SV); SEQ ID NO: 5901, TP63_SV (SV); SEQ ID NO: 5902, TP63_SV (SV); SEQ ID NO: 5903, TP63_SV (SV); SEQ ID NO: 5904, TP63_SV (SV); SEQ ID NO: 5905, TP63_SV (SV); SEQ ID NO: 5906, TP63_SV (SV); SEQ ID NO: 5907, TP63_SV (SV); SEQ ID NO: 5908, TP63_SV (SV); SEQ ID NO: 5909, TP63_SV (SV); SEQ ID NO: 5910, TP63_SV (SV); SEQ ID NO: 5911, TP63_SV (SV); SEQ ID NO: 5912, TP63_SV (SV); SEQ ID NO: 5913, TP63_SV (SV); SEQ ID NO: 5914, TP63_SV (SV); SEQ ID NO: 5915, TP63_SV (SV); SEQ ID NO: 5916, TP63_SV (SV); SEQ ID NO: 5917, TP63_SV (SV); SEQ ID NO: 5918, TP63_SV (SV); SEQ ID NO: 5919, TP63_SV (SV); SEQ ID NO: 5920, TP63_SV (SV); SEQ ID NO: 5921, TP63_SV (SV); SEQ ID NO: 5922, TP63_SV (SV); SEQ ID NO: 5923, TP63_SV (SV); SEQ ID NO: 5924, TP63_SV (SV); SEQ ID NO: 5925, TP63_SV (SV); SEQ ID NO: 5926, TP63_SV (SV); SEQ ID NO: 5927, TP63_SV (SV); SEQ ID NO: 5928, TP63_SV (SV); SEQ ID NO: 5929, TP63_SV (SV); SEQ ID NO: 5930, TP63_SV (SV); SEQ ID NO: 5931, TP63_SV (SV); SEQ ID NO: 5932, TP63_SV (SV); SEQ ID NO: 5933, TP63_SV (SV); SEQ ID NO: 5934, TP63_SV (SV); SEQ ID NO: 5935, TP63_SV (SV); SEQ ID NO: 5936, TP63_SV (SV); SEQ ID NO: 5937, TP63_SV (SV); SEQ ID NO: 5938, TP63_SV (SV); SEQ ID NO: 5939, TP63_SV (SV); SEQ ID NO: 5940, TP63_SV (SV); SEQ ID NO: 5941, TP63_SV (SV); SEQ ID NO: 5942, TP63_SV (SV); SEQ ID NO: 5943, TP63_SV (SV); SEQ ID NO: 5944, TP63_SV (SV); SEQ ID NO: 5945, TP63_SV (SV); SEQ ID NO: 5946, TP63_SV (SV); SEQ ID NO: 5947, TP63_SV (SV); SEQ ID NO: 5948, TP63_SV (SV); SEQ ID NO: 5949, TP63_SV (SV); SEQ ID NO: 5950, TP63_SV (SV); SEQ ID NO: 5951, TP63_SV (SV); SEQ ID NO: 5952, TP63_SV (SV); SEQ ID NO: 5953, TP63_SV (SV); SEQ ID NO: 5954, TP63_SV (SV); SEQ ID NO: 5955, TP63_SV (SV); SEQ ID NO: 5956, TP63_SV (SV); SEQ ID NO: 5957, TP63_SV (SV); SEQ ID NO: 5958, TP63_SV (SV); SEQ ID NO: 5959, TP63_SV (SV); SEQ ID NO: 5960, TP63_SV (SV); SEQ ID NO: 5961, TP63_SV (SV); SEQ ID NO: 5962, TP63_SV (SV); SEQ ID NO: 5963, TP63_SV (SV); SEQ ID NO: 5964, TP63_SV (SV); SEQ ID NO: 5965, TP63_SV (SV); SEQ ID NO: 5966, TP63_SV (SV); SEQ ID NO: 5967, TP63_SV (SV); SEQ ID NO: 5968, TP63_SV (SV); SEQ ID NO: 5969, TP63_SV (SV); SEQ ID NO: 5970, TP63_SV (SV); SEQ ID NO: 5971, TP63_SV (SV); SEQ ID NO: 5972, TP63_SV (SV); SEQ ID NO: 5973, TP63_SV (SV); SEQ ID NO: 5974, TP63_SV (SV); SEQ ID NO: 5975, TP63_SV (SV); SEQ ID NO: 5976, TP63_SV (SV); SEQ ID NO: 5977, TP63_SV (SV); SEQ ID NO: 5978, TP63_SV (SV); SEQ ID NO: 5979, TP63_SV (SV); SEQ ID NO: 5980, TP63_SV (SV); SEQ ID NO: 5981, TP63_SV (SV); SEQ ID NO: 5982, TP63_SV (SV); SEQ ID NO: 5983, TP63_SV (SV); SEQ ID NO: 5984, TP63_SV (SV); SEQ ID NO: 5985, TP63_SV (SV); SEQ ID NO: 5986, TP63_SV (SV); SEQ ID NO: 5987, TP63_SV (SV); SEQ ID NO: 5988, TP63_SV (SV); SEQ ID NO: 5989, TP63_SV (SV); SEQ ID NO: 5990, TP63_SV (SV); SEQ ID NO: 5991, TP63_SV (SV); SEQ ID NO: 5992, TP63_SV (SV); SEQ ID NO: 5993, TP63_SV (SV); SEQ ID NO: 5994, TP63_SV (SV); SEQ ID NO: 5995, TP63_SV (SV); SEQ ID NO: 5996, TP63_SV (SV); SEQ ID NO: 5997, TP63_SV (SV); SEQ ID NO: 5998, TP63_SV (SV); SEQ ID NO: 5999, TP63_SV (SV); SEQ ID NO: 6000, TP63_SV (SV); SEQ ID NO: 6001, TP63_SV (SV); SEQ ID NO: 6002, TP63_SV (SV); SEQ ID NO: 6003, TP63 (Gene); SEQ ID NO: 6004, TP63 (Gene); SEQ ID NO: 6005, TP63 (Gene); SEQ ID NO: 6006, TP63 (Gene); SEQ ID NO: 6007, TP63 (Gene); SEQ ID NO: 6008, TP63 (Gene); SEQ ID NO: 6009, TP63 (Gene); SEQ ID NO: 6010, TP63 (Gene); SEQ ID NO: 6011, TP63 (Gene); SEQ ID NO: 6012, TP63 (Gene); SEQ ID NO: 6013, TP63 (Gene); SEQ ID NO: 6014, 3q28_DLBCL (FOCAL); SEQ ID NO: 6015, 3q28_DLBCL (FOCAL); SEQ ID NO: 6016, 3q28 DLBCL (FOCAL); SEQ ID NO: 6017, 3q28 DLBCL (FOCAL); SEQ ID NO: 6018, 3q28_DLBCL (FOCAL); SEQ ID NO: 6019, 3q28_DLBCL (FOCAL); SEQ ID NO: 6020, 3q28_DLBCL (FOCAL); SEQ ID NO: 6021, 3q28_DLBCL (FOCAL); SEQ ID NO: 6022, 3q28_DLBCL (FOCAL); SEQ ID NO: 6023, 3q28_DLBCL (FOCAL); SEQ ID NO: 6024, 3q28_DLBCL (FOCAL); SEQ ID NO: 6025, 3q28_DLBCL (FOCAL); SEQ ID NO: 6026, 3q28_DLBCL (FOCAL); SEQ ID NO: 6027, 3q28_DLBCL (FOCAL); SEQ ID NO: 6028, 3q28_DLBCL (FOCAL); SEQ ID NO: 6029, 3q28_DLBCL (FOCAL); SEQ ID NO: 6030, 3q28_DLBCL (FOCAL); SEQ ID NO: 6031, 3q28_DLBCL (FOCAL); SEQ ID NO: 6032, 3q28_DLBCL (FOCAL); SEQ ID NO: 6033, 3q28_DLBCL (FOCAL); SEQ ID NO: 6034, 3q28_DLBCL (FOCAL); SEQ ID NO: 6035, 3q28_DLBCL (FOCAL); SEQ ID NO: 6036, 3q28_DLBCL (FOCAL); SEQ ID NO: 6037, 3q28 DLBCL (FOCAL); SEQ ID NO: 6038, 3q28_DLBCL (FOCAL); SEQ ID NO: 6039, 3q28_DLBCL (FOCAL); SEQ ID NO: 6040, 3q28_DLBCL (FOCAL); SEQ ID NO: 6041, 3q28_DLBCL (FOCAL); SEQ ID NO: 6042, 3q28_DLBCL (FOCAL); SEQ ID NO: 6043, 3q28_DLBCL (FOCAL); SEQ ID NO: 6044, 3q28_DLBCL (FOCAL); SEQ ID NO: 6045, 3q28_DLBCL (FOCAL); SEQ ID NO: 6046, 3q28_DLBCL (FOCAL); SEQ ID NO: 6047, 3q28 DLBCL (FOCAL); SEQ ID NO: 6048, 3q28_DLBCL (FOCAL); SEQ ID NO: 6049, 3q28_DLBCL (FOCAL); SEQ ID NO: 6050, 3q28_DLBCL (FOCAL); SEQ ID NO: 6051, 3q28_DLBCL (FOCAL); SEQ ID NO: 6052, 3q28_DLBCL (FOCAL); SEQ ID NO: 6053, 4P (ARM); SEQ ID NO: 6054, NSD2 (Gene); SEQ ID NO: 6055, NSD2 (Gene); SEQ ID NO: 6056, NSD2 (Gene); SEQ ID NO: 6057, NSD2 (Gene); SEQ ID NO: 6058, NSD2 (Gene); SEQ ID NO: 6059, NSD2 (Gene); SEQ ID NO: 6060, NSD2 (Gene); SEQ ID NO: 6061, NSD2 (Gene); SEQ ID NO: 6062, NSD2 (Gene); SEQ ID NO: 6063, NSD2 (Gene); SEQ ID NO: 6064, NSD2 (Gene); SEQ ID NO: 6065, NSD2 (Gene); SEQ ID NO: 6066, NSD2 (Gene); SEQ ID NO: 6067, NSD2 (Gene); SEQ ID NO: 6068, NSD2 (Gene); SEQ ID NO: 6069, NSD2 (Gene); SEQ ID NO: 6070, NSD2 (Gene); SEQ ID NO: 6071, NSD2 (Gene); SEQ ID NO: 6072, NSD2 (Gene); SEQ ID NO: 6073, NSD2 (Gene); SEQ ID NO: 6074, NSD2 (Gene); SEQ ID NO: 6075, NSD2 (Gene); SEQ ID NO: 6076, NSD2 (Gene); SEQ ID NO: 6077, NSD2 (Gene); SEQ ID NO: 6078, NSD2 (Gene); SEQ ID NO: 6079, MSI (MSI); SEQ ID NO: 6080, MSI (MSI); SEQ ID NO: 6081, 4P (ARM); SEQ ID NO: 6082, 4P (ARM); SEQ ID NO: 6083, 4P (ARM); SEQ ID NO: 6084, 4P (ARM); SEQ ID NO: 6085, 4P (ARM); SEQ ID NO: 6086, 4P (ARM); SEQ ID NO: 6087, 4P (ARM); SEQ ID NO: 6088, 4P (ARM); SEQ ID NO: 6089, FP (FP); SEQ ID NO: 6090, 4P (ARM); SEQ ID NO: 6091, 4P (ARM); SEQ ID NO: 6092, 4P (ARM); SEQ ID NO: 6093, 4P (ARM); SEQ ID NO: 6094, 4P (ARM); SEQ ID NO: 6095, MSI (MSI); SEQ ID NO: 6096, MSI (MSI); SEQ ID NO: 6097, 4P (ARM); SEQ ID NO: 6098, 4P (ARM); SEQ ID NO: 6099, 4P (ARM); SEQ ID NO: 6100, 4P (ARM); SEQ ID NO: 6101, 4P (ARM); SEQ ID NO: 6102, 4P (ARM); SEQ ID NO: 6103, 4P (ARM); SEQ ID NO: 6104, 4P (ARM); SEQ ID NO: 6105, 4P (ARM); SEQ ID NO: 6106, SEL1L3 (Gene); SEQ ID NO: 6107, SEL1L3 (Gene); SEQ ID NO: 6108, SEL1L3 (Gene); SEQ ID NO: 6109, SEL1L3 (Gene); SEQ ID NO: 6110, SEL1L3 (Gene); SEQ ID NO: 6111, SEL1L3 (Gene); SEQ ID NO: 6112, SEL1L3 (Gene); SEQ ID NO: 6113, SEL1L3 (Gene); SEQ ID NO: 6114, SEL1L3 (Gene); SEQ ID NO: 6115, SEL1L3 (Gene); SEQ ID NO: 6116, SEL1L3 (Gene); SEQ ID NO: 6117, SEL1L3 (Gene); SEQ ID NO: 6118, SEL1L3 (Gene); SEQ ID NO: 6119, SEL1L3 (Gene); SEQ ID NO: 6120, SEL1L3 (Gene); SEQ ID NO: 6121, SEL1L3 (Gene); SEQ ID NO: 6122, SEL1L3 (Gene); SEQ ID NO: 6123, SEL1L3 (Gene); SEQ ID NO: 6124, SEL1L3 (Gene); SEQ ID NO: 6125, SEL1L3 (Gene); SEQ ID NO: 6126, SEL1L3 (Gene); SEQ ID NO: 6127, SEL1L3 (Gene); SEQ ID NO: 6128, SEL1L3 (Gene); SEQ ID NO: 6129, SEL1L3 (Gene); SEQ ID NO: 6130, SEL1L3 (Gene); SEQ ID NO: 6131, SEL1L3 (Gene); SEQ ID NO: 6132, 4P (ARM); SEQ ID NO: 6133, FP (FP); SEQ ID NO: 6134, 4P (ARM); SEQ ID NO: 6135, 4P (ARM); SEQ ID NO: 6136, 4P (ARM); SEQ ID NO: 6137, 4P (ARM); SEQ ID NO: 6138, 4P (ARM); SEQ ID NO: 6139, 4P (ARM); SEQ ID NO: 6140, 4P (ARM); SEQ ID NO: 6141, 4P (ARM); SEQ ID NO: 6142, 4P (ARM); SEQ ID NO: 6143, 4P (ARM); SEQ ID NO: 6144, 4P (ARM); SEQ ID NO: 6145, 4P (ARM); SEQ ID NO: 6146, 4P (ARM); SEQ ID NO: 6147, 4P (ARM); SEQ ID NO: 6148, 4P (ARM); SEQ ID NO: 6149, 4P (ARM); SEQ ID NO: 6150, 4P (ARM); SEQ ID NO: 6151, 4P (ARM); SEQ ID NO: 6152, 4P (ARM); SEQ ID NO: 6153, KIT (Gene); SEQ ID NO: 6154, KIT (Gene); SEQ ID NO: 6155, KIT (Gene); SEQ ID NO: 6156, KIT (Gene); SEQ ID NO: 6157, KIT (Gene); SEQ ID NO: 6158, KIT (Gene); SEQ ID NO: 6159, KIT (Gene); SEQ ID NO: 6160, KIT (Gene); SEQ ID NO: 6161, KIT (Gene); SEQ ID NO: 6162, KIT (Gene); SEQ ID NO: 6163, KIT (Gene); SEQ ID NO: 6164, KIT (Gene); SEQ ID NO: 6165, KIT (Gene); SEQ ID NO: 6166, KIT (Gene); SEQ ID NO: 6167, KIT (Gene); SEQ ID NO: 6168, KIT (Gene); SEQ ID NO: 6169, KIT (Gene); SEQ ID NO: 6170, KIT (Gene); SEQ ID NO: 6171, KIT (Gene); SEQ ID NO: 6172, KIT (Gene); SEQ ID NO: 6173, KIT (Gene); SEQ ID NO: 6174, 4P (ARM); SEQ ID NO: 6175, 4P (ARM); SEQ ID NO: 6176, 4P (ARM); SEQ ID NO: 6177, 4P (ARM); SEQ ID NO: 6178, 4P (ARM); SEQ ID NO: 6179, 4P (ARM); SEQ ID NO: 6180, 4P (ARM); SEQ ID NO: 6181, FP (FP); SEQ ID NO: 6182, 4q21.22_DLBCL (FOCAL); SEQ ID NO: 6183, 4q21.22_DLBCL (FOCAL); SEQ ID NO: 6184, 4q21.22_DLBCL (FOCAL); SEQ ID NO: 6185, 4q21.22_DLBCL (FOCAL); SEQ ID NO: 6186, 4q21.22_DLBCL (FOCAL); SEQ ID NO: 6187, 4q21.22_DLBCL (FOCAL); SEQ ID NO: 6188, 4q21.22_DLBCL (FOCAL); SEQ ID NO: 6189, 4q21.22_DLBCL (FOCAL); SEQ ID NO: 6190, 4q21.22_DLBCL (FOCAL); SEQ ID NO: 6191, 4q21.22_DLBCL (FOCAL); SEQ ID NO: 6192, 4q21.22_DLBCL (FOCAL); SEQ ID NO: 6193, 4q21.22_DLBCL (FOCAL); SEQ ID NO: 6194, 4q21.22_DLBCL (FOCAL); SEQ ID NO: 6195, 4q21.22_DLBCL (FOCAL); SEQ ID NO: 6196, 4q21.22_DLBCL (FOCAL); SEQ ID NO: 6197, 4q21.22_DLBCL (FOCAL); SEQ ID NO: 6198, 4q21.22_DLBCL (FOCAL); SEQ ID NO: 6199, 4q21.22_DLBCL (FOCAL); SEQ ID NO: 6200, 4q21.22_DLBCL (FOCAL); SEQ ID NO: 6201, 4q21.22_DLBCL (FOCAL); SEQ ID NO: 6202, 4q21.22_DLBCL (FOCAL); SEQ ID NO: 6203, 4q21.22_DLBCL (FOCAL); SEQ ID NO: 6204, 4q21.22_DLBCL (FOCAL); SEQ ID NO: 6205, 4q21.22_DLBCL (FOCAL); SEQ ID NO: 6206, 4q21.22_DLBCL (FOCAL); SEQ ID NO: 6207, 4q21.22_DLBCL (FOCAL); SEQ ID NO: 6208, 4q21.22_DLBCL (FOCAL); SEQ ID NO: 6209, 4q21.22_DLBCL (FOCAL); SEQ ID NO: 6210, 4q21.22_DLBCL (FOCAL); SEQ ID NO: 6211, 4q21.22_DLBCL (FOCAL); SEQ ID NO: 6212, 4q21.22_DLBCL (FOCAL); SEQ ID NO: 6213, 4q21.22_DLBCL (FOCAL); SEQ ID NO: 6214, 4q21.22_DLBCL (FOCAL); SEQ ID NO: 6215, 4q21.22_DLBCL (FOCAL); SEQ ID NO: 6216, 4q21.22_DLBCL (FOCAL); SEQ ID NO: 6217, 4q21.22_DLBCL (FOCAL); SEQ ID NO: 6218, HNRNPD (Gene); SEQ ID NO: 6219, HNRNPD (Gene); SEQ ID NO: 6220, HNRNPD (Gene); SEQ ID NO: 6221, HNRNPD (Gene); SEQ ID NO: 6222, HNRNPD (Gene); SEQ ID NO: 6223, HNRNPD (Gene); SEQ ID NO: 6224, HNRNPD (Gene); SEQ ID NO: 6225, HNRNPD (Gene); SEQ ID NO: 6226, 4q21.22_DLBCL (FOCAL); SEQ ID NO: 6227, 4q21.22_DLBCL (FOCAL); SEQ ID NO: 6228, 4q21.22_DLBCL (FOCAL); SEQ ID NO: 6229, MSI (MSI); SEQ ID NO: 6230, MSI (MSI); SEQ ID NO: 6231, 4q21.22_DLBCL (FOCAL); SEQ ID NO: 6232, 4q21.22_DLBCL (FOCAL); SEQ ID NO: 6233, 4q21.22_DLBCL (FOCAL); SEQ ID NO: 6234, 4q21.22_DLBCL (FOCAL); SEQ ID NO: 6235, 4q21.22_DLBCL (FOCAL); SEQ ID NO: 6236, 4q21.22_DLBCL (FOCAL); SEQ ID NO: 6237, 4q21.22_DLBCL (FOCAL); SEQ ID NO: 6238, 4q21.22_DLBCL (FOCAL); SEQ ID NO: 6239, 4q21.22_DLBCL (FOCAL); SEQ ID NO: 6240, WDFY3 (Gene); SEQ ID NO: 6241, WDFY3 (Gene); SEQ ID NO: 6242, WDFY3 (Gene); SEQ ID NO: 6243, WDFY3 (Gene); SEQ ID NO: 6244, WDFY3 (Gene); SEQ ID NO: 6245, WDFY3 (Gene); SEQ ID NO: 6246, WDFY3 (Gene); SEQ ID NO: 6247, WDFY3 (Gene); SEQ ID NO: 6248, WDFY3 (Gene); SEQ ID NO: 6249, WDFY3 (Gene); SEQ ID NO: 6250, WDFY3 (Gene); SEQ ID NO: 6251, WDFY3 (Gene); SEQ ID NO: 6252, WDFY3 (Gene); SEQ ID NO: 6253, WDFY3 (Gene); SEQ ID NO: 6254, WDFY3 (Gene); SEQ ID NO: 6255, WDFY3 (Gene); SEQ ID NO: 6256, WDFY3 (Gene); SEQ ID NO: 6257, WDFY3 (Gene); SEQ ID NO: 6258, WDFY3 (Gene); SEQ ID NO: 6259, WDFY3 (Gene); SEQ ID NO: 6260, WDFY3 (Gene); SEQ ID NO: 6261, WDFY3 (Gene); SEQ ID NO: 6262, WDFY3 (Gene); SEQ ID NO: 6263, WDFY3 (Gene); SEQ ID NO: 6264, WDFY3 (Gene); SEQ ID NO: 6265, WDFY3 (Gene); SEQ ID NO: 6266, WDFY3 (Gene); SEQ ID NO: 6267, WDFY3 (Gene); SEQ ID NO: 6268, WDFY3 (Gene); SEQ ID NO: 6269, WDFY3 (Gene); SEQ ID NO: 6270, WDFY3 (Gene); SEQ ID NO: 6271, WDFY3 (Gene); SEQ ID NO: 6272, WDFY3 (Gene); SEQ ID NO: 6273, WDFY3 (Gene); SEQ ID NO: 6274, WDFY3 (Gene); SEQ ID NO: 6275, WDFY3 (Gene); SEQ ID NO: 6276, WDFY3 (Gene); SEQ ID NO: 6277, WDFY3 (Gene); SEQ ID NO: 6278, WDFY3 (Gene); SEQ ID NO: 6279, WDFY3 (Gene); SEQ ID NO: 6280, WDFY3 (Gene); SEQ ID NO: 6281, WDFY3 (Gene); SEQ ID NO: 6282, WDFY3 (Gene); SEQ ID NO: 6283, WDFY3 (Gene); SEQ ID NO: 6284, WDFY3 (Gene); SEQ ID NO: 6285, WDFY3 (Gene); SEQ ID NO: 6286, WDFY3 (Gene); SEQ ID NO: 6287, WDFY3 (Gene); SEQ ID NO: 6288, WDFY3 (Gene); SEQ ID NO: 6289, WDFY3 (Gene); SEQ ID NO: 6290, WDFY3 (Gene); SEQ ID NO: 6291, WDFY3 (Gene); SEQ ID NO: 6292, WDFY3 (Gene); SEQ ID NO: 6293, WDFY3 (Gene); SEQ ID NO: 6294, WDFY3 (Gene); SEQ ID NO: 6295, WDFY3 (Gene); SEQ ID NO: 6296, WDFY3 (Gene); SEQ ID NO: 6297, WDFY3 (Gene); SEQ ID NO: 6298, WDFY3 (Gene); SEQ ID NO: 6299, WDFY3 (Gene); SEQ ID NO: 6300, WDFY3 (Gene); SEQ ID NO: 6301, WDFY3 (Gene); SEQ ID NO: 6302, WDFY3 (Gene); SEQ ID NO: 6303, WDFY3 (Gene); SEQ ID NO: 6304, WDFY3 (Gene); SEQ ID NO: 6305, 4q21.22_DLBCL (FOCAL); SEQ ID NO: 6306, 4q21.22_DLBCL (FOCAL); SEQ ID NO: 6307, 4q21.22_DLBCL (FOCAL); SEQ ID NO: 6308, 4q21.22_DLBCL (FOCAL); SEQ ID NO: 6309, 4q21.22_DLBCL (FOCAL); SEQ ID NO: 6310, 4q21.22_DLBCL (FOCAL); SEQ ID NO: 6311, 4q21.22_DLBCL (FOCAL); SEQ ID NO: 6312, 4q21.22_DLBCL (FOCAL); SEQ ID NO: 6313, 4q21.22_DLBCL (FOCAL); SEQ ID NO: 6314, 4q21.22_DLBCL (FOCAL); SEQ ID NO: 6315, 4q21.22_DLBCL (FOCAL); SEQ ID NO: 6316, 4q21.22_DLBCL (FOCAL); SEQ ID NO: 6317, 4q21.22_DLBCL (FOCAL); SEQ ID NO: 6318, 4q21.22_DLBCL (FOCAL); SEQ ID NO: 6319, 4q21.22_DLBCL (FOCAL); SEQ ID NO: 6320, 4q21.22_DLBCL (FOCAL); SEQ ID NO: 6321, 4q21.22_DLBCL (FOCAL); SEQ ID NO: 6322, 4q21.22_DLBCL (FOCAL); SEQ ID NO: 6323, 4q21.22_DLBCL (FOCAL); SEQ ID NO: 6324, 4q21.22_DLBCL (FOCAL); SEQ ID NO: 6325, 4q21.22_DLBCL (FOCAL); SEQ ID NO: 6326, 4q21.22_DLBCL (FOCAL); SEQ ID NO: 6327, 4q21.22_DLBCL (FOCAL); SEQ ID NO: 6328, 4q21.22_DLBCL (FOCAL); SEQ ID NO: 6329, 4q21.22_DLBCL (FOCAL); SEQ ID NO: 6330, 4q21.22_DLBCL (FOCAL); SEQ ID NO: 6331, 4q21.22_DLBCL (FOCAL); SEQ ID NO: 6332, 4q21.22_DLBCL (FOCAL); SEQ ID NO: 6333, 4q21.22_DLBCL (FOCAL); SEQ ID NO: 6334, 4q21.22_DLBCL (FOCAL); SEQ ID NO: 6335, 4q21.22_DLBCL (FOCAL); SEQ ID NO: 6336, 4q21.22_DLBCL (FOCAL); SEQ ID NO: 6337, 4q21.22_DLBCL (FOCAL); SEQ ID NO: 6338, 4q21.22_DLBCL (FOCAL); SEQ ID NO: 6339, 4q21.22_DLBCL (FOCAL); SEQ ID NO: 6340, 4q21.22_DLBCL (FOCAL); SEQ ID NO: 6341, 4q21.22_DLBCL (FOCAL); SEQ ID NO: 6342, 4q21.22_DLBCL (FOCAL); SEQ ID NO: 6343, 4q21.22_DLBCL (FOCAL); SEQ ID NO: 6344, 4q21.22_DLBCL (FOCAL); SEQ ID NO: 6345, 4q21.22_DLBCL (FOCAL); SEQ ID NO: 6346, 4q21.22_DLBCL (FOCAL); SEQ ID NO: 6347, 4P (ARM); SEQ ID NO: 6348, 4P (ARM); SEQ ID NO: 6349, 4P (ARM); SEQ ID NO: 6350, 4P (ARM); SEQ ID NO: 6351, 4P (ARM); SEQ ID NO: 6352, 4P (ARM); SEQ ID NO: 6353, 4Q (ARM); SEQ ID NO: 6354, 4Q (ARM); SEQ ID NO: 6355, 4Q (ARM); SEQ ID NO: 6356, 4Q (ARM); SEQ ID NO: 6357, 4Q (ARM); SEQ ID NO: 6358, TET2 (Gene); SEQ ID NO: 6359, TET2 (Gene); SEQ ID NO: 6360, TET2 (Gene); SEQ ID NO: 6361, TET2 (Gene); SEQ ID NO: 6362, TET2 (Gene); SEQ ID NO: 6363, TET2 (Gene); SEQ ID NO: 6364, TET2 (Gene); SEQ ID NO: 6365, TET2 (Gene); SEQ ID NO: 6366, TET2 (Gene); SEQ ID NO: 6367, TET2 (Gene); SEQ ID NO: 6368, TET2 (Gene); SEQ ID NO: 6369, TET2 (Gene); SEQ ID NO: 6370, TET2 (Gene); SEQ ID NO: 6371, TET2 (Gene); SEQ ID NO: 6372, TET2 (Gene); SEQ ID NO: 6373, TET2 (Gene); SEQ ID NO: 6374, TET2 (Gene); SEQ ID NO: 6375, TET2 (Gene); SEQ ID NO: 6376, TET2 (Gene); SEQ ID NO: 6377, 4Q (ARM); SEQ ID NO: 6378, 4Q (ARM); SEQ ID NO: 6379, 4Q (ARM); SEQ ID NO: 6380, 4Q (ARM); SEQ ID NO: 6381, 4Q (ARM); SEQ ID NO: 6382, 4Q (ARM); SEQ ID NO: 6383, 4Q (ARM); SEQ ID NO: 6384, 4Q (ARM); SEQ ID NO: 6385, 4Q (ARM); SEQ ID NO: 6386, 4Q (ARM); SEQ ID NO: 6387, 4Q (ARM); SEQ ID NO: 6388, 4Q (ARM); SEQ ID NO: 6389, ANKRD50 (Gene); SEQ ID NO: 6390, ANKRD50 (Gene); SEQ ID NO: 6391, ANKRD50 (Gene); SEQ ID NO: 6392, ANKRD50 (Gene); SEQ ID NO: 6393, ANKRD50 (Gene); SEQ ID NO: 6394, ANKRD50 (Gene); SEQ ID NO: 6395, ANKRD50 (Gene); SEQ ID NO: 6396, ANKRD50 (Gene); SEQ ID NO: 6397, ANKRD50 (Gene); SEQ ID NO: 6398, ANKRD50 (Gene); SEQ ID NO: 6399, ANKRD50 (Gene); SEQ ID NO: 6400, 4Q (ARM); SEQ ID NO: 6401, 4Q (ARM); SEQ ID NO: 6402, 4Q (ARM); SEQ ID NO: 6403, 4Q (ARM); SEQ ID NO: 6404, 4Q (ARM); SEQ ID NO: 6405, 4Q (ARM); SEQ ID NO: 6406, 4Q (ARM); SEQ ID NO: 6407, 4Q (ARM); SEQ ID NO: 6408, 4Q (ARM); SEQ ID NO: 6409, 4Q (ARM); SEQ ID NO: 6410, 4Q (ARM); SEQ ID NO: 6411, 4q35.1_DLBCL (FOCAL); SEQ ID NO: 6412, 4q35.1_DLBCL (FOCAL); SEQ ID NO: 6413, 4q35.1_DLBCL (FOCAL); SEQ ID NO: 6414, 4q35.1_DLBCL (FOCAL); SEQ ID NO: 6415, 4q35.1_DLBCL (FOCAL); SEQ ID NO: 6416, 4q35.1_DLBCL (FOCAL); SEQ ID NO: 6417, 4q35.1_DLBCL (FOCAL); SEQ ID NO: 6418, 4q35.1_DLBCL (FOCAL); SEQ ID NO: 6419, 4q35.1_DLBCL (FOCAL); SEQ ID NO: 6420, 4q35.1_DLBCL (FOCAL); SEQ ID NO: 6421, 4q35.1_DLBCL (FOCAL); SEQ ID NO: 6422, 4q35.1_DLBCL (FOCAL); SEQ ID NO: 6423, 4q35.1_DLBCL (FOCAL); SEQ ID NO: 6424, 4q35.1_DLBCL (FOCAL); SEQ ID NO: 6425, 4q35.1_DLBCL (FOCAL); SEQ ID NO: 6426, 4q35.1_DLBCL (FOCAL); SEQ ID NO: 6427, 4q35.1_DLBCL (FOCAL); SEQ ID NO: 6428, 4q35.1_DLBCL (FOCAL); SEQ ID NO: 6429, 4q35.1_DLBCL (FOCAL); SEQ ID NO: 6430, 4q35.1_DLBCL (FOCAL); SEQ ID NO: 6431, 4q35.1_DLBCL (FOCAL); SEQ ID NO: 6432, 4q35.1_DLBCL (FOCAL); SEQ ID NO: 6433, 4q35.1_DLBCL (FOCAL); SEQ ID NO: 6434, 4q35.1_DLBCL (FOCAL); SEQ ID NO: 6435, 4q35.1_DLBCL (FOCAL); SEQ ID NO: 6436, 4q35.1_DLBCL (FOCAL); SEQ ID NO: 6437, 4q35.1_DLBCL (FOCAL); SEQ ID NO: 6438, 4q35.1_DLBCL (FOCAL); SEQ ID NO: 6439, 4q35.1_DLBCL (FOCAL); SEQ ID NO: 6440, 4q35.1_DLBCL (FOCAL); SEQ ID NO: 6441, 4q35.1_DLBCL (FOCAL); SEQ ID NO: 6442, 4q35.1_DLBCL (FOCAL); SEQ ID NO: 6443, 4q35.1_DLBCL (FOCAL); SEQ ID NO: 6444, 4q35.1_DLBCL (FOCAL); SEQ ID NO: 6445, 4q35.1_DLBCL (FOCAL); SEQ ID NO: 6446, FBXW7 (Gene); SEQ ID NO: 6447, FBXW7 (Gene); SEQ ID NO: 6448, FBXW7 (Gene); SEQ ID NO: 6449, FBXW7 (Gene); SEQ ID NO: 6450, FBXW7 (Gene); SEQ ID NO: 6451, FBXW7 (Gene); SEQ ID NO: 6452, FBXW7 (Gene); SEQ ID NO: 6453, FBXW7 (Gene); SEQ ID NO: 6454, FBXW7 (Gene); SEQ ID NO: 6455, FBXW7 (Gene); SEQ ID NO: 6456, FBXW7 (Gene); SEQ ID NO: 6457, FBXW7 (Gene); SEQ ID NO: 6458, FBXW7 (Gene); SEQ ID NO: 6459, FBXW7 (Gene); SEQ ID NO: 6460, 4q35.1_DLBCL (FOCAL); SEQ ID NO: 6461, 4q35.1_DLBCL (FOCAL); SEQ ID NO: 6462, 4q35.1_DLBCL (FOCAL); SEQ ID NO: 6463, 4q35.1_DLBCL (FOCAL); SEQ ID NO: 6464, 4q35.1_DLBCL (FOCAL); SEQ ID NO: 6465, TLR2 (Gene); SEQ ID NO: 6466, TLR2 (Gene); SEQ ID NO: 6467, TLR2 (Gene); SEQ ID NO: 6468, TLR2 (Gene); SEQ ID NO: 6469, TLR2 (Gene); SEQ ID NO: 6470, 4q35.1_DLBCL (FOCAL); SEQ ID NO: 6471, 4q35.1_DLBCL (FOCAL); SEQ ID NO: 6472, 4q35.1_DLBCL (FOCAL); SEQ ID NO: 6473, 4q35.1_DLBCL (FOCAL); SEQ ID NO: 6474, 4q35.1_DLBCL (FOCAL); SEQ ID NO: 6475, 4q35.1_DLBCL (FOCAL); SEQ ID NO: 6476, 4q35.1_DLBCL (FOCAL); SEQ ID NO: 6477, 4q35.1_DLBCL (FOCAL); SEQ ID NO: 6478, 4q35.1_DLBCL (FOCAL); SEQ ID NO: 6479, 4q35.1_DLBCL (FOCAL); SEQ ID NO: 6480, 4q35.1_DLBCL (FOCAL); SEQ ID NO: 6481, 4q35.1_DLBCL (FOCAL); SEQ ID NO: 6482, 4q35.1_DLBCL (FOCAL); SEQ ID NO: 6483, 4q35.1_DLBCL (FOCAL); SEQ ID NO: 6484, 4q35.1_DLBCL (FOCAL); SEQ ID NO: 6485, 4q35.1_DLBCL (FOCAL); SEQ ID NO: 6486, 4q35.1_DLBCL (FOCAL); SEQ ID NO: 6487, 4q35.1_DLBCL (FOCAL); SEQ ID NO: 6488, 4q35.1_DLBCL (FOCAL); SEQ ID NO: 6489, 4q35.1_DLBCL (FOCAL); SEQ ID NO: 6490, 4q35.1_DLBCL (FOCAL); SEQ ID NO: 6491, 4q35.1_DLBCL (FOCAL); SEQ ID NO: 6492, 4q35.1_DLBCL (FOCAL); SEQ ID NO: 6493, 4q35.1_DLBCL (FOCAL); SEQ ID NO: 6494, 4q35.1_DLBCL (FOCAL); SEQ ID NO: 6495, 4q35.1_DLBCL (FOCAL); SEQ ID NO: 6496, 4q35.1_DLBCL (FOCAL); SEQ ID NO: 6497, 4q35.1_DLBCL (FOCAL); SEQ ID NO: 6498, 4q35.1_DLBCL (FOCAL); SEQ ID NO: 6499, 4q35.1_DLBCL (FOCAL); SEQ ID NO: 6500, 4q35.1_DLBCL (FOCAL); SEQ ID NO: 6501, 4q35.1_DLBCL (FOCAL); SEQ ID NO: 6502, 4q35.1_DLBCL (FOCAL); SEQ ID NO: 6503, 4q35.1_DLBCL (FOCAL); SEQ ID NO: 6504, 4q35.1_DLBCL (FOCAL); SEQ ID NO: 6505, 4q35.1_DLBCL (FOCAL); SEQ ID NO: 6506, 4q35.1_DLBCL (FOCAL); SEQ ID NO: 6507, 4q35.1_DLBCL (FOCAL); SEQ ID NO: 6508, 4q35.1_DLBCL (FOCAL); SEQ ID NO: 6509, 4q35.1_DLBCL (FOCAL); SEQ ID NO: 6510, 4q35.1_DLBCL (FOCAL); SEQ ID NO: 6511, 4q35.1_DLBCL (FOCAL); SEQ ID NO: 6512, 4q35.1_DLBCL (FOCAL); SEQ ID NO: 6513, 4q35.1_DLBCL (FOCAL); SEQ ID NO: 6514, 4q35.1_DLBCL (FOCAL); SEQ ID NO: 6515, 4q35.1_DLBCL (FOCAL); SEQ ID NO: 6516, 4q35.1_DLBCL (FOCAL); SEQ ID NO: 6517, 4q35.1_DLBCL (FOCAL); SEQ ID NO: 6518, 4q35.1_DLBCL (FOCAL); SEQ ID NO: 6519, 4q35.1_DLBCL (FOCAL); SEQ ID NO: 6520, 4q35.1_DLBCL (FOCAL); SEQ ID NO: 6521, 4q35.1_DLBCL (FOCAL); SEQ ID NO: 6522, 4q35.1_DLBCL (FOCAL); SEQ ID NO: 6523, 4q35.1_DLBCL (FOCAL); SEQ ID NO: 6524, 4q35.1_DLBCL (FOCAL); SEQ ID NO: 6525, 4q35.1_DLBCL (FOCAL); SEQ ID NO: 6526, 4q35.1_DLBCL (FOCAL); SEQ ID NO: 6527, 4q35.1_DLBCL (FOCAL); SEQ ID NO: 6528, 4q35.1_DLBCL (FOCAL); SEQ ID NO: 6529, 4q35.1_DLBCL (FOCAL); SEQ ID NO: 6530, 4q35.1_DLBCL (FOCAL); SEQ ID NO: 6531, 4q35.1_DLBCL (FOCAL); SEQ ID NO: 6532, 4q35.1_DLBCL (FOCAL); SEQ ID NO: 6533, 4q35.1_DLBCL (FOCAL); SEQ ID NO: 6534, 4q35.1_DLBCL (FOCAL); SEQ ID NO: 6535, 4q35.1_DLBCL (FOCAL); SEQ ID NO: 6536, 4q35.1_DLBCL (FOCAL); SEQ ID NO: 6537, 4q35.1_DLBCL (FOCAL); SEQ ID NO: 6538, 4q35.1_DLBCL (FOCAL); SEQ ID NO: 6539, 4q35.1_DLBCL (FOCAL); SEQ ID NO: 6540, 4q35.1_DLBCL (FOCAL); SEQ ID NO: 6541, 4q35.1_DLBCL (FOCAL); SEQ ID NO: 6542, 4q35.1_DLBCL (FOCAL); SEQ ID NO: 6543, 4q35.1_DLBCL (FOCAL); SEQ ID NO: 6544, 4q35.1_DLBCL (FOCAL); SEQ ID NO: 6545, 4q35.1_DLBCL (FOCAL); SEQ ID NO: 6546, 4q35.1_DLBCL (FOCAL); SEQ ID NO: 6547, 4q35.1_DLBCL (FOCAL); SEQ ID NO: 6548, 4q35.1_DLBCL (FOCAL); SEQ ID NO: 6549, 4q35.1_DLBCL (FOCAL); SEQ ID NO: 6550, 4q35.1_DLBCL (FOCAL); SEQ ID NO: 6551, 4q35.1_DLBCL (FOCAL); SEQ ID NO: 6552, 4q35.1_DLBCL (FOCAL); SEQ ID NO: 6553, 4q35.1_DLBCL (FOCAL); SEQ ID NO: 6554, 4q35.1_DLBCL (FOCAL); SEQ ID NO: 6555, 4q35.1_DLBCL (FOCAL); SEQ ID NO: 6556, 4q35.1_DLBCL (FOCAL); SEQ ID NO: 6557, 4q35.1_DLBCL (FOCAL); SEQ ID NO: 6558, 4q35.1_DLBCL (FOCAL); SEQ ID NO: 6559, 4q35.1_DLBCL (FOCAL); SEQ ID NO: 6560, 4q35.1_DLBCL (FOCAL); SEQ ID NO: 6561, 4q35.1_DLBCL (FOCAL); SEQ ID NO: 6562, 4q35.1_DLBCL (FOCAL); SEQ ID NO: 6563, 4q35.1_DLBCL (FOCAL); SEQ ID NO: 6564, 4q35.1_DLBCL (FOCAL); SEQ ID NO: 6565, 4q35.1_DLBCL (FOCAL); SEQ ID NO: 6566, 4q35.1_DLBCL (FOCAL); SEQ ID NO: 6567, 4q35.1_DLBCL (FOCAL); SEQ ID NO: 6568, 4q35.1_DLBCL (FOCAL); SEQ ID NO: 6569, 4q35.1_DLBCL (FOCAL); SEQ ID NO: 6570, 4q35.1_DLBCL (FOCAL); SEQ ID NO: 6571, 4q35.1_DLBCL (FOCAL); SEQ ID NO: 6572, 4q35.1_DLBCL (FOCAL); SEQ ID NO: 6573, 4q35.1_DLBCL (FOCAL); SEQ ID NO: 6574, 4q35.1_DLBCL (FOCAL); SEQ ID NO: 6575, 4q35.1_DLBCL (FOCAL); SEQ ID NO: 6576, 4q35.1_DLBCL (FOCAL); SEQ ID NO: 6577, 4q35.1_DLBCL (FOCAL); SEQ ID NO: 6578, 4q35.1_DLBCL (FOCAL); SEQ ID NO: 6579, 4q35.1_DLBCL (FOCAL); SEQ ID NO: 6580, 4q35.1_DLBCL (FOCAL); SEQ ID NO: 6581, 4q35.1_DLBCL (FOCAL); SEQ ID NO: 6582, 4q35.1_DLBCL (FOCAL); SEQ ID NO: 6583, 4q35.1_DLBCL (FOCAL); SEQ ID NO: 6584, 4q35.1_DLBCL (FOCAL); SEQ ID NO: 6585, 4q35.1_DLBCL (FOCAL); SEQ ID NO: 6586, 4q35.1_DLBCL (FOCAL); SEQ ID NO: 6587, 4q35.1_DLBCL (FOCAL); SEQ ID NO: 6588, 4q35.1_DLBCL (FOCAL); SEQ ID NO: 6589, 4q35.1_DLBCL (FOCAL); SEQ ID NO: 6590, 4q35.1_DLBCL (FOCAL); SEQ ID NO: 6591, 4q35.1_DLBCL (FOCAL); SEQ ID NO: 6592, 4q35.1_DLBCL (FOCAL); SEQ ID NO: 6593, 4q35.1_DLBCL (FOCAL); SEQ ID NO: 6594, 4q35.1_DLBCL (FOCAL); SEQ ID NO: 6595, 4q35.1_DLBCL (FOCAL); SEQ ID NO: 6596, 4q35.1_DLBCL (FOCAL); SEQ ID NO: 6597, 4q35.1_DLBCL (FOCAL); SEQ ID NO: 6598, 4q35.1_DLBCL (FOCAL); SEQ ID NO: 6599, 4q35.1_DLBCL (FOCAL); SEQ ID NO: 6600, 4q35.1_DLBCL (FOCAL); SEQ ID NO: 6601, 4q35.1_DLBCL (FOCAL); SEQ ID NO: 6602, 4q35.1_DLBCL (FOCAL); SEQ ID NO: 6603, 4q35.1_DLBCL (FOCAL); SEQ ID NO: 6604, 4q35.1_DLBCL (FOCAL); SEQ ID NO: 6605, 4q35.1_DLBCL (FOCAL); SEQ ID NO: 6606, 4q35.1_DLBCL (FOCAL); SEQ ID NO: 6607, 4q35.1_DLBCL (FOCAL); SEQ ID NO: 6608, 4q35.1_DLBCL (FOCAL); SEQ ID NO: 6609, 4q35.1_DLBCL (FOCAL); SEQ ID NO: 6610, 4q35.1_DLBCL (FOCAL); SEQ ID NO: 6611, 4q35.1_DLBCL (FOCAL); SEQ ID NO: 6612, 4q35.1_DLBCL (FOCAL); SEQ ID NO: 6613, 4q35.1_DLBCL (FOCAL); SEQ ID NO: 6614, 4q35.1_DLBCL (FOCAL); SEQ ID NO: 6615, 4q35.1_DLBCL (FOCAL); SEQ ID NO: 6616, 4q35.1_DLBCL (FOCAL); SEQ ID NO: 6617, 4q35.1_DLBCL (FOCAL); SEQ ID NO: 6618, 4q35.1_DLBCL (FOCAL); SEQ ID NO: 6619, 4q35.1_DLBCL (FOCAL); SEQ ID NO: 6620, 4q35.1_DLBCL (FOCAL); SEQ ID NO: 6621, MSI (MSI); SEQ ID NO: 6622, MSI (MSI); SEQ ID NO: 6623, 4q35.1_DLBCL (FOCAL); SEQ ID NO: 6624, 4q35.1_DLBCL (FOCAL); SEQ ID NO: 6625, 4q35.1_DLBCL (FOCAL); SEQ ID NO: 6626, 4q35.1_DLBCL (FOCAL); SEQ ID NO: 6627, 4q35.1_DLBCL (FOCAL); SEQ ID NO: 6628, 4q35.1_DLBCL (FOCAL); SEQ ID NO: 6629, 4q35.1_DLBCL (FOCAL); SEQ ID NO: 6630, 4q35.1_DLBCL (FOCAL); SEQ ID NO: 6631, 4q35.1_DLBCL (FOCAL); SEQ ID NO: 6632, 4q35.1_DLBCL (FOCAL); SEQ ID NO: 6633, 4q35.1_DLBCL (FOCAL); SEQ ID NO: 6634, 4q35.1_DLBCL (FOCAL); SEQ ID NO: 6635, 4q35.1_DLBCL (FOCAL); SEQ ID NO: 6636, 4q35.1_DLBCL (FOCAL); SEQ ID NO: 6637, 4q35.1_DLBCL (FOCAL); SEQ ID NO: 6638, FP (FP); SEQ ID NO: 6639, TERT promoter (SV); SEQ ID NO: 6640, 5P_DLBCL (ARM); SEQ ID NO: 6641, 5P_DLBCL (ARM); SEQ ID NO: 6642, 5P_DLBCL (ARM); SEQ ID NO: 6643, FP (FP); SEQ ID NO: 6644, 5P_DLBCL (ARM); SEQ ID NO: 6645, 5P_DLBCL (ARM); SEQ ID NO: 6646, 5P_DLBCL (ARM); SEQ ID NO: 6647, 5P_DLBCL (ARM); SEQ ID NO: 6648, 5P_DLBCL (ARM); SEQ ID NO: 6649, FP (FP); SEQ ID NO: 6650, 5P_DLBCL (ARM); SEQ ID NO: 6651, 5P_DLBCL (ARM); SEQ ID NO: 6652, 5P_DLBCL (ARM); SEQ ID NO: 6653, 5P_DLBCL (ARM); SEQ ID NO: 6654, 5P_DLBCL (ARM); SEQ ID NO: 6655, 5P_DLBCL (ARM); SEQ ID NO: 6656, 5P_DLBCL (ARM); SEQ ID NO: 6657, 5P_DLBCL (ARM); SEQ ID NO: 6658, 5P_DLBCL (ARM); SEQ ID NO: 6659, 5P_DLBCL (ARM); SEQ ID NO: 6660, 5P_DLBCL (ARM); SEQ ID NO: 6661, 5P_DLBCL (ARM); SEQ ID NO: 6662, 5P_DLBCL (ARM); SEQ ID NO: 6663, 5P_DLBCL (ARM); SEQ ID NO: 6664, 5P_DLBCL (ARM); SEQ ID NO: 6665, FP (FP); SEQ ID NO: 6666, 5P_DLBCL (ARM); SEQ ID NO: 6667, 5P_DLBCL (ARM); SEQ ID NO: 6668, 5P_DLBCL (ARM); SEQ ID NO: 6669, 5P_DLBCL (ARM); SEQ ID NO: 6670, 5P_DLBCL (ARM); SEQ ID NO: 6671, 5P_DLBCL (ARM); SEQ ID NO: 6672, 5P_DLBCL (ARM); SEQ ID NO: 6673, 5P_DLBCL (ARM); SEQ ID NO: 6674, 5P_DLBCL (ARM); SEQ ID NO: 6675, 5P_DLBCL (ARM); SEQ ID NO: 6676, 5P_DLBCL (ARM); SEQ ID NO: 6677, 5P_DLBCL (ARM); SEQ ID NO: 6678, 5P_DLBCL (ARM); SEQ ID NO: 6679, 5P_DLBCL (ARM); SEQ ID NO: 6680, 5P_DLBCL (ARM); SEQ ID NO: 6681, 5P_DLBCL (ARM); SEQ ID NO: 6682, 5P_DLBCL (ARM); SEQ ID NO: 6683, 5Q_DLBCL (ARM); SEQ ID NO: 6684, 5Q_DLBCL (ARM); SEQ ID NO: 6685, 5Q_DLBCL (ARM); SEQ ID NO: 6686, 5Q_DLBCL (ARM); SEQ ID NO: 6687, 5Q_DLBCL (ARM); SEQ ID NO: 6688, 5Q_DLBCL (ARM); SEQ ID NO: 6689, 5Q_DLBCL (ARM); SEQ ID NO: 6690, 5Q_DLBCL (ARM); SEQ ID NO: 6691, 5Q_DLBCL (ARM); SEQ ID NO: 6692, 5Q_DLBCL (ARM); SEQ ID NO: 6693, 5Q_DLBCL (ARM); SEQ ID NO: 6694, 5Q_DLBCL (ARM); SEQ ID NO: 6695, 5Q_DLBCL (ARM); SEQ ID NO: 6696, 5Q_DLBCL (ARM); SEQ ID NO: 6697, 5Q_DLBCL (ARM); SEQ ID NO: 6698, 5Q_DLBCL (ARM); SEQ ID NO: 6699, 5Q_DLBCL (ARM); SEQ ID NO: 6700, 5Q_DLBCL (ARM); SEQ ID NO: 6701, 5Q_DLBCL (ARM); SEQ ID NO: 6702, 5Q_DLBCL (ARM); SEQ ID NO: 6703, 5Q_DLBCL (ARM); SEQ ID NO: 6704, 5Q_DLBCL (ARM); SEQ ID NO: 6705, 5Q_DLBCL (ARM); SEQ ID NO: 6706, 5Q_DLBCL (ARM); SEQ ID NO: 6707, 5Q_DLBCL (ARM); SEQ ID NO: 6708, FP (FP); SEQ ID NO: 6709, 5Q_DLBCL (ARM); SEQ ID NO: 6710, MSI (MSI); SEQ ID NO: 6711, MSI (MSI); SEQ ID NO: 6712, 5Q_DLBCL (ARM); SEQ ID NO: 6713, 5Q_DLBCL (ARM); SEQ ID NO: 6714, 5Q_DLBCL (ARM); SEQ ID NO: 6715, 5Q_DLBCL (ARM); SEQ ID NO: 6716, 5Q_DLBCL (ARM); SEQ ID NO: 6717, FP (FP); SEQ ID NO: 6718, ADGRV1 (Gene); SEQ ID NO: 6719, ADGRV1 (Gene); SEQ ID NO: 6720, ADGRV1 (Gene); SEQ ID NO: 6721, ADGRV1 (Gene); SEQ ID NO: 6722, ADGRV1 (Gene); SEQ ID NO: 6723, ADGRV1 (Gene); SEQ ID NO: 6724, ADGRV1 (Gene); SEQ ID NO: 6725, ADGRV1 (Gene); SEQ ID NO: 6726, ADGRV1 (Gene); SEQ ID NO: 6727, ADGRV1 (Gene); SEQ ID NO: 6728, ADGRV1 (Gene); SEQ ID NO: 6729, ADGRV1 (Gene); SEQ ID NO: 6730, ADGRV1 (Gene); SEQ ID NO: 6731, ADGRV1 (Gene); SEQ ID NO: 6732, ADGRV1 (Gene); SEQ ID NO: 6733, ADGRV1 (Gene); SEQ ID NO: 6734, ADGRV1 (Gene); SEQ ID NO: 6735, ADGRV1 (Gene); SEQ ID NO: 6736, ADGRV1 (Gene); SEQ ID NO: 6737, ADGRV1 (Gene); SEQ ID NO: 6738, ADGRV1 (Gene); SEQ ID NO: 6739, ADGRV1 (Gene); SEQ ID NO: 6740, ADGRV1 (Gene); SEQ ID NO: 6741, ADGRV1 (Gene); SEQ ID NO: 6742, ADGRV1 (Gene); SEQ ID NO: 6743, ADGRV1 (Gene); SEQ ID NO: 6744, ADGRV1 (Gene); SEQ ID NO: 6745, ADGRV1 (Gene); SEQ ID NO: 6746, ADGRV1 (Gene); SEQ ID NO: 6747, ADGRV1 (Gene); SEQ ID NO: 6748, ADGRV1 (Gene); SEQ ID NO: 6749, ADGRV1 (Gene); SEQ ID NO: 6750, ADGRV1 (Gene); SEQ ID NO: 6751, ADGRV1 (Gene); SEQ ID NO: 6752, ADGRV1 (Gene); SEQ ID NO: 6753, ADGRV1 (Gene); SEQ ID NO: 6754, ADGRV1 (Gene); SEQ ID NO: 6755, ADGRV1 (Gene); SEQ ID NO: 6756, ADGRV1 (Gene); SEQ ID NO: 6757, ADGRV1 (Gene); SEQ ID NO: 6758, ADGRV1 (Gene); SEQ ID NO: 6759, ADGRV1 (Gene); SEQ ID NO: 6760, ADGRV1 (Gene); SEQ ID NO: 6761, ADGRV1 (Gene); SEQ ID NO: 6762, ADGRV1 (Gene); SEQ ID NO: 6763, ADGRV1 (Gene); SEQ ID NO: 6764, ADGRV1 (Gene); SEQ ID NO: 6765, ADGRV1 (Gene); SEQ ID NO: 6766, ADGRV1 (Gene); SEQ ID NO: 6767, ADGRV1 (Gene); SEQ ID NO: 6768, ADGRV1 (Gene); SEQ ID NO: 6769, ADGRV1 (Gene); SEQ ID NO: 6770, ADGRV1 (Gene); SEQ ID NO: 6771, ADGRV1 (Gene); SEQ ID NO: 6772, ADGRV1 (Gene); SEQ ID NO: 6773, ADGRV1 (Gene); SEQ ID NO: 6774, ADGRV1 (Gene); SEQ ID NO: 6775, ADGRV1 (Gene); SEQ ID NO: 6776, ADGRV1 (Gene); SEQ ID NO: 6777, ADGRV1 (Gene); SEQ ID NO: 6778, ADGRV1 (Gene); SEQ ID NO: 6779, ADGRV1 (Gene); SEQ ID NO: 6780, ADGRV1 (Gene); SEQ ID NO: 6781, ADGRV1 (Gene); SEQ ID NO: 6782, ADGRV1 (Gene); SEQ ID NO: 6783, ADGRV1 (Gene); SEQ ID NO: 6784, ADGRV1 (Gene); SEQ ID NO: 6785, ADGRV1 (Gene); SEQ ID NO: 6786, ADGRV1 (Gene); SEQ ID NO: 6787, ADGRV1 (Gene); SEQ ID NO: 6788, ADGRV1 (Gene); SEQ ID NO: 6789, ADGRV1 (Gene); SEQ ID NO: 6790, ADGRV1 (Gene); SEQ ID NO: 6791, ADGRV1 (Gene); SEQ ID NO: 6792, ADGRV1 (Gene); SEQ ID NO: 6793, ADGRV1 (Gene); SEQ ID NO: 6794, ADGRV1 (Gene); SEQ ID NO: 6795, ADGRV1 (Gene); SEQ ID NO: 6796, ADGRV1 (Gene); SEQ ID NO: 6797, ADGRV1 (Gene); SEQ ID NO: 6798, ADGRV1 (Gene); SEQ ID NO: 6799, ADGRV1 (Gene); SEQ ID NO: 6800, ADGRV1 (Gene); SEQ ID NO: 6801, ADGRV1 (Gene); SEQ ID NO: 6802, ADGRV1 (Gene); SEQ ID NO: 6803, ADGRV1 (Gene); SEQ ID NO: 6804, ADGRV1 (Gene); SEQ ID NO: 6805, ADGRV1 (Gene); SEQ ID NO: 6806, ADGRV1 (Gene); SEQ ID NO: 6807, ADGRV1 (Gene); SEQ ID NO: 6808, ADGRV1 (Gene); SEQ ID NO: 6809, ADGRV1 (Gene); SEQ ID NO: 6810, ADGRV1 (Gene); SEQ ID NO: 6811, ADGRV1 (Gene); SEQ ID NO: 6812, ADGRV1 (Gene); SEQ ID NO: 6813, ADGRV1 (Gene); SEQ ID NO: 6814, ADGRV1 (Gene); SEQ ID NO: 6815, ADGRV1 (Gene); SEQ ID NO: 6816, ADGRV1 (Gene); SEQ ID NO: 6817, ADGRV1 (Gene); SEQ ID NO: 6818, ADGRV1 (Gene); SEQ ID NO: 6819, ADGRV1 (Gene); SEQ ID NO: 6820, 5Q_DLBCL (ARM); SEQ ID NO: 6821, 5Q_DLBCL (ARM); SEQ ID NO: 6822, 5Q_DLBCL (ARM); SEQ ID NO: 6823, 5Q_DLBCL (ARM); SEQ ID NO: 6824, 5Q_DLBCL (ARM); SEQ ID NO: 6825, 5Q_DLBCL (ARM); SEQ ID NO: 6826, 5Q_DLBCL (ARM); SEQ ID NO: 6827, 5Q_DLBCL (ARM); SEQ ID NO: 6828, 5Q_DLBCL (ARM); SEQ ID NO: 6829, 5Q_DLBCL (ARM); SEQ ID NO: 6830, 5Q_DLBCL (ARM); SEQ ID NO: 6831, 5Q_DLBCL (ARM); SEQ ID NO: 6832, 5Q_DLBCL (ARM); SEQ ID NO: 6833, 5Q_DLBCL (ARM); SEQ ID NO: 6834, 5Q_DLBCL (ARM); SEQ ID NO: 6835, 5Q_DLBCL (ARM); SEQ ID NO: 6836, 5Q_DLBCL (ARM); SEQ ID NO: 6837, 5Q_DLBCL (ARM); SEQ ID NO: 6838, 5Q_DLBCL (ARM); SEQ ID NO: 6839, 5Q_DLBCL (ARM); SEQ ID NO: 6840, 5Q_DLBCL (ARM); SEQ ID NO: 6841, 5Q_DLBCL (ARM); SEQ ID NO: 6842, 5Q_DLBCL (ARM); SEQ ID NO: 6843, 5Q_DLBCL (ARM); SEQ ID NO: 6844, 5Q_DLBCL (ARM); SEQ ID NO: 6845, ZNF608 (Gene); SEQ ID NO: 6846, ZNF608 (Gene); SEQ ID NO: 6847, ZNF608 (Gene); SEQ ID NO: 6848, ZNF608 (Gene); SEQ ID NO: 6849, ZNF608 (Gene); SEQ ID NO: 6850, ZNF608 (Gene); SEQ ID NO: 6851, ZNF608 (Gene); SEQ ID NO: 6852, ZNF608 (Gene); SEQ ID NO: 6853, ZNF608 (Gene); SEQ ID NO: 6854, ZNF608 (Gene); SEQ ID NO: 6855, ZNF608 (Gene); SEQ ID NO: 6856, ZNF608 (Gene); SEQ ID NO: 6857, ZNF608 (Gene); SEQ ID NO: 6858, ZNF608 (Gene); SEQ ID NO: 6859, ZNF608 (Gene); SEQ ID NO: 6860, 5Q_DLBCL (ARM); SEQ ID NO: 6861, 5Q_DLBCL (ARM); SEQ ID NO: 6862, 5Q_DLBCL (ARM); SEQ ID NO: 6863, 5Q_DLBCL (ARM); SEQ ID NO: 6864, 5Q_DLBCL (ARM); SEQ ID NO: 6865, MSI (MSI); SEQ ID NO: 6866, MSI (MSI); SEQ ID NO: 6867, 5Q_DLBCL (ARM); SEQ ID NO: 6868, 5Q_DLBCL (ARM); SEQ ID NO: 6869, 5Q_DLBCL (ARM); SEQ ID NO: 6870, TGFBI (Gene); SEQ ID NO: 6871, TGFBI (Gene); SEQ ID NO: 6872, TGFBI (Gene); SEQ ID NO: 6873, TGFBI (Gene); SEQ ID NO: 6874, TGFBI (Gene); SEQ ID NO: 6875, TGFBI (Gene); SEQ ID NO: 6876, TGFBI (Gene); SEQ ID NO: 6877, TGFBI (Gene); SEQ ID NO: 6878, TGFBI (Gene); SEQ ID NO: 6879, TGFBI (Gene); SEQ ID NO: 6880, TGFBI (Gene); SEQ ID NO: 6881, TGFBI (Gene); SEQ ID NO: 6882, TGFBI (Gene); SEQ ID NO: 6883, TGFBI (Gene); SEQ ID NO: 6884, TGFBI (Gene); SEQ ID NO: 6885, TGFBI (Gene); SEQ ID NO: 6886, TGFBI (Gene); SEQ ID NO: 6887, 5Q_DLBCL (ARM); SEQ ID NO: 6888, 5Q_DLBCL (ARM); SEQ ID NO: 6889, 5Q_DLBCL (ARM); SEQ ID NO: 6890, MSI (MSI); SEQ ID NO: 6891, MSI (MSI); SEQ ID NO: 6892, 5Q_DLBCL (ARM); SEQ ID NO: 6893, MSI (MSI); SEQ ID NO: 6894, MSI (MSI); SEQ ID NO: 6895, 5Q_DLBCL (ARM); SEQ ID NO: 6896, 5Q_DLBCL (ARM); SEQ ID NO: 6897, 5Q_DLBCL (ARM); SEQ ID NO: 6898, 5Q_DLBCL (ARM); SEQ ID NO: 6899, 5Q_DLBCL (ARM); SEQ ID NO: 6900, 5Q_DLBCL (ARM); SEQ ID NO: 6901, 5Q_DLBCL (ARM); SEQ ID NO: 6902, PDGFRB (Gene); SEQ ID NO: 6903, PDGFRB (Gene); SEQ ID NO: 6904, PDGFRB (Gene); SEQ ID NO: 6905, PDGFRB (Gene); SEQ ID NO: 6906, PDGFRB (Gene); SEQ ID NO: 6907, PDGFRB (Gene); SEQ ID NO: 6908, PDGFRB (Gene); SEQ ID NO: 6909, PDGFRB (Gene); SEQ ID NO: 6910, PDGFRB (Gene); SEQ ID NO: 6911, PDGFRB (Gene); SEQ ID NO: 6912, PDGFRB (Gene); SEQ ID NO: 6913, PDGFRB (Gene); SEQ ID NO: 6914, PDGFRB (Gene); SEQ ID NO: 6915, PDGFRB (Gene); SEQ ID NO: 6916, PDGFRB (Gene); SEQ ID NO: 6917, PDGFRB (Gene); SEQ ID NO: 6918, PDGFRB (Gene); SEQ ID NO: 6919, PDGFRB (Gene); SEQ ID NO: 6920, PDGFRB (Gene); SEQ ID NO: 6921, PDGFRB (Gene); SEQ ID NO: 6922, PDGFRB (Gene); SEQ ID NO: 6923, PDGFRB (Gene); SEQ ID NO: 6924, TNIP1 (Gene); SEQ ID NO: 6925, TNIP1 (Gene); SEQ ID NO: 6926, TNIP1 (Gene); SEQ ID NO: 6927, TNIP1 (Gene); SEQ ID NO: 6928, TNIP1 (Gene); SEQ ID NO: 6929, TNIP1 (Gene); SEQ ID NO: 6930, TNIP1 (Gene); SEQ ID NO: 6931, TNIP1 (Gene); SEQ ID NO: 6932, TNIP1 (Gene); SEQ ID NO: 6933, TNIP1 (Gene); SEQ ID NO: 6934, 5Q_DLBCL (ARM); SEQ ID NO: 6935, TNIP1 (Gene); SEQ ID NO: 6936, TNIP1 (Gene); SEQ ID NO: 6937, TNIP1 (Gene); SEQ ID NO: 6938, TNIP1 (Gene); SEQ ID NO: 6939, TNIP1 (Gene); SEQ ID NO: 6940, TNIP1 (Gene); SEQ ID NO: 6941, TNIP1 (Gene); SEQ ID NO: 6942, 5Q_DLBCL (ARM); SEQ ID NO: 6943, 5Q_DLBCL (ARM); SEQ ID NO: 6944, 5Q_DLBCL (ARM); SEQ ID NO: 6945, 5Q_DLBCL (ARM); SEQ ID NO: 6946, HAVCR2 (Gene); SEQ ID NO: 6947, HAVCR2 (Gene); SEQ ID NO: 6948, HAVCR2 (Gene); SEQ ID NO: 6949, HAVCR2 (Gene); SEQ ID NO: 6950, HAVCR2 (Gene); SEQ ID NO: 6951, HAVCR2 (Gene); SEQ ID NO: 6952, HAVCR2 (Gene); SEQ ID NO: 6953, ITK (Gene); SEQ ID NO: 6954, ITK (Gene); SEQ ID NO: 6955, ITK (Gene); SEQ ID NO: 6956, ITK (Gene); SEQ ID NO: 6957, ITK (Gene); SEQ ID NO: 6958, ITK (Gene); SEQ ID NO: 6959, ITK (Gene); SEQ ID NO: 6960, ITK (Gene); SEQ ID NO: 6961, ITK (Gene); SEQ ID NO: 6962, ITK (Gene); SEQ ID NO: 6963, ITK (Gene); SEQ ID NO: 6964, ITK (Gene); SEQ ID NO: 6965, ITK (Gene); SEQ ID NO: 6966, ITK (Gene); SEQ ID NO: 6967, ITK (Gene); SEQ ID NO: 6968, 5Q_DLBCL (ARM); SEQ ID NO: 6969, 5Q_DLBCL (ARM); SEQ ID NO: 6970, EBF1 (Gene); SEQ ID NO: 6971, EBF1 (Gene); SEQ ID NO: 6972, EBF1 (Gene); SEQ ID NO: 6973, EBF1 (Gene); SEQ ID NO: 6974, EBF1 (Gene); SEQ ID NO: 6975, EBF1 (Gene); SEQ ID NO: 6976, EBF1 (Gene); SEQ ID NO: 6977, EBF1 (Gene); SEQ ID NO: 6978, EBF1 (Gene); SEQ ID NO: 6979, EBF1 (Gene); SEQ ID NO: 6980, EBF1 (Gene); SEQ ID NO: 6981, 5Q_DLBCL (ARM); SEQ ID NO: 6982, EBF1 (Gene); SEQ ID NO: 6983, EBF1 (Gene); SEQ ID NO: 6984, EBF1 (Gene); SEQ ID NO: 6985, EBF1 (Gene); SEQ ID NO: 6986, EBF1 (Gene); SEQ ID NO: 6987, EBF1 (Gene); SEQ ID NO: 6988, EBF1 (Gene); SEQ ID NO: 6989, 5Q_DLBCL (ARM); SEQ ID NO: 6990, 5Q_DLBCL (ARM); SEQ ID NO: 6991, 5Q_DLBCL (ARM); SEQ ID NO: 6992, 5Q_DLBCL (ARM); SEQ ID NO: 6993, 5Q_DLBCL (ARM); SEQ ID NO: 6994, 5Q_DLBCL (ARM); SEQ ID NO: 6995, 5Q_DLBCL (ARM); SEQ ID NO: 6996, 5Q_DLBCL (ARM); SEQ ID NO: 6997, 5Q_DLBCL (ARM); SEQ ID NO: 6998, 5Q_DLBCL (ARM); SEQ ID NO: 6999, 5Q_DLBCL (ARM); SEQ ID NO: 7000, 5Q_DLBCL (ARM); SEQ ID NO: 7001, 5Q_DLBCL (ARM); SEQ ID NO: 7002, 5Q_DLBCL (ARM); SEQ ID NO: 7003, 5Q_DLBCL (ARM); SEQ ID NO: 7004, FP (FP); SEQ ID NO: 7005, 5Q_DLBCL (ARM); SEQ ID NO: 7006, 5Q_DLBCL (ARM); SEQ ID NO: 7007, 5Q_DLBCL (ARM); SEQ ID NO: 7008, 5Q_DLBCL (ARM); SEQ ID NO: 7009, 5Q_DLBCL (ARM); SEQ ID NO: 7010, DUSP22 (Gene); SEQ ID NO: 7011, DUSP22 (Gene); SEQ ID NO: 7012, DUSP22 (Gene); SEQ ID NO: 7013, DUSP22 (Gene); SEQ ID NO: 7014, DUSP22 (Gene); SEQ ID NO: 7015, DUSP22 (Gene); SEQ ID NO: 7016, IRF4 (Gene); SEQ ID NO: 7017, IRF4 (Gene); SEQ ID NO: 7018, IRF4 (Gene); SEQ ID NO: 7019, IRF4 (Gene); SEQ ID NO: 7020, IRF4 (Gene); SEQ ID NO: 7021, IRF4 (Gene); SEQ ID NO: 7022, IRF4 (Gene); SEQ ID NO: 7023, IRF4 (Gene); SEQ ID NO: 7024, 6P_DLBCL (ARM); SEQ ID NO: 7025, FP (FP); SEQ ID NO: 7026, 6P_DLBCL (ARM); SEQ ID NO: 7027, FOXC1 (Gene); SEQ ID NO: 7028, FOXC1 (Gene); SEQ ID NO: 7029, FOXC1 (Gene); SEQ ID NO: 7030, FOXC1 (Gene); SEQ ID NO: 7031, 6P_DLBCL (ARM); SEQ ID NO: 7032, 6P_DLBCL (ARM); SEQ ID NO: 7033, 6P_DLBCL (ARM); SEQ ID NO: 7034, 6P_DLBCL (ARM); SEQ ID NO: 7035, 6P_DLBCL (ARM); SEQ ID NO: 7036, 6P_DLBCL (ARM); SEQ ID NO: 7037, 6P_DLBCL (ARM); SEQ ID NO: 7038, 6P_DLBCL (ARM); SEQ ID NO: 7039, 6P_DLBCL (ARM); SEQ ID NO: 7040, 6P_DLBCL (ARM); SEQ ID NO: 7041, 6P_DLBCL (ARM); SEQ ID NO: 7042, 6P_DLBCL (ARM); SEQ ID NO: 7043, CD83 (Gene); SEQ ID NO: 7044, CD83 (Gene); SEQ ID NO: 7045, CD83 (Gene); SEQ ID NO: 7046, CD83 (Gene); SEQ ID NO: 7047, CD83 (Gene); SEQ ID NO: 7048, 6P_DLBCL (ARM); SEQ ID NO: 7049, 6P_DLBCL (ARM); SEQ ID NO: 7050, 6P_DLBCL (ARM); SEQ ID NO: 7051, 6P_DLBCL (ARM); SEQ ID NO: 7052, 6P_DLBCL (ARM); SEQ ID NO: 7053, FP (FP); SEQ ID NO: 7054, 6P_DLBCL (ARM); SEQ ID NO: 7055, 6P_DLBCL (ARM); SEQ ID NO: 7056, 6P_DLBCL (ARM); SEQ ID NO: 7057, 6P_DLBCL (ARM); SEQ ID NO: 7058, 6P_DLBCL (ARM); SEQ ID NO: 7059, FP (FP); SEQ ID NO: 7060, 6P_DLBCL (ARM); SEQ ID NO: 7061, HIST1H3B (Gene); SEQ ID NO: 7062, HIST1H1C (Gene); SEQ ID NO: 7063, HIST1H1C (Gene); SEQ ID NO: 7064, HIST1H2BC (Gene); SEQ ID NO: 7065, HIST1H2AC (Gene); SEQ ID NO: 7066, HIST1H1E (Gene); SEQ ID NO: 7067, HIST1H1E (Gene); SEQ ID NO: 7068, HIST1H1D (Gene); SEQ ID NO: 7069, HIST1H1D (Gene); SEQ ID NO: 7070, HIST1H2BK (Gene); SEQ ID NO: 7071, HIST1H1B (Gene); SEQ ID NO: 7072, HIST1H1B (Gene); SEQ ID NO: 7073, HIST1H2AM (Gene); SEQ ID NO: 7074, 6P_DLBCL (ARM); SEQ ID NO: 7075, 6p21_HLA (FOCAL); SEQ ID NO: 7076, 6p21 HLA (FOCAL); SEQ ID NO: 7077, HLA-A (Gene); SEQ ID NO: 7078, HLA-A (Gene); SEQ ID NO: 7079, HLA-A (Gene); SEQ ID NO: 7080, HLA-A (Gene); SEQ ID NO: 7081, HLA-A (Gene); SEQ ID NO: 7082, HLA-A (Gene); SEQ ID NO: 7083, HLA-A (Gene); SEQ ID NO: 7084, HLA-A (Gene); SEQ ID NO: 7085, 6p21_HLA (FOCAL); SEQ ID NO: 7086, 6p21_HLA (FOCAL); SEQ ID NO: 7087, 6p21_HLA (FOCAL); SEQ ID NO: 7088, 6p21_HLA (FOCAL); SEQ ID NO: 7089, 6p21_HLA (FOCAL); SEQ ID NO: 7090, 6p21_HLA (FOCAL); SEQ ID NO: 7091, HLA-C (Gene); SEQ ID NO: 7092, HLA-C (Gene); SEQ ID NO: 7093, HLA-C (Gene); SEQ ID NO: 7094, HLA-C (Gene); SEQ ID NO: 7095, HLA-C (Gene); SEQ ID NO: 7096, HLA-C (Gene); SEQ ID NO: 7097, HLA-C (Gene); SEQ ID NO: 7098, HLA-C (Gene); SEQ ID NO: 7099, HLA-B (Gene); SEQ ID NO: 7100, HLA-B (Gene); SEQ ID NO: 7101, HLA-B (Gene); SEQ ID NO: 7102, HLA-B (Gene); SEQ ID NO: 7103, HLA-B (Gene); SEQ ID NO: 7104, HLA-B (Gene); SEQ ID NO: 7105, HLA-B (Gene); SEQ ID NO: 7106, 6p21_HLA (FOCAL); SEQ ID NO: 7107, LTB (Gene); SEQ ID NO: 7108, LTB (Gene); SEQ ID NO: 7109, LTB (Gene); SEQ ID NO: 7110, LTB (Gene); SEQ ID NO: 7111, 6p21_HLA (FOCAL); SEQ ID NO: 7112, 6p21_HLA (FOCAL); SEQ ID NO: 7113, 6p21_HLA (FOCAL); SEQ ID NO: 7114, 6p21_HLA (FOCAL); SEQ ID NO: 7115, HLA-DRA (Gene); SEQ ID NO: 7116, HLA-DRA (Gene); SEQ ID NO: 7117, HLA-DRA (Gene); SEQ ID NO: 7118, HLA-DRA (Gene); SEQ ID NO: 7119, HLA-DRA (Gene); SEQ ID NO: 7120, 6p21_HLA (FOCAL); SEQ ID NO: 7121, HLA-DMB (Gene); SEQ ID NO: 7122, HLA-DMB (Gene); SEQ ID NO: 7123, HLA-DMB (Gene); SEQ ID NO: 7124, HLA-DMB (Gene); SEQ ID NO: 7125, HLA-DMB (Gene); SEQ ID NO: 7126, HLA-DMB (Gene); SEQ ID NO: 7127, HLA-DMA (Gene); SEQ ID NO: 7128, HLA-DMA (Gene); SEQ ID NO: 7129, HLA-DMA (Gene); SEQ ID NO: 7130, HLA-DMA (Gene); SEQ ID NO: 7131, HLA-DMA (Gene); SEQ ID NO: 7132, 6p21 HLA (FOCAL); SEQ ID NO: 7133, 6p21 HLA (FOCAL); SEQ ID NO: 7134, 6P_DLBCL (ARM); SEQ ID NO: 7135, FANCE (Gene); SEQ ID NO: 7136, FANCE (Gene); SEQ ID NO: 7137, FANCE (Gene); SEQ ID NO: 7138, FANCE (Gene); SEQ ID NO: 7139, FANCE (Gene); SEQ ID NO: 7140, FANCE (Gene); SEQ ID NO: 7141, FANCE (Gene); SEQ ID NO: 7142, FANCE (Gene); SEQ ID NO: 7143, FANCE (Gene); SEQ ID NO: 7144, FANCE (Gene); SEQ ID NO: 7145, FANCE (Gene); SEQ ID NO: 7146, FANCE (Gene); SEQ ID NO: 7147, FANCE (Gene); SEQ ID NO: 7148, FANCE (Gene); SEQ ID NO: 7149, FANCE (Gene); SEQ ID NO: 7150, 6P_DLBCL (ARM); SEQ ID NO: 7151, CDKN1A (Gene); SEQ ID NO: 7152, CDKN1A (Gene); SEQ ID NO: 7153, CDKN1A (Gene); SEQ ID NO: 7154, PIM1 (Gene); SEQ ID NO: 7155, PIM1 (Gene); SEQ ID NO: 7156, PIM1 (Gene); SEQ ID NO: 7157, PIM1 (Gene); SEQ ID NO: 7158, PIM1 (Gene); SEQ ID NO: 7159, PIM1 (Gene); SEQ ID NO: 7160, PIM1 (Gene); SEQ ID NO: 7161, PIM1 (Gene); SEQ ID NO: 7162, 6P_DLBCL (ARM); SEQ ID NO: 7163, 6P_DLBCL (ARM); SEQ ID NO: 7164, 6P_DLBCL (ARM); SEQ ID NO: 7165, 6P_DLBCL (ARM); SEQ ID NO: 7166, 6P_DLBCL (ARM); SEQ ID NO: 7167, CCND3 (Gene); SEQ ID NO: 7168, CCND3 (Gene); SEQ ID NO: 7169, CCND3 (Gene); SEQ ID NO: 7170, CCND3 (Gene); SEQ ID NO: 7171, CCND3 (Gene); SEQ ID NO: 7172, CCND3 (Gene); SEQ ID NO: 7173, CCND3 upstream (SV); SEQ ID NO: 7174, CCND3 upstream (SV); SEQ ID NO: 7175, CCND3 upstream (SV); SEQ ID NO: 7176, CCND3 upstream (SV); SEQ ID NO: 7177, CCND3 upstream (SV); SEQ ID NO: 7178, CCND3 upstream (SV); SEQ ID NO: 7179, CCND3 upstream (SV); SEQ ID NO: 7180, CCND3 upstream (SV); SEQ ID NO: 7181, CCND3 upstream (SV); SEQ ID NO: 7182, CCND3 upstream (SV); SEQ ID NO: 7183, CCND3 upstream (SV); SEQ ID NO: 7184, CCND3 upstream (SV); SEQ ID NO: 7185, CCND3 upstream (SV); SEQ ID NO: 7186, CCND3 upstream (SV); SEQ ID NO: 7187, CCND3 upstream (SV); SEQ ID NO: 7188, CCND3 upstream (SV); SEQ ID NO: 7189, CCND3 upstream (SV); SEQ ID NO: 7190, CCND3 upstream (SV); SEQ ID NO: 7191, CCND3 upstream (SV); SEQ ID NO: 7192, 6P_DLBCL (ARM); SEQ ID NO: 7193, NFKBIE (Gene); SEQ ID NO: 7194, NFKBIE (Gene); SEQ ID NO: 7195, NFKBIE (Gene); SEQ ID NO: 7196, NFKBIE (Gene); SEQ ID NO: 7197, NFKBIE (Gene); SEQ ID NO: 7198, NFKBIE (Gene); SEQ ID NO: 7199, NFKBIE (Gene); SEQ ID NO: 7200, NFKBIE (Gene); SEQ ID NO: 7201, 6P_DLBCL (ARM); SEQ ID NO: 7202, 6P_DLBCL (ARM); SEQ ID NO: 7203, 6P_DLBCL (ARM); SEQ ID NO: 7204, 6P_DLBCL (ARM); SEQ ID NO: 7205, 6P_DLBCL (ARM); SEQ ID NO: 7206, 6P_DLBCL (ARM); SEQ ID NO: 7207, 6P_DLBCL (ARM); SEQ ID NO: 7208, 6P_DLBCL (ARM); SEQ ID NO: 7209, 6P_DLBCL (ARM); SEQ ID NO: 7210, 6P_DLBCL (ARM); SEQ ID NO: 7211, 6P_DLBCL (ARM); SEQ ID NO: 7212, 6Q_DLBCL (ARM); SEQ ID NO: 7213, FP (FP); SEQ ID NO: 7214, 6q12_HL_custom (FOCAL); SEQ ID NO: 7215, 6q12_HL_custom (FOCAL); SEQ ID NO: 7216, 6q12_HL_custom (FOCAL); SEQ ID NO: 7217, 6q12_HL_custom (FOCAL); SEQ ID NO: 7218, 6q12_HL_custom (FOCAL); SEQ ID NO: 7219, 6q12 HL_custom (FOCAL); SEQ ID NO: 7220, 6q12_HL_custom (FOCAL); SEQ ID NO: 7221, 6q12_HL_custom (FOCAL); SEQ ID NO: 7222, 6q12_HL_custom (FOCAL); SEQ ID NO: 7223, 6q12_HL_custom (FOCAL); SEQ ID NO: 7224, 6q12_HL_custom (FOCAL); SEQ ID NO: 7225, 6q12_HL_custom (FOCAL); SEQ ID NO: 7226, 6q12_HL_custom (FOCAL); SEQ ID NO: 7227, 6q12_HL_custom (FOCAL); SEQ ID NO: 7228, 6q12_HL_custom (FOCAL); SEQ ID NO: 7229, 6q12_HL_custom (FOCAL); SEQ ID NO: 7230, 6q12 HL_custom (FOCAL); SEQ ID NO: 7231, 6q12_HL_custom (FOCAL); SEQ ID NO: 7232, 6q12_HL_custom (FOCAL); SEQ ID NO: 7233, 6q12_HL_custom (FOCAL); SEQ ID NO: 7234, 6Q_DLBCL (ARM); SEQ ID NO: 7235, 6Q_DLBCL (ARM); SEQ ID NO: 7236, EEF1A1 (Gene); SEQ ID NO: 7237, EEF1A1 (Gene); SEQ ID NO: 7238, EEF1A1 (Gene); SEQ ID NO: 7239, EEF1A1 (Gene); SEQ ID NO: 7240, EEF1A1 (Gene); SEQ ID NO: 7241, EEF1A1 (Gene); SEQ ID NO: 7242, EEF1A1 (Gene); SEQ ID NO: 7243, 6q14.1_DLBCL (FOCAL); SEQ ID NO: 7244, 6q14.1_DLBCL (FOCAL); SEQ ID NO: 7245, 6q14.1_DLBCL (FOCAL); SEQ ID NO: 7246, 6q14.1_DLBCL (FOCAL); SEQ ID NO: 7247, 6q14.1_DLBCL (FOCAL); SEQ ID NO: 7248, 6q14.1_DLBCL (FOCAL); SEQ ID NO: 7249, 6q14.1_DLBCL (FOCAL); SEQ ID NO: 7250, 6q14.1_DLBCL (FOCAL); SEQ ID NO: 7251, TMEM30A (Gene); SEQ ID NO: 7252, TMEM30A (Gene); SEQ ID NO: 7253, TMEM30A (Gene); SEQ ID NO: 7254, TMEM30A (Gene); SEQ ID NO: 7255, TMEM30A (Gene); SEQ ID NO: 7256, TMEM30A (Gene); SEQ ID NO: 7257, TMEM30A (Gene); SEQ ID NO: 7258, 6q14.1_DLBCL (FOCAL); SEQ ID NO: 7259, 6q14.1_DLBCL (FOCAL); SEQ ID NO: 7260, 6q14.1_DLBCL (FOCAL); SEQ ID NO: 7261, 6q14.1_DLBCL (FOCAL); SEQ ID NO: 7262, 6Q_DLBCL (ARM); SEQ ID NO: 7263, FP (FP); SEQ ID NO: 7264, 6Q_DLBCL (ARM); SEQ ID NO: 7265, 6Q_DLBCL (ARM); SEQ ID NO: 7266, 6Q_DLBCL (ARM); SEQ ID NO: 7267, 6Q_DLBCL (ARM); SEQ ID NO: 7268, MSI (MSI); SEQ ID NO: 7269, MSI (MSI); SEQ ID NO: 7270, 6Q_DLBCL (ARM); SEQ ID NO: 7271, 6Q_DLBCL (ARM); SEQ ID NO: 7272, ZNF292 (Gene); SEQ ID NO: 7273, ZNF292 (Gene); SEQ ID NO: 7274, ZNF292 (Gene); SEQ ID NO: 7275, ZNF292 (Gene); SEQ ID NO: 7276, ZNF292 (Gene); SEQ ID NO: 7277, ZNF292 (Gene); SEQ ID NO: 7278, ZNF292 (Gene); SEQ ID NO: 7279, ZNF292 (Gene); SEQ ID NO: 7280, ZNF292 (Gene); SEQ ID NO: 7281, ZNF292 (Gene); SEQ ID NO: 7282, ZNF292 (Gene); SEQ ID NO: 7283, ZNF292 (Gene); SEQ ID NO: 7284, ZNF292 (Gene); SEQ ID NO: 7285, ZNF292 (Gene); SEQ ID NO: 7286, ZNF292 (Gene); SEQ ID NO: 7287, ZNF292 (Gene); SEQ ID NO: 7288, ZNF292 (Gene); SEQ ID NO: 7289, ZNF292 (Gene); SEQ ID NO: 7290, ZNF292 (Gene); SEQ ID NO: 7291, ZNF292 (Gene); SEQ ID NO: 7292, ZNF292 (Gene); SEQ ID NO: 7293, ZNF292 (Gene); SEQ ID NO: 7294, 6Q_DLBCL (ARM); SEQ ID NO: 7295, 6Q_DLBCL (ARM); SEQ ID NO: 7296, MSI (MSI); SEQ ID NO: 7297, MSI (MSI); SEQ ID NO: 7298, 6Q_DLBCL (ARM); SEQ ID NO: 7299, 6Q_DLBCL (ARM); SEQ ID NO: 7300, 6Q_DLBCL (ARM); SEQ ID NO: 7301, 6Q_DLBCL (ARM); SEQ ID NO: 7302, 6Q_DLBCL (ARM); SEQ ID NO: 7303, 6q21_DLBCL (FOCAL); SEQ ID NO: 7304, 6q21_DLBCL (FOCAL); SEQ ID NO: 7305, 6q21_DLBCL (FOCAL); SEQ ID NO: 7306, 6q21_DLBCL (FOCAL); SEQ ID NO: 7307, 6q21_DLBCL (FOCAL); SEQ ID NO: 7308, 6q21_DLBCL (FOCAL); SEQ ID NO: 7309, 6q21_DLBCL (FOCAL); SEQ ID NO: 7310, 6q21_DLBCL (FOCAL); SEQ ID NO: 7311, 6q21_DLBCL (FOCAL); SEQ ID NO: 7312, 6q21_DLBCL (FOCAL); SEQ ID NO: 7313, 6q21_DLBCL (FOCAL); SEQ ID NO: 7314, 6q21_DLBCL (FOCAL); SEQ ID NO: 7315, 6q21_DLBCL (FOCAL); SEQ ID NO: 7316, 6q21_DLBCL (FOCAL); SEQ ID NO: 7317, MSI (MSI); SEQ ID NO: 7318, MSI (MSI); SEQ ID NO: 7319, 6q21_DLBCL (FOCAL); SEQ ID NO: 7320, 6q21_DLBCL (FOCAL); SEQ ID NO: 7321, 6q21_DLBCL (FOCAL); SEQ ID NO: 7322, 6q21_DLBCL (FOCAL); SEQ ID NO: 7323, 6q21_DLBCL (FOCAL); SEQ ID NO: 7324, 6q21_DLBCL (FOCAL); SEQ ID NO: 7325, 6q21_DLBCL (FOCAL); SEQ ID NO: 7326, 6q21_DLBCL (FOCAL); SEQ ID NO: 7327, 6q21_DLBCL (FOCAL); SEQ ID NO: 7328, 6q21_DLBCL (FOCAL); SEQ ID NO: 7329, 6q21_DLBCL (FOCAL); SEQ ID NO: 7330, 6q21_DLBCL (FOCAL); SEQ ID NO: 7331, 6q21_DLBCL (FOCAL); SEQ ID NO: 7332, 6q21_DLBCL (FOCAL); SEQ ID NO: 7333, 6q21_DLBCL (FOCAL); SEQ ID NO: 7334, 6q21_DLBCL (FOCAL); SEQ ID NO: 7335, 6q21_DLBCL (FOCAL); SEQ ID NO: 7336, 6q21_DLBCL (FOCAL); SEQ ID NO: 7337, 6q21_DLBCL (FOCAL); SEQ ID NO: 7338, 6q21_DLBCL (FOCAL); SEQ ID NO: 7339, 6q21_DLBCL (FOCAL); SEQ ID NO: 7340, 6q21_DLBCL (FOCAL); SEQ ID NO: 7341, 6q21_DLBCL (FOCAL); SEQ ID NO: 7342, 6q21_DLBCL (FOCAL); SEQ ID NO: 7343, 6q21_DLBCL (FOCAL); SEQ ID NO: 7344, 6q21_DLBCL (FOCAL); SEQ ID NO: 7345, 6q21_DLBCL (FOCAL); SEQ ID NO: 7346, 6q21_DLBCL (FOCAL); SEQ ID NO: 7347, 6q21_DLBCL (FOCAL); SEQ ID NO: 7348, PRDM1 (Gene); SEQ ID NO: 7349, PRDM1 (Gene); SEQ ID NO: 7350, PRDM1 (Gene); SEQ ID NO: 7351, PRDM1 (Gene); SEQ ID NO: 7352, PRDM1 (Gene); SEQ ID NO: 7353, PRDM1 (Gene); SEQ ID NO: 7354, PRDM1 (Gene); SEQ ID NO: 7355, PRDM1 (Gene); SEQ ID NO: 7356, PRDM1 (Gene); SEQ ID NO: 7357, PRDM1 (Gene); SEQ ID NO: 7358, PRDM1 (Gene); SEQ ID NO: 7359, PRDM1 (Gene); SEQ ID NO: 7360, 6q21_DLBCL (FOCAL); SEQ ID NO: 7361, 6q21_DLBCL (FOCAL); SEQ ID NO: 7362, 6q21_DLBCL (FOCAL); SEQ ID NO: 7363, 6q21_DLBCL (FOCAL); SEQ ID NO: 7364, 6q21_DLBCL (FOCAL); SEQ ID NO: 7365, 6q21_DLBCL (FOCAL); SEQ ID NO: 7366, 6q21_DLBCL (FOCAL); SEQ ID NO: 7367, 6q21_DLBCL (FOCAL); SEQ ID NO: 7368, 6q21_DLBCL (FOCAL); SEQ ID NO: 7369, 6q21_DLBCL (FOCAL); SEQ ID NO: 7370, 6q21_DLBCL (FOCAL); SEQ ID NO: 7371, 6q21_DLBCL (FOCAL); SEQ ID NO: 7372, 6q21_DLBCL (FOCAL); SEQ ID NO: 7373, 6q21_DLBCL (FOCAL); SEQ ID NO: 7374, 6q21_DLBCL (FOCAL); SEQ ID NO: 7375, 6q21_DLBCL (FOCAL); SEQ ID NO: 7376, FP (FP); SEQ ID NO: 7377, 6q21_DLBCL (FOCAL); SEQ ID NO: 7378, 6q21_DLBCL (FOCAL); SEQ ID NO: 7379, 6q21_DLBCL (FOCAL); SEQ ID NO: 7380, 6q21_DLBCL (FOCAL); SEQ ID NO: 7381, 6q21_DLBCL (FOCAL); SEQ ID NO: 7382, 6q21_DLBCL (FOCAL); SEQ ID NO: 7383, FYN (Gene); SEQ ID NO: 7384, FYN (Gene); SEQ ID NO: 7385, FYN (Gene); SEQ ID NO: 7386, FYN (Gene); SEQ ID NO: 7387, FYN (Gene); SEQ ID NO: 7388, FYN (Gene); SEQ ID NO: 7389, FYN (Gene); SEQ ID NO: 7390, FYN (Gene); SEQ ID NO: 7391, FYN (Gene); SEQ ID NO: 7392, FYN (Gene); SEQ ID NO: 7393, FYN (Gene); SEQ ID NO: 7394, FYN (Gene); SEQ ID NO: 7395, FYN (Gene); SEQ ID NO: 7396, 6Q_DLBCL (ARM); SEQ ID NO: 7397, 6Q_DLBCL (ARM); SEQ ID NO: 7398, 6Q_DLBCL (ARM); SEQ ID NO: 7399, 6Q_DLBCL (ARM); SEQ ID NO: 7400, 6Q_DLBCL (ARM); SEQ ID NO: 7401, 6Q_DLBCL (ARM); SEQ ID NO: 7402, 6Q_DLBCL (ARM); SEQ ID NO: 7403, 6Q_DLBCL (ARM); SEQ ID NO: 7404, 6Q_DLBCL (ARM); SEQ ID NO: 7405, PTPRK (Gene); SEQ ID NO: 7406, PTPRK (Gene); SEQ ID NO: 7407, PTPRK (Gene); SEQ ID NO: 7408, PTPRK (Gene); SEQ ID NO: 7409, PTPRK (Gene); SEQ ID NO: 7410, PTPRK (Gene); SEQ ID NO: 7411, PTPRK (Gene); SEQ ID NO: 7412, PTPRK (Gene); SEQ ID NO: 7413, PTPRK (Gene); SEQ ID NO: 7414, PTPRK (Gene); SEQ ID NO: 7415, PTPRK (Gene); SEQ ID NO: 7416, PTPRK (Gene); SEQ ID NO: 7417, PTPRK (Gene); SEQ ID NO: 7418, PTPRK (Gene); SEQ ID NO: 7419, PTPRK (Gene); SEQ ID NO: 7420, PTPRK (Gene); SEQ ID NO: 7421, PTPRK (Gene); SEQ ID NO: 7422, PTPRK (Gene); SEQ ID NO: 7423, PTPRK (Gene); SEQ ID NO: 7424, PTPRK (Gene); SEQ ID NO: 7425, PTPRK (Gene); SEQ ID NO: 7426, PTPRK (Gene); SEQ ID NO: 7427, PTPRK (Gene); SEQ ID NO: 7428, PTPRK (Gene); SEQ ID NO: 7429, PTPRK (Gene); SEQ ID NO: 7430, PTPRK (Gene); SEQ ID NO: 7431, PTPRK (Gene); SEQ ID NO: 7432, PTPRK (Gene); SEQ ID NO: 7433, PTPRK (Gene); SEQ ID NO: 7434, PTPRK (Gene); SEQ ID NO: 7435, PTPRK (Gene); SEQ ID NO: 7436, PTPRK (Gene); SEQ ID NO: 7437, PTPRK (Gene); SEQ ID NO: 7438, PTPRK (Gene); SEQ ID NO: 7439, PTPRK (Gene); SEQ ID NO: 7440, PTPRK (Gene); SEQ ID NO: 7441, 6Q_DLBCL (ARM); SEQ ID NO: 7442, 6Q_DLBCL (ARM); SEQ ID NO: 7443, 6Q_DLBCL (ARM); SEQ ID NO: 7444, 6Q_DLBCL (ARM); SEQ ID NO: 7445, 6q23.3_HL_wide (FOCAL); SEQ ID NO: 7446, 6q23.3_HL_wide (FOCAL); SEQ ID NO: 7447, 6q23.3_HL_wide (FOCAL); SEQ ID NO: 7448, 6q23.3_HL_wide (FOCAL); SEQ ID NO: 7449, 6q23.3_HL_wide (FOCAL); SEQ ID NO: 7450, 6q23.3_HL_wide (FOCAL); SEQ ID NO: 7451, SGK1 (Gene); SEQ ID NO: 7452, SGK1 (Gene); SEQ ID NO: 7453, SGK1 (Gene); SEQ ID NO: 7454, SGK1 (Gene); SEQ ID NO: 7455, SGK1 (Gene); SEQ ID NO: 7456, SGK1 (Gene); SEQ ID NO: 7457, SGK1 (Gene); SEQ ID NO: 7458, SGK1 (Gene); SEQ ID NO: 7459, SGK1 (Gene); SEQ ID NO: 7460, SGK1 (Gene); SEQ ID NO: 7461, SGK1 (Gene); SEQ ID NO: 7462, SGK1 (Gene); SEQ ID NO: 7463, SGK1 (Gene); SEQ ID NO: 7464, SGK1 (Gene); SEQ ID NO: 7465, SGK1 (Gene); SEQ ID NO: 7466, SGK1 (Gene); SEQ ID NO: 7467, SGK1 (Gene); SEQ ID NO: 7468, 6q23.3_HL_wide (FOCAL); SEQ ID NO: 7469, 6q23.3_HL_wide (FOCAL); SEQ ID NO: 7470, 6q23.3_HL_wide (FOCAL); SEQ ID NO: 7471, 6q23.3_HL_wide (FOCAL); SEQ ID NO: 7472, 6q23.3_HL_wide (FOCAL); SEQ ID NO: 7473, 6q23.3_HL_wide (FOCAL); SEQ ID NO: 7474, 6q23.3_HL_wide (FOCAL); SEQ ID NO: 7475, 6q23.3_HL_wide (FOCAL); SEQ ID NO: 7476, 6q23.3_HL_wide (FOCAL); SEQ ID NO: 7477, 6q23.3_HL_wide (FOCAL); SEQ ID NO: 7478, 6q23.3_HL_wide (FOCAL); SEQ ID NO: 7479, 6q23.3_HL_wide (FOCAL); SEQ ID NO: 7480, 6q23.3 HL_wide (FOCAL); SEQ ID NO: 7481, 6q23.3_HL_wide (FOCAL); SEQ ID NO: 7482, 6q23.3_HL_wide (FOCAL); SEQ ID NO: 7483, 6q23.3_HL_wide (FOCAL); SEQ ID NO: 7484, TNFAIP3 (Gene); SEQ ID NO: 7485, TNFAIP3 (Gene); SEQ ID NO: 7486, TNFAIP3 (Gene); SEQ ID NO: 7487, TNFAIP3 (Gene); SEQ ID NO: 7488, TNFAIP3 (Gene); SEQ ID NO: 7489, TNFAIP3 (Gene); SEQ ID NO: 7490, TNFAIP3 (Gene); SEQ ID NO: 7491, TNFAIP3 (Gene); SEQ ID NO: 7492, TNFAIP3 (Gene); SEQ ID NO: 7493, 6q23.3_HL_wide (FOCAL); SEQ ID NO: 7494, 6q23.3_HL_wide (FOCAL); SEQ ID NO: 7495, 6q23.3_HL_wide (FOCAL); SEQ ID NO: 7496, 6q23.3_HL_wide (FOCAL); SEQ ID NO: 7497, MSI (MSI); SEQ ID NO: 7498, MSI (MSI); SEQ ID NO: 7499, ECT2L (Gene); SEQ ID NO: 7500, ECT2L (Gene); SEQ ID NO: 7501, ECT2L (Gene); SEQ ID NO: 7502, ECT2L (Gene); SEQ ID NO: 7503, ECT2L (Gene); SEQ ID NO: 7504, ECT2L (Gene); SEQ ID NO: 7505, ECT2L (Gene); SEQ ID NO: 7506, ECT2L (Gene); SEQ ID NO: 7507, ECT2L (Gene); SEQ ID NO: 7508, ECT2L (Gene); SEQ ID NO: 7509, ECT2L (Gene); SEQ ID NO: 7510, ECT2L (Gene); SEQ ID NO: 7511, ECT2L (Gene); SEQ ID NO: 7512, ECT2L (Gene); SEQ ID NO: 7513, ECT2L (Gene); SEQ ID NO: 7514, ECT2L (Gene); SEQ ID NO: 7515, ECT2L (Gene); SEQ ID NO: 7516, ECT2L (Gene); SEQ ID NO: 7517, ECT2L (Gene); SEQ ID NO: 7518, ECT2L (Gene); SEQ ID NO: 7519, 6q23.3_HL_wide (FOCAL); SEQ ID NO: 7520, 6q23.3_HL_wide (FOCAL); SEQ ID NO: 7521, 6q23.3_HL_wide (FOCAL); SEQ ID NO: 7522, 6q23.3_HL_wide (FOCAL); SEQ ID NO: 7523, 6q23.3_HL_wide (FOCAL); SEQ ID NO: 7524, 6q23.3_HL_wide (FOCAL); SEQ ID NO: 7525, 6q23.3_HL_wide (FOCAL); SEQ ID NO: 7526, 6q23.3_HL_wide (FOCAL); SEQ ID NO: 7527, 6q23.3_HL_wide (FOCAL); SEQ ID NO: 7528, 6q23.3_HL_wide (FOCAL); SEQ ID NO: 7529, 6q23.3_HL_wide (FOCAL); SEQ ID NO: 7530, 6q23.3_HL_wide (FOCAL); SEQ ID NO: 7531, 6q23.3_HL_wide (FOCAL); SEQ ID NO: 7532, 6q23.3_HL_wide (FOCAL); SEQ ID NO: 7533, 6q23.3_HL_wide (FOCAL); SEQ ID NO: 7534, ADGRG6 (Gene); SEQ ID NO: 7535, ADGRG6 (Gene); SEQ ID NO: 7536, ADGRG6 (Gene); SEQ ID NO: 7537, ADGRG6 (Gene); SEQ ID NO: 7538, ADGRG6 (Gene); SEQ ID NO: 7539, ADGRG6 (Gene); SEQ ID NO: 7540, ADGRG6 (Gene); SEQ ID NO: 7541, ADGRG6 (Gene); SEQ ID NO: 7542, ADGRG6 (Gene); SEQ ID NO: 7543, ADGRG6 (Gene); SEQ ID NO: 7544, ADGRG6 (Gene); SEQ ID NO: 7545, ADGRG6 (Gene); SEQ ID NO: 7546, ADGRG6 (Gene); SEQ ID NO: 7547, ADGRG6 (Gene); SEQ ID NO: 7548, ADGRG6 (Gene); SEQ ID NO: 7549, ADGRG6 (Gene); SEQ ID NO: 7550, ADGRG6 (Gene); SEQ ID NO: 7551, ADGRG6 (Gene); SEQ ID NO: 7552, ADGRG6 (Gene); SEQ ID NO: 7553, ADGRG6 (Gene); SEQ ID NO: 7554, ADGRG6 (Gene); SEQ ID NO: 7555, ADGRG6 (Gene); SEQ ID NO: 7556, ADGRG6 (Gene); SEQ ID NO: 7557, ADGRG6 (Gene); SEQ ID NO: 7558, ADGRG6 (Gene); SEQ ID NO: 7559, ADGRG6 (Gene); SEQ ID NO: 7560, ADGRG6 (Gene); SEQ ID NO: 7561, 6q23.3_HL_wide (FOCAL); SEQ ID NO: 7562, 6q23.3_HL_wide (FOCAL); SEQ ID NO: 7563, 6q23.3_HL_wide (FOCAL); SEQ ID NO: 7564, 6q23.3_HL_wide (FOCAL); SEQ ID NO: 7565, 6q23.3_HL_wide (FOCAL); SEQ ID NO: 7566, 6q23.3_HL_wide (FOCAL); SEQ ID NO: 7567, 6q23.3_HL_wide (FOCAL); SEQ ID NO: 7568, 6q23.3_HL_wide (FOCAL); SEQ ID NO: 7569, 6q23.3_HL_wide (FOCAL); SEQ ID NO: 7570, 6q23.3_HL_wide (FOCAL); SEQ ID NO: 7571, 6q23.3_HL_wide (FOCAL); SEQ ID NO: 7572, 6q23.3_HL_wide (FOCAL); SEQ ID NO: 7573, 6q23.3_HL_wide (FOCAL); SEQ ID NO: 7574, 6q23.3_HL_wide (FOCAL); SEQ ID NO: 7575, 6q23.3_HL_wide (FOCAL); SEQ ID NO: 7576, 6q23.3_HL_wide (FOCAL); SEQ ID NO: 7577, 6q23.3_HL_wide (FOCAL); SEQ ID NO: 7578, 6q23.3_HL_wide (FOCAL); SEQ ID NO: 7579, 6q23.3_HL_wide (FOCAL); SEQ ID NO: 7580, 6q23.3_HL_wide (FOCAL); SEQ ID NO: 7581, 6q23.3_HL_wide (FOCAL); SEQ ID NO: 7582, 6q23.3_HL_wide (FOCAL); SEQ ID NO: 7583, 6q23.3_HL_wide (FOCAL); SEQ ID NO: 7584, 6q23.3_HL_wide (FOCAL); SEQ ID NO: 7585, 6q23.3_HL_wide (FOCAL); SEQ ID NO: 7586, 6Q_DLBCL (ARM); SEQ ID NO: 7587, 6Q_DLBCL (ARM); SEQ ID NO: 7588, FP (FP); SEQ ID NO: 7589, 6Q_DLBCL (ARM); SEQ ID NO: 7590, 6Q_DLBCL (ARM); SEQ ID NO: 7591, 6Q_DLBCL (ARM); SEQ ID NO: 7592, 6Q_DLBCL (ARM); SEQ ID NO: 7593, 6Q_DLBCL (ARM); SEQ ID NO: 7594, 6Q_DLBCL (ARM); SEQ ID NO: 7595, ARID1B (Gene); SEQ ID NO: 7596, ARID1B (Gene); SEQ ID NO: 7597, ARID1B (Gene); SEQ ID NO: 7598, ARID1B (Gene); SEQ ID NO: 7599, ARID1B (Gene); SEQ ID NO: 7600, ARID1B (Gene); SEQ ID NO: 7601, ARID1B (Gene); SEQ ID NO: 7602, ARID1B (Gene); SEQ ID NO: 7603, 6Q_DLBCL (ARM); SEQ ID NO: 7604, ARID1B (Gene); SEQ ID NO: 7605, ARID1B (Gene); SEQ ID NO: 7606, ARID1B (Gene); SEQ ID NO: 7607, ARID1B (Gene); SEQ ID NO: 7608, ARID1B (Gene); SEQ ID NO: 7609, ARID1B (Gene); SEQ ID NO: 7610, ARID1B (Gene); SEQ ID NO: 7611, ARID1B (Gene); SEQ ID NO: 7612, ARID1B (Gene); SEQ ID NO: 7613, ARID1B (Gene); SEQ ID NO: 7614, ARID1B (Gene); SEQ ID NO: 7615, ARID1B (Gene); SEQ ID NO: 7616, ARID1B (Gene); SEQ ID NO: 7617, ARID1B (Gene); SEQ ID NO: 7618, ARID1B (Gene); SEQ ID NO: 7619, ARID1B (Gene); SEQ ID NO: 7620, ARID1B (Gene); SEQ ID NO: 7621, ARID1B (Gene); SEQ ID NO: 7622, ARID1B (Gene); SEQ ID NO: 7623, ARID1B (Gene); SEQ ID NO: 7624, ARID1B (Gene); SEQ ID NO: 7625, MSI (MSI); SEQ ID NO: 7626, MSI (MSI); SEQ ID NO: 7627, 6Q_DLBCL (ARM); SEQ ID NO: 7628, 6Q_DLBCL (ARM); SEQ ID NO: 7629, 6Q_DLBCL (ARM); SEQ ID NO: 7630, 6Q_DLBCL (ARM); SEQ ID NO: 7631, 6Q_DLBCL (ARM); SEQ ID NO: 7632, 6Q_DLBCL (ARM); SEQ ID NO: 7633, 6Q_DLBCL (ARM); SEQ ID NO: 7634, 6Q_DLBCL (ARM); SEQ ID NO: 7635, MSI (MSI); SEQ ID NO: 7636, MSI (MSI); SEQ ID NO: 7637, 6Q_DLBCL (ARM); SEQ ID NO: 7638, FP (FP); SEQ ID NO: 7639, 6Q_DLBCL (ARM); SEQ ID NO: 7640, 6Q_DLBCL (ARM); SEQ ID NO: 7641, 6Q_DLBCL (ARM); SEQ ID NO: 7642, 6Q_DLBCL (ARM); SEQ ID NO: 7643, 6Q_DLBCL (ARM); SEQ ID NO: 7644, 7P_DLBCL (ARM); SEQ ID NO: 7645, CARD11 (Gene); SEQ ID NO: 7646, CARD11 (Gene); SEQ ID NO: 7647, CARD11 (Gene); SEQ ID NO: 7648, CARD11 (Gene); SEQ ID NO: 7649, CARD11 (Gene); SEQ ID NO: 7650, CARD11 (Gene); SEQ ID NO: 7651, CARD11 (Gene); SEQ ID NO: 7652, CARD11 (Gene); SEQ ID NO: 7653, CARD11 (Gene); SEQ ID NO: 7654, CARD11 (Gene); SEQ ID NO: 7655, CARD11 (Gene); SEQ ID NO: 7656, CARD11 (Gene); SEQ ID NO: 7657, CARD11 (Gene); SEQ ID NO: 7658, CARD11 (Gene); SEQ ID NO: 7659, CARD11 (Gene); SEQ ID NO: 7660, CARD11 (Gene); SEQ ID NO: 7661, CARD11 (Gene); SEQ ID NO: 7662, CARD11 (Gene); SEQ ID NO: 7663, CARD11 (Gene); SEQ ID NO: 7664, CARD11 (Gene); SEQ ID NO: 7665, CARD11 (Gene); SEQ ID NO: 7666, CARD11 (Gene); SEQ ID NO: 7667, CARD11 (Gene); SEQ ID NO: 7668, CARD11 (Gene); SEQ ID NO: 7669, CARD11 (Gene); SEQ ID NO: 7670, 7P_DLBCL (ARM); SEQ ID NO: 7671, 7P_DLBCL (ARM); SEQ ID NO: 7672, 7P_DLBCL (ARM); SEQ ID NO: 7673, ACTB (Gene); SEQ ID NO: 7674, ACTB (Gene); SEQ ID NO: 7675, ACTB (Gene); SEQ ID NO: 7676, ACTB (Gene); SEQ ID NO: 7677, ACTB (Gene); SEQ ID NO: 7678, 7P_DLBCL (ARM); SEQ ID NO: 7679, MSI (MSI); SEQ ID NO: 7680, MSI (MSI); SEQ ID NO: 7681, 7P_DLBCL (ARM); SEQ ID NO: 7682, 7P_DLBCL (ARM); SEQ ID NO: 7683, 7P_DLBCL (ARM); SEQ ID NO: 7684, 7P_DLBCL (ARM); SEQ ID NO: 7685, 7P_DLBCL (ARM); SEQ ID NO: 7686, 7P_DLBCL (ARM); SEQ ID NO: 7687, 7P_DLBCL (ARM); SEQ ID NO: 7688, 7P_DLBCL (ARM); SEQ ID NO: 7689, 7P_DLBCL (ARM); SEQ ID NO: 7690, 7P_DLBCL (ARM); SEQ ID NO: 7691, 7P_DLBCL (ARM); SEQ ID NO: 7692, FP (FP); SEQ ID NO: 7693, 7P_DLBCL (ARM); SEQ ID NO: 7694, 7P_DLBCL (ARM); SEQ ID NO: 7695, 7P_DLBCL (ARM); SEQ ID NO: 7696, 7P_DLBCL (ARM); SEQ ID NO: 7697, IL6 (Gene); SEQ ID NO: 7698, IL6 (Gene); SEQ ID NO: 7699, IL6 (Gene); SEQ ID NO: 7700, IL6 (Gene); SEQ ID NO: 7701, IL6 (Gene); SEQ ID NO: 7702, 7P_DLBCL (ARM); SEQ ID NO: 7703, 7P_DLBCL (ARM); SEQ ID NO: 7704, 7P_DLBCL (ARM); SEQ ID NO: 7705, 7P_DLBCL (ARM); SEQ ID NO: 7706, 7P_DLBCL (ARM); SEQ ID NO: 7707, 7P_DLBCL (ARM); SEQ ID NO: 7708, 7P_DLBCL (ARM); SEQ ID NO: 7709, 7P_DLBCL (ARM); SEQ ID NO: 7710, 7P_DLBCL (ARM); SEQ ID NO: 7711, 7P_DLBCL (ARM); SEQ ID NO: 7712, 7P_DLBCL (ARM); SEQ ID NO: 7713, 7P_DLBCL (ARM); SEQ ID NO: 7714, 7P_DLBCL (ARM); SEQ ID NO: 7715, 7P_DLBCL (ARM); SEQ ID NO: 7716, 7P_DLBCL (ARM); SEQ ID NO: 7717, 7P_DLBCL (ARM); SEQ ID NO: 7718, 7P_DLBCL (ARM); SEQ ID NO: 7719, 7P_DLBCL (ARM); SEQ ID NO: 7720, 7P_DLBCL (ARM); SEQ ID NO: 7721, 7P_DLBCL (ARM); SEQ ID NO: 7722, 7P_DLBCL (ARM); SEQ ID NO: 7723, 7P_DLBCL (ARM); SEQ ID NO: 7724, 7P_DLBCL (ARM); SEQ ID NO: 7725, 7P_DLBCL (ARM); SEQ ID NO: 7726, 7P_DLBCL (ARM); SEQ ID NO: 7727, 7P_DLBCL (ARM); SEQ ID NO: 7728, 7P_DLBCL (ARM); SEQ ID NO: 7729, 7P_DLBCL (ARM); SEQ ID NO: 7730, 7P_DLBCL (ARM); SEQ ID NO: 7731, 7P_DLBCL (ARM); SEQ ID NO: 7732, 7P_DLBCL (ARM); SEQ ID NO: 7733, 7P_DLBCL (ARM); SEQ ID NO: 7734, 7P_DLBCL (ARM); SEQ ID NO: 7735, 7P_DLBCL (ARM); SEQ ID NO: 7736, 7Q_DLBCL (ARM); SEQ ID NO: 7737, 7Q_DLBCL (ARM); SEQ ID NO: 7738, 7Q_DLBCL (ARM); SEQ ID NO: 7739, 7Q_DLBCL (ARM); SEQ ID NO: 7740, 7Q_DLBCL (ARM); SEQ ID NO: 7741, 7Q_DLBCL (ARM); SEQ ID NO: 7742, MSI (MSI); SEQ ID NO: 7743, MSI (MSI); SEQ ID NO: 7744, 7Q_DLBCL (ARM); SEQ ID NO: 7745, 7Q_DLBCL (ARM); SEQ ID NO: 7746, 7Q_DLBCL (ARM); SEQ ID NO: 7747, 7Q_DLBCL (ARM); SEQ ID NO: 7748, 7Q_DLBCL (ARM); SEQ ID NO: 7749, 7Q_DLBCL (ARM); SEQ ID NO: 7750, 7Q_DLBCL (ARM); SEQ ID NO: 7751, FP (FP); SEQ ID NO: 7752, 7Q_DLBCL (ARM); SEQ ID NO: 7753, 7Q_DLBCL (ARM); SEQ ID NO: 7754, 7Q_DLBCL (ARM); SEQ ID NO: 7755, 7Q_DLBCL (ARM); SEQ ID NO: 7756, 7Q_DLBCL (ARM); SEQ ID NO: 7757, 7q22.1_DLBCL (FOCAL); SEQ ID NO: 7758, 7q22.1_DLBCL (FOCAL); SEQ ID NO: 7759, 7q22.1_DLBCL (FOCAL); SEQ ID NO: 7760, 7q22.1_DLBCL (FOCAL); SEQ ID NO: 7761, 7q22.1_DLBCL (FOCAL); SEQ ID NO: 7762, 7q22.1_DLBCL (FOCAL); SEQ ID NO: 7763, TRRAP (Gene); SEQ ID NO: 7764, TRRAP (Gene); SEQ ID NO: 7765, TRRAP (Gene); SEQ ID NO: 7766, TRRAP (Gene); SEQ ID NO: 7767, TRRAP (Gene); SEQ ID NO: 7768, TRRAP (Gene); SEQ ID NO: 7769, TRRAP (Gene); SEQ ID NO: 7770, TRRAP (Gene); SEQ ID NO: 7771, TRRAP (Gene); SEQ ID NO: 7772, TRRAP (Gene); SEQ ID NO: 7773, TRRAP (Gene); SEQ ID NO: 7774, TRRAP (Gene); SEQ ID NO: 7775, TRRAP (Gene); SEQ ID NO: 7776, TRRAP (Gene); SEQ ID NO: 7777, TRRAP (Gene); SEQ ID NO: 7778, TRRAP (Gene); SEQ ID NO: 7779, TRRAP (Gene); SEQ ID NO: 7780, TRRAP (Gene); SEQ ID NO: 7781, TRRAP (Gene); SEQ ID NO: 7782, TRRAP (Gene); SEQ ID NO: 7783, TRRAP (Gene); SEQ ID NO: 7784, TRRAP (Gene); SEQ ID NO: 7785, TRRAP (Gene); SEQ ID NO: 7786, TRRAP (Gene); SEQ ID NO: 7787, TRRAP (Gene); SEQ ID NO: 7788, TRRAP (Gene); SEQ ID NO: 7789, TRRAP (Gene); SEQ ID NO: 7790, TRRAP (Gene); SEQ ID NO: 7791, TRRAP (Gene); SEQ ID NO: 7792, TRRAP (Gene); SEQ ID NO: 7793, TRRAP (Gene); SEQ ID NO: 7794, TRRAP (Gene); SEQ ID NO: 7795, TRRAP (Gene); SEQ ID NO: 7796, TRRAP (Gene); SEQ ID NO: 7797, TRRAP (Gene); SEQ ID NO: 7798, TRRAP (Gene); SEQ ID NO: 7799, TRRAP (Gene); SEQ ID NO: 7800, TRRAP (Gene); SEQ ID NO: 7801, TRRAP (Gene); SEQ ID NO: 7802, TRRAP (Gene); SEQ ID NO: 7803, TRRAP (Gene); SEQ ID NO: 7804, TRRAP (Gene); SEQ ID NO: 7805, TRRAP (Gene); SEQ ID NO: 7806, TRRAP (Gene); SEQ ID NO: 7807, TRRAP (Gene); SEQ ID NO: 7808, TRRAP (Gene); SEQ ID NO: 7809, TRRAP (Gene); SEQ ID NO: 7810, TRRAP (Gene); SEQ ID NO: 7811, TRRAP (Gene); SEQ ID NO: 7812, TRRAP (Gene); SEQ ID NO: 7813, TRRAP (Gene); SEQ ID NO: 7814, TRRAP (Gene); SEQ ID NO: 7815, TRRAP (Gene); SEQ ID NO: 7816, TRRAP (Gene); SEQ ID NO: 7817, TRRAP (Gene); SEQ ID NO: 7818, TRRAP (Gene); SEQ ID NO: 7819, TRRAP (Gene); SEQ ID NO: 7820, TRRAP (Gene); SEQ ID NO: 7821, TRRAP (Gene); SEQ ID NO: 7822, TRRAP (Gene); SEQ ID NO: 7823, TRRAP (Gene); SEQ ID NO: 7824, TRRAP (Gene); SEQ ID NO: 7825, TRRAP (Gene); SEQ ID NO: 7826, TRRAP (Gene); SEQ ID NO: 7827, TRRAP (Gene); SEQ ID NO: 7828, TRRAP (Gene); SEQ ID NO: 7829, TRRAP (Gene); SEQ ID NO: 7830, TRRAP (Gene); SEQ ID NO: 7831, TRRAP (Gene); SEQ ID NO: 7832, TRRAP (Gene); SEQ ID NO: 7833, TRRAP (Gene); SEQ ID NO: 7834, TRRAP (Gene); SEQ ID NO: 7835, TRRAP (Gene); SEQ ID NO: 7836, 7q22.1_DLBCL (FOCAL); SEQ ID NO: 7837, 7q22.1_DLBCL (FOCAL); SEQ ID NO: 7838, 7q22.1_DLBCL (FOCAL); SEQ ID NO: 7839, 7q22.1_DLBCL (FOCAL); SEQ ID NO: 7840, 7q22.1_DLBCL (FOCAL); SEQ ID NO: 7841, 7q22.1_DLBCL (FOCAL); SEQ ID NO: 7842, 7q22.1_DLBCL (FOCAL); SEQ ID NO: 7843, 7q22.1_DLBCL (FOCAL); SEQ ID NO: 7844, GNB2 (Gene); SEQ ID NO: 7845, GNB2 (Gene); SEQ ID NO: 7846, GNB2 (Gene); SEQ ID NO: 7847, GNB2 (Gene); SEQ ID NO: 7848, GNB2 (Gene); SEQ ID NO: 7849, GNB2 (Gene); SEQ ID NO: 7850, GNB2 (Gene); SEQ ID NO: 7851, GNB2 (Gene); SEQ ID NO: 7852, GNB2 (Gene); SEQ ID NO: 7853, 7q22.1_DLBCL (FOCAL); SEQ ID NO: 7854, 7q22.1_DLBCL (FOCAL); SEQ ID NO: 7855, 7q22.1_DLBCL (FOCAL); SEQ ID NO: 7856, MSI (MSI); SEQ ID NO: 7857, MSI (MSI); SEQ ID NO: 7858, 7q22.1_DLBCL (FOCAL); SEQ ID NO: 7859, 7q22.1_DLBCL (FOCAL); SEQ ID NO: 7860, 7q22.1_DLBCL (FOCAL); SEQ ID NO: 7861, 7q22.1_DLBCL (FOCAL); SEQ ID NO: 7862, 7q22.1_DLBCL (FOCAL); SEQ ID NO: 7863, 7q22.1_DLBCL (FOCAL); SEQ ID NO: 7864, 7q22.1_DLBCL (FOCAL); SEQ ID NO: 7865, 7q22.1_DLBCL (FOCAL); SEQ ID NO: 7866, 7q22.1_DLBCL (FOCAL); SEQ ID NO: 7867, RELN (Gene); SEQ ID NO: 7868, FP (FP); SEQ ID NO: 7869, RELN (Gene); SEQ ID NO: 7870, RELN (Gene); SEQ ID NO: 7871, RELN (Gene); SEQ ID NO: 7872, RELN (Gene); SEQ ID NO: 7873, RELN (Gene); SEQ ID NO: 7874, RELN (Gene); SEQ ID NO: 7875, RELN (Gene); SEQ ID NO: 7876, RELN (Gene); SEQ ID NO: 7877, RELN (Gene); SEQ ID NO: 7878, RELN (Gene); SEQ ID NO: 7879, RELN (Gene); SEQ ID NO: 7880, RELN (Gene); SEQ ID NO: 7881, RELN (Gene); SEQ ID NO: 7882, RELN (Gene); SEQ ID NO: 7883, RELN (Gene); SEQ ID NO: 7884, RELN (Gene); SEQ ID NO: 7885, RELN (Gene); SEQ ID NO: 7886, RELN (Gene); SEQ ID NO: 7887, RELN (Gene); SEQ ID NO: 7888, RELN (Gene); SEQ ID NO: 7889, RELN (Gene); SEQ ID NO: 7890, RELN (Gene); SEQ ID NO: 7891, RELN (Gene); SEQ ID NO: 7892, RELN (Gene); SEQ ID NO: 7893, RELN (Gene); SEQ ID NO: 7894, RELN (Gene); SEQ ID NO: 7895, RELN (Gene); SEQ ID NO: 7896, RELN (Gene); SEQ ID NO: 7897, RELN (Gene); SEQ ID NO: 7898, RELN (Gene); SEQ ID NO: 7899, RELN (Gene); SEQ ID NO: 7900, RELN (Gene); SEQ ID NO: 7901, RELN (Gene); SEQ ID NO: 7902, RELN (Gene); SEQ ID NO: 7903, RELN (Gene); SEQ ID NO: 7904, RELN (Gene); SEQ ID NO: 7905, RELN (Gene); SEQ ID NO: 7906, RELN (Gene); SEQ ID NO: 7907, RELN (Gene); SEQ ID NO: 7908, RELN (Gene); SEQ ID NO: 7909, RELN (Gene); SEQ ID NO: 7910, RELN (Gene); SEQ ID NO: 7911, RELN (Gene); SEQ ID NO: 7912, RELN (Gene); SEQ ID NO: 7913, RELN (Gene); SEQ ID NO: 7914, RELN (Gene); SEQ ID NO: 7915, RELN (Gene); SEQ ID NO: 7916, RELN (Gene); SEQ ID NO: 7917, RELN (Gene); SEQ ID NO: 7918, RELN (Gene); SEQ ID NO: 7919, RELN (Gene); SEQ ID NO: 7920, RELN (Gene); SEQ ID NO: 7921, RELN (Gene); SEQ ID NO: 7922, RELN (Gene); SEQ ID NO: 7923, RELN (Gene); SEQ ID NO: 7924, RELN (Gene); SEQ ID NO: 7925, RELN (Gene); SEQ ID NO: 7926, RELN (Gene); SEQ ID NO: 7927, RELN (Gene); SEQ ID NO: 7928, RELN (Gene); SEQ ID NO: 7929, RELN (Gene); SEQ ID NO: 7930, RELN (Gene); SEQ ID NO: 7931, RELN (Gene); SEQ ID NO: 7932, RELN (Gene); SEQ ID NO: 7933, RELN (Gene); SEQ ID NO: 7934, 7q22.1_DLBCL (FOCAL); SEQ ID NO: 7935, RELN (Gene); SEQ ID NO: 7936, 7q22.1_DLBCL (FOCAL); SEQ ID NO: 7937, 7q22.1_DLBCL (FOCAL); SEQ ID NO: 7938, 7q22.1_DLBCL (FOCAL); SEQ ID NO: 7939, 7q22.1_DLBCL (FOCAL); SEQ ID NO: 7940, 7q22.1_DLBCL (FOCAL); SEQ ID NO: 7941, 7q22.1_DLBCL (FOCAL); SEQ ID NO: 7942, 7q22.1_DLBCL (FOCAL); SEQ ID NO: 7943, 7q22.1_DLBCL (FOCAL); SEQ ID NO: 7944, 7q22.1_DLBCL (FOCAL); SEQ ID NO: 7945, 7q22.1_DLBCL (FOCAL); SEQ ID NO: 7946, 7Q_DLBCL (ARM); SEQ ID NO: 7947, 7Q_DLBCL (ARM); SEQ ID NO: 7948, 7Q_DLBCL (ARM); SEQ ID NO: 7949, 7Q_DLBCL (ARM); SEQ ID NO: 7950, 7Q_DLBCL (ARM); SEQ ID NO: 7951, 7Q_DLBCL (ARM); SEQ ID NO: 7952, 7Q_DLBCL (ARM); SEQ ID NO: 7953, 7Q_DLBCL (ARM); SEQ ID NO: 7954, 7Q_DLBCL (ARM); SEQ ID NO: 7955, 7Q_DLBCL (ARM); SEQ ID NO: 7956, 7Q_DLBCL (ARM); SEQ ID NO: 7957, 7Q_DLBCL (ARM); SEQ ID NO: 7958, POT1 (Gene); SEQ ID NO: 7959, POT1 (Gene); SEQ ID NO: 7960, POT1 (Gene); SEQ ID NO: 7961, POT1 (Gene); SEQ ID NO: 7962, POT1 (Gene); SEQ ID NO: 7963, POT1 (Gene); SEQ ID NO: 7964, POT1 (Gene); SEQ ID NO: 7965, POT1 (Gene); SEQ ID NO: 7966, POT1 (Gene); SEQ ID NO: 7967, POT1 (Gene); SEQ ID NO: 7968, POT1 (Gene); SEQ ID NO: 7969, POT1 (Gene); SEQ ID NO: 7970, POT1 (Gene); SEQ ID NO: 7971, POT1 (Gene); SEQ ID NO: 7972, POT1 (Gene); SEQ ID NO: 7973, 7Q_DLBCL (ARM); SEQ ID NO: 7974, 7Q_DLBCL (ARM); SEQ ID NO: 7975, 7Q_DLBCL (ARM); SEQ ID NO: 7976, 7Q_DLBCL (ARM); SEQ ID NO: 7977, 7Q_DLBCL (ARM); SEQ ID NO: 7978, 7Q_DLBCL (ARM); SEQ ID NO: 7979, 7Q_DLBCL (ARM); SEQ ID NO: 7980, 7Q_DLBCL (ARM); SEQ ID NO: 7981, 7Q_DLBCL (ARM); SEQ ID NO: 7982, 7Q_DLBCL (ARM); SEQ ID NO: 7983, 7Q_DLBCL (ARM); SEQ ID NO: 7984, 7Q_DLBCL (ARM); SEQ ID NO: 7985, 7Q_DLBCL (ARM); SEQ ID NO: 7986, 7Q_DLBCL (ARM); SEQ ID NO: 7987, 7Q_DLBCL (ARM); SEQ ID NO: 7988, BRAF (Gene); SEQ ID NO: 7989, BRAF (Gene); SEQ ID NO: 7990, BRAF (Gene); SEQ ID NO: 7991, BRAF (Gene); SEQ ID NO: 7992, BRAF (Gene); SEQ ID NO: 7993, BRAF (Gene); SEQ ID NO: 7994, BRAF (Gene); SEQ ID NO: 7995, BRAF (Gene); SEQ ID NO: 7996, BRAF (Gene); SEQ ID NO: 7997, BRAF (Gene); SEQ ID NO: 7998, BRAF (Gene); SEQ ID NO: 7999, BRAF (Gene); SEQ ID NO: 8000, BRAF (Gene); SEQ ID NO: 8001, BRAF (Gene); SEQ ID NO: 8002, BRAF (Gene); SEQ ID NO: 8003, BRAF (Gene); SEQ ID NO: 8004, BRAF (Gene); SEQ ID NO: 8005, BRAF (Gene); SEQ ID NO: 8006, BRAF (Gene); SEQ ID NO: 8007, 7Q_DLBCL (ARM); SEQ ID NO: 8008, 7Q_DLBCL (ARM); SEQ ID NO: 8009, 7Q_DLBCL (ARM); SEQ ID NO: 8010, 7Q_DLBCL (ARM); SEQ ID NO: 8011, 7Q_DLBCL (ARM); SEQ ID NO: 8012, 7Q_DLBCL (ARM); SEQ ID NO: 8013, 7Q_DLBCL (ARM); SEQ ID NO: 8014, EZH2 (Gene); SEQ ID NO: 8015, EZH2 (Gene); SEQ ID NO: 8016, EZH2 (Gene); SEQ ID NO: 8017, EZH2 (Gene); SEQ ID NO: 8018, EZH2 (Gene); SEQ ID NO: 8019, EZH2 (Gene); SEQ ID NO: 8020, EZH2 (Gene); SEQ ID NO: 8021, EZH2 (Gene); SEQ ID NO: 8022, EZH2 (Gene); SEQ ID NO: 8023, EZH2 (Gene); SEQ ID NO: 8024, EZH2 (Gene); SEQ ID NO: 8025, EZH2 (Gene); SEQ ID NO: 8026, EZH2 (Gene); SEQ ID NO: 8027, EZH2 (Gene); SEQ ID NO: 8028, EZH2 (Gene); SEQ ID NO: 8029, EZH2 (Gene); SEQ ID NO: 8030, EZH2 (Gene); SEQ ID NO: 8031, EZH2 (Gene); SEQ ID NO: 8032, EZH2 (Gene); SEQ ID NO: 8033, EZH2 (Gene); SEQ ID NO: 8034, 7Q_DLBCL (ARM); SEQ ID NO: 8035, 7Q_DLBCL (ARM); SEQ ID NO: 8036, 7Q_DLBCL (ARM); SEQ ID NO: 8037, KMT2C (Gene); SEQ ID NO: 8038, KMT2C (Gene); SEQ ID NO: 8039, KMT2C (Gene); SEQ ID NO: 8040, KMT2C (Gene); SEQ ID NO: 8041, KMT2C (Gene); SEQ ID NO: 8042, KMT2C (Gene); SEQ ID NO: 8043, KMT2C (Gene); SEQ ID NO: 8044, KMT2C (Gene); SEQ ID NO: 8045, KMT2C (Gene); SEQ ID NO: 8046, KMT2C (Gene); SEQ ID NO: 8047, KMT2C (Gene); SEQ ID NO: 8048, KMT2C (Gene); SEQ ID NO: 8049, KMT2C (Gene); SEQ ID NO: 8050, KMT2C (Gene); SEQ ID NO: 8051, KMT2C (Gene); SEQ ID NO: 8052, KMT2C (Gene); SEQ ID NO: 8053, KMT2C (Gene); SEQ ID NO: 8054, KMT2C (Gene); SEQ ID NO: 8055, KMT2C (Gene); SEQ ID NO: 8056, KMT2C (Gene); SEQ ID NO: 8057, KMT2C (Gene); SEQ ID NO: 8058, KMT2C (Gene); SEQ ID NO: 8059, KMT2C (Gene); SEQ ID NO: 8060, KMT2C (Gene); SEQ ID NO: 8061, KMT2C (Gene); SEQ ID NO: 8062, KMT2C (Gene); SEQ ID NO: 8063, KMT2C (Gene); SEQ ID NO: 8064, KMT2C (Gene); SEQ ID NO: 8065, KMT2C (Gene); SEQ ID NO: 8066, KMT2C (Gene); SEQ ID NO: 8067, KMT2C (Gene); SEQ ID NO: 8068, KMT2C (Gene); SEQ ID NO: 8069, KMT2C (Gene); SEQ ID NO: 8070, KMT2C (Gene); SEQ ID NO: 8071, KMT2C (Gene); SEQ ID NO: 8072, KMT2C (Gene); SEQ ID NO: 8073, KMT2C (Gene); SEQ ID NO: 8074, KMT2C (Gene); SEQ ID NO: 8075, KMT2C (Gene); SEQ ID NO: 8076, KMT2C (Gene); SEQ ID NO: 8077, KMT2C (Gene); SEQ ID NO: 8078, KMT2C (Gene); SEQ ID NO: 8079, KMT2C (Gene); SEQ ID NO: 8080, KMT2C (Gene); SEQ ID NO: 8081, KMT2C (Gene); SEQ ID NO: 8082, KMT2C (Gene); SEQ ID NO: 8083, KMT2C (Gene); SEQ ID NO: 8084, KMT2C (Gene); SEQ ID NO: 8085, KMT2C (Gene); SEQ ID NO: 8086, KMT2C (Gene); SEQ ID NO: 8087, KMT2C (Gene); SEQ ID NO: 8088, KMT2C (Gene); SEQ ID NO: 8089, KMT2C (Gene); SEQ ID NO: 8090, KMT2C (Gene); SEQ ID NO: 8091, KMT2C (Gene); SEQ ID NO: 8092, KMT2C (Gene); SEQ ID NO: 8093, KMT2C (Gene); SEQ ID NO: 8094, KMT2C (Gene); SEQ ID NO: 8095, KMT2C (Gene); SEQ ID NO: 8096, KMT2C (Gene); SEQ ID NO: 8097, KMT2C (Gene); SEQ ID NO: 8098, KMT2C (Gene); SEQ ID NO: 8099, KMT2C (Gene); SEQ ID NO: 8100, KMT2C (Gene); SEQ ID NO: 8101, KMT2C (Gene); SEQ ID NO: 8102, KMT2C (Gene); SEQ ID NO: 8103, KMT2C (Gene); SEQ ID NO: 8104, KMT2C (Gene); SEQ ID NO: 8105, KMT2C (Gene); SEQ ID NO: 8106, KMT2C (Gene); SEQ ID NO: 8107, KMT2C (Gene); SEQ ID NO: 8108, KMT2C (Gene); SEQ ID NO: 8109, 7Q_DLBCL (ARM); SEQ ID NO: 8110, 7Q_DLBCL (ARM); SEQ ID NO: 8111, 7Q_DLBCL (ARM); SEQ ID NO: 8112, 7Q_DLBCL (ARM); SEQ ID NO: 8113, 7Q_DLBCL (ARM); SEQ ID NO: 8114, FP (FP); SEQ ID NO: 8115, 7Q_DLBCL (ARM); SEQ ID NO: 8116, 7Q_DLBCL (ARM); SEQ ID NO: 8117, 8P_CLL (ARM); SEQ ID NO: 8118, 8P_CLL (ARM); SEQ ID NO: 8119, FP (FP); SEQ ID NO: 8120, 8P_CLL (ARM); SEQ ID NO: 8121, 8P_CLL (ARM); SEQ ID NO: 8122, 8P_CLL (ARM); SEQ ID NO: 8123, 8P_CLL (ARM); SEQ ID NO: 8124, 8P_CLL (ARM); SEQ ID NO: 8125, 8P_CLL (ARM); SEQ ID NO: 8126, 8P_CLL (ARM); SEQ ID NO: 8127, 8P_CLL (ARM); SEQ ID NO: 8128, 8P_CLL (ARM); SEQ ID NO: 8129, 8P_CLL (ARM); SEQ ID NO: 8130, 8P_CLL (ARM); SEQ ID NO: 8131, 8P_CLL (ARM); SEQ ID NO: 8132, 8P_CLL (ARM); SEQ ID NO: 8133, 8P_CLL (ARM); SEQ ID NO: 8134, 8P_CLL (ARM); SEQ ID NO: 8135, 8P_CLL (ARM); SEQ ID NO: 8136, 8P_CLL (ARM); SEQ ID NO: 8137, 8P_CLL (ARM); SEQ ID NO: 8138, 8P_CLL (ARM); SEQ ID NO: 8139, 8P_CLL (ARM); SEQ ID NO: 8140, 8P_CLL (ARM); SEQ ID NO: 8141, 8P_CLL (ARM); SEQ ID NO: 8142, 8P_CLL (ARM); SEQ ID NO: 8143, 8P_CLL (ARM); SEQ ID NO: 8144, 8P_CLL (ARM); SEQ ID NO: 8145, 8P_CLL (ARM); SEQ ID NO: 8146, TNFRSF10B (Gene); SEQ ID NO: 8147, TNFRSF10B (Gene); SEQ ID NO: 8148, TNFRSF10B (Gene); SEQ ID NO: 8149, TNFRSF10B (Gene); SEQ ID NO: 8150, TNFRSF10B (Gene); SEQ ID NO: 8151, TNFRSF10B (Gene); SEQ ID NO: 8152, TNFRSF10B (Gene); SEQ ID NO: 8153, TNFRSF10B (Gene); SEQ ID NO: 8154, TNFRSF10B (Gene); SEQ ID NO: 8155, 8P_CLL (ARM); SEQ ID NO: 8156, TNFRSF10A (Gene); SEQ ID NO: 8157, TNFRSF10A (Gene); SEQ ID NO: 8158, TNFRSF10A (Gene); SEQ ID NO: 8159, TNFRSF10A (Gene); SEQ ID NO: 8160, TNFRSF10A (Gene); SEQ ID NO: 8161, TNFRSF10A (Gene); SEQ ID NO: 8162, TNFRSF10A (Gene); SEQ ID NO: 8163, TNFRSF10A (Gene); SEQ ID NO: 8164, TNFRSF10A (Gene); SEQ ID NO: 8165, TNFRSF10A (Gene); SEQ ID NO: 8166, 8P_CLL (ARM); SEQ ID NO: 8167, 8P_CLL (ARM); SEQ ID NO: 8168, 8P_CLL (ARM); SEQ ID NO: 8169, 8P_CLL (ARM); SEQ ID NO: 8170, 8P_CLL (ARM); SEQ ID NO: 8171, 8P_CLL (ARM); SEQ ID NO: 8172, 8P_CLL (ARM); SEQ ID NO: 8173, 8P_CLL (ARM); SEQ ID NO: 8174, 8P_CLL (ARM); SEQ ID NO: 8175, 8P_CLL (ARM); SEQ ID NO: 8176, MSI (MSI); SEQ ID NO: 8177, MSI (MSI); SEQ ID NO: 8178, 8P_CLL (ARM); SEQ ID NO: 8179, FP (FP); SEQ ID NO: 8180, 8P_CLL (ARM); SEQ ID NO: 8181, IKBKB (Gene); SEQ ID NO: 8182, IKBKB (Gene); SEQ ID NO: 8183, IKBKB (Gene); SEQ ID NO: 8184, IKBKB (Gene); SEQ ID NO: 8185, IKBKB (Gene); SEQ ID NO: 8186, IKBKB (Gene); SEQ ID NO: 8187, IKBKB (Gene); SEQ ID NO: 8188, IKBKB (Gene); SEQ ID NO: 8189, IKBKB (Gene); SEQ ID NO: 8190, IKBKB (Gene); SEQ ID NO: 8191, IKBKB (Gene); SEQ ID NO: 8192, IKBKB (Gene); SEQ ID NO: 8193, IKBKB (Gene); SEQ ID NO: 8194, IKBKB (Gene); SEQ ID NO: 8195, IKBKB (Gene); SEQ ID NO: 8196, IKBKB (Gene); SEQ ID NO: 8197, IKBKB (Gene); SEQ ID NO: 8198, IKBKB (Gene); SEQ ID NO: 8199, IKBKB (Gene); SEQ ID NO: 8200, IKBKB (Gene); SEQ ID NO: 8201, IKBKB (Gene); SEQ ID NO: 8202, 8P (ARM); SEQ ID NO: 8203, 8P (ARM); SEQ ID NO: 8204, 8P (ARM); SEQ ID NO: 8205, LYN (Gene); SEQ ID NO: 8206, LYN (Gene); SEQ ID NO: 8207, LYN (Gene); SEQ ID NO: 8208, LYN (Gene); SEQ ID NO: 8209, LYN (Gene); SEQ ID NO: 8210, LYN (Gene); SEQ ID NO: 8211, LYN (Gene); SEQ ID NO: 8212, LYN (Gene); SEQ ID NO: 8213, LYN (Gene); SEQ ID NO: 8214, LYN (Gene); SEQ ID NO: 8215, LYN (Gene); SEQ ID NO: 8216, LYN (Gene); SEQ ID NO: 8217, 8P (ARM); SEQ ID NO: 8218, 8P (ARM); SEQ ID NO: 8219, 8q12.1_DLBCL (FOCAL); SEQ ID NO: 8220, TOX (Gene); SEQ ID NO: 8221, TOX (Gene); SEQ ID NO: 8222, TOX (Gene); SEQ ID NO: 8223, TOX (Gene); SEQ ID NO: 8224, TOX (Gene); SEQ ID NO: 8225, TOX (Gene); SEQ ID NO: 8226, TOX (Gene); SEQ ID NO: 8227, TOX (Gene); SEQ ID NO: 8228, TOX (Gene); SEQ ID NO: 8229, TOX (Gene); SEQ ID NO: 8230, 8q12.1_DLBCL (FOCAL); SEQ ID NO: 8231, 8q12.1_DLBCL (FOCAL); SEQ ID NO: 8232, 8q12.1_DLBCL (FOCAL); SEQ ID NO: 8233, 8q12.1_DLBCL (FOCAL); SEQ ID NO: 8234, 8q12.1_DLBCL (FOCAL); SEQ ID NO: 8235, 8q12.1_DLBCL (FOCAL); SEQ ID NO: 8236, 8q12.1_DLBCL (FOCAL); SEQ ID NO: 8237, 8q12.1_DLBCL (FOCAL); SEQ ID NO: 8238, 8P (ARM); SEQ ID NO: 8239, 8P (ARM); SEQ ID NO: 8240, 8P (ARM); SEQ ID NO: 8241, 8P (ARM); SEQ ID NO: 8242, FP (FP); SEQ ID NO: 8243, 8P (ARM); SEQ ID NO: 8244, 8P (ARM); SEQ ID NO: 8245, 8P (ARM); SEQ ID NO: 8246, 8P (ARM); SEQ ID NO: 8247, 8P (ARM); SEQ ID NO: 8248, 8P (ARM); SEQ ID NO: 8249, 8P (ARM); SEQ ID NO: 8250, 8P (ARM); SEQ ID NO: 8251, 8P (ARM); SEQ ID NO: 8252, 8P (ARM); SEQ ID NO: 8253, 8P (ARM); SEQ ID NO: 8254, 8P (ARM); SEQ ID NO: 8255, 8P (ARM); SEQ ID NO: 8256, 8P (ARM); SEQ ID NO: 8257, 8P (ARM); SEQ ID NO: 8258, 8P (ARM); SEQ ID NO: 8259, 8P (ARM); SEQ ID NO: 8260, MSI (MSI); SEQ ID NO: 8261, MSI (MSI); SEQ ID NO: 8262, 8P (ARM); SEQ ID NO: 8263, 8P (ARM); SEQ ID NO: 8264, 8P (ARM); SEQ ID NO: 8265, 8P (ARM); SEQ ID NO: 8266, 8Q (ARM); SEQ ID NO: 8267, 8Q (ARM); SEQ ID NO: 8268, 8Q (ARM); SEQ ID NO: 8269, UBR5 (Gene); SEQ ID NO: 8270, UBR5 (Gene); SEQ ID NO: 8271, UBR5 (Gene); SEQ ID NO: 8272, UBR5 (Gene); SEQ ID NO: 8273, UBR5 (Gene); SEQ ID NO: 8274, UBR5 (Gene); SEQ ID NO: 8275, UBR5 (Gene); SEQ ID NO: 8276, UBR5 (Gene); SEQ ID NO: 8277, UBR5 (Gene); SEQ ID NO: 8278, UBR5 (Gene); SEQ ID NO: 8279, UBR5 (Gene); SEQ ID NO: 8280, UBR5 (Gene); SEQ ID NO: 8281, UBR5 (Gene); SEQ ID NO: 8282, UBR5 (Gene); SEQ ID NO: 8283, UBR5 (Gene); SEQ ID NO: 8284, UBR5 (Gene); SEQ ID NO: 8285, UBR5 (Gene); SEQ ID NO: 8286, UBR5 (Gene); SEQ ID NO: 8287, UBR5 (Gene); SEQ ID NO: 8288, UBR5 (Gene); SEQ ID NO: 8289, UBR5 (Gene); SEQ ID NO: 8290, UBR5 (Gene); SEQ ID NO: 8291, UBR5 (Gene); SEQ ID NO: 8292, UBR5 (Gene); SEQ ID NO: 8293, UBR5 (Gene); SEQ ID NO: 8294, UBR5 (Gene); SEQ ID NO: 8295, UBR5 (Gene); SEQ ID NO: 8296, UBR5 (Gene); SEQ ID NO: 8297, UBR5 (Gene); SEQ ID NO: 8298, UBR5 (Gene); SEQ ID NO: 8299, UBR5 (Gene); SEQ ID NO: 8300, UBR5 (Gene); SEQ ID NO: 8301, UBR5 (Gene); SEQ ID NO: 8302, UBR5 (Gene); SEQ ID NO: 8303, UBR5 (Gene); SEQ ID NO: 8304, UBR5 (Gene); SEQ ID NO: 8305, UBR5 (Gene); SEQ ID NO: 8306, UBR5 (Gene); SEQ ID NO: 8307, UBR5 (Gene); SEQ ID NO: 8308, UBR5 (Gene); SEQ ID NO: 8309, UBR5 (Gene); SEQ ID NO: 8310, UBR5 (Gene); SEQ ID NO: 8311, UBR5 (Gene); SEQ ID NO: 8312, UBR5 (Gene); SEQ ID NO: 8313, UBR5 (Gene); SEQ ID NO: 8314, UBR5 (Gene); SEQ ID NO: 8315, UBR5 (Gene); SEQ ID NO: 8316, UBR5 (Gene); SEQ ID NO: 8317, UBR5 (Gene); SEQ ID NO: 8318, UBR5 (Gene); SEQ ID NO: 8319, UBR5 (Gene); SEQ ID NO: 8320, UBR5 (Gene); SEQ ID NO: 8321, UBR5 (Gene); SEQ ID NO: 8322, UBR5 (Gene); SEQ ID NO: 8323, UBR5 (Gene); SEQ ID NO: 8324, UBR5 (Gene); SEQ ID NO: 8325, UBR5 (Gene); SEQ ID NO: 8326, UBR5 (Gene); SEQ ID NO: 8327, UBR5 (Gene); SEQ ID NO: 8328, UBR5 (Gene); SEQ ID NO: 8329, 8Q (ARM); SEQ ID NO: 8330, 8Q (ARM); SEQ ID NO: 8331, 8Q (ARM); SEQ ID NO: 8332, 8Q (ARM); SEQ ID NO: 8333, 8Q (ARM); SEQ ID NO: 8334, 8Q (ARM); SEQ ID NO: 8335, 8Q (ARM); SEQ ID NO: 8336, 8Q (ARM); SEQ ID NO: 8337, 8Q (ARM); SEQ ID NO: 8338, 8Q (ARM); SEQ ID NO: 8339, 8Q (ARM); SEQ ID NO: 8340, 8Q (ARM); SEQ ID NO: 8341, 8Q (ARM); SEQ ID NO: 8342, 8Q (ARM); SEQ ID NO: 8343, 8Q (ARM); SEQ ID NO: 8344, 8Q (ARM); SEQ ID NO: 8345, 8Q (ARM); SEQ ID NO: 8346, 8Q (ARM); SEQ ID NO: 8347, 8Q (ARM); SEQ ID NO: 8348, 8Q (ARM); SEQ ID NO: 8349, 8Q (ARM); SEQ ID NO: 8350, MYC_SV (SV); SEQ ID NO: 8351, MYC_SV (SV); SEQ ID NO: 8352, MYC_SV (SV); SEQ ID NO: 8353, MYC_SV (SV); SEQ ID NO: 8354, MYC_SV (SV); SEQ ID NO: 8355, MYC_SV (SV); SEQ ID NO: 8356, MYC_SV (SV); SEQ ID NO: 8357, MYC_SV (SV); SEQ ID NO: 8358, MYC_SV (SV); SEQ ID NO: 8359, MYC_SV (SV); SEQ ID NO: 8360, MYC_SV (SV); SEQ ID NO: 8361, MYC_SV (SV); SEQ ID NO: 8362, MYC_SV (SV); SEQ ID NO: 8363, MYC_SV (SV); SEQ ID NO: 8364, MYC_SV (SV); SEQ ID NO: 8365, 8Q (ARM); SEQ ID NO: 8366, 8Q (ARM); SEQ ID NO: 8367, 8Q (ARM); SEQ ID NO: 8368, 8Q (ARM); SEQ ID NO: 8369, 8Q (ARM); SEQ ID NO: 8370, 8q24.22_DLBCL (FOCAL); SEQ ID NO: 8371, 8q24.22_DLBCL (FOCAL); SEQ ID NO: 8372, 8q24.22_DLBCL (FOCAL); SEQ ID NO: 8373, 8q24.22_DLBCL (FOCAL); SEQ ID NO: 8374, 8q24.22_DLBCL (FOCAL); SEQ ID NO: 8375, 8q24.22_DLBCL (FOCAL); SEQ ID NO: 8376, 8q24.22_DLBCL (FOCAL); SEQ ID NO: 8377, 8q24.22_DLBCL (FOCAL); SEQ ID NO: 8378, 8q24.22_DLBCL (FOCAL); SEQ ID NO: 8379, 8Q (ARM); SEQ ID NO: 8380, 8Q (ARM); SEQ ID NO: 8381, 8Q (ARM); SEQ ID NO: 8382, 8Q (ARM); SEQ ID NO: 8383, 8Q (ARM); SEQ ID NO: 8384, 8Q (ARM); SEQ ID NO: 8385, 8Q (ARM); SEQ ID NO: 8386, 8Q (ARM); SEQ ID NO: 8387, 8Q (ARM); SEQ ID NO: 8388, FP (FP); SEQ ID NO: 8389, 8Q (ARM); SEQ ID NO: 8390, 8Q (ARM); SEQ ID NO: 8391, 8Q (ARM); SEQ ID NO: 8392, 8Q (ARM); SEQ ID NO: 8393, 8Q (ARM); SEQ ID NO: 8394, 8Q (ARM); SEQ ID NO: 8395, 8Q (ARM); SEQ ID NO: 8396, 8Q (ARM); SEQ ID NO: 8397, 9P (ARM); SEQ ID NO: 8398, FP (FP); SEQ ID NO: 8399, 9P (ARM); SEQ ID NO: 8400, 9P (ARM); SEQ ID NO: 8401, 9p24.1_DLBCL (FOCAL); SEQ ID NO: 8402, 9p24.1_DLBCL (FOCAL); SEQ ID NO: 8403, 9p24.1_DLBCL (FOCAL); SEQ ID NO: 8404, 9p24.1_DLBCL (FOCAL); SEQ ID NO: 8405, JAK2 (Gene); SEQ ID NO: 8406, JAK2 (Gene); SEQ ID NO: 8407, JAK2 (Gene); SEQ ID NO: 8408, JAK2 (Gene); SEQ ID NO: 8409, JAK2 (Gene); SEQ ID NO: 8410, JAK2 (Gene); SEQ ID NO: 8411, JAK2 (Gene); SEQ ID NO: 8412, JAK2 (Gene); SEQ ID NO: 8413, JAK2 (Gene); SEQ ID NO: 8414, JAK2 (Gene); SEQ ID NO: 8415, JAK2 (Gene); SEQ ID NO: 8416, JAK2 (Gene); SEQ ID NO: 8417, JAK2 (Gene); SEQ ID NO: 8418, JAK2 (Gene); SEQ ID NO: 8419, JAK2 (Gene); SEQ ID NO: 8420, JAK2 (Gene); SEQ ID NO: 8421, JAK2 (Gene); SEQ ID NO: 8422, JAK2 (Gene); SEQ ID NO: 8423, JAK2 (Gene); SEQ ID NO: 8424, JAK2 (Gene); SEQ ID NO: 8425, JAK2 (Gene); SEQ ID NO: 8426, JAK2 (Gene); SEQ ID NO: 8427, JAK2 (Gene); SEQ ID NO: 8428, 9p24.1_DLBCL (FOCAL); SEQ ID NO: 8429, 9p24.1_DLBCL (FOCAL); SEQ ID NO: 8430, CD274_SV (SV); SEQ ID NO: 8431, CD274_SV (SV); SEQ ID NO: 8432, CD274_SV (SV); SEQ ID NO: 8433, CD274_SV (SV); SEQ ID NO: 8434, CD274_SV (SV); SEQ ID NO: 8435, CD274_SV (SV); SEQ ID NO: 8436, CD274_SV (SV); SEQ ID NO: 8437, CD274_SV (SV); SEQ ID NO: 8438, CD274_SV (SV); SEQ ID NO: 8439, CD274_SV (SV); SEQ ID NO: 8440, CD274_SV (SV); SEQ ID NO: 8441, CD274_SV (SV); SEQ ID NO: 8442, CD274 (Gene); SEQ ID NO: 8443, CD274 (Gene); SEQ ID NO: 8444, CD274 (Gene); SEQ ID NO: 8445, CD274 (Gene); SEQ ID NO: 8446, CD274_3UTR (SV); SEQ ID NO: 8447, CD274_3UTR (SV); SEQ ID NO: 8448, CD274_3UTR (SV); SEQ ID NO: 8449, CD274_3UTR (SV); SEQ ID NO: 8450, CD274_3UTR (SV); SEQ ID NO: 8451, CD274_3UTR (SV); SEQ ID NO: 8452, CD274_3UTR (SV); SEQ ID NO: 8453, CD274-PDCD1LG2 intergenic region_SV (SV); SEQ ID NO: 8454, CD274-PDCD1LG2 intergenic region_SV (SV); SEQ ID NO: 8455, CD274-PDCD1LG2 intergenic region_SV (SV); SEQ ID NO: 8456, CD274-PDCD1LG2 intergenic region_SV (SV); SEQ ID NO: 8457, CD274-PDCD1LG2 intergenic region_SV (SV); SEQ ID NO: 8458, CD274-PDCD1LG2 intergenic region_SV (SV); SEQ ID NO: 8459, CD274-PDCD1LG2 intergenic region_SV (SV); SEQ ID NO: 8460, CD274-PDCD1LG2 intergenic region_SV (SV); SEQ ID NO: 8461, CD274-PDCD1LG2 intergenic region_SV (SV); SEQ ID NO: 8462, CD274-PDCD1LG2 intergenic region_SV (SV); SEQ ID NO: 8463, CD274-PDCD1LG2 intergenic region_SV (SV); SEQ ID NO: 8464, CD274-PDCD1LG2 intergenic region_SV (SV); SEQ ID NO: 8465, CD274-PDCD1LG2 intergenic region_SV (SV); SEQ ID NO: 8466, CD274-PDCD1LG2 intergenic region_SV (SV); SEQ ID NO: 8467, CD274-PDCD1LG2 intergenic region_SV (SV); SEQ ID NO: 8468, CD274-PDCD1LG2 intergenic region_SV (SV); SEQ ID NO: 8469, CD274-PDCD1LG2 intergenic region_SV (SV); SEQ ID NO: 8470, CD274-PDCD1LG2 intergenic region_SV (SV); SEQ ID NO: 8471, CD274-PDCD1LG2 intergenic region_SV (SV); SEQ ID NO: 8472, CD274-PDCD1LG2 intergenic region_SV (SV); SEQ ID NO: 8473, CD274-PDCD1LG2 intergenic region_SV (SV); SEQ ID NO: 8474, CD274-PDCD1LG2 intergenic region_SV (SV); SEQ ID NO: 8475, CD274-PDCD1LG2 intergenic region_SV (SV); SEQ ID NO: 8476, CD274-PDCD1LG2 intergenic region_SV (SV); SEQ ID NO: 8477, CD274-PDCD1LG2 intergenic region_SV (SV); SEQ ID NO: 8478, CD274-PDCD1LG2 intergenic region_SV (SV); SEQ ID NO: 8479, CD274-PDCD1LG2 intergenic region_SV (SV); SEQ ID NO: 8480, CD274-PDCD1LG2 intergenic region_SV (SV); SEQ ID NO: 8481, CD274-PDCD1LG2 intergenic region_SV (SV); SEQ ID NO: 8482, CD274-PDCD1LG2 intergenic region_SV (SV); SEQ ID NO: 8483, CD274-PDCD1LG2 intergenic region_SV (SV); SEQ ID NO: 8484, CD274-PDCD1LG2 intergenic region_SV (SV); SEQ ID NO: 8485, CD274-PDCD1LG2 intergenic region_SV (SV); SEQ ID NO: 8486, CD274-PDCD1LG2 intergenic region_SV (SV); SEQ ID NO: 8487, CD274-PDCD1LG2 intergenic region_SV (SV); SEQ ID NO: 8488, CD274-PDCD1LG2 intergenic region_SV (SV); SEQ ID NO: 8489, CD274-PDCD1LG2 intergenic region_SV (SV); SEQ ID NO: 8490, CD274-PDCD1LG2 intergenic region_SV (SV); SEQ ID NO: 8491, CD274-PDCD1LG2 intergenic region_SV (SV); SEQ ID NO: 8492, CD274-PDCD1LG2 intergenic region_SV (SV); SEQ ID NO: 8493, CD274-PDCD1LG2 intergenic region_SV (SV); SEQ ID NO: 8494, CD274-PDCD1LG2 intergenic region_SV (SV); SEQ ID NO: 8495, CD274-PDCD1LG2 intergenic region_SV (SV); SEQ ID NO: 8496, CD274-PDCD1LG2 intergenic region_SV (SV); SEQ ID NO: 8497, CD274-PDCD1LG2 intergenic region_SV (SV); SEQ ID NO: 8498, CD274-PDCD1LG2 intergenic region_SV (SV); SEQ ID NO: 8499, CD274-PDCD1LG2 intergenic region_SV (SV); SEQ ID NO: 8500, CD274-PDCD1LG2 intergenic region_SV (SV); SEQ ID NO: 8501, CD274-PDCD1LG2 intergenic region_SV (SV); SEQ ID NO: 8502, CD274-PDCD1LG2 intergenic region_SV (SV); SEQ ID NO: 8503, CD274-PDCD1LG2 intergenic region_SV (SV); SEQ ID NO: 8504, CD274-PDCD1LG2 intergenic region_SV (SV); SEQ ID NO: 8505, CD274-PDCD1LG2 intergenic region_SV (SV); SEQ ID NO: 8506, CD274-PDCD1LG2 intergenic region_SV (SV); SEQ ID NO: 8507, CD274-PDCD1LG2 intergenic region_SV (SV); SEQ ID NO: 8508, CD274-PDCD1LG2 intergenic region_SV (SV); SEQ ID NO: 8509, CD274-PDCD1LG2 intergenic region_SV (SV); SEQ ID NO: 8510, CD274-PDCD1LG2 intergenic region_SV (SV); SEQ ID NO: 8511, CD274-PDCD1LG2 intergenic region_SV (SV); SEQ ID NO: 8512, CD274-PDCD1LG2 intergenic region_SV (SV); SEQ ID NO: 8513, CD274-PDCD1LG2 intergenic region_SV (SV); SEQ ID NO: 8514, CD274-PDCD1LG2 intergenic region_SV (SV); SEQ ID NO: 8515, CD274-PDCD1LG2 intergenic region_SV (SV); SEQ ID NO: 8516, CD274-PDCD1LG2 intergenic region_SV (SV); SEQ ID NO: 8517, CD274-PDCD1LG2 intergenic region_SV (SV); SEQ ID NO: 8518, CD274-PDCD1LG2 intergenic region_SV (SV); SEQ ID NO: 8519, CD274-PDCD1LG2 intergenic region_SV (SV); SEQ ID NO: 8520, CD274-PDCD1LG2 intergenic region_SV (SV); SEQ ID NO: 8521, CD274-PDCD1LG2 intergenic region_SV (SV); SEQ ID NO: 8522, CD274-PDCD1LG2 intergenic region_SV (SV); SEQ ID NO: 8523, CD274-PDCD1LG2 intergenic region_SV (SV); SEQ ID NO: 8524, CD274-PDCD1LG2 intergenic region_SV (SV); SEQ ID NO: 8525, CD274-PDCD1LG2 intergenic region_SV (SV); SEQ ID NO: 8526, CD274-PDCD1LG2 intergenic region_SV (SV); SEQ ID NO: 8527, CD274-PDCD1LG2 intergenic region_SV (SV); SEQ ID NO: 8528, CD274-PDCD1LG2 intergenic region_SV (SV); SEQ ID NO: 8529, CD274-PDCD1LG2 intergenic region_SV (SV); SEQ ID NO: 8530, CD274-PDCD1LG2 intergenic region_SV (SV); SEQ ID NO: 8531, CD274-PDCD1LG2 intergenic region_SV (SV); SEQ ID NO: 8532, CD274-PDCD1LG2 intergenic region_SV (SV); SEQ ID NO: 8533, CD274-PDCD1LG2 intergenic region_SV (SV); SEQ ID NO: 8534, CD274-PDCD1LG2 intergenic region_SV (SV); SEQ ID NO: 8535, CD274-PDCD1LG2 intergenic region_SV (SV); SEQ ID NO: 8536, CD274-PDCD1LG2 intergenic region_SV (SV); SEQ ID NO: 8537, CD274-PDCD1LG2 intergenic region_SV (SV); SEQ ID NO: 8538, CD274-PDCD1LG2 intergenic region_SV (SV); SEQ ID NO: 8539, CD274-PDCD1LG2 intergenic region_SV (SV); SEQ ID NO: 8540, CD274-PDCD1LG2 intergenic region_SV (SV); SEQ ID NO: 8541, CD274-PDCD1LG2 intergenic region_SV (SV); SEQ ID NO: 8542, CD274-PDCD1LG2 intergenic region_SV (SV); SEQ ID NO: 8543, CD274-PDCD1LG2 intergenic region_SV (SV); SEQ ID NO: 8544, CD274-PDCD1LG2 intergenic region_SV (SV); SEQ ID NO: 8545, CD274-PDCD1LG2 intergenic region_SV (SV); SEQ ID NO: 8546, CD274-PDCD1LG2 intergenic region_SV (SV); SEQ ID NO: 8547, CD274-PDCD1LG2 intergenic region_SV (SV); SEQ ID NO: 8548, PDCD1LG2_SV (SV); SEQ ID NO: 8549, PDCD1LG2_SV (SV); SEQ ID NO: 8550, PDCD1LG2_SV (SV); SEQ ID NO: 8551, PDCD1LG2_SV (SV); SEQ ID NO: 8552, PDCD1LG2_SV (SV); SEQ ID NO: 8553, PDCD1LG2_SV (SV); SEQ ID NO: 8554, PDCD1LG2_SV (SV); SEQ ID NO: 8555, PDCD1LG2_SV (SV); SEQ ID NO: 8556, PDCD1LG2_SV (SV); SEQ ID NO: 8557, PDCD1LG2_SV (SV); SEQ ID NO: 8558, PDCD1LG2_SV (SV); SEQ ID NO: 8559, PDCD1LG2_SV (SV); SEQ ID NO: 8560, PDCD1LG2_SV (SV); SEQ ID NO: 8561, PDCD1LG2_SV (SV); SEQ ID NO: 8562, PDCD1LG2_SV (SV); SEQ ID NO: 8563, PDCD1LG2_SV (SV); SEQ ID NO: 8564, PDCD1LG2_SV (SV); SEQ ID NO: 8565, PDCD1LG2_SV (SV); SEQ ID NO: 8566, PDCD1LG2_SV (SV); SEQ ID NO: 8567, PDCD1LG2_SV (SV); SEQ ID NO: 8568, PDCD1LG2_SV (SV); SEQ ID NO: 8569, PDCD1LG2_SV (SV); SEQ ID NO: 8570, PDCD1LG2_SV (SV); SEQ ID NO: 8571, PDCD1LG2_SV (SV); SEQ ID NO: 8572, PDCD1LG2_SV (SV); SEQ ID NO: 8573, PDCD1LG2_SV (SV); SEQ ID NO: 8574, PDCD1LG2_SV (SV); SEQ ID NO: 8575, PDCD1LG2_SV (SV); SEQ ID NO: 8576, PDCD1LG2_SV (SV); SEQ ID NO: 8577, PDCD1LG2_SV (SV); SEQ ID NO: 8578, PDCD1LG2_SV (SV); SEQ ID NO: 8579, PDCD1LG2_SV (SV); SEQ ID NO: 8580, PDCD1LG2_SV (SV); SEQ ID NO: 8581, PDCD1LG2 (Gene); SEQ ID NO: 8582, PDCD1LG2 (Gene); SEQ ID NO: 8583, PDCD1LG2 (Gene); SEQ ID NO: 8584, PDCD1LG2_SV (SV); SEQ ID NO: 8585, PDCD1LG2_SV (SV); SEQ ID NO: 8586, PDCD1LG2_SV (SV); SEQ ID NO: 8587, PDCD1LG2_SV (SV); SEQ ID NO: 8588, PDCD1LG2_SV (SV); SEQ ID NO: 8589, PDCD1LG2_SV (SV); SEQ ID NO: 8590, PDCD1LG2_SV (SV); SEQ ID NO: 8591, PDCD1LG2_SV (SV); SEQ ID NO: 8592, PDCD1LG2_SV (SV); SEQ ID NO: 8593, PDCD1LG2_SV (SV); SEQ ID NO: 8594, PDCD1LG2_SV (SV); SEQ ID NO: 8595, PDCD1LG2_SV (SV); SEQ ID NO: 8596, PDCD1LG2_SV (SV); SEQ ID NO: 8597, PDCD1LG2_SV (SV); SEQ ID NO: 8598, PDCD1LG2_SV (SV); SEQ ID NO: 8599, PDCD1LG2_3UTR (SV); SEQ ID NO: 8600, PDCD1LG2_SV (SV); SEQ ID NO: 8601, PDCD1LG2_SV (SV); SEQ ID NO: 8602, PDCD1LG2_SV (SV); SEQ ID NO: 8603, PDCD1LG2_SV (SV); SEQ ID NO: 8604, PDCD1LG2_SV (SV); SEQ ID NO: 8605, PDCD1LG2_SV (SV); SEQ ID NO: 8606, PDCD1LG2_SV (SV); SEQ ID NO: 8607, PDCD1LG2_SV (SV); SEQ ID NO: 8608, PDCD1LG2_SV (SV); SEQ ID NO: 8609, PDCD1LG2_SV (SV); SEQ ID NO: 8610, 9p24.1_DLBCL (FOCAL); SEQ ID NO: 8611, 9p24.1_DLBCL (FOCAL); SEQ ID NO: 8612, 9P (ARM); SEQ ID NO: 8613, 9P (ARM); SEQ ID NO: 8614, 9P (ARM); SEQ ID NO: 8615, 9P (ARM); SEQ ID NO: 8616, 9P (ARM); SEQ ID NO: 8617, 9P (ARM); SEQ ID NO: 8618, 9P (ARM); SEQ ID NO: 8619, 9P (ARM); SEQ ID NO: 8620, 9P (ARM); SEQ ID NO: 8621, 9P (ARM); SEQ ID NO: 8622, 9P (ARM); SEQ ID NO: 8623, 9P (ARM); SEQ ID NO: 8624, 9P (ARM); SEQ ID NO: 8625, 9P (ARM); SEQ ID NO: 8626, 9P (ARM); SEQ ID NO: 8627, 9P (ARM); SEQ ID NO: 8628, 9P (ARM); SEQ ID NO: 8629, 9p21.3_DLBCL (FOCAL); SEQ ID NO: 8630, 9p21.3_DLBCL (FOCAL); SEQ ID NO: 8631, 9p21.3_DLBCL (FOCAL); SEQ ID NO: 8632, 9p21.3_DLBCL (FOCAL); SEQ ID NO: 8633, 9p21.3_DLBCL (FOCAL); SEQ ID NO: 8634, 9p21.3_DLBCL (FOCAL); SEQ ID NO: 8635, 9p21.3_DLBCL (FOCAL); SEQ ID NO: 8636, 9p21.3_DLBCL (FOCAL); SEQ ID NO: 8637, CDKN2A (Gene); SEQ ID NO: 8638, CDKN2A (Gene); SEQ ID NO: 8639, CDKN2A (Gene); SEQ ID NO: 8640, CDKN2A (Gene); SEQ ID NO: 8641, CDKN2A (Gene); SEQ ID NO: 8642, CDKN2B (Gene); SEQ ID NO: 8643, CDKN2B (Gene); SEQ ID NO: 8644, 9p21.3_DLBCL (FOCAL); SEQ ID NO: 8645, 9p21.3_DLBCL (FOCAL); SEQ ID NO: 8646, 9p21.3_DLBCL (FOCAL); SEQ ID NO: 8647, 9p21.3_DLBCL (FOCAL); SEQ ID NO: 8648, 9p21.3_DLBCL (FOCAL); SEQ ID NO: 8649, 9p21.3_DLBCL (FOCAL); SEQ ID NO: 8650, 9p21.3_DLBCL (FOCAL); SEQ ID NO: 8651, 9p21.3_DLBCL (FOCAL); SEQ ID NO: 8652, 9p21.3_DLBCL (FOCAL); SEQ ID NO: 8653, 9p21.3_DLBCL (FOCAL); SEQ ID NO: 8654, 9p21.3_DLBCL (FOCAL); SEQ ID NO: 8655, 9p21.3_DLBCL (FOCAL); SEQ ID NO: 8656, 9p21.3_DLBCL (FOCAL); SEQ ID NO: 8657, 9p21.3_DLBCL (FOCAL); SEQ ID NO: 8658, 9p21.3_DLBCL (FOCAL); SEQ ID NO: 8659, 9p21.3_DLBCL (FOCAL); SEQ ID NO: 8660, 9p21.3_DLBCL (FOCAL); SEQ ID NO: 8661, 9p21.3_DLBCL (FOCAL); SEQ ID NO: 8662, 9p21.3_DLBCL (FOCAL); SEQ ID NO: 8663, 9p21.3_DLBCL (FOCAL); SEQ ID NO: 8664, 9p21.3_DLBCL (FOCAL); SEQ ID NO: 8665, 9p21.3_DLBCL (FOCAL); SEQ ID NO: 8666, 9p21.3_DLBCL (FOCAL); SEQ ID NO: 8667, 9P (ARM); SEQ ID NO: 8668, 9P (ARM); SEQ ID NO: 8669, 9P (ARM); SEQ ID NO: 8670, 9P (ARM); SEQ ID NO: 8671, 9P (ARM); SEQ ID NO: 8672, MSI (MSI); SEQ ID NO: 8673, MSI (MSI); SEQ ID NO: 8674, 9P (ARM); SEQ ID NO: 8675, PAX5 (Gene); SEQ ID NO: 8676, PAX5 (Gene); SEQ ID NO: 8677, PAX5 (Gene); SEQ ID NO: 8678, PAX5 (Gene); SEQ ID NO: 8679, PAX5 (Gene); SEQ ID NO: 8680, 9P (ARM); SEQ ID NO: 8681, PAX5 (Gene); SEQ ID NO: 8682, PAX5 (Gene); SEQ ID NO: 8683, PAX5 (Gene); SEQ ID NO: 8684, PAX5 (Gene); SEQ ID NO: 8685, PAX5 (Gene); SEQ ID NO: 8686, PAX5_enhancer (SV); SEQ ID NO: 8687, PAX5_enhancer (SV); SEQ ID NO: 8688, PAX5_enhancer (SV); SEQ ID NO: 8689, PAX5_enhancer (SV); SEQ ID NO: 8690, PAX5_enhancer (SV); SEQ ID NO: 8691, PAX5_enhancer (SV); SEQ ID NO: 8692, PAX5_enhancer (SV); SEQ ID NO: 8693, PAX5_enhancer (SV); SEQ ID NO: 8694, PAX5_enhancer (SV); SEQ ID NO: 8695, PAX5_enhancer (SV); SEQ ID NO: 8696, PAX5_enhancer (SV); SEQ ID NO: 8697, PAX5_enhancer (SV); SEQ ID NO: 8698, PAX5_enhancer (SV); SEQ ID NO: 8699, PAX5_enhancer (SV); SEQ ID NO: 8700, PAX5_enhancer (SV); SEQ ID NO: 8701, PAX5_enhancer (SV); SEQ ID NO: 8702, PAX5_enhancer (SV); SEQ ID NO: 8703, PAX5_enhancer (SV); SEQ ID NO: 8704, PAX5_enhancer (SV); SEQ ID NO: 8705, PAX5_enhancer (SV); SEQ ID NO: 8706, GRHPR (Gene); SEQ ID NO: 8707, GRHPR (Gene); SEQ ID NO: 8708, GRHPR (Gene); SEQ ID NO: 8709, GRHPR (Gene); SEQ ID NO: 8710, GRHPR (Gene); SEQ ID NO: 8711, GRHPR (Gene); SEQ ID NO: 8712, GRHPR (Gene); SEQ ID NO: 8713, GRHPR (Gene); SEQ ID NO: 8714, GRHPR (Gene); SEQ ID NO: 8715, 9P (ARM); SEQ ID NO: 8716, 9P (ARM); SEQ ID NO: 8717, 9q21.13_DLBCL_trim (FOCAL); SEQ ID NO: 8718, 9q21.13_DLBCL_trim (FOCAL); SEQ ID NO: 8719, 9q21.13_DLBCL_trim (FOCAL); SEQ ID NO: 8720, 9q21.13_DLBCL_trim (FOCAL); SEQ ID NO: 8721, 9q21.13_DLBCL_trim (FOCAL); SEQ ID NO: 8722, 9q21.13_DLBCL_trim (FOCAL); SEQ ID NO: 8723, 9q21.13_DLBCL_trim (FOCAL); SEQ ID NO: 8724, 9q21.13_DLBCL_trim (FOCAL); SEQ ID NO: 8725, 9q21.13_DLBCL_trim (FOCAL); SEQ ID NO: 8726, 9q21.13_DLBCL_trim (FOCAL); SEQ ID NO: 8727, 9q21.13_DLBCL_trim (FOCAL); SEQ ID NO: 8728, 9q21.13_DLBCL_trim (FOCAL); SEQ ID NO: 8729, 9q21.13_DLBCL_trim (FOCAL); SEQ ID NO: 8730, 9q21.13_DLBCL_trim (FOCAL); SEQ ID NO: 8731, 9q21.13_DLBCL_trim (FOCAL); SEQ ID NO: 8732, 9q21.13_DLBCL_trim (FOCAL); SEQ ID NO: 8733, 9q21.13_DLBCL_trim (FOCAL); SEQ ID NO: 8734, 9q21.13_DLBCL_trim (FOCAL); SEQ ID NO: 8735, 9q21.13_DLBCL_trim (FOCAL); SEQ ID NO: 8736, 9q21.13_DLBCL_trim (FOCAL); SEQ ID NO: 8737, 9q21.13_DLBCL_trim (FOCAL); SEQ ID NO: 8738, 9q21.13_DLBCL_trim (FOCAL); SEQ ID NO: 8739, 9q21.13_DLBCL_trim (FOCAL); SEQ ID NO: 8740, 9q21.13_DLBCL_trim (FOCAL); SEQ ID NO: 8741, 9q21.13_DLBCL_trim (FOCAL); SEQ ID NO: 8742, 9q21.13_DLBCL_trim (FOCAL); SEQ ID NO: 8743, 9q21.13_DLBCL_trim (FOCAL); SEQ ID NO: 8744, 9q21.13_DLBCL_trim (FOCAL); SEQ ID NO: 8745, 9q21.13_DLBCL_trim (FOCAL); SEQ ID NO: 8746, 9q21.13_DLBCL_trim (FOCAL); SEQ ID NO: 8747, 9q21.13_DLBCL_trim (FOCAL); SEQ ID NO: 8748, 9q21.13_DLBCL_trim (FOCAL); SEQ ID NO: 8749, 9q21.13_DLBCL_trim (FOCAL); SEQ ID NO: 8750, 9q21.13_DLBCL_trim (FOCAL); SEQ ID NO: 8751, 9q21.13_DLBCL_trim (FOCAL); SEQ ID NO: 8752, 9q21.13_DLBCL_trim (FOCAL); SEQ ID NO: 8753, 9q21.13_DLBCL_trim (FOCAL); SEQ ID NO: 8754, 9q21.13_DLBCL_trim (FOCAL); SEQ ID NO: 8755, 9q21.13_DLBCL_trim (FOCAL); SEQ ID NO: 8756, PRUNE2 (Gene); SEQ ID NO: 8757, PRUNE2 (Gene); SEQ ID NO: 8758, PRUNE2 (Gene); SEQ ID NO: 8759, PRUNE2 (Gene); SEQ ID NO: 8760, PRUNE2 (Gene); SEQ ID NO: 8761, PRUNE2 (Gene); SEQ ID NO: 8762, PRUNE2 (Gene); SEQ ID NO: 8763, FP (FP); SEQ ID NO: 8764, PRUNE2 (Gene); SEQ ID NO: 8765, PRUNE2 (Gene); SEQ ID NO: 8766, PRUNE2 (Gene); SEQ ID NO: 8767, PRUNE2 (Gene); SEQ ID NO: 8768, PRUNE2 (Gene); SEQ ID NO: 8769, PRUNE2 (Gene); SEQ ID NO: 8770, PRUNE2 (Gene); SEQ ID NO: 8771, PRUNE2 (Gene); SEQ ID NO: 8772, PRUNE2 (Gene); SEQ ID NO: 8773, PRUNE2 (Gene); SEQ ID NO: 8774, PRUNE2 (Gene); SEQ ID NO: 8775, PRUNE2 (Gene); SEQ ID NO: 8776, PRUNE2 (Gene); SEQ ID NO: 8777, PRUNE2 (Gene); SEQ ID NO: 8778, PRUNE2 (Gene); SEQ ID NO: 8779, PRUNE2 (Gene); SEQ ID NO: 8780, PRUNE2 (Gene); SEQ ID NO: 8781, PRUNE2 (Gene); SEQ ID NO: 8782, PRUNE2 (Gene); SEQ ID NO: 8783, PRUNE2 (Gene); SEQ ID NO: 8784, PRUNE2 (Gene); SEQ ID NO: 8785, PRUNE2 (Gene); SEQ ID NO: 8786, PRUNE2 (Gene); SEQ ID NO: 8787, PRUNE2 (Gene); SEQ ID NO: 8788, PRUNE2 (Gene); SEQ ID NO: 8789, PRUNE2 (Gene); SEQ ID NO: 8790, PRUNE2 (Gene); SEQ ID NO: 8791, PRUNE2 (Gene); SEQ ID NO: 8792, PRUNE2 (Gene); SEQ ID NO: 8793, PRUNE2 (Gene); SEQ ID NO: 8794, 9q21.13_DLBCL_trim (FOCAL); SEQ ID NO: 8795, PRUNE2 (Gene); SEQ ID NO: 8796, 9q21.13_DLBCL_trim (FOCAL); SEQ ID NO: 8797, 9q21.13_DLBCL_trim (FOCAL); SEQ ID NO: 8798, 9q21.13_DLBCL_trim (FOCAL); SEQ ID NO: 8799, 9q21.13_DLBCL_trim (FOCAL); SEQ ID NO: 8800, 9q21.13_DLBCL_trim (FOCAL); SEQ ID NO: 8801, 9q21.13_DLBCL_trim (FOCAL); SEQ ID NO: 8802, 9q21.13_DLBCL_trim (FOCAL); SEQ ID NO: 8803, 9q21.13_DLBCL_trim (FOCAL); SEQ ID NO: 8804, 9q21.13_DLBCL_trim (FOCAL); SEQ ID NO: 8805, 9q21.13_DLBCL_trim (FOCAL); SEQ ID NO: 8806, 9q21.13_DLBCL_trim (FOCAL); SEQ ID NO: 8807, 9q21.13_DLBCL_trim (FOCAL); SEQ ID NO: 8808, 9q21.13_DLBCL_trim (FOCAL); SEQ ID NO: 8809, 9q21.13_DLBCL_trim (FOCAL); SEQ ID NO: 8810, 9q21.13_DLBCL_trim (FOCAL); SEQ ID NO: 8811, 9q21.13_DLBCL_trim (FOCAL); SEQ ID NO: 8812, 9q21.13_DLBCL_trim (FOCAL); SEQ ID NO: 8813, 9q21.13_DLBCL_trim (FOCAL); SEQ ID NO: 8814, 9q21.13_DLBCL_trim (FOCAL); SEQ ID NO: 8815, 9q21.13_DLBCL_trim (FOCAL); SEQ ID NO: 8816, 9q21.13_DLBCL_trim (FOCAL); SEQ ID NO: 8817, 9q21.13_DLBCL_trim (FOCAL); SEQ ID NO: 8818, 9q21.13_DLBCL_trim (FOCAL); SEQ ID NO: 8819, 9q21.13_DLBCL_trim (FOCAL); SEQ ID NO: 8820, 9q21.13_DLBCL_trim (FOCAL); SEQ ID NO: 8821, 9q21.13_DLBCL_trim (FOCAL); SEQ ID NO: 8822, 9q21.13_DLBCL_trim (FOCAL); SEQ ID NO: 8823, 9q21.13_DLBCL_trim (FOCAL); SEQ ID NO: 8824, 9q21.13_DLBCL_trim (FOCAL); SEQ ID NO: 8825, 9q21.13_DLBCL_trim (FOCAL); SEQ ID NO: 8826, 9q21.13_DLBCL_trim (FOCAL); SEQ ID NO: 8827, 9q21.13_DLBCL_trim (FOCAL); SEQ ID NO: 8828, 9q21.13_DLBCL_trim (FOCAL); SEQ ID NO: 8829, 9q21.13_DLBCL_trim (FOCAL); SEQ ID NO: 8830, 9q21.13_DLBCL_trim (FOCAL); SEQ ID NO: 8831, 9q21.13_DLBCL_trim (FOCAL); SEQ ID NO: 8832, 9q21.13_DLBCL_trim (FOCAL); SEQ ID NO: 8833, 9q21.13_DLBCL_trim (FOCAL); SEQ ID NO: 8834, 9q21.13_DLBCL_trim (FOCAL); SEQ ID NO: 8835, 9q21.13_DLBCL_trim (FOCAL); SEQ ID NO: 8836, 9q21.13_DLBCL_trim (FOCAL); SEQ ID NO: 8837, 9q21.13_DLBCL_trim (FOCAL); SEQ ID NO: 8838, 9q21.13_DLBCL_trim (FOCAL); SEQ ID NO: 8839, 9q21.13_DLBCL_trim (FOCAL); SEQ ID NO: 8840, 9q21.13_DLBCL_trim (FOCAL); SEQ ID NO: 8841, 9q21.13_DLBCL_trim (FOCAL); SEQ ID NO: 8842, 9q21.13_DLBCL_trim (FOCAL); SEQ ID NO: 8843, 9q21.13_DLBCL_trim (FOCAL); SEQ ID NO: 8844, 9q21.13_DLBCL_trim (FOCAL); SEQ ID NO: 8845, 9q21.13_DLBCL_trim (FOCAL); SEQ ID NO: 8846, 9q21.13_DLBCL_trim (FOCAL); SEQ ID NO: 8847, 9q21.13_DLBCL_trim (FOCAL); SEQ ID NO: 8848, 9q21.13_DLBCL_trim (FOCAL); SEQ ID NO: 8849, 9q21.13_DLBCL_trim (FOCAL); SEQ ID NO: 8850, 9q21.13_DLBCL_trim (FOCAL); SEQ ID NO: 8851, 9q21.13_DLBCL_trim (FOCAL); SEQ ID NO: 8852, 9q21.13_DLBCL_trim (FOCAL); SEQ ID NO: 8853, 9q21.13_DLBCL_trim (FOCAL); SEQ ID NO: 8854, 9q21.13_DLBCL_trim (FOCAL); SEQ ID NO: 8855, 9q21.13_DLBCL_trim (FOCAL); SEQ ID NO: 8856, 9q21.13_DLBCL_trim (FOCAL); SEQ ID NO: 8857, 9q21.13_DLBCL_trim (FOCAL); SEQ ID NO: 8858, 9q21.13_DLBCL_trim (FOCAL); SEQ ID NO: 8859, 9q21.13_DLBCL_trim (FOCAL); SEQ ID NO: 8860, 9q21.13_DLBCL_trim (FOCAL); SEQ ID NO: 8861, 9q21.13_DLBCL_trim (FOCAL); SEQ ID NO: 8862, 9q21.13_DLBCL_trim (FOCAL); SEQ ID NO: 8863, 9q21.13_DLBCL_trim (FOCAL); SEQ ID NO: 8864, 9q21.13_DLBCL_trim (FOCAL); SEQ ID NO: 8865, 9q21.13_DLBCL_trim (FOCAL); SEQ ID NO: 8866, 9q21.13_DLBCL_trim (FOCAL); SEQ ID NO: 8867, 9q21.13_DLBCL_trim (FOCAL); SEQ ID NO: 8868, 9q21.13_DLBCL_trim (FOCAL); SEQ ID NO: 8869, 9q21.13_DLBCL_trim (FOCAL); SEQ ID NO: 8870, 9q21.13_DLBCL_trim (FOCAL); SEQ ID NO: 8871, 9q21.13_DLBCL_trim (FOCAL); SEQ ID NO: 8872, 9q21.13_DLBCL_trim (FOCAL); SEQ ID NO: 8873, 9q21.13_DLBCL_trim (FOCAL); SEQ ID NO: 8874, 9q21.13_DLBCL_trim (FOCAL); SEQ ID NO: 8875, 9q21.13_DLBCL_trim (FOCAL); SEQ ID NO: 8876, 9q21.13_DLBCL_trim (FOCAL); SEQ ID NO: 8877, 9q21.13_DLBCL_trim (FOCAL); SEQ ID NO: 8878, PTCH1 (Gene); SEQ ID NO: 8879, PTCH1 (Gene); SEQ ID NO: 8880, PTCH1 (Gene); SEQ ID NO: 8881, PTCH1 (Gene); SEQ ID NO: 8882, PTCH1 (Gene); SEQ ID NO: 8883, PTCH1 (Gene); SEQ ID NO: 8884, PTCH1 (Gene); SEQ ID NO: 8885, PTCH1 (Gene); SEQ ID NO: 8886, PTCH1 (Gene); SEQ ID NO: 8887, PTCH1 (Gene); SEQ ID NO: 8888, PTCH1 (Gene); SEQ ID NO: 8889, PTCH1 (Gene); SEQ ID NO: 8890, 9q21.13_DLBCL_trim (FOCAL); SEQ ID NO: 8891, PTCH1 (Gene); SEQ ID NO: 8892, PTCH1 (Gene); SEQ ID NO: 8893, PTCH1 (Gene); SEQ ID NO: 8894, PTCH1 (Gene); SEQ ID NO: 8895, PTCH1 (Gene); SEQ ID NO: 8896, PTCH1 (Gene); SEQ ID NO: 8897, PTCH1 (Gene); SEQ ID NO: 8898, PTCH1 (Gene); SEQ ID NO: 8899, PTCH1 (Gene); SEQ ID NO: 8900, PTCH1 (Gene); SEQ ID NO: 8901, PTCH1 (Gene); SEQ ID NO: 8902, PTCH1 (Gene); SEQ ID NO: 8903, PTCH1 (Gene); SEQ ID NO: 8904, 9q21.13_DLBCL_trim (FOCAL); SEQ ID NO: 8905, 9q21.13_DLBCL_trim (FOCAL); SEQ ID NO: 8906, 9q21.13_DLBCL_trim (FOCAL); SEQ ID NO: 8907, 9q21.13_DLBCL_trim (FOCAL); SEQ ID NO: 8908, 9q21.13_DLBCL_trim (FOCAL); SEQ ID NO: 8909, 9q21.13_DLBCL_trim (FOCAL); SEQ ID NO: 8910, 9q21.13_DLBCL_trim (FOCAL); SEQ ID NO: 8911, 9q21.13_DLBCL_trim (FOCAL); SEQ ID NO: 8912, 9q21.13_DLBCL_trim (FOCAL); SEQ ID NO: 8913, 9q21.13_DLBCL_trim (FOCAL); SEQ ID NO: 8914, 9q21.13_DLBCL_trim (FOCAL); SEQ ID NO: 8915, 9q21.13_DLBCL_trim (FOCAL); SEQ ID NO: 8916, 9q21.13_DLBCL_trim (FOCAL); SEQ ID NO: 8917, 9q21.13_DLBCL_trim (FOCAL); SEQ ID NO: 8918, 9q21.13_DLBCL_trim (FOCAL); SEQ ID NO: 8919, 9q21.13_DLBCL_trim (FOCAL); SEQ ID NO: 8920, 9q21.13_DLBCL_trim (FOCAL); SEQ ID NO: 8921, 9q21.13_DLBCL_trim (FOCAL); SEQ ID NO: 8922, 9q21.13_DLBCL_trim (FOCAL); SEQ ID NO: 8923, 9q21.13_DLBCL_trim (FOCAL); SEQ ID NO: 8924, 9q21.13_DLBCL_trim (FOCAL); SEQ ID NO: 8925, 9Q_DLBCL (ARM); SEQ ID NO: 8926, 9Q_DLBCL (ARM); SEQ ID NO: 8927, 9Q_DLBCL (ARM); SEQ ID NO: 8928, 9Q_DLBCL (ARM); SEQ ID NO: 8929, 9Q_DLBCL (ARM); SEQ ID NO: 8930, 9Q_DLBCL (ARM); SEQ ID NO: 8931, 9Q_DLBCL (ARM); SEQ ID NO: 8932, 9Q_DLBCL (ARM); SEQ ID NO: 8933, 9Q_DLBCL (ARM); SEQ ID NO: 8934, 9Q_DLBCL (ARM); SEQ ID NO: 8935, 9Q_DLBCL (ARM); SEQ ID NO: 8936, 9Q_DLBCL (ARM); SEQ ID NO: 8937, 9Q_DLBCL (ARM); SEQ ID NO: 8938, 9Q_DLBCL (ARM); SEQ ID NO: 8939, 9Q_DLBCL (ARM); SEQ ID NO: 8940, 9Q_DLBCL (ARM); SEQ ID NO: 8941, 9Q_DLBCL (ARM); SEQ ID NO: 8942, 9Q_DLBCL (ARM); SEQ ID NO: 8943, 9Q_DLBCL (ARM); SEQ ID NO: 8944, 9Q_DLBCL (ARM); SEQ ID NO: 8945, 9Q_DLBCL (ARM); SEQ ID NO: 8946, 9Q_DLBCL (ARM); SEQ ID NO: 8947, 9Q_DLBCL (ARM); SEQ ID NO: 8948, 9Q_DLBCL (ARM); SEQ ID NO: 8949, 9Q_DLBCL (ARM); SEQ ID NO: 8950, 9Q_DLBCL (ARM); SEQ ID NO: 8951, FP (FP); SEQ ID NO: 8952, 9Q_DLBCL (ARM); SEQ ID NO: 8953, 9Q_DLBCL (ARM); SEQ ID NO: 8954, 9Q_DLBCL (ARM); SEQ ID NO: 8955, 9Q_DLBCL (ARM); SEQ ID NO: 8956, 9Q_DLBCL (ARM); SEQ ID NO: 8957, FP (FP); SEQ ID NO: 8958, 9Q_DLBCL (ARM); SEQ ID NO: 8959, 9Q_DLBCL (ARM); SEQ ID NO: 8960, 9Q_DLBCL (ARM); SEQ ID NO: 8961, ABL1 (Gene); SEQ ID NO: 8962, ABL1 (Gene); SEQ ID NO: 8963, ABL1 (Gene); SEQ ID NO: 8964, ABL1 (Gene); SEQ ID NO: 8965, ABL1 (Gene); SEQ ID NO: 8966, ABL1 (Gene); SEQ ID NO: 8967, ABL1 (Gene); SEQ ID NO: 8968, ABL1 (Gene); SEQ ID NO: 8969, ABL1 (Gene); SEQ ID NO: 8970, ABL1 (Gene); SEQ ID NO: 8971, ABL1 (Gene); SEQ ID NO: 8972, ABL1 (Gene); SEQ ID NO: 8973, ABL1 (Gene); SEQ ID NO: 8974, ABL1 (Gene); SEQ ID NO: 8975, ABL1 (Gene); SEQ ID NO: 8976, 9Q_DLBCL (ARM); SEQ ID NO: 8977, MSI (MSI); SEQ ID NO: 8978, MSI (MSI); SEQ ID NO: 8979, 9Q_DLBCL (ARM); SEQ ID NO: 8980, 9Q_DLBCL (ARM); SEQ ID NO: 8981, NOTCH1_3UTR (SV); SEQ ID NO: 8982, NOTCH1 (Gene); SEQ ID NO: 8983, NOTCH1 (Gene); SEQ ID NO: 8984, NOTCH1 (Gene); SEQ ID NO: 8985, NOTCH1 (Gene); SEQ ID NO: 8986, NOTCH1 (Gene); SEQ ID NO: 8987, NOTCH1 (Gene); SEQ ID NO: 8988, NOTCH1 (Gene); SEQ ID NO: 8989, NOTCH1 (Gene); SEQ ID NO: 8990, NOTCH1 (Gene); SEQ ID NO: 8991, NOTCH1 (Gene); SEQ ID NO: 8992, NOTCH1 (Gene); SEQ ID NO: 8993, NOTCH1 (Gene); SEQ ID NO: 8994, NOTCH1 (Gene); SEQ ID NO: 8995, NOTCH1 (Gene); SEQ ID NO: 8996, NOTCH1 (Gene); SEQ ID NO: 8997, NOTCH1 (Gene); SEQ ID NO: 8998, NOTCH1 (Gene); SEQ ID NO: 8999, NOTCH1 (Gene); SEQ ID NO: 9000, NOTCH1 (Gene); SEQ ID NO: 9001, NOTCH1 (Gene); SEQ ID NO: 9002, NOTCH1 (Gene); SEQ ID NO: 9003, NOTCH1 (Gene); SEQ ID NO: 9004, NOTCH1 (Gene); SEQ ID NO: 9005, NOTCH1 (Gene); SEQ ID NO: 9006, NOTCH1 (Gene); SEQ ID NO: 9007, NOTCH1 (Gene); SEQ ID NO: 9008, NOTCH1 (Gene); SEQ ID NO: 9009, NOTCH1 (Gene); SEQ ID NO: 9010, NOTCH1 (Gene); SEQ ID NO: 9011, NOTCH1 (Gene); SEQ ID NO: 9012, NOTCH1 (Gene); SEQ ID NO: 9013, NOTCH1 (Gene); SEQ ID NO: 9014, NOTCH1 (Gene); SEQ ID NO: 9015, NOTCH1 (Gene); SEQ ID NO: 9016, NOTCH1 (Gene); SEQ ID NO: 9017, NOTCH1 (Gene); SEQ ID NO: 9018, NOTCH1 (Gene); SEQ ID NO: 9019, NOTCH1 (Gene); SEQ ID NO: 9020, TRAF2 (Gene); SEQ ID NO: 9021, TRAF2 (Gene); SEQ ID NO: 9022, TRAF2 (Gene); SEQ ID NO: 9023, TRAF2 (Gene); SEQ ID NO: 9024, TRAF2 (Gene); SEQ ID NO: 9025, TRAF2 (Gene); SEQ ID NO: 9026, TRAF2 (Gene); SEQ ID NO: 9027, TRAF2 (Gene); SEQ ID NO: 9028, TRAF2 (Gene); SEQ ID NO: 9029, TRAF2 (Gene); SEQ ID NO: 9030, XP (ARM); SEQ ID NO: 9031, P2RY8 (Gene); SEQ ID NO: 9032, P2RY8 (Gene); SEQ ID NO: 9033, P2RY8 (Gene); SEQ ID NO: 9034, XP (ARM); SEQ ID NO: 9035, XP (ARM); SEQ ID NO: 9036, XP (ARM); SEQ ID NO: 9037, XP (ARM); SEQ ID NO: 9038, XP (ARM); SEQ ID NO: 9039, XP (ARM); SEQ ID NO: 9040, XP (ARM); SEQ ID NO: 9041, XP (ARM); SEQ ID NO: 9042, XP (ARM); SEQ ID NO: 9043, MSI (MSI); SEQ ID NO: 9044, MSI (MSI); SEQ ID NO: 9045, XP (ARM); SEQ ID NO: 9046, XP (ARM); SEQ ID NO: 9047, TMSB4X (Gene); SEQ ID NO: 9048, TMSB4X (Gene); SEQ ID NO: 9049, XP (ARM); SEQ ID NO: 9050, MSI (MSI); SEQ ID NO: 9051, MSI (MSI); SEQ ID NO: 9052, XP (ARM); SEQ ID NO: 9053, XP (ARM); SEQ ID NO: 9054, XP (ARM); SEQ ID NO: 9055, XP (ARM); SEQ ID NO: 9056, XP (ARM); SEQ ID NO: 9057, XP (ARM); SEQ ID NO: 9058, XP (ARM); SEQ ID NO: 9059, XP (ARM); SEQ ID NO: 9060, XP (ARM); SEQ ID NO: 9061, XP (ARM); SEQ ID NO: 9062, XP (ARM); SEQ ID NO: 9063, XP (ARM); SEQ ID NO: 9064, MSI (MSI); SEQ ID NO: 9065, MSI (MSI); SEQ ID NO: 9066, XP (ARM); SEQ ID NO: 9067, XP (ARM); SEQ ID NO: 9068, XP (ARM); SEQ ID NO: 9069, DDX3X (Gene); SEQ ID NO: 9070, DDX3X (Gene); SEQ ID NO: 9071, DDX3X (Gene); SEQ ID NO: 9072, DDX3X (Gene); SEQ ID NO: 9073, DDX3X (Gene); SEQ ID NO: 9074, DDX3X (Gene); SEQ ID NO: 9075, DDX3X (Gene); SEQ ID NO: 9076, DDX3X (Gene); SEQ ID NO: 9077, DDX3X (Gene); SEQ ID NO: 9078, DDX3X (Gene); SEQ ID NO: 9079, DDX3X (Gene); SEQ ID NO: 9080, DDX3X (Gene); SEQ ID NO: 9081, DDX3X (Gene); SEQ ID NO: 9082, DDX3X (Gene); SEQ ID NO: 9083, DDX3X (Gene); SEQ ID NO: 9084, DDX3X (Gene); SEQ ID NO: 9085, DDX3X (Gene); SEQ ID NO: 9086, XQ (ARM); SEQ ID NO: 9087, KDM6A (Gene); SEQ ID NO: 9088, KDM6A (Gene); SEQ ID NO: 9089, KDM6A (Gene); SEQ ID NO: 9090, KDM6A (Gene); SEQ ID NO: 9091, KDM6A (Gene); SEQ ID NO: 9092, KDM6A (Gene); SEQ ID NO: 9093, KDM6A (Gene); SEQ ID NO: 9094, KDM6A (Gene); SEQ ID NO: 9095, KDM6A (Gene); SEQ ID NO: 9096, KDM6A (Gene); SEQ ID NO: 9097, KDM6A (Gene); SEQ ID NO: 9098, KDM6A (Gene); SEQ ID NO: 9099, KDM6A (Gene); SEQ ID NO: 9100, KDM6A (Gene); SEQ ID NO: 9101, KDM6A (Gene); SEQ ID NO: 9102, KDM6A (Gene); SEQ ID NO: 9103, KDM6A (Gene); SEQ ID NO: 9104, KDM6A (Gene); SEQ ID NO: 9105, KDM6A (Gene); SEQ ID NO: 9106, KDM6A (Gene); SEQ ID NO: 9107, KDM6A (Gene); SEQ ID NO: 9108, KDM6A (Gene); SEQ ID NO: 9109, KDM6A (Gene); SEQ ID NO: 9110, KDM6A (Gene); SEQ ID NO: 9111, KDM6A (Gene); SEQ ID NO: 9112, KDM6A (Gene); SEQ ID NO: 9113, KDM6A (Gene); SEQ ID NO: 9114, KDM6A (Gene); SEQ ID NO: 9115, KDM6A (Gene); SEQ ID NO: 9116, KDM6A (Gene); SEQ ID NO: 9117, KDM6A (Gene); SEQ ID NO: 9118, XQ (ARM); SEQ ID NO: 9119, XQ (ARM); SEQ ID NO: 9120, PIM2 (Gene); SEQ ID NO: 9121, PIM2 (Gene); SEQ ID NO: 9122, PIM2 (Gene); SEQ ID NO: 9123, PIM2 (Gene); SEQ ID NO: 9124, PIM2 (Gene); SEQ ID NO: 9125, PIM2 (Gene); SEQ ID NO: 9126, XQ (ARM); SEQ ID NO: 9127, XQ (ARM); SEQ ID NO: 9128, XQ (ARM); SEQ ID NO: 9129, TAF1 (Gene); SEQ ID NO: 9130, TAF1 (Gene); SEQ ID NO: 9131, TAF1 (Gene); SEQ ID NO: 9132, TAF1 (Gene); SEQ ID NO: 9133, TAF1 (Gene); SEQ ID NO: 9134, TAF1 (Gene); SEQ ID NO: 9135, TAF1 (Gene); SEQ ID NO: 9136, TAF1 (Gene); SEQ ID NO: 9137, TAF1 (Gene); SEQ ID NO: 9138, TAF1 (Gene); SEQ ID NO: 9139, TAF1 (Gene); SEQ ID NO: 9140, TAF1 (Gene); SEQ ID NO: 9141, TAF1 (Gene); SEQ ID NO: 9142, TAF1 (Gene); SEQ ID NO: 9143, TAF1 (Gene); SEQ ID NO: 9144, TAF1 (Gene); SEQ ID NO: 9145, TAF1 (Gene); SEQ ID NO: 9146, TAF1 (Gene); SEQ ID NO: 9147, TAF1 (Gene); SEQ ID NO: 9148, TAF1 (Gene); SEQ ID NO: 9149, TAF1 (Gene); SEQ ID NO: 9150, TAF1 (Gene); SEQ ID NO: 9151, TAF1 (Gene); SEQ ID NO: 9152, TAF1 (Gene); SEQ ID NO: 9153, TAF1 (Gene); SEQ ID NO: 9154, TAF1 (Gene); SEQ ID NO: 9155, TAF1 (Gene); SEQ ID NO: 9156, TAF1 (Gene); SEQ ID NO: 9157, TAF1 (Gene); SEQ ID NO: 9158, TAF1 (Gene); SEQ ID NO: 9159, TAF1 (Gene); SEQ ID NO: 9160, TAF1 (Gene); SEQ ID NO: 9161, TAF1 (Gene); SEQ ID NO: 9162, TAF1 (Gene); SEQ ID NO: 9163, TAF1 (Gene); SEQ ID NO: 9164, TAF1 (Gene); SEQ ID NO: 9165, TAF1 (Gene); SEQ ID NO: 9166, TAF1 (Gene); SEQ ID NO: 9167, TAF1 (Gene); SEQ ID NO: 9168, MAGT1 (Gene); SEQ ID NO: 9169, MAGT1 (Gene); SEQ ID NO: 9170, MAGT1 (Gene); SEQ ID NO: 9171, MAGT1 (Gene); SEQ ID NO: 9172, MAGT1 (Gene); SEQ ID NO: 9173, MAGT1 (Gene); SEQ ID NO: 9174, MAGT1 (Gene); SEQ ID NO: 9175, MAGT1 (Gene); SEQ ID NO: 9176, MAGT1 (Gene); SEQ ID NO: 9177, MAGT1 (Gene); SEQ ID NO: 9178, XQ (ARM); SEQ ID NO: 9179, XQ (ARM); SEQ ID NO: 9180, XQ (ARM); SEQ ID NO: 9181, XQ (ARM); SEQ ID NO: 9182, XQ (ARM); SEQ ID NO: 9183, XQ (ARM); SEQ ID NO: 9184, XQ (ARM); SEQ ID NO: 9185, XQ (ARM); SEQ ID NO: 9186, BTK (Gene); SEQ ID NO: 9187, BTK (Gene); SEQ ID NO: 9188, BTK (Gene); SEQ ID NO: 9189, BTK (Gene); SEQ ID NO: 9190, BTK (Gene); SEQ ID NO: 9191, BTK (Gene); SEQ ID NO: 9192, BTK (Gene); SEQ ID NO: 9193, BTK (Gene); SEQ ID NO: 9194, BTK (Gene); SEQ ID NO: 9195, BTK (Gene); SEQ ID NO: 9196, BTK (Gene); SEQ ID NO: 9197, BTK (Gene); SEQ ID NO: 9198, BTK (Gene); SEQ ID NO: 9199, BTK (Gene); SEQ ID NO: 9200, BTK (Gene); SEQ ID NO: 9201, BTK (Gene); SEQ ID NO: 9202, BTK (Gene); SEQ ID NO: 9203, BTK (Gene); SEQ ID NO: 9204, BTK (Gene); SEQ ID NO: 9205, XQ (ARM); SEQ ID NO: 9206, MSI (MSI); SEQ ID NO: 9207, MSI (MSI); SEQ ID NO: 9208, XQ (ARM); SEQ ID NO: 9209, XQ (ARM); SEQ ID NO: 9210, XQ (ARM); SEQ ID NO: 9211, XQ (ARM); SEQ ID NO: 9212, XQ (ARM); SEQ ID NO: 9213, XQ (ARM); SEQ ID NO: 9214, UBE2A (Gene); SEQ ID NO: 9215, UBE2A (Gene); SEQ ID NO: 9216, UBE2A (Gene); SEQ ID NO: 9217, UBE2A (Gene); SEQ ID NO: 9218, UBE2A (Gene); SEQ ID NO: 9219, UBE2A (Gene); SEQ ID NO: 9220, XQ (ARM); SEQ ID NO: 9221, XQ (ARM); SEQ ID NO: 9222, XQ (ARM); SEQ ID NO: 9223, XQ (ARM); SEQ ID NO: 9224, XQ (ARM); SEQ ID NO: 9225, MSI (MSI); SEQ ID NO: 9226, MSI (MSI); SEQ ID NO: 9227, XQ (ARM); SEQ ID NO: 9228, XQ (ARM); SEQ ID NO: 9229, XQ (ARM); SEQ ID NO: 9230, XQ (ARM); SEQ ID NO: 9231, XQ (ARM); SEQ ID NO: 9232, XQ (ARM); SEQ ID NO: 9233, XQ (ARM); SEQ ID NO: 9234, XQ (ARM); SEQ ID NO: 9235, XQ (ARM); SEQ ID NO: 9236, XQ (ARM); SEQ ID NO: 9237, XQ (ARM); SEQ ID NO: 9238, XQ (ARM); SEQ ID NO: 9239, XQ (ARM); SEQ ID NO: 9240, XQ (ARM); SEQ ID NO: 9241, XQ (ARM); SEQ ID NO: 9242, XQ (ARM); SEQ ID NO: 9243, FP (FP); SEQ ID NO: 9244, P2RY8 (Gene).

Baits suitable for use in embodiments of the invention can include a set of polynucleotides selected from SEQ ID NOs: 1-9244. The set of polynucleotides (i.e., bait set) can include all or a sub-set of polynucleotides identified as targeting a particular variant. The set of polynucleotides can include all or a sub-set of polynucleotides targeting a particular variant class and/or variant (i.e., target) type. The set of polynucleotides can include all or a sub-set of polynucleotides with sequences provided in the sequence listing as any one or more of SEQ ID NOs: 1-9244 and targeting all of the variants listed in Table 3. The set of polynucleotides can include polynucleotides complementary or identical to about or at least about 1%, 2%, 3%, 4%, 5%, 10%, 25%, 50%, 75%, 80%, 85%, 90%, 95%, or 100% of regions collectively defined/targeted by a set of polynucleotides with sequences provided in the sequence listing as any one or more of SEQ ID NOs: 1-9244. The set of polynucleotides can include sequences having about or at least about 1%, 2%, 3%, 4%, 5%, 0%, 25%, 50%, 75%, 80%, 85%, 90%, 95%, or 100% sequence identity to any one or more of SEQ ID NOs: 1-9244. The sequence identity can be calculated across the full contiguous span of bases contained by a sequence(s) listed in SEQ ID NOs: 1-9244, or across 1%, 2%, 3%, 4%, 5%, 10%, 25%, 50%, 75%, 80%, 85%, 90%, 95% of a contiguous span of bases contained by a sequence(s) listed in SEQ ID NOs: 1-9244, or across about or at least about 5, 10, 15, 20, 25, 30, 35, 40, 45, 50, 55, 60, 65, 70 75, 80, 85, 90, 95, 100, 200, 300, 400, 500, or 1000 bp of a sequence(s) listed in SEQ ID NOs: 1-9244. The polynucleotides in the set of polynucleotides can individually include sequences complementary or identical to at least 10, 20, 30, 40, 50, 60, 70, 80, 90, 100, 120, 150, 200, 300, or 500 contiguous, and optionally terminal, base pairs of a set of polynucleotides selected from those polynucleotides listed in SEQ ID NOs: 1-9244. The polynucleotides in the set of polynucleotides can individually include contiguous sequences, optionally terminal sequences, that are complementary to chromosomal regions adjacent or proximal to (i.e., within about or at least about 10 bp, 50 bp, 100 bp, 500 bp, or 1000 bp of a terminal extent of a targeted region) those chromosomal regions targeted by sequences listed in SEQ ID NOs: 1-9244, where the contiguous sequences can be about or at least about 5, 10, 15, 20, 25, 30, 35, 40, 45, 50, 55, 60, 65, 70 75, 80, 85, 90, 95, 100, 200, 300, 400, 500, or 1000 bp in length, and/or no more than about 5, 10, 15, 20, 25, 30, 35, 40, 45, 50, 55, 60, 65, 70 75, 80, 85, 90, 95, 100, 200, 300, 400, 500, or 1000 bp in length.

In embodiments, the bait sets include oligonucleotides that can detect microsatellite instability (see MSI Target sequences indicated in the above List of Sequence Descriptions), tumor mutational burden, and/or Epstein Barr virus (see sequences provided in Table 2). Representative baits suitable for detection of Epstein Barr virus in a sample are provided in Table 2 below and as SEQ ID NOs: 9245-9316 in the Sequence Listing. The bait sets can be used to determine tumor mutational burden in a subject or for quantifying levels of circulating tumor DNA in a subject. The bait sets capture alterations necessary and sufficient to classify DLBCLs as belonging to C1-C5. The bait sets can also be used to measure microsatellite instability and/or tumor mutational burden.

TABLE 2
Baits for detection of the Epstein-Barr virus.

| SEQ | |
|---|---|
| ID NO | Bait Description |
| 9245 | EBV_LMP1_NC_007605_1_167702_169016_Human_gammaherpesvirus_4_0_1200 |
| 9246 | EBV_LMP1_AY961628_3_167613_to_168357_Human_herpesvirus_4_strain_GD1_0_720 |
| 9247 | EBV_LMP1_HQ020558_1_c163432_162195_Human_herpesvirus_4_strain_GD2_0_600 |
| 9248 | EBV_LMP1_NC_007605_1_167702_169016_Human_gammaherpesvirus_4_0_1200 |
| 9249 | EBV_LMP1_DQ279927_1_c169948_169188_Human_herpesvirus_4_strain_AG876_0_720 |
| 9250 | EBV_LMP1_NC_007605_1_167702_169016_Human_gammaherpesvirus_4_0_1200 |
| 9251 | EBV_EBNA1_DQ279927_1_96492_98417_Huma_herpesvirus_4_strain_AG876_1200_1920 |
| 9252 | EBV_LMP1_NC_007605_1_167702_169016_Human_gammaherpesvirus_4_0_1200 |
| 9253 | EBV_EBNA1_JQ009376_2_95779_97209Human_herpesvirus_4_strain_HKNPC1_480_1320 |
| 9254 | EBV_EBNA1_JQ009376_2_95779_97209Human_herpesvirus_4_strain_HKNPC1_480_1320 |
| 9255 | EBV_EBNA1_JQ009376_2_95779_97209Human_herpesvirus_4_strain_HKNPC1_480_1320 |
| 9256 | EBV_LMP1_AY961628_3_167613_to_168357_Human_herpesvirus_4_strain_GD1_0_720 |
| 9257 | EBV_LMP1_HQ020558_1_c163432_162195_Human_herpesvirus_4_strain_GD2_0_600 |
| 9258 | EBV_LMP1_AY961628_3_167613_to_168357_Human_herpesvirus_4_strain_GD1_0_720 |
| 9259 | EBV_LMP1_HQ020558_1_c163432_162195_Human_herpesvirus_4_strain_GD2_960_1200 |
| 9260 | EBV_LMP1_DQ279927_1_c169948_169188_Human_herpesvirus_4_strain_AG876_0_720 |
| 9261 | EBV_LMP1_JQ009376_2_168596_167294_Human_herpesvirus_4_strain_HKNPC1_960_1200 |
| 9262 | EBV_LMP1_JQ009376_2_c168596_167294_Human_herpesvirus_4_strain_HKNPC1_0_1200 |
| 9263 | EBV_LMP1_NC_007605_1_167702_169016_Human_gammaherpesvirus_4_0_1200 |
| 9264 | EBV_LMP1_JQ009376_2_168596_167294_Human_herpesvirus_4_strain_HKNPC1_960_1200 |
| 9265 | EBV_EBNA1_HQ020558_1_93224_94907_Human_herpesvirus_4_strain_GD2_840_1200 |
| 9266 | EBV_EBNA1_DQ279927_1_96492_98417_Huma_herpesvirus_4_strain_AG876_1200_1920 |
| 9267 | EBV_LMP1_NC_007605_1_167702_169016_Human_gammaherpesvirus_4_0_1200 |
| 9268 | EBV_LMP1_HQ020558_1_c163432_162195_Human_herpesvirus_4_strain_GD2_960_1200 |
| 9269 | EBV_EBNA1_DQ279927_1_96492_98417_Huma_herpesvirus_4_strain_AG876_0_240 |
| 9270 | EBV_LMP1_DQ279927_1_c169948_169188_Human_herpesvirus_4_strain_AG876_0_720 |
| 9271 | EBV_LMP1_NC_007605_1_167702_169016_Human_gammaherpesvirus_4_0_1200 |
| 9272 | EBV_EBNA1_DQ279927_1_96492_98417_Huma_herpesvirus_4_strain_AG876_1200_1920 |
| 9273 | EBV_EBNA1_HQ020558_1_93224_94907_Human_herpesvirus_4_strain_GD2_840_1200 |
| 9274 | EBV_EBNA1_HQ020558_1_93224_94907_Human_herpesvirus_4_strain_GD2_0_240 |
| 9275 | EBV_LMP1_DQ279927_1_c169948_169188_Human_herpesvirus_4_strain_AG876_0_720 |
| 9276 | EBV_EBNA1_V01555_2_107950_109875_Epstein_Barr_virus_EBV_genome_strain_B95_8_1440_1920 |
| 9277 | EBV_EBNA1_AY961628_3_95580_97505_Human_herpesvirus_4_strain_GD1_1320_1560 |
| 9278 | EBV_EBNA1_DQ279927_1_96492_98417_Huma_herpesvirus_4_strain_AG876_1200_1920 |
| 9279 | EBV_EBNA1_V01555_2_107950_109875_Epstein_Barr_virus_EBV_genome_strain_B95_8_1440_1920 |
| 9280 | EBV_EBNA1_JQ009376_2_95779_97209Human_herpesvirus_4_strain_HKNPC1_480_1320 |
| 9281 | EBV_EBNA1_JQ009376_2_95779_97209Human_herpesvirus_4_strain_HKNPC1_480_1320 |
| 9282 | EBV_LMP1_DQ279927_1_c169948_169188_Human_herpesvirus_4_strain_AG876_0_720 |
| 9283 | EBV_LMP1_NC_007605_1_167702_169016_Human_gammaherpesvirus_4_0_1200 |
| 9284 | EBV_EBNA1_DQ279927_1_96492_98417_Huma_herpesvirus_4_strain_AG876_0_240 |
| 9285 | EBV_LMP1_JQ009376_2_c168596_167294_Human_herpesvirus_4_strain_HKNPC1_0_1200 |
| 9286 | EBV_LMP1_AY961628_3_167613_to_168357_Human_herpesvirus_4_strain_GD1_0_720 |
| 9287 | EBV_LMP1_HQ020558_1_c163432_162195_Human_herpesvirus_4_strain_GD2_0_600 |
| 9288 | EBV_EBNA1_V01555_2_107950_109875_Epstein_Barr_virus_EBV_genome_strain_B95_8_1440_1920 |
| 9289 | EBV_EBNA1_JQ009376_2_95779_97209Human_herpesvirus_4_strain_HKNPC1_480_1320 |
| 9290 | EBV_LMP1_V01555_2_168286_168964_Human_herpesvirus_4_strain_B95_8_0_240 |
| 9291 | EBV_LMP1_V01555_2_168286_168964_Human_herpesvirus_4_strain_B95_8_0_240 |
| 9292 | EBV_EBNA1_AY961628_3_95580_97505_Human_herpesvirus_4_strain_GD1_1320_1560 |
| 9293 | EBV_EBNA1_V01555_2_107950_109875_Epstein_Barr_virus_EBV_genome_strain_B95_8_1440_1920 |
| 9294 | EBV_LMP1_DQ279927_1_c169948_169188_Human_herpesvirus_4_strain_AG876_0_720 |
| 9295 | EBV_LMP1_AY961628_3_167613_to_168357_Human_herpesvirus_4_strain_GD1_0_720 |
| 9296 | EBV_LMP1_HQ020558_1_c163432_162195_Human_herpesvirus_4_strain_GD2_0_600 |
| 9297 | EBV_LMP1_AY961628_3_167613_to_168357_Human_herpesvirus_4_strain_GD1_0_720 |
| 9298 | EBV_EBNA1_HQ020558_1_93224_94907_Human_herpesvirus_4_strain_GD2_0_240 |
| 9299 | EBV_LMP1_DQ279927_1_c170457_170190_Human_herpesvirus_4_strain_AG876_0_240 |
| 9300 | EBV_LMP1_HQ020558_1_c163432_162195_Human_herpesvirus_4_strain_GD2_0_600 |
| 9301 | EBV_EBNA1_JQ009376_2_95779_97209Human_herpesvirus_4_strain_HKNPC1_480_1320 |
| 9302 | EBV_LMP1_DQ279927_1_c170457_170190_Human_herpesvirus_4_strain_AG876_0_240 |
| 9303 | EBV_LMP1_NC_007605_1_167702_169016_Human_gammaherpesvirus_4_0_1200 |
| 9304 | EBV_EBNA1_DQ279927_1_96492_98417_Huma_herpesvirus_4_strain_AG876_1200_1920 |
| 9305 | EBV_LMP1_HQ020558_1_c163432_162195_Human_herpesvirus_4_strain_GD2_720_840 |
| 9306 | EBV_EBNA1_HQ020558_1_93224_94907_Human_herpesvirus_4_strain_GD2_1320_1440 |
| 9307 | EBV_LMP1_V01555_2_168286_168964_Human_herpesvirus_4_strain_B95_8_480_600 |
| 9308 | EBV_EBNA1_V01555_2_107950_109875_Epstein_Barr_virus_{——}EBV_genome_strain_B95_8_0_120 |
| 9309 | EBV_EBNA1_NC_007605_1_95662_97587_Human_gammaherpesvirus_4_1080_1920 |
| 9310 | EBV_EBNA1_NC_007605_1_95662_97587_Human_gammaherpesvirus_4_0_240 |
| 9311 | EBV_EBNA1_V01555_2_107950_109875_Epstein_Barr_virus_EBV_genome_strain_B95_8_1080_1200 |
| 9312 | EBV_EBNA1_AY961628_3_95580_97505_Human_herpesvirus_4_strain_GD1_1080_1200 |
| 9313 | EBV_LMP1_JQ009376_2_c168596_167294_Human_herpesvirus_4_strain_HKNPC1_0_1200 |
| 9314 | EBV_EBNA1_AY961628_3_95580_97505_Human_herpesvirus_4_strain_GD1_1680_1920 |
| 9315 | EBV_LMP1_AY961628_3_168601_to_168868_Human_herpesvirus_4_strain_GD1_0_240 |
| 9316 | EBV_LMP1_JQ009376_2_168596_167294_Human_herpesvirus_4_strain_HKNPC1_600_720 |

### Treatments

The methods described herein can be used for selecting, and then optionally administering, an optimal treatment for a subject. Thus the methods described herein include methods for the treatment of cancer, particularly DLBCL. Generally, the methods include administering a therapeutically effective amount of a treatment as described herein, to a subject who is in need of, or who has been determined to be in need of, such treatment. The treatments can be selected based upon the subtype (C1-C5) of a DLBCL.

The distinct genetic subtypes (C1-C5) of DLBCLs may differ in their responses to immunotherapies. For example, C3 includes EZH2 mutations and has, thus, been associated with decreased MHC class I and II expression and reduced T-cell infiltrates. MHC expression can be restored by inhibiting EZH2 in EZH2-mutated (e.g., C3) DLBCLs. Also, differences in abundance of conventional targets (e.g., CD20) in the different categories of DLBCL may be useful in predicting efficacy of immunotherapy (e.g., anti-CD20 therapy). An example of an anti-CD20 drug used in immunotherapy is Rituxan. Thus, the present invention provides a method for suggesting treatment targets.

As used in this context, to “treat” means to ameliorate at least one symptom of the cancer. For example, a treatment can result in a reduction in tumor size, tumor growth, cancer cell number, cancer cell growth, or metastasis or risk of metastasis.

For example, the methods can include selecting and/or administering a treatment that includes a therapeutically effective amount of a BCR/TLR signaling inhibitor and/or a BCL2 inhibitor, to a subject having a select DLBCL cancer/tumor (e.g., a DLBCL of the C5 class, as defined herein).

Therapeutic agents specifically implicated for administration in using the instant DLBCL classifier include inhibitors of the following genetic targets:

### BCL-2

B-cell lymphoma 2 (BCL-2) is the founding member of the Bcl-2 family of regulator proteins that regulate apoptosis. Bcl-2 is considered an important anti-apoptotic protein but not a proto-oncogene. Targeted selective BCL-2 inhibitors for clinical use include oblimersen, ABT-263, and Venetoclax (ABT-199).

### BCL-6

B-cell lymphoma 6 (BCL-6) is an evolutionarily conserved zinc finger transcription factor and contains an N-terminal POZ/BTB domain. This protein acts as a sequence-specific repressor of transcription and has been shown to modulate the STAT-dependent Interleukin 4 (IL-4) responses of B cells. This gene is found to be frequently translocated and hypermutated in diffuse large B cell lymphoma (DLBCL) and contributes to the pathogenesis of DLBCL. An exemplary BCL6 inhibitor is 79-6-Calbiochem from Millipore.

### BCR/TLR Pathway

The B-cell receptor (BCR) protein controls the activation of B-cells. Toll-like receptors (TLRs) are a class of proteins that play a key role in the innate immune system. TLR's are single, membrane-spanning, non-catalytic receptors typically expressed on cells such as macrophages and dendritic cells. TLR's recognize structurally conserved molecules derived from microbes. BCR and TLR signaling synergizes for induction of activation-induced cytidine deaminase (AID) and immunoglobulin class-switching.

### Notch

The Notch signaling pathway is a highly conserved cell signaling system present in most multicellular organisms. Mammals possess four different notch receptors, NOTCH1, NOTCH2, NOTCH3, and NOTCH4. Notch signaling promotes proliferative signaling during neurogenesis, and its activity is inhibited by Numb to promote neural differentiation. NOTCH also plays a major role in the regulation of embryonic development. No approved NOTCH small molecule inhibitors are currently in use.

### PI3K

Phosphatidylinositol-4,5-bisphosphate 3-kinases, also called phosphatidylinositol-3-kinases (PI3K), are enzymes involved in cellular functions such as cell growth, proliferation, differentiation, motility, survival and intracellular trafficking. PI3Ks perform their functions through phosphorylating the 3 position hydroxyl group of the inositol ring of phosphatidylinositol. All PI3-kinases are inhibited by the drugs wortmannin and LY294002.

### EZH2

Enhancer of zeste homolog 2 (EZH2) is a histone-lysine N-methyltransferase enzyme (EC 2.1.1.43) that participates in histone methylation and, ultimately, transcriptional repression. Methylation activity of EZH2 facilitates heterochromatin formation thereby silences gene function. EZH2 is also the functional enzymatic component of the Polycomb Repressive Complex 2 (PRC2), which is responsible for healthy embryonic development through the epigenetic maintenance of genes responsible for regulating development and differentiation. Several inhibitors of EZH2 have been developed as of 2015, including 3-deazaneplanocin A (DZNep), EPZ005687, Ell, GSK126, and UNC1999. UNC1999 was developed as an analogue of GSK126, and was the first orally bioavailable EZH2 inhibitor to show activity. However, UNC1999 also binds to EZH1 and may have off-target effects.

### CREBBP

CREB-binding protein (CREBBP), is a protein that carries out its function by activating transcription. Interaction with transcription factors is managed by one or more CREB domains: the nuclear receptor interaction domain (RID), the KIX domain (CREB and MYB interaction domain), the cysteine/histidine regions (TAZ1/CH1 and TAZ2/CH3) and the interferon response binding domain (IBiD). A small molecule inhibitor (I-CBP112) binding to the bromodomain domain of CBP/p300 has been developed for leukemia therapy.

### JAK/STAT

The Janus kinase/signal transducer and activator of transcription (JAK/STAT) signaling pathway transmits information from extracellular chemical signals to the nucleus resulting in DNA transcription and expression of genes involved in immunity, proliferation, differentiation, apoptosis and oncogenesis. JAK-STAT signaling consists of three main components: a cell surface receptor, a JAK and two STAT proteins. An example JAK/STAT small molecule inhibitors is ruxolitinib.

### BRAF/MEK1 Blockade

BRAF is a human gene that encodes a protein called B-Raf. The gene is also referred to as proto-oncogene B-Raf, v-Raf murine sarcoma viral oncogene homolog B, and serine/threonine-protein kinase B-Raf. B-Raf protein is involved in internal cell signaling that directs cell growth.

Mitogen-activated protein kinase (MEK1 or MAP2K1), is a chromosome-axis-associated kinase that slows sister chromatid recombination. BRAF/MEK1 inhibition is possible with Vemurafenib and Cobimetinib, small molecule inhibitors currently used for advanced melanoma treatment.

Further examples of methods, and agents and combinations thereof suitable for treating a DLBCL are described in International Patent Applications No. WO2016189014A1, WO2012041800A1, WO2014068114A1, WO2001087337A1, WO2013024097A1, WO2013024095A1, WO2018078123A1, WO2017207574A1, WO2017032679A1, WO2013004806A1, WO2018220040A1, WO2020225196A1, WO2020120730A1, and WO2020055040A1, and in European Patent Application Publications No. EP2799451A1, and EP1667717A2, the disclosures of all of which are incorporated herein by reference in their entirety for all purposes.

An effective amount of an agent can be administered in one or more administrations, applications or dosages. A therapeutically effective amount of a therapeutic compound (i.e., an effective dosage) depends on the therapeutic compounds selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Determining Course (Progression/Remission) of Disease

In one embodiment, this invention provides methods for determining the course of disease in a subject. Disease course refers to changes in disease status over time, including disease progression (worsening) and disease regression (improvement). Determining the course of disease in a subject can involve measuring levels of circulating tumor DNA (ctDNA) in the subject. A decrease in ctDNA levels is generally indicative of disease regression.

### Reporting the Status

Additional embodiments of the invention relate to the communication of assay results or diagnoses or both to technicians, physicians or patients, for example. In certain embodiments, computers will be used to communicate assay results or diagnoses or both to interested parties, e.g., physicians and their patients. In some embodiments, the assays will be performed or the assay results analyzed in a country or jurisdiction which differs from the country or jurisdiction to which the results or diagnoses are communicated.

In a preferred embodiment of the invention, a diagnosis is communicated to the subject as soon as possible after the diagnosis is obtained. The diagnosis may be communicated to the subject by the subject's treating physician. Alternatively, the diagnosis may be sent to a subject by email or communicated to the subject by phone. A computer may be used to communicate the diagnosis by email or phone. In certain embodiments, the message containing results of a diagnostic test may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

### Subject Management

In certain embodiments, the methods of the invention involve managing subject treatment based on disease status. Such management includes referral, for example, to qualified specialist (e.g., an oncologist). In one embodiment, if a physician makes a diagnosis of a neoplasm or cancer, then a certain regime of treatment, such as prescription or administration of therapeutic agent might follow. Alternatively, a diagnosis of non-caner might be followed with further testing to determine a specific disease that might the patient might be suffering from. Also, if the diagnostic test gives an inconclusive result on cancer status, further tests may be called for.

Additional embodiments of the invention relate to the communication of assay results or diagnoses or both to technicians, physicians, or patients. In certain embodiments, computers will be used to communicate assay results or diagnoses or both to interested parties, e.g., physicians and their patients. In some embodiments, the assays will be performed, or the assay results analyzed in a country or jurisdiction which differs from the country or jurisdiction to which the results or diagnoses are communicated.

### Pharmaceutical Compositions

Agents of the present disclosure can be incorporated into a variety of formulations for therapeutic use (e.g., by administration) or in the manufacture of a medicament (e.g., for treating or preventing a DLBCL cancer) by combining the agents with appropriate pharmaceutically acceptable carriers or diluents, and may be formulated into preparations in solid, semi-solid, liquid or gaseous forms. Examples of such formulations include, without limitation, tablets, capsules, powders, granules, ointments, solutions, suppositories, injections, inhalants, gels, microspheres, and aerosols.

Pharmaceutical compositions can include, depending on the formulation desired, pharmaceutically-acceptable, non-toxic carriers of diluents, which are vehicles commonly used to formulate pharmaceutical compositions for animal or human administration The diluent is selected so as not to affect the biological activity of the combination. Examples of such diluents include. without limitation, distilled water, buffered water, physiological saline, PBS, Ringer's solution, dextrose solution, and Hank's solution. A pharmaceutical composition or formulation of the present disclosure can further include other carriers, adjuvants, or non-toxic, nontherapeutic, nonimmunogenic stabilizers, excipients and the like. The compositions can also include additional substances to approximate physiological conditions, such as pH adjusting and buffering agents. toxicity adjusting agents, wetting agents and detergents.

Further examples of formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249: 1527-1533 (1990).

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink.

Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents. stabilizers, and preservatives.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J Pharmaceutical Sciences 66 (1977): 1-19, incorporated herein by reference. The salts can be prepared in situ during the final isolation and purification of the compounds (e.g., FDA-approved compounds) of the application, or separately by reacting a free base or free acid function with a suitable reagent, as described generally below. For example, a free base function can be reacted with a suitable acid. Furthermore, where the compounds to be administered of the application carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters that hydrolyze in vivo and include those that break down readily in the human body to leave the parent compound (e.g., an FDA-approved compound where administered to a human subject) or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms.

Examples of particular esters include formates, acetates, propionates, butyrates, acrylates and ethylsuccinates.

Furthermore, the term “pharmaceutically acceptable prodrugs” as used herein refers to those prodrugs of the certain compounds of the present application which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the application. The term “prodrug” refers to compounds that are rapidly transformed in vivo to yield the parent compound of an agent of the instant disclosure, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, (1987), both of which are incorporated herein by reference.

The components used to formulate the pharmaceutical compositions are preferably of high purity and are substantially free of potentially harmful contaminants (e.g., at least National Food (NF) grade, generally at least analytical grade, and more typically at least pharmaceutical grade) Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Formulations may be optimized for retention and stabilization in a subject and/or tissue of a subject, e.g., to prevent rapid clearance of a formulation by the subject. Stabilization techniques include cross-linking. multimerizing, or linking to groups such as polyethylene glycol. polyacrylamide, neutral protein carriers, etc. in order to achieve an increase in molecular weight.

Other strategies for increasing retention include the entrapment of the agent, such as a BCL2 inhibitor, PI3K inhibitor, BCR/TLR signaling inhibitor, JAK/STAT inhibitor, etc., in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation Implants may be particles, sheets, patches, plaques, fibers, microcapsules and the like and may be of any size or shape compatible with the selected site of insertion.

The implants may be monolithic, i.e. having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment.

Biodegradable polymeric compositions which may be employed may be organic esters or ethers, which when degraded result in physiologically acceptable degradation products, including the monomers Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the individual instant disclosure. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

### Pharmaceutical Dosages

Pharmaceutical compositions of the present disclosure containing an agent described herein may be used (e.g., administered to an individual, such as a human individual, in need of treatment with a BCL2 inhibitor, PI3K inhibitor, BCR/TLR signaling inhibitor, JAK/STAT inhibitor, etc.) in accord with known methods, such as oral administration, intravenous administration as a bolus or by continuous infusion over a period of time, by intramuscular, intraperitoneal, intracerobrospinal, intracranial, intraspinal, subcutaneous, intraarticular, intrasynovial, intrathecal, topical, or inhalation routes.

Dosages and desired drug concentration of pharmaceutical compositions of the present disclosure may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles described in Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

For in vivo administration of any of the agents of the present disclosure, normal dosage amounts may vary from about 10 ng/kg up to about 100 mg/kg of an individual's and/or subject's body weight or more per day, depending upon the route of administration. In some embodiments, the dose amount is about 1 mg/kg/day to 10 mg/kg/day. For repeated administrations over several days or longer, depending on the severity of the disease, disorder, or condition to be treated, the treatment is sustained until a desired suppression of symptoms is achieved.

An effective amount of an agent of the instant disclosure may vary, e.g., from about 0.001 mg/kg to about 1000 mg/kg or more in one or more dose administrations for one or several days (depending on the mode of administration). In certain embodiments, the effective amount per dose varies from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, and from about 10.0 mg/kg to about 150 mg/kg.

An exemplary dosing regimen may include administering an initial dose of an agent of the disclosure of about 200 μg/kg, followed by a weekly maintenance dose of about 100 μg/kg every other week. Other dosage regimens may be useful, depending on the pattern of pharmacokinetic decay that the physician wishes to achieve. For example, dosing an individual from one to twenty-one times a week is contemplated herein. In certain embodiments, dosing ranging from about 3 μg/kg to about 2 mg/kg (such as about 3 μg/kg, about 10 μg/kg, about 30 μg/kg. about 100 μg/kg, about 300 μg/kg, about 1 mg/kg. or about 2 mg/kg) may be used. In certain embodiments, dosing frequency is three times per day, twice per day, once per day. once every other day. once weekly, once every two weeks, once every four weeks, once every five weeks, once every six weeks, once every seven weeks, once every eight weeks, once every nine weeks, once every ten weeks, or once monthly, once every two months, once every three months, or longer. Progress of the therapy is easily monitored by conventional techniques and assays. The dosing regimen, including the agent(s) administered, can vary over time independently of the dose used.

Pharmaceutical compositions described herein can be prepared by any method known in the art of pharmacology. In general, such preparatory methods include the steps of bringing the agent or compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, Poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methyl cellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl@.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of an agent (e.g., a BCL2 inhibitor, PI3K inhibitor, BCR/TLR signaling inhibitor, JAK/STAT inhibitor, etc.) described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required.

Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration.

Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid nonionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

FDA-approved drugs provided herein are typically formulated in dosage unit form for ease of administration and uniformity of dosage. It will be understood, however, that the total daily usage of the agents described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The agents and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the agent or pharmaceutical composition described herein is suitable for topical administration to the eye of a subject.

The exact amount of an agent required to achieve an effective amount will vary from subject to subject, depending, for example, on species, age, and general condition of a subject, severity of the side effects or disorder, identity of the particular agent, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of an agent (e.g., a BCL2 inhibitor, PI3K inhibitor, BCR/TLR signaling inhibitor, JAK/STAT inhibitor, etc.) described herein.

As noted elsewhere herein, a drug of the instant disclosure may be administered via a number of routes of administration, including but not limited to: subcutaneous, intravenous, intrathecal, intramuscular, intranasal, oral, transepidermal, parenteral, by inhalation, or intracerebroventricular.

The term “injection” or “injectable” as used herein refers to a bolus injection (administration of a discrete amount of an agent for raising its concentration in a bodily fluid), slow bolus injection over several minutes, or prolonged infusion, or several consecutive injections/infusions that are given at spaced apart intervals.

In some embodiments of the present disclosure, a formulation as herein defined is administered to the subject by bolus administration.

The FDA-approved drug or other therapy is administered to the subject in an amount sufficient to achieve a desired effect at a desired site (e.g., reduction of cancer size, cancer cell abundance, symptoms, etc.) determined by a skilled clinician to be effective. In some embodiments of the disclosure, the agent is administered at least once a year. In other embodiments of the disclosure, the agent is administered at least once a day. In other embodiments of the disclosure, the agent is administered at least once a week. In some embodiments of the disclosure, the agent is administered at least once a month.

Additional exemplary doses for administration of an agent of the disclosure to a subject include, but are not limited to, the following: 1-20 mg/kg/day, 2-15 mg/kg/day, 5-12 mg/kg/day, 10 mg/kg/day, 1-500 mg/kg/day, 2-250 mg/kg/day, 5-150 mg/kg/day, 20-125 mg/kg/day, 50-120 mg/kg/day, 100 mg/kg/day, at least 10 μg/kg/day, at least 100 μg/kg/day, at least 250 μg/kg/day, at least 500 μg/kg/day, at least 1 mg/kg/day, at least 2 mg/kg/day, at least 5 mg/kg/day, at least 10 mg/kg/day, at least 20 mg/kg/day, at least 50 mg/kg/day, at least 75 mg/kg/day, at least 100 mg/kg/day, at least 200 mg/kg/day, at least 500 mg/kg/day, at least 1 g/kg/day, and a therapeutically effective dose that is less than 500 mg/kg/day, less than 200 mg/kg/day, less than 100 mg/kg/day, less than 50 mg/kg/day, less than 20 mg/kg/day, less than 10 mg/kg/day, less than 5 mg/kg/day, less than 2 mg/kg/day, less than 1 mg/kg/day, less than 500 μg/kg/day, and less than 500 μg/kg/day.

In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of an agent (e.g., a BCL2 inhibitor, PI3K inhibitor, BCR/TLR signaling inhibitor, JAK/STAT inhibitor, etc.) described herein.

In certain embodiments, a dose described herein includes independently between 1 mg and 3 mg, inclusive, of an agent (e.g., a BCL2 inhibitor, PI3K inhibitor, BCR/TLR signaling inhibitor, JAK/STAT inhibitor, etc.) described herein. In certain embodiments, a dose described herein includes independently between 3 mg and 10 mg, inclusive, of an agent (e.g., a BCL2 inhibitor, PI3K inhibitor, BCR/TLR signaling inhibitor, JAK/STAT inhibitor, etc.) described herein. In certain embodiments, a dose described herein includes independently between 10 mg and 30 mg, inclusive, of an agent (e.g., a BCL2 inhibitor, PI3K inhibitor, BCR/TLR signaling inhibitor, JAK/STAT inhibitor, etc.) described herein. In certain embodiments, a dose described herein includes independently between 30 mg and 100 mg, inclusive, of an agent (e.g., a BCL2 inhibitor, PI3K inhibitor, BCR/TLR signaling inhibitor, JAK/STAT inhibitor, etc.) described herein.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

It will be also appreciated that an agent (e.g., a BCL2 inhibitor, PI3K inhibitor, BCR/TLR signaling inhibitor, JAK/STAT inhibitor, etc.) or composition, as described herein, can be administered in combination with one or more additional pharmaceutical agents (e.g., therapeutically and/or prophylactically active agents), which are different from the agent or composition and may be useful as, e.g., combination therapies. The agents or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk of developing a disease in a subject in need thereof, in inhibiting the replication of a virus, in killing a virus, etc. in a subject or cell. In certain embodiments, a pharmaceutical composition described herein including an agent (e.g., a BCL2 inhibitor, PI3K inhibitor, BCR/TLR signaling inhibitor, JAK/STAT inhibitor, etc.) described herein and an additional pharmaceutical agent shows a synergistic effect that is absent in a pharmaceutical composition including one of the agent and the additional pharmaceutical agent, but not both.

In some embodiments of the disclosure, a therapeutic agent distinct from a first therapeutic agent of the disclosure is administered prior to, in combination with, at the same time, or after administration of the agent of the disclosure. In some embodiments, the second therapeutic agent is selected from the group consisting of a chemotherapeutic, an antioxidant, an anti-inflammatory agent, an antimicrobial, a steroid, etc.

The agent or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease described herein. Each additional pharmaceutical agent may be administered at a dose and/or on a time schedule determined for that pharmaceutical agent. The additional pharmaceutical agents may also be administered together with each other and/or with the agent or composition described herein in a single dose or administered separately in different doses. The particular combination to employ in a regimen will take into account compatibility of the agent described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, additional BCL2 inhibitors, PI3K inhibitors, BCR/TLR signaling inhibitors, JAK/STAT inhibitors, other epigenetic modifier inhibitors, etc., other anti-cancer agents, immunomodulatory agents, anti-proliferative agents, cytotoxic agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, and pain-relieving agents. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine kinase inhibitors), modulators of protein stability (e.g., proteasome inhibitors), Hsp90 inhibitors, glucocorticoids, all-trans retinoic acids, and other agents that promote differentiation. In certain embodiments, the agents described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy.

Dosages for a particular agent of the instant disclosure may be determined empirically in individuals who have been given one or more administrations of the agent.

Administration of an agent of the present disclosure can be continuous or intermittent, depending, for example, on the recipient's physiological condition, whether the purpose of the administration is therapeutic or prophylactic, and other factors known to skilled practitioners. The administration of an agent may be essentially continuous over a preselected period of time or may be in a series of spaced doses.

Guidance regarding particular dosages and methods of delivery is provided in the literature; see, for example, U.S. Pat. Nos. 4,657,760; 5,206,344; or 5,225,212. It is within the scope of the instant disclosure that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

### Kits

The instant disclosure also provides kits containing agents of this disclosure for use in the methods of the present disclosure. Kits of the instant disclosure may include one or more containers comprising an agent for treatment of a DLBCL and/or may contain agents (e.g., oligonucleotide primers, probes, etc.) for identifying a cancer or subject as possessing one or more variant sequences. In some embodiments, the kits further include instructions for use in accordance with the methods of this disclosure. In some embodiments, these instructions comprise a description of use of the agent to treat or diagnose, e.g., a DLBCL, according to any of the methods of this disclosure. In some embodiments, the instructions comprise a description of how to detect a DLBCL class of cancer, for example in an individual, in a tissue sample, or in a cell. The kit may further comprise a description of treatments suggested for an individual as suitable for treatment based on identifying whether that subject has a specific class of DLBCL.

Instructions supplied in the kits of the instant disclosure are typically written instructions on a label or package insert (e.g., a paper sheet included in the kit), but machine-readable instructions (e.g., instructions carried on a magnetic or optical storage disk) are also acceptable. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Kits may optionally provide additional components such as buffers and interpretive information. Normally, the kit comprises a container and a label or package insert(s) on or associated with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Genetic Analyses of DLBCL

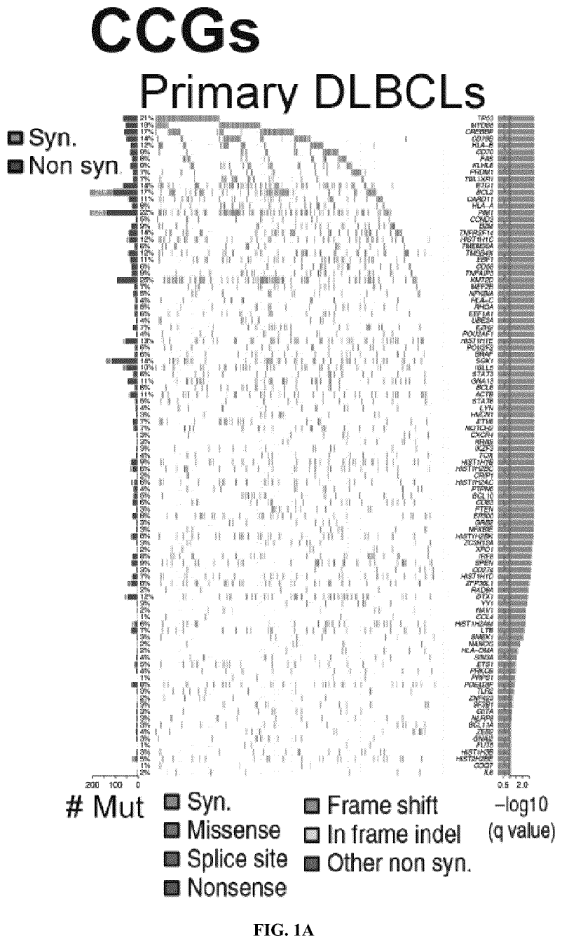

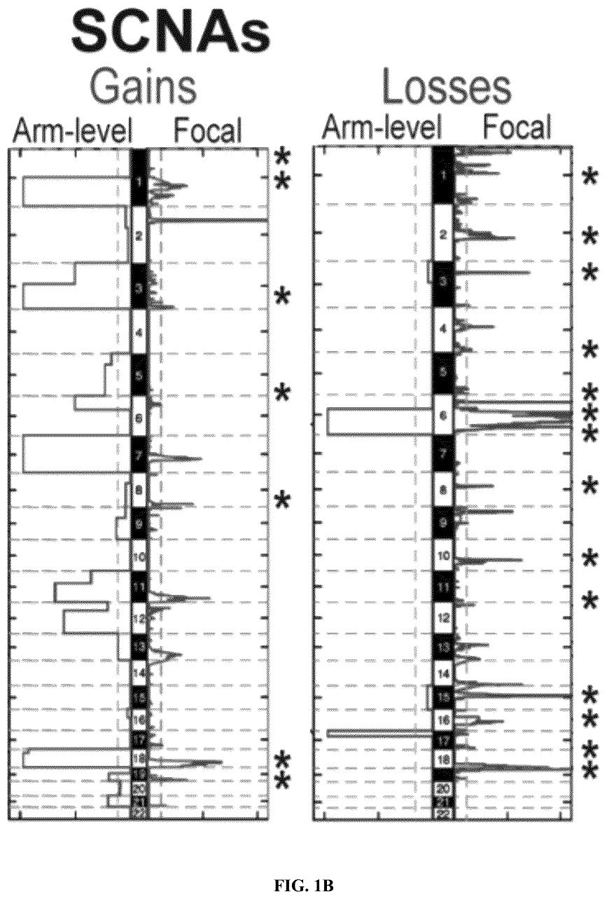

To define the genetic features of discrete diffuse large B-cell lymphoma (DLBCL) subsets, recurrent mutations, somatic copy number alterations (SCNAs), and structural variants (SVs) were identified in 304 newly diagnosed DLBCLs from patients who were treated with state-of-the-art combination chemotherapy (Rituximab, cyclophosphamide adriamycin, vincristine, prednisone, R-CHOP) and had long term follow up (FIGS. 1A and 1B) (Goy A. Succeeding in Breaking the R-CHOP Ceiling in DLBCL: Learning From Negative Trials. J Clin Oncol. 2017; 35(31):3519-3522). To accomplish these goals, new computational methods were developed to characterize 1) formalin-fixed paraffin embedded tumor biopsies; 2) tumor specimens without paired normal DNAs; and 3) structural variants (SVs) using a 4-algorithm pipeline. Significantly mutated candidate cancer genes (CCGs) were found to be highly likely to reside within focal SCNAs (Fisher Exact test, p=1e-44) indicating that these driver genes may have been perturbed by multiple mechanisms (FIG. 1B).

### Example 2: Identification of Genetically Distinct DLBCL Subsets

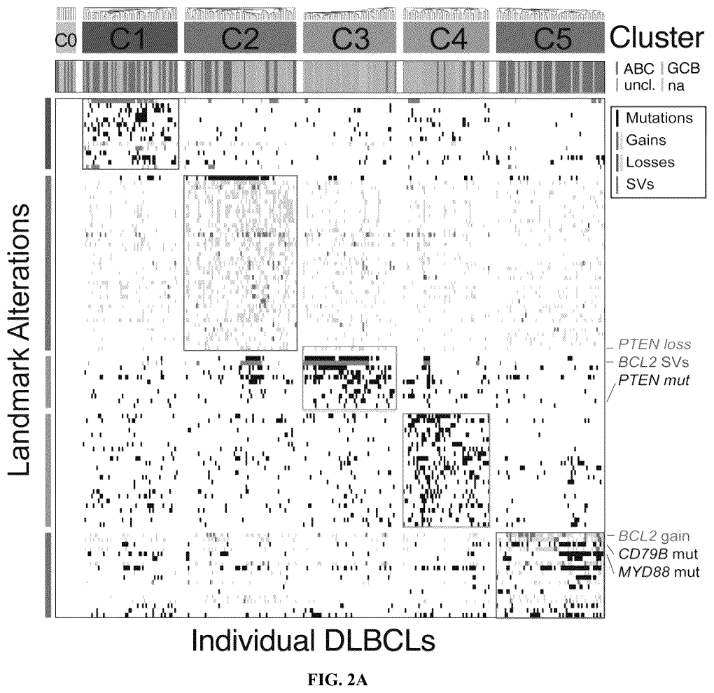

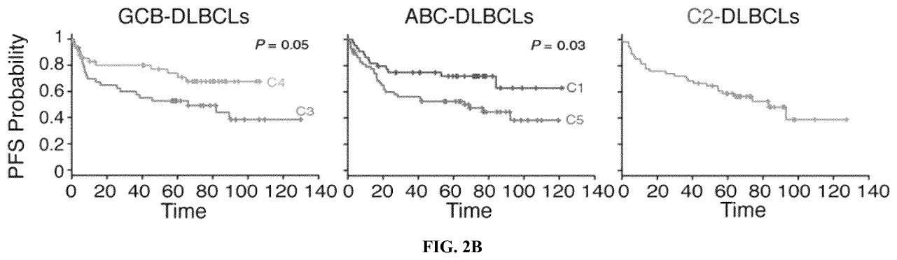

Next, using the genetic information obtained in Example 1, subsets of DLBCL were defined. Non-negative matrix factorization (NMF) consensus clustering was applied to identified recurrent driver alterations, and 5 subsets of tumors (clusters) were defined with discrete genetic signatures (C1-C5; 51 to 72 samples each). These clusters included the following, which were each associated with the indicated biologic features: 1) high-risk ABC-DLBCLs with near-uniform BCL2 copy gain, frequent activating MYD88^{L265P} and CD79B mutations and extranodal tropism (C5); 2) previously unappreciated low-risk ABC-DLBCLs with genetic features of an extrafollicular, possibly marginal zone, origin (C1); 3) high-risk GCB-DLBCLs with BCL2 SVs, inactivating mutations and/or copy loss of PTEN and alterations of epigenetic enzymes (C3); 4) a newly defined group of low-risk GCB-DLBCLs with distinct alterations in JAK/STAT and BRAF pathway components and multiple histones (C4); and 5) an ABC/GCB-independent group of tumors with biallelic inactivation of TP53, 9p21.3/CDKN2A copy loss and associated genomic instability (C2) (FIG. 2A) (the biologic features mentioned here as well as further biologic features associated with each subtype are presented in Chapuy B, Stewart C, Dunford A J, et al. Molecular subtypes of diffuse large B cell lymphoma are associated with distinct pathogenic mechanisms and outcomes. Nat Med. 2018; 24(5):679-690). The C2 subtype was associated with increased genomic instability and arm level and focal SCNAs. Patients with the genetically distinct transcriptional subtypes (C1 and C5 ABC DLBCLs; C3 and C4 GCB DLBCLs) had marked differences in progression-free survival (PFS) with significantly higher risks for relapse in C5 ABC- and C3 GCB-DLBCLs, and patients with C2 DLBCLs had a distinct trajectory and steady rate of progression over time (FIG. 2B). Thus, the genetically defined clusters identified separate high- and low-risk categories of the transcriptionally-defined subsets “ABC” (C5 and C1, respectively) and “GCB” (Clusters C3 and C4, respectively) subsets. C2 was an additional category of DLBCLs unrelated to the cell-of-origin classification; therefore, the genetically defined clusters superseded the previously proposed transcriptional clusters (ABC and GCB), which have proven unsuccessful in guiding therapy.

Genetic alterations identified as characterizing each cluster revealed multiple complementary mechanisms used by specific categories (clusters) of DLBCLs. These complementary mechanisms may be targeted together to treat a particular category of DLBCL. For example, aberrant B-cell receptor signaling and deregulated BCL-2 expression (associated with decreased apoptosis) has been associated with C3 and C5 and this can, therefore, inform selection of a therapy for DLBCLs falling into these categories.

### Example 3: Validation of the Genetic DLBCL Substructure in an Independent Dataset

To increase the clinical utility of the genetic subclasses identified in Example 2, the genetic substructure was validated in an independent dataset. Then a robust molecular classifier was developed that allowed prediction in new samples.

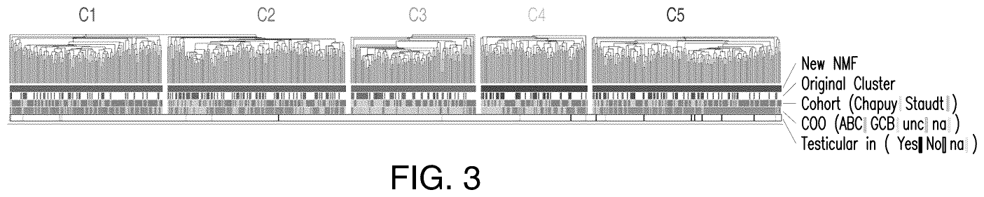

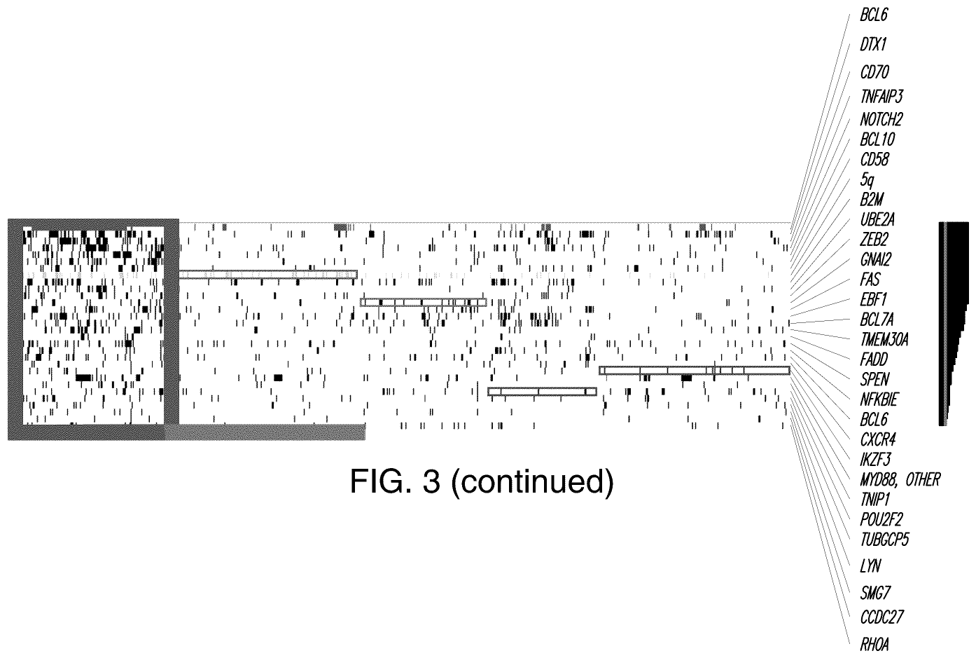

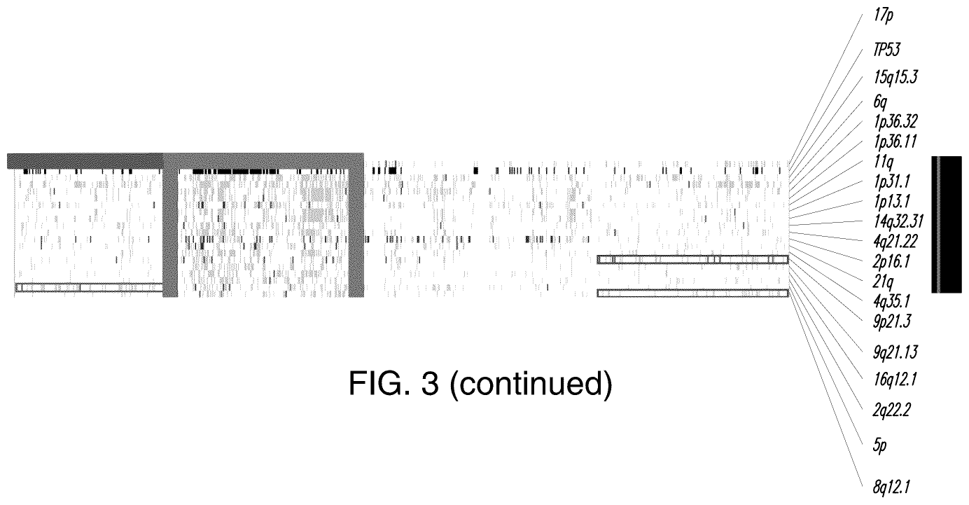

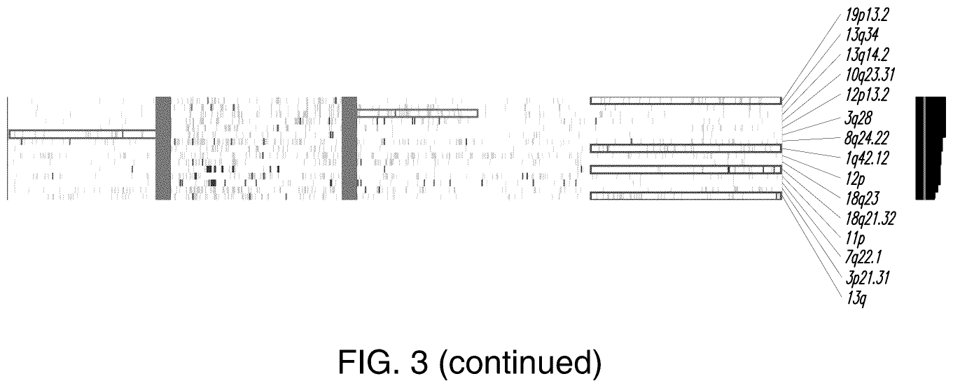

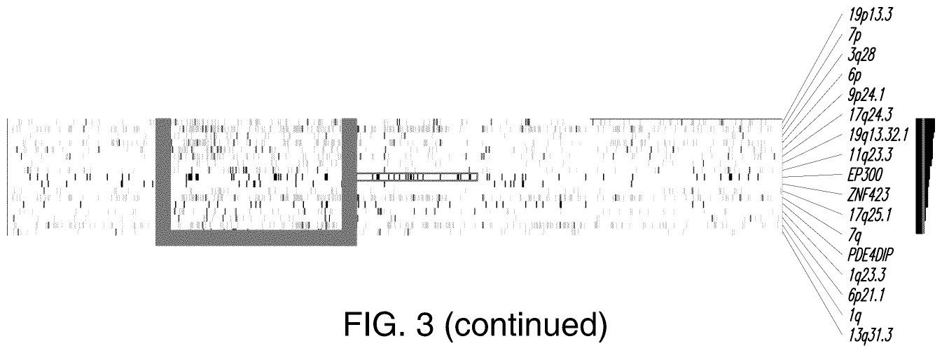

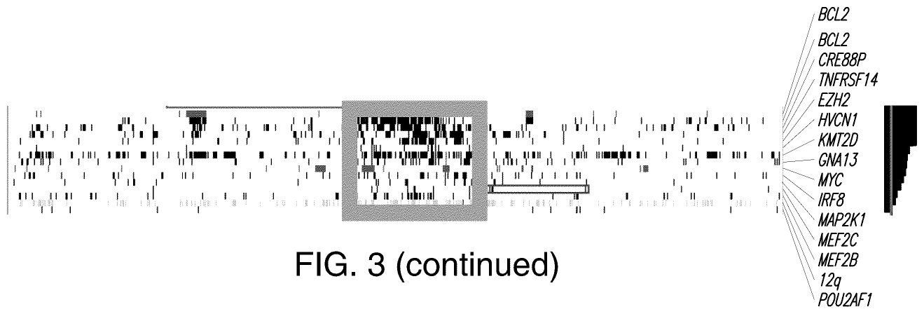

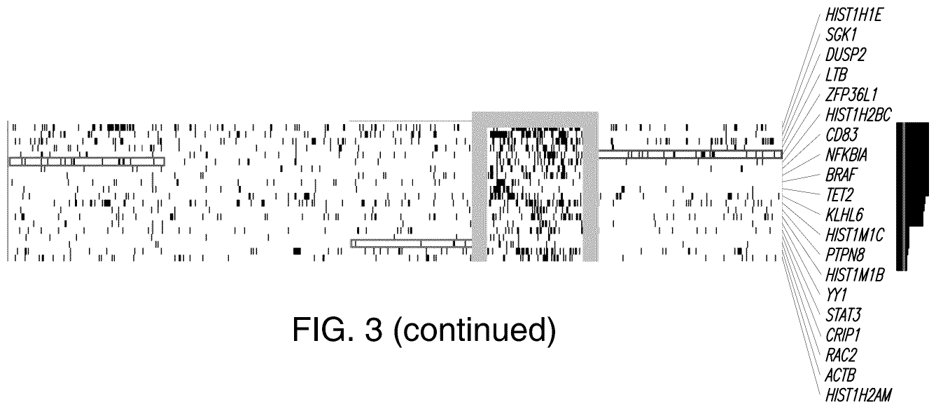

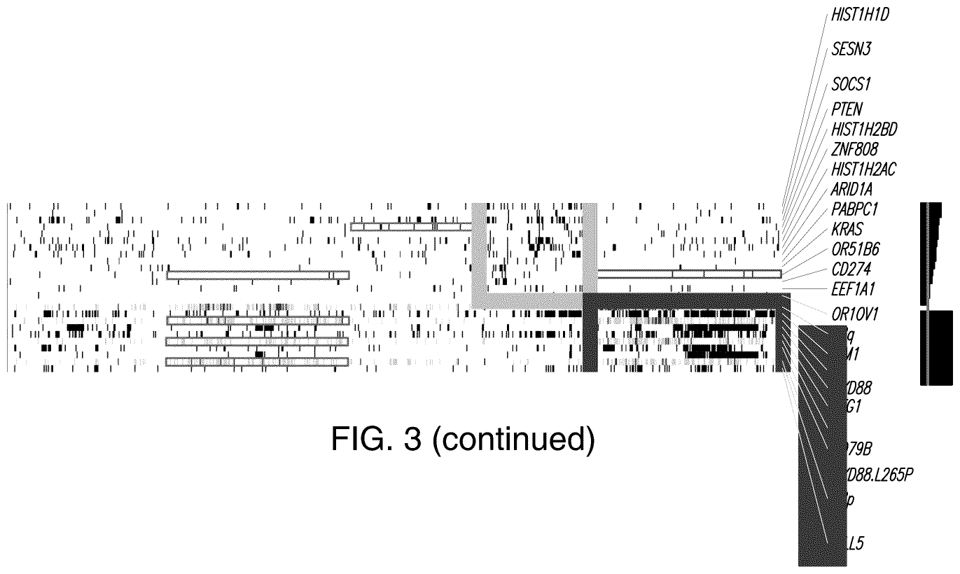

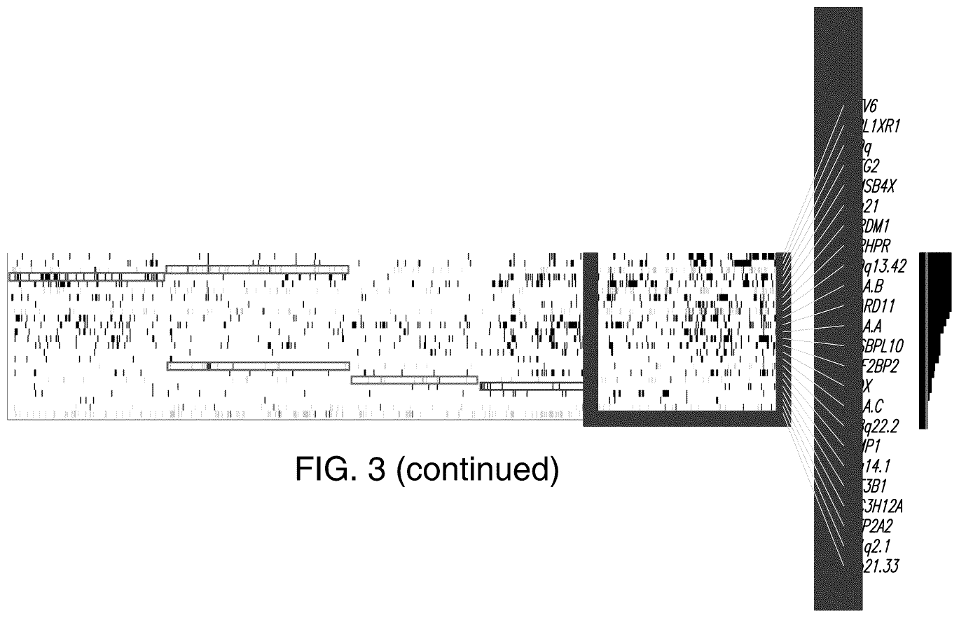

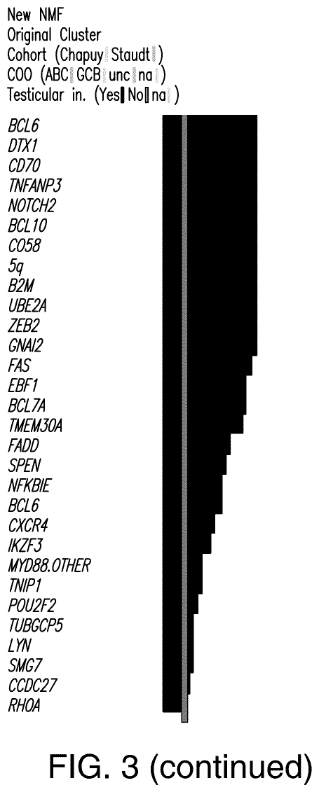

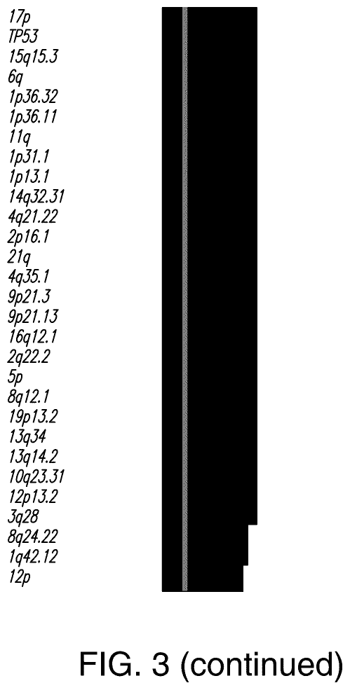

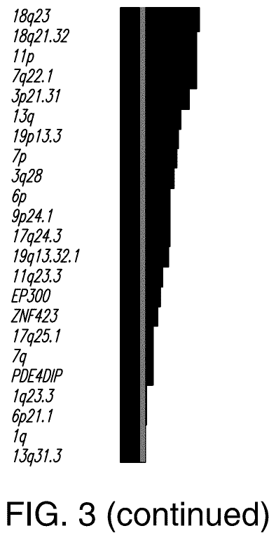

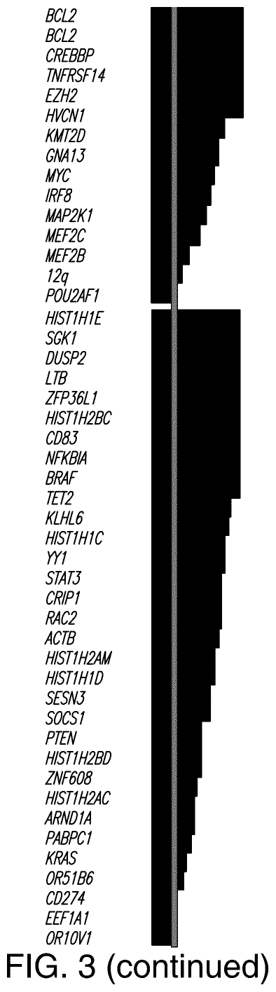

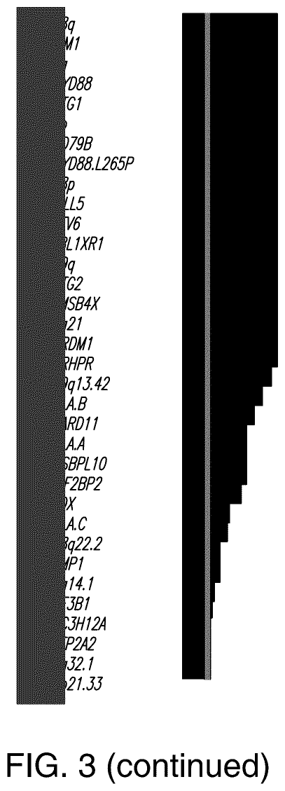

To validate the genetic substructure, raw genetic data (mutations, SCNAs, and SVs) was obtained from a recently published independent series of patients with newly diagnosed DLBCLs who were treated with state-of-the-art therapy (Schmitz, et al. Genetics and Pathogenesis of Diffuse Large B-Cell Lymphoma. N Engl J Med. 2018; 378(15):1396-1407). To avoid batch effects, mutations and SCNAs were reanalyzed using published pipelines (Chapuy et al. Molecular subtypes of diffuse large B cell lymphoma are associated with distinct pathogenic mechanisms and outcomes. Nat Med. 2018; 24(5):679-690) including a newly generated tumor-only algorithm to harmonize datasets and avoid batch effects. In the combined set, low purity cases and samples with missing data were removed and non-negative matrix factorization (NMF) clustering was applied to all samples of the combined set using the previously characterized 158 genetic drivers (FIG. 3). NMF clustering in the combined set (n=721) identified 5 clusters. Notably, samples of each series contributed to each cluster in the same proportion (FIG. 3). It was confirmed that: 1) C1 and C5 DLBCLs were enriched in the ABC transcriptional subtype (C1 p=0.02; C5 p=2e-33); 2) C3 and C4 DLBCLs were enriched in GCB (C3 p=4e-26; C4 p=2e-6), and 3) C2 DLBCLs were ABC/GCB independent (p=ns).

The biologic features of specific clusters were also validated using orthogonal evidence. Specifically, it was found that MYD88^{L265P} mutations were enriched in C5 DLBCLs (q=8e-23), MYD88^{non-L26P} mutations were predominantly found in C1 (q=0.07) and genome doublings were enriched in C2 DLBCLs (q=2e-18). Also, outcome differences for the respective clusters were preserved (logrank p=0.011). In summary, identity, associated marker genes, and biology of the C1-C5 DLBCL clusters in the combined cohort were confirmed, which served with its C1-C5 labels as a gold standard for the development of a molecular classifier.

### Example 4: Development of the DLBCL Molecular Classifier

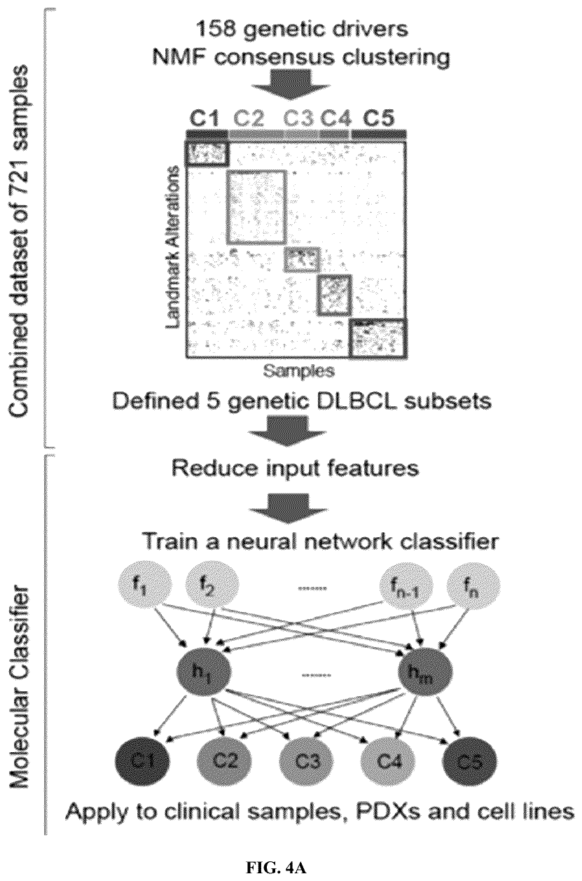

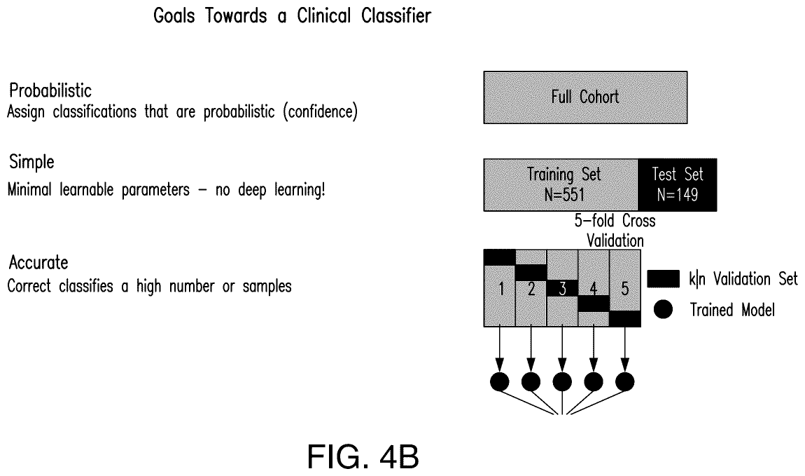

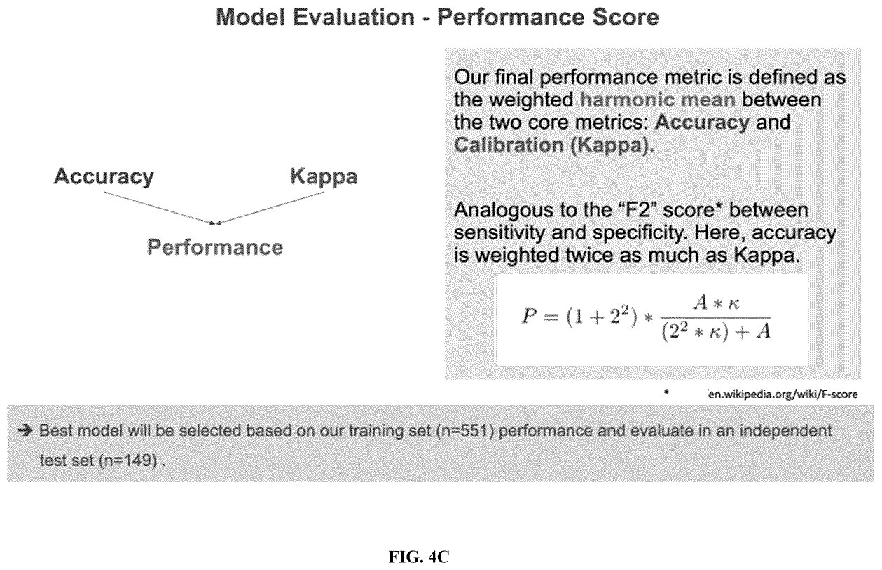

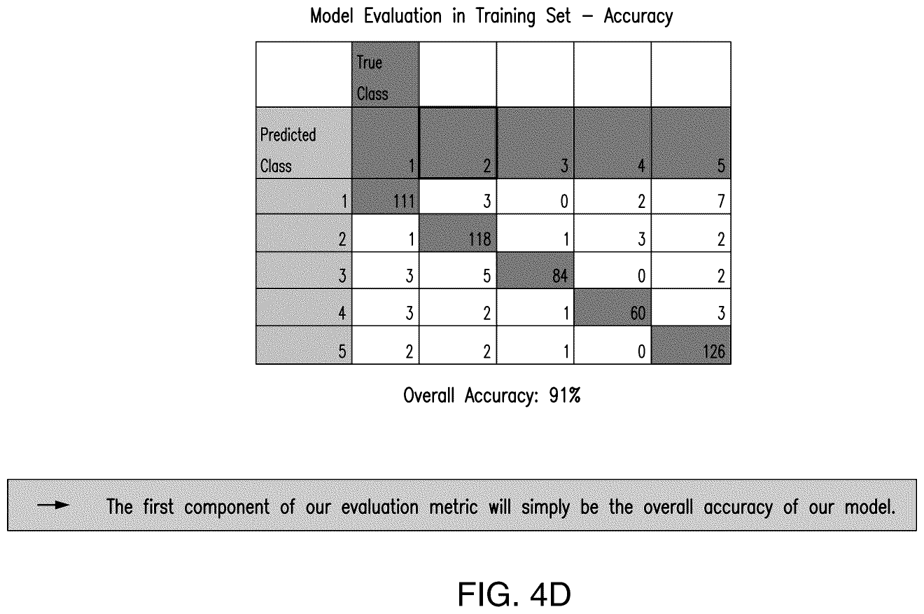

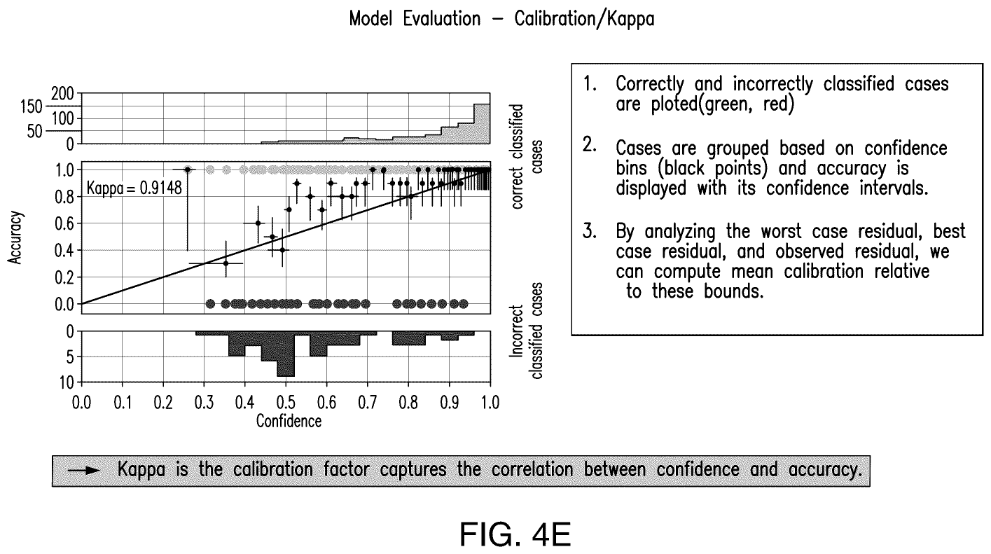

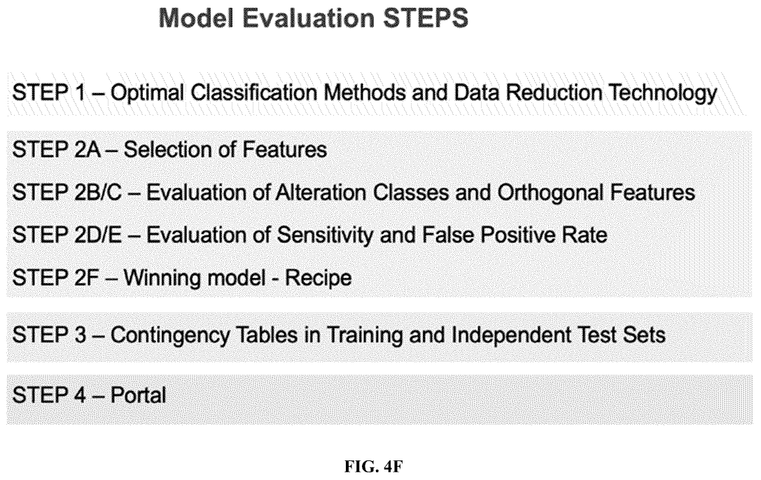

To match high-risk DLBCL patients with appropriate combination therapies, a robust molecular classifier was developed that can prospectively identify the newly defined tumor subsets. The classifier assigned single samples to specific clusters using a minimum number of easy-to-measure input features and designated as many samples as possible with high confidence. The labels from the combined set, composed of recently published DLBCL subtypes (Chapuy B, Stewart C, Dunford A J, et al. Molecular subtypes of diffuse large B cell lymphoma are associated with distinct pathogenic mechanisms and outcomes. Nat Med. 2018; 24(5):679-690) and the independent dataset (Schmitz R, Wright G W, Huang D W, et al. Genetics and Pathogenesis of Diffuse Large B-Cell Lymphoma. N Engl J Med. 2018; 378(15):1396-1407) served as a gold standard training and test dataset (FIG. 3 and the top of FIG. 4A).

The workflow and evaluation criteria for the DLBCL molecular classifier are presented in FIGS. 4A-4F. The overall design of the molecular classifier is presented in FIGS. 4A and 4B. To develop a genetic/molecular classifier, the combined datasets of DLBCL tumors from FIG. 3 was used as gold-standard to define the C1-C5 DLBCL classes. This cohort was divided into a training set (n=551) and a test set (n=149). Different feature (input variables) reduction technologies were evaluated and different computer-based classification methods were explored and optimized in the training set. The final model was evaluated in an independent test set suitable for application to different new datasets. The overall goals of the classifier were that it needed to be easy/simple (minimal learnable parameters), robust and probabilistic (assign classifications that are probabilistic (confidence)), and accurate for a large number of samples allowing an assignment into a given C1-C5 class and defining the specific probability (“certainty”) for the classification. FIG. 4C provides an overview of a performance score used to evaluate the molecular classifier. The evaluation of different classifier models leveraged a newly constructed “performance matrix”. This performance (P) matrix was defined as a weighted harmonic mean between two core metrics (accuracy and kappa), with accuracy (A) being weighted twice as much as the calibration factor, kappa (κ):

$$P = ( 1 + {2}^{2} ) * \frac{A * κ}{( {2}^{2} * κ ) + A} .$$

The performance metric was defined as the harmonic mean between accuracy and calibration (kappa) and was analogous to the “F2” score between sensitivity and specificity. To calculate the accuracy (FIG. 4D), correctly assigned cases were divided though all classified cases. An example of an high accuracy model is shown in the matrix of FIG. 4D. Kappa is a calibration factor that captures the correlation between confidence and accuracy (FIG. 4E). To calculate kappa, first correctly and incorrectly classified cases were plotted and grouped based on confidence bins. By analyzing the worst case residual, best case residual, and observed residual, mean calibration could be calculated relative to these bounds. A visualization of a model with good performance is shown in FIG. 4E.

FIG. 4F provides an overview of the steps used during the model construction and evaluation: 1) Optimal classification methods and data reduction technology, 2A) Selection of features, 2B/C) Evaluation of alteration classes and orthogonal features, 2D/E) Evaluation of sensitivity and false positive rate, 2F) Winning model—Recipe, 3) Contingency tables in training and independent test sets, 4) Portal (described further below).

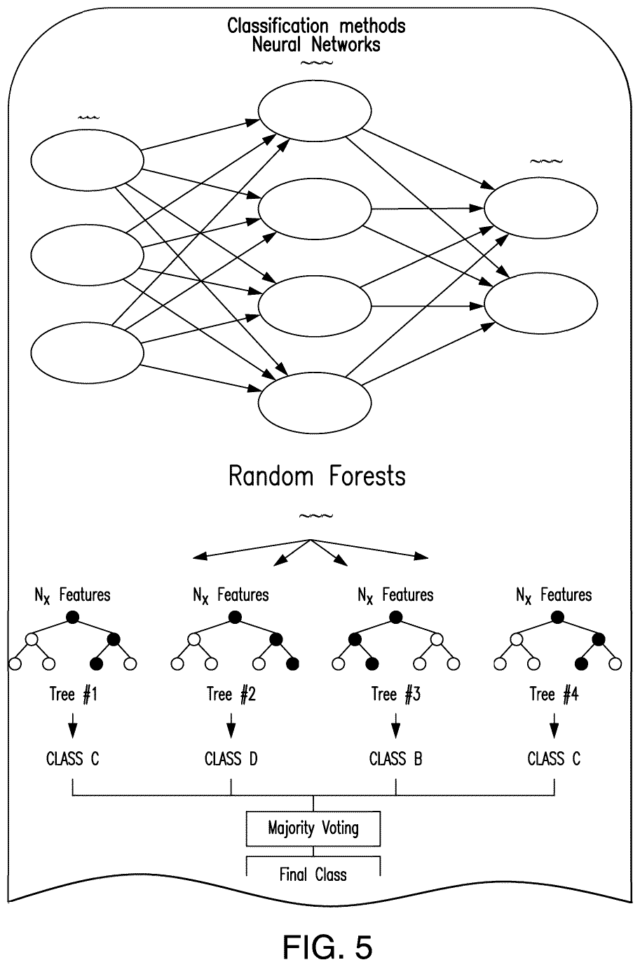

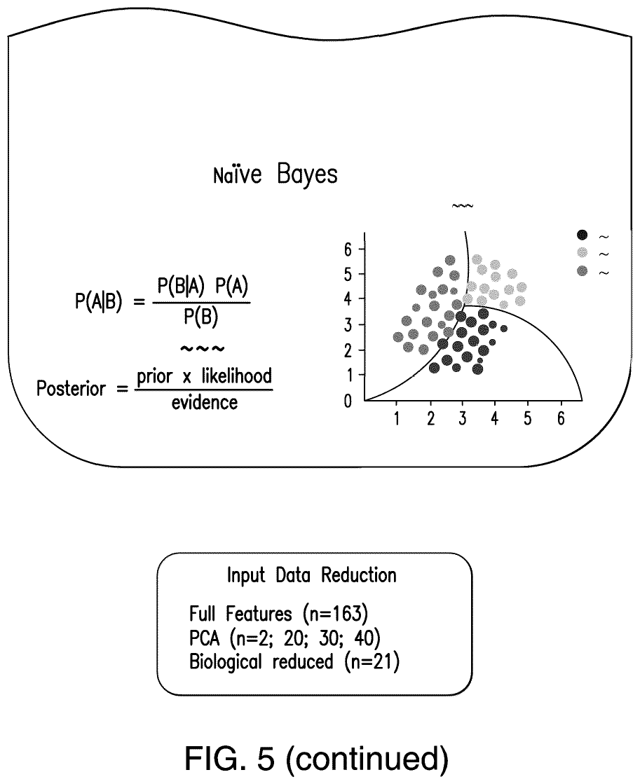

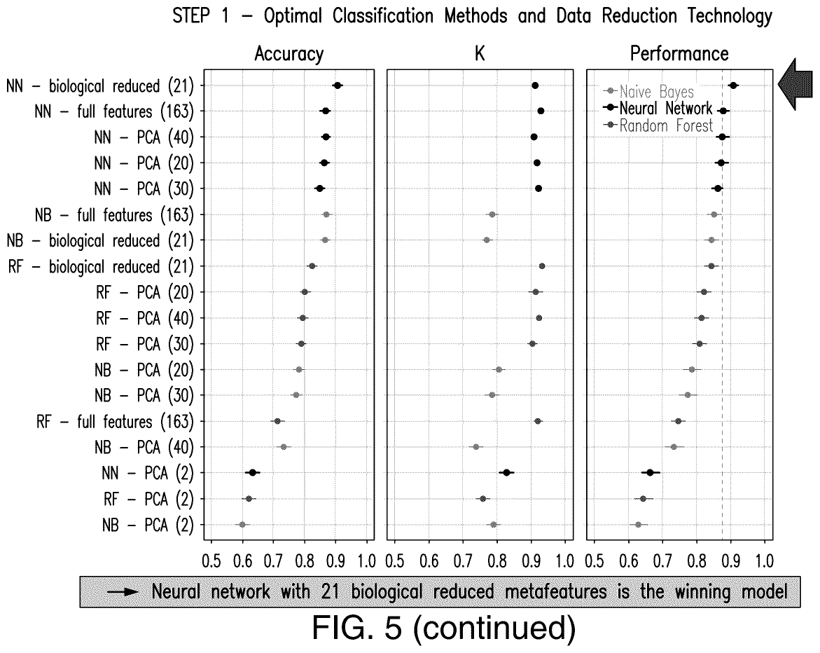

### Step 1: Selecting Optimal Classification Method and Data Reduction Technology (FIG. 5)

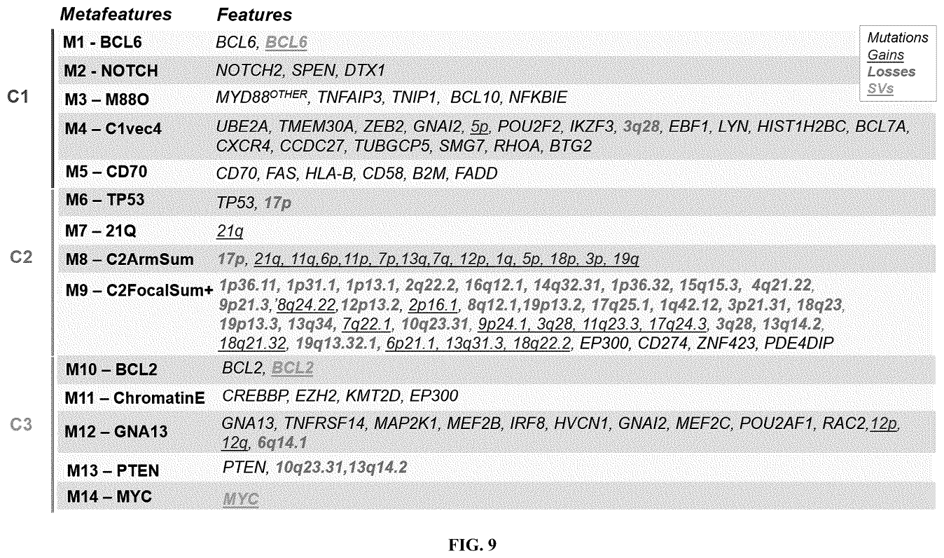

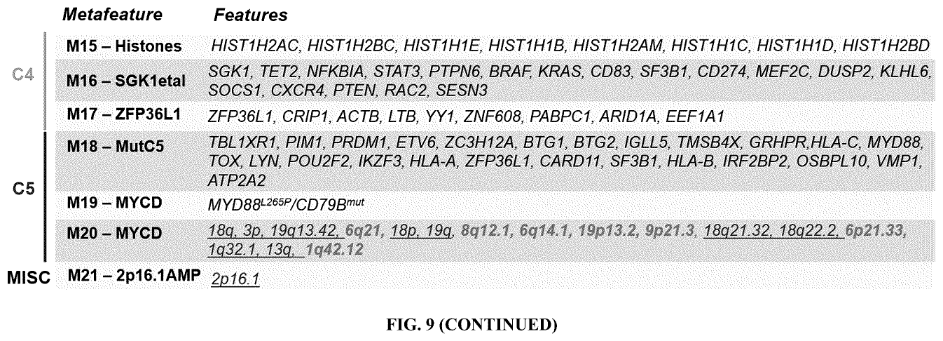

Different classification methods, including artificial neural networks, random forest and naïve Bayes classifying methods, were evaluated. The input features/variables were either used as full features (n=163) or reduced in their complexity by different dimensionality reduction technologies (e.g., PCA, biological reduction (summing of biologically relevant groups)). The Accuracy, kappa (κ) and the computed performance was calculated for each combination and ranked by performance. A model with good performance was an artificial neural network that learned the classification from a biological reduced input feature set (metafeatures, see Table 3 and FIG. 9).

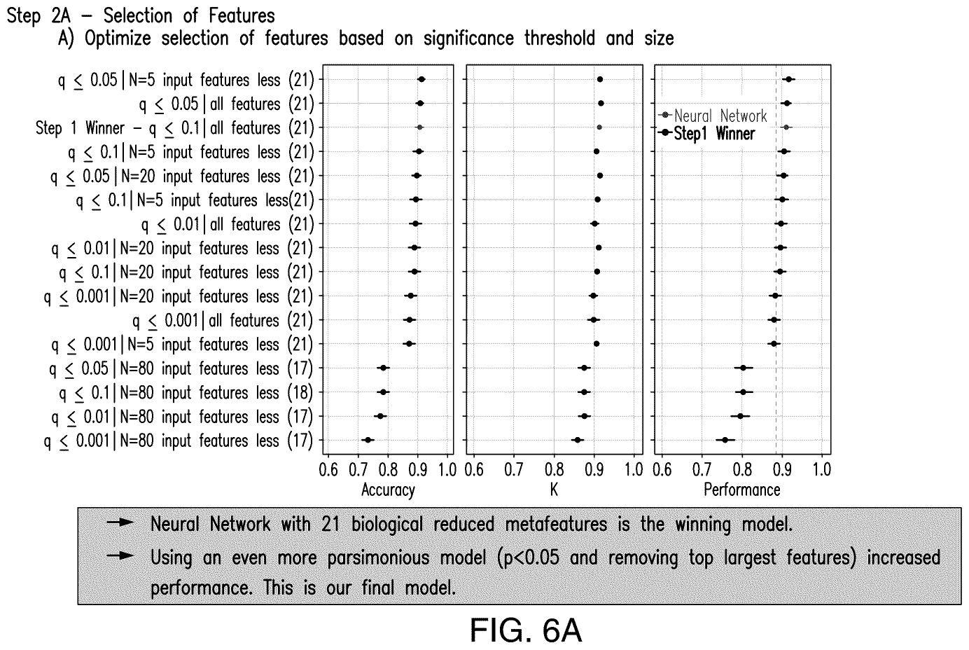

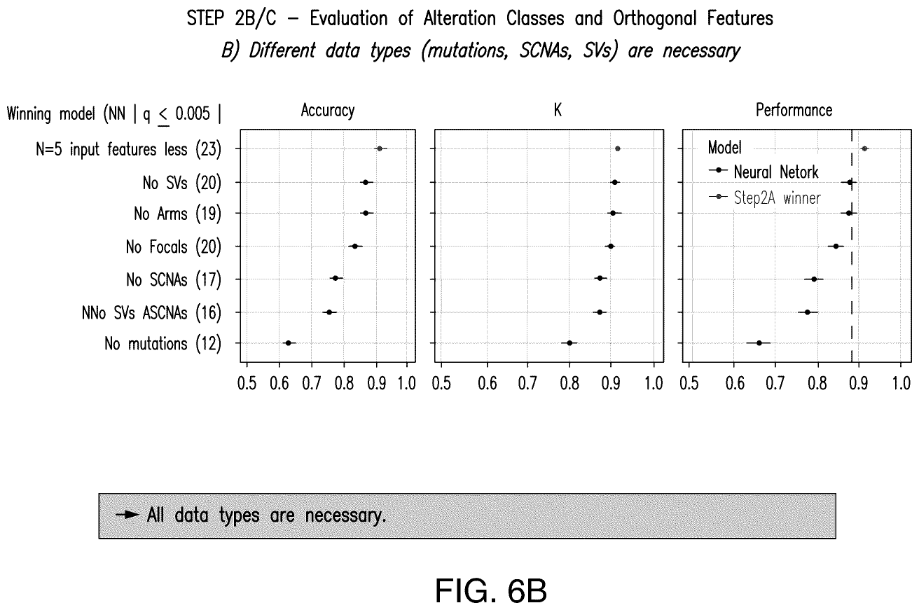

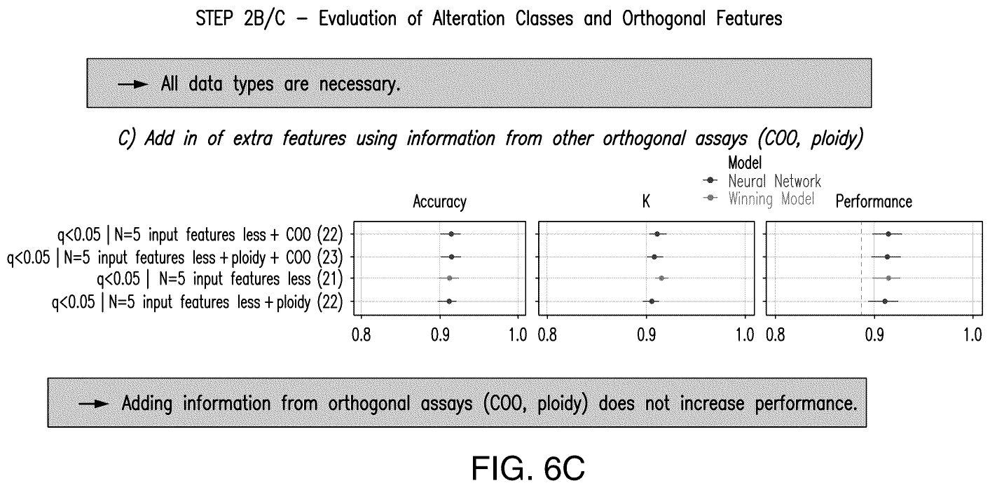

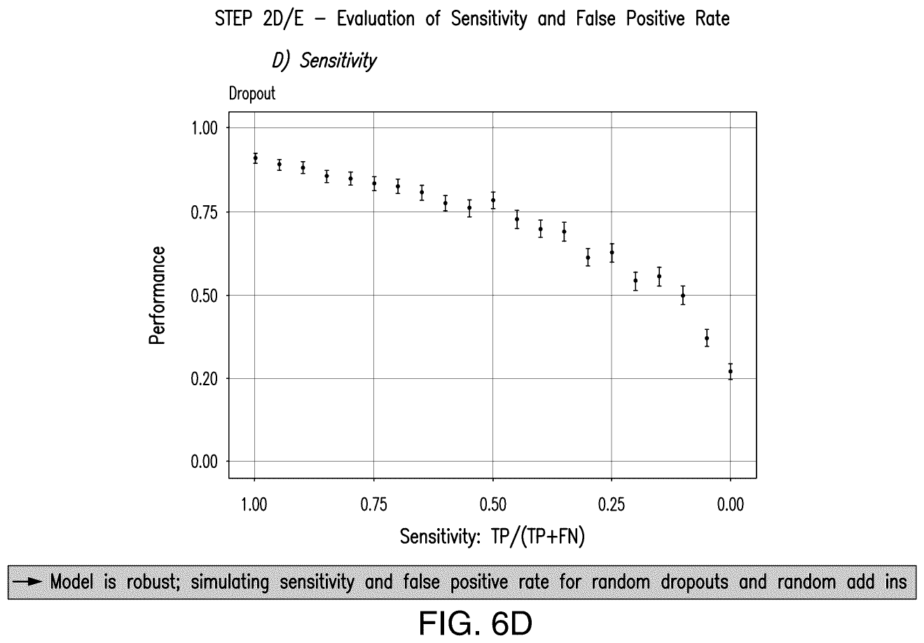

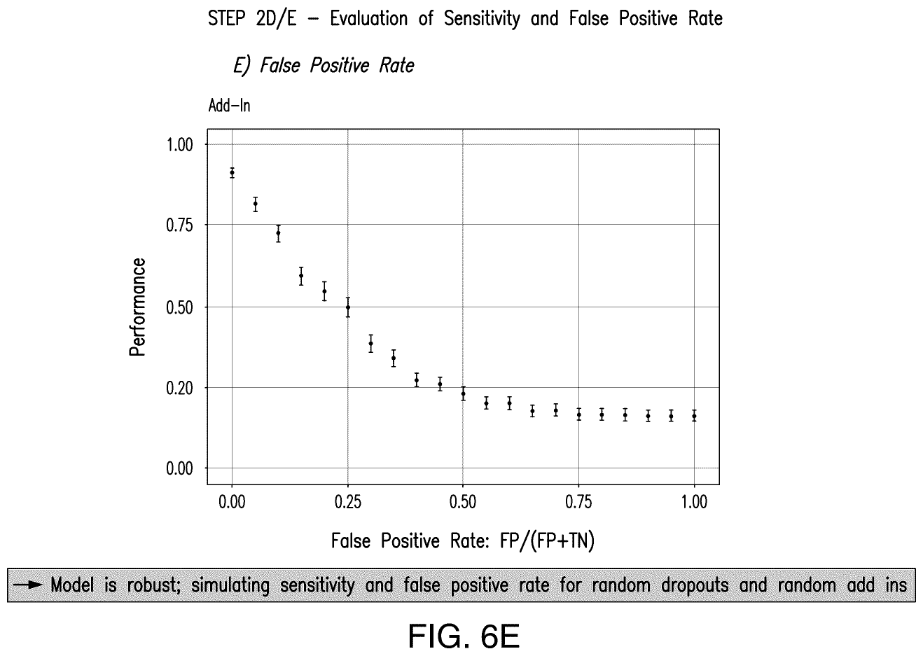

### Steps 2A-2E: Further Optimization and Testing of Robustness of the Molecular Classifier (FIGS. 6A-6E)

A model performing well in the prior step (FIG. 5) was further optimized. Features (i.e., metafeatures) were optimized by significance and size (FIG. 6A). It was explored if a more stringent selection of marker genes with increased q-value thresholds and/or the reduction of features increased the performance of the molecular classifier. Features were dropped by size of the feature in the genome space (larger features were removed first). Notably, with this step a final q-value at 0.05 was able to be determined and 5 features were dropped. The final list of metafeatures is provided in Table 3 and FIG. 9. It was next endeavored to determine if all datatypes (mutations, copy number alterations and structural variants) were necessary (FIG. 6B). Notably, all data types were important to use in combination because the removal of features or classes of alterations deteriorated the performance of the model. A further reduction of features (i.e., metafeatures) reduced the performance of the molecular classifier. The addition of orthogonal assays to capture molecular complexity in DLBCL, such as the cell of origin defined transcriptional subtypes and/or ploidy, did not significantly improve the performance of the molecular classifier (FIG. 6C). For this reason, the decision was made not to include these features in the molecular classifier. By simulating loss of input data (FIG. 6D) or adding of random noise/artifacts (FIG. 6E), the robustness of the model was evaluated and sensitivity and false positive rate of the model were determined.

As shown in Table 3 below and in FIG. 9, the metafeatures correspond to a compression of several measured features (i.e., the indicated variants). Each metafeature was calculated as a sum of variant classification-specific weighted values (see Table 1) determined based upon the measurement of each variant included in the metafeature. For example, the metafeature BCL6.C1 could range from 0 (no mutation and no SV) to 5 (non-synonymous mutation and SV). Typically, all of the variants and the indicated classifications were measured, see Table 3 and FIG. 9.

### Steps 3: Contingency Tables in Training and Independent Test Sets (FIG. 7)

The classification output of the molecular classifier as confusion matrix for the training and independent test sets is provided in FIG. 7. Since the classifier was probabilistic, it allowed for defining post-hoc thresholds on the confidence. In FIG. 7 the accuracy in training and test cohorts (top and bottom row, respectively) for no confidence threshold (left column, all cases are classified), the top 70% confident cases (middle panel) and above 90% confident cases, right column) are exemplified. The different confidence thresholds can be used to apply the classifier in a scenario in which all cases need to be classified with no confidence limit threshold. In a scenario in which the classifier is used to assign patients to a clinical trial, a high confidence threshold (e.g., 70% 75%, 80%, 85%, 90%, 95%, or 99%) might be used with the caveat of not classifying all patients.

TABLE 3
Input Variables (metafeatures) (see FIG. 9). The indicated 21 metafeatures, M1-M21, were composed of
the listed genetic features. The specific features are noted and the alteration types are indicated.

| Metafeature | | Associated | | Variant |
|---|---|---|---|---|
| No. | Metafeature value name | DLBCL Cluster | Variant | Classification |
| M1 | BCL6.C1.vec | C1 | BCL6 | Mutation |
| | | | BCL6 | SV |
| M2 | NOTCH2.C1.vec | C1 | NOTCH2 | Mutation |
| | | | SPEN | Mutation |
| | | | DTX1 | Mutation |
| M3 | NFKB.in.C1.vec | C1 | MYD88^{OTHER} | Mutation |
| | | | TNFAIP3 | Mutation |
| | | | TNIP1 | Mutation |
| | | | BCL10 | Mutation |
| | | | NFKBIE | Mutation |
| M4 | Ambiv.C1.vec | C1 | UBE2A | Mutation |
| | | | TMEM30A | Mutation |
| | | | ZEB2 | Mutation |
| | | | GNAI2 | Mutation |
| | | | 5p | SCNA.Arm.Gain |
| | | | POU2F2 | Mutation |
| | | | IKZF3 | Mutation |
| | | | 3q28 | SCNA.Focal.Loss |
| | | | EBF1 | Mutation |
| | | | LYN | Mutation |
| | | | HIST1H2BC | Mutation |
| | | | BCL7A | Mutation |
| | | | CXCR4 | Mutation |
| | | | CCDC27 | Mutation |
| | | | TUBGCP5 | Mutation |
| | | | SMG7 | Mutation |
| | | | RHOA | Mutation |
| | | | BTG2 | Mutation |
| M5 | Immunescape.C1.vec | C1 | CD70 | Mutation |
| | | | FAS | Mutation |
| | | | HLA-B | Mutation |
| | | | CD58 | Mutation |
| | | | B2M | Mutation |
| | | | FADD | Mutation |
| M6 | TP53.C2.vec | C2 | TP53 | Mutation |
| | | | 17p | SCNA.Arm.Loss |
| M7 | 21q.C2.vec | C2 | 21q | SCNA.Arm.Gain |
| M8 | C2.sumCN.arm.C2.vec | C2 | 17p | SCNA.Arm.Loss |
| | | | 21q | SCNA.Arm.Gain |
| | | | 11q | SCNA.Arm.Gain |
| | | | 6p | SCNA.Arm.Gain |
| | | | 11p | SCNA.Arm.Gain |
| | | | 7p | SCNA.Arm.Gain |
| | | | 13q | SCNA.Arm.Gain |
| | | | 7q | SCNA.Arm.Gain |
| | | | 12p | SCNA.Gain |
| | | | 1q | SCNA.Arm.Gain |
| | | | 5p | SCNA.Arm.Gain |
| | | | 18p | SCNA.Arm.Gain |
| | | | 3p | SCNA.Arm.Gain |
| | | | 19q | SCNA.Arm.Gain |
| M9 | C2.sumCN.focal.C2.vec | C2 | 1p36.11 | SCNA.Focal.Loss |
| | | | 1p31.1 | SCNA.Focal.Loss |
| | | | 1p13.1 | SCNA.Focal.Loss |
| | | | 2q22.2 | SCNA.Focal.Loss |
| | | | 16q12.1 | SCNA.Focal.Loss |
| | | | 14q32.31 | SCNA.Focal.Loss |
| | | | 1p36.32 | SCNA.Focal.Loss |
| | | | 4q35.1 | SCNA.Focal.Loss |
| | | | 9q21.13 | SCNA.Focal.Loss |
| | | | 15q15.3 | SCNA.Focal.Loss |
| | | | 4q21.22 | SCNA.Focal.Loss |
| | | | 9p21.3 | SCNA.Focal.Loss |
| | | | 8q24.22 | SCNA.Focal.Gain |
| | | | 12p13.2 | SCNA.Focal.Loss |
| | | | 2p16.1 | SCNA.Focal.Gain |
| | | | 8q12.1 | SCNA.Focal.Loss |
| | | | 19p13.2 | SCNA.Focal.Loss |
| | | | 17q25.1 | SCNA.Focal.Loss |
| | | | 1q42.12 | SCNA.Focal.Loss |
| | | | 3p21.31 | SCNA.Focal.Loss |
| | | | 18q23 | SCNA.Focal.Loss |
| | | | 19p13.3 | SCNA.Focal.Loss |
| | | | 13q34 | SCNA.Focal.Loss |
| | | | 7q22.1 | SCNA.Focal.Gain |
| | | | 10q23.31 | SCNA.Focal.Loss |
| | | | 9p24.1 | SCNA.Focal.Gain |
| | | | 1q23.3 | SCNA.Focal.Gain |
| | | | 3q28 | SCNA.Focal.Gain |
| | | | 11q23.3 | SCNA.Focal.Gain |
| | | | 17q24.3 | SCNA.Focal.Gain |
| | | | 3q28 | SCNA.Focal.Loss |
| | | | 13q14.2 | SCNA.Focal.Loss |
| | | | 18q21.32 | SCNA.Gain |
| | | | 19q13.32.1 | SCNA.Loss |
| | | | 6p21.1 | SCNA.Gain |
| | | | 13q31.3 | SCNA.Gain |
| | | | 18q22.2 | SCNA.Gain |
| | | | EP300 | Mutation |
| | | | CD274 | Mutation |
| | | | ZNF423 | Mutation |
| | | | PDE4DIP | Mutation |
| M10 | BCL2.C3.vec | C3 | BCL2 | Mutation |
| | | | BCL2 | SV |
| M11 | ChromatinModifier.C3.vec | C3 | CREBBP | Mutation |
| | | | EZH2 | Mutation |
| | | | KMT2D | Mutation |
| | | | EP300 | Mutation |
| M12 | Ambiv.C3.vec | C3 | GNA13 | Mutation |
| | | | TNFRSF14 | Mutation |
| | | | MAP2K1 | Mutation |
| | | | MEF2B | Mutation |
| | | | IRF8 | Mutation |
| | | | HVCN1 | Mutation |
| | | | GNAI2 | Mutation |
| | | | MEF2C | Mutation |
| | | | POU2AF1 | Mutation |
| | | | RAC2 | Mutation |
| | | | 12p | SCNA.Gain |
| | | | 12q | SCNA.Gain |
| | | | 6q14.1 | SCNA.Loss |
| M13 | PTENplus.C3.vec | C3 | PTEN | Mutation |
| | | | 10Q23.31 | SCNA.Focal.Loss |
| | | | 13Q14.2 | SCNA.Focal.Loss |
| M14 | MYC.C3.vec | C3 | MYC | SV |
| M15 | HIST.C4.vec | C4 | HIST1H2AC | Mutation |
| | | | HIST1H2BC | Mutation |
| | | | HIST1H1E | Mutation |
| | | | HIST1H1B | Mutation |
| | | | HIST1H2AM | Mutation |
| | | | HIST1H1C | Mutation |
| | | | HIST1H1D | Mutation |
| | | | HIST1H2BD | Mutation |
| M16 | SGK1.RAS.JAK.Immunescape.C4.vec | C4 | SGK1 | Mutation |
| | | | TET2 | Mutation |
| | | | NFKBIA | Mutation |
| | | | STAT3 | Mutation |
| | | | PTPN6 | Mutation |
| | | | BRAF | Mutation |
| | | | KRAS | Mutation |
| | | | CD83 | Mutation |
| | | | SF3B1 | Mutation |
| | | | CD274 | Mutation |
| | | | MEF2C | Mutation |
| | | | DUSP2 | Mutation |
| | | | KLHL6 | Mutation |
| | | | SOCS1 | Mutation |
| | | | CXCR4 | Mutation |
| | | | PTEN | Mutation |
| | | | RAC2 | Mutation |
| | | | SESN3 | Mutation |
| M17 | Ambiv.C4.vec | C4 | ZFP36L1 | Mutation |
| | | | CRIP1 | Mutation |
| | | | ACTB | Mutation |
| | | | LTB | Mutation |
| | | | YY1 | Mutation |
| | | | ZNF608 | Mutation |
| | | | PABPC1 | Mutation |
| | | | ARID1A | Mutation |
| | | | EEF1A1 | Mutation |
| M18 | Ambiv.C5.vec | C5 | TBL1XR1 | Mutation |
| | | | PIM1 | Mutation |
| | | | PRDM1 | Mutation |
| | | | ETV6 | Mutation |
| | | | ZC3H12A | Mutation |
| | | | BTG1 | Mutation |
| | | | BTG2 | Mutation |
| | | | IGLL5 | Mutation |
| | | | TMSB4X | Mutation |
| | | | GRHPR | Mutation |
| | | | HLA-C | Mutation |
| | | | MYD88 | Mutation |
| | | | TOX | Mutation |
| | | | LYN | Mutation |
| | | | POU2F2 | Mutation |
| | | | IKZF3 | Mutation |
| | | | HLA-A | Mutation |
| | | | ZFP36L1 | Mutation |
| | | | CARD11 | Mutation |
| | | | SF3B1 | Mutation |
| | | | HLA-B | Mutation |
| | | | IRF2BP2 | Mutation |
| | | | OSBPL10 | Mutation |
| | | | VMP1 | Mutation |
| | | | ATP2A2 | Mutation |
| M19 | MYD88CD79B | C5 | MYD88^{L265P} | Mutation |
| | | | CD79B^{mut} | Mutation |
| M20 | SumCN.C5.vec | C5 | 18q | SCNA.Arm.Gain |
| | | | 3p | SCNA.Arm.Gain |
| | | | 19q13.42 | SCNA.Focal.Gain |
| | | | 6q21 | SCNA.Focal.Loss |
| | | | 18p | SCNA.Arm.Gain |
| | | | 19q | SCNA.Arm.Gain |
| | | | 8q12.1 | SCNA.Focal.Loss |
| | | | 6q14.1 | SCNA.Focal.Loss |
| | | | 19p13.2 | SCNA.Focal.Loss |
| | | | 9p21.3 | SCNA.Loss |
| | | | 18q21.32 | SCNA.Gain |
| | | | 18q22.2 | SCNA.Gain |
| | | | 6p21.33 | SCNA.Loss |
| | | | 1q32.1 | SCNA.Gain |
| | | | 13q | SCNA.Gain |
| | | | 1q42.12 | SCNA.Loss |
| M21 | 2p16.1.vec | Miscellaneous | 2p16.1 | SCNA.Focal.Gain |

### Step 4: Development of an Online Tool

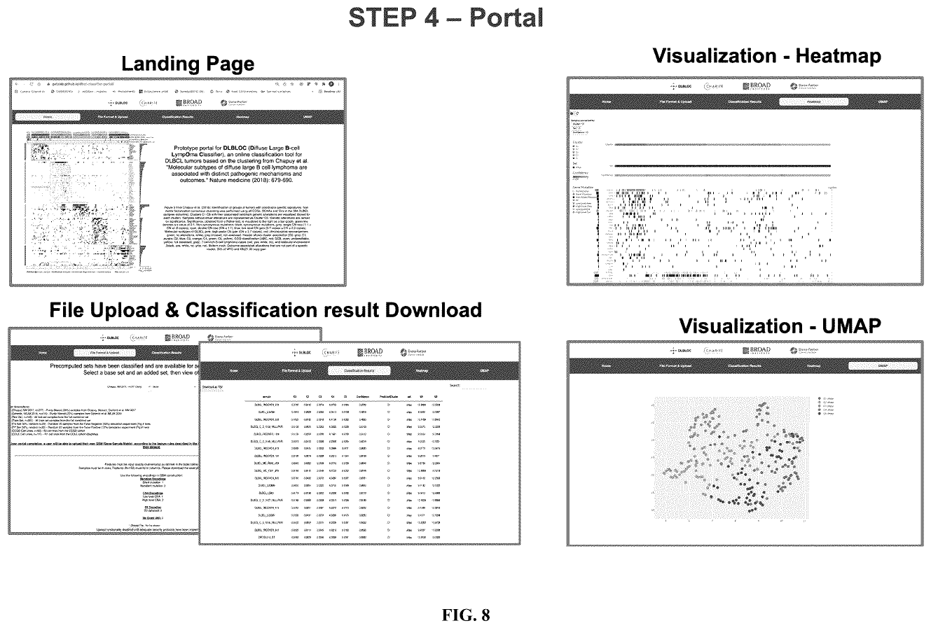

As one example of a tool that allows the commutation of the classification results for new cases, a web-portal was designed. The web portal (FIG. 8) was password protected, had a landing page, allowed for file/data upload, allowed for classification using the molecular classifier, and allowed for download of classification results (e.g., class and probability/confidence). In addition, the portal allowed visualization of the data in context to published, including the generation of heatmaps and UMAPs.

The web-portal was but one example of a tool that allows communication of classification results. Other tools might be apps for smartphones, etc.

### Example 5: Pilot Study

To match high-risk DLBCL patients with appropriate combination therapies, a robust molecular classifier was developed that can prospectively identify the newly defined tumor subsets. The classifier assigned single samples to specific clusters using a minimum number of easy-to-measure input features and designated as many samples as possible with high confidence. The labels from the combined set, composed of recently published DLBCL subtypes (Chapuy B, Stewart C, Dunford A J, et al. Molecular subtypes of diffuse large B cell lymphoma are associated with distinct pathogenic mechanisms and outcomes. Nat Med. 2018; 24(5):679-690) and the independent dataset (Schmitz R, Wright G W, Huang D W, et al. Genetics and Pathogenesis of Diffuse Large B-Cell Lymphoma. N Engl J Med. 2018; 378(15):1396-1407) served as a gold standard training and test dataset (FIG. 3 and the top of FIG. 4A). To develop a genetic/molecular classifier, the informative cases with C1-C5 assignments and full genetic marker availability were split into training and validation sets as well as an independent test set, composed of 50%, 25% and 25% of cases, respectively. During the supervised and iterative processes of classifier optimization, the training set and the validation set were used with k-fold cross validation. The test set was only used to measure the final performance of the candidate classifier.

### Input Features and Pilot Artificial Neural Network (ANN) Classifier

To avoid overtraining the classifier, the number of free parameters (input features, f1-n and hidden layers, h1-m) were limited to as few as possible while retaining classifier performance (FIG. 4A, bottom). In an initial pilot study, biological insights into distinguishing molecular features (e.g., the biologic features noted in Example 2 above) were used to reduce the 158 genetic features from NMF clustering (Chapuy B, Stewart C, Dunford A J, et al. Molecular subtypes of diffuse large B cell lymphoma are associated with distinct pathogenic mechanisms and outcomes. Nat Med. 2018; 24(5):679-690) to 21 input variables, see Table 3. As shown in Table 3 below, the 21 input variables each corresponded to a metafeature corresponding to a binning/compression of several measured features (i.e., the indicated variants). Each metafeature was calculated as a sum of variant classification-specific weighted values (see Table 1) determined based upon the measurement of each variant included in the metafeature. For example, the metafeature BCL6.C1 could range from 0 (no mutation and no SV) to 5 (non-synonymous mutation and SV). All of the variants and the indicated classifications were measured, see Table 3.

With the 21 input parameters, DLBCLs with known non-negative matrix factorization (NMF) clusters were visualized in uniform manifold approximation and projection (UMAP), a technique for dimensionality reduction. These 21 variables, which clearly separated the NMF-defined clusters, were used to train an artificial neural network (ANN) classifier. This pilot classifier was 99% and 95% accurate in assigning high confidence DLBCLs (>0.9 confidence) in the training/validation and test sets, respectively. Notably, the classifier output was designed to be probabilistic to reflect the heterogeneity in biology between patients. The probability estimate provided a cluster designation and the probability of accurate assignment, which are essential for treatment decisions.

### Optimization of the Classifier

The performance of the pilot ANN classifier with input parameters chosen for biological significance was compared to a classifier in which input parameters were selected solely by principal component analysis, by a neural network autoencoder, or by a Q-value-based method. The value of adding a confidence measurement of each assigned DLBCL cluster to the sample training vector was also assessed. The output of the ANN classifier was compared and benchmarked to that of other classifier algorithms (i.e. random forest, multinomial naïve Bayes). The robustness of the molecular classifier was assessed against various clinical scenarios (e.g., loss of sensitivity, loss of specificity, detection of only some of the alteration types, etc.). A significant deterioration of model accuracy was observed when variant classes were omitted, which confirmed that it was necessary to include all 3 classes of genetic alterations (mutations, SCNAs, and SV) to accurately predict C1-C5 DLBCLs.

### Example 6: Development of a Targeted Sequencing Assay

The next step towards the development of a broad-based clinical assay was to design a targeted sequencing panel that captured all of the genetic alterations needed to classify DLBCLs as clusters C1-C5 with the molecular classifier. The first version of this targeted bait set was designed using TWIST Biosciences chemistry. In additional to capturing the recurrent genetic alterations needed for the molecular classifier, the targeted sequencing assay also included bait sets to measure microsatellite instability, to measure tumor mutational burden, and detect Epstein Barr virus.

The sequencing assay and molecular classifier can be used as companion diagnostics and CLIA-approved tests for broader clinical use.

### Example 7: Targeted Sequencing Assay

The present example describes how the targeted sequencing panel is used to measure genetic alterations (variants) needed to classify DLBCLs as belonging to one of clusters C1-C5 using the molecular classifier.

### Library Construction

An aliquot of genomic DNA (100-250 ng in 50 μL) is used as the input into DNA fragmentation (aka shearing). Shearing is performed acoustically using a Covaris focused-ultrasonicator, targeting 150 bp fragments. For cfDNA (cell free DNA; e.g., circulating tumor DNA), no shearing is performed and 5-50 ng of DNA is used.

Library preparation is performed using a commercially available kit provided by KAPA Biosystems (KAPA HyperPrep Kit with Library Amplification product KK8504) and IDT's duplex UMI adapters. Unique 8-base dual index sequences embedded within the p5 and p7 primers (from IDT) are added during PCR. Enzymatic clean-ups are performed using Beckman Coultier AMPure XP beads with elution volumes reduced to 30 μL to maximize library concentration.

Following library construction, library quantification is performed using the Invitrogen Quant-It broad range dsDNA quantification assay kit (Thermo Scientific Catalog: Q33130) with a 1:200 PicoGreen dilution. Following quantification, each library is normalized to a set concentration (e.g., 35 ng/μL), using Tris-HCl, 10 mM, pH 8.0. All steps performed during the library construction process and library quantification process are performed on the Agilent Bravo liquid handling system.

### In-Solution Hybrid Selection

After library construction, hybridization and capture are performed using IDT's XGen hybridization and wash kit following the manufacturer's suggested protocol, with some alterations. A set of 12-plex pre-hybridization pools is created. These pre-hybridization pools are created by equivolume pooling of the normalized libraries, Human Cot-1, and IDT XGen blocking oligos. The pre-hybridization pools undergo lyophilization using the Biotage SPE-DRY. Post lyophilization, the targeted sequencing panel (TWIST Biosciences) along with hybridization mastermix is added to the lyophilized pool prior to resuspension. Samples are incubated overnight. Library normalization and hybridization setup are performed on a Hamilton Starlet liquid handling platform, while target capture is performed on the Agilent Bravo automated platform. Post capture, a PCR is performed to amplify captured DNA.

### Preparation of Libraries for Cluster Amplification and Sequencing

After post-capture enrichment, library pools are quantified using qPCR (automated assay on the Agilent Bravo), using a kit from KAPA Biosystems with probes specific to the ends of the adapters. Based on qPCR quantification, pools are normalized using a Hamilton Starlet to the required loading concentration. Up to 24 samples are loaded per flowcell lane for Illumina's NovaSeq S4 sequencing technology.

### Cluster Amplification and Sequencing

Cluster amplification of library pools is performed according to the manufacturer's protocol (Illumina) using Exclusion Amplification cluster chemistry and NovaSeq S4 flowcells. Flowcells are sequenced using Sequencing-by-Synthesis chemistry for NovaSeq S4 flowcells using paired 151 bp runs.

### Other Embodiments

From the foregoing description, it will be apparent that variations and modifications may be made to the invention described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The present disclosure may be related to U.S. Patent Application Publication No. 2019/0292602 A1, published Sep. 26, 2019, the disclosure of which is incorporated herein by reference for all purposes. All patents and publications mentioned in this specification are herein incorporated by reference to the same extent as if each independent patent and publication was specifically and individually indicated to be incorporated by reference.

What is claimed is:

1. A method for characterizing a diffuse large B-cell lymphoma (DLBCL) in a subject, the method comprising:
(a) characterizing variants in a biological sample from the subject, wherein the variants are selected from the group consisting of 10q23.31, 11p, 11q, 11q23.3, 12p, 12p13.2, 12q, 13q, 13q14.2, 13q31.3, 13q34, 14q32.31, 15q15.3, 16q12.1, 17p, 17q24.3, 17q25.1, 18p, 18q, 18q21.32, 18q22.2, 18q23, 19p13.2, 19p13.3, 19q, 19q13.32.1, 19q13.42, 1p13.1, 1p31.1, 1p36.11, 1p36.32, 1q, 1932.1, 1942.12, 21q, 2p16.1, 2q22.2, 3p, 3p21.31, 3q28, 4q21.22, 5p, 6p, 6p21.1, 6p21.33, 6q14.1, 6q21, 7p, 7q, 7q22.1, 8q12.1, 8q24.22, 9p21.3, 9p24.1, ACTB, ARID1A, ATP2A2, B2M, BCL10, BCL2, BCL6, BCL7A, BRAF, BTG1, BTG2, CARD11, CCDC27, CD274, CD58, CD70, CD79B^{mut}, CD83, CREBBP, CRIP1, CXCR4, DTX1, DUSP2, EBF1, EEF1A1, EP300, ETV6, EZH2, FADD, FAS, GNA13, GNAI2, GRHPR, HIST1H1B, HIST1H1C, HIST1H1D, HIST1H1E, HIST1H2AC, HIST1H2AM, HIST1H2BC, HIST1H2BD, HLA-A, HLA-B, HLA-C, HVCN1, IGLL5, IKZF3, IRF2BP2, IRF8, KLHL6, KMT2D, KRAS, LTB, LYN, MAP2K1, MEF2B, MEF2C, MYC, MYD88, MYD88^{L265P}, MYD88^{OTHER}, NFKBIA, NFKBIE, NOTCH2, OSBPL10, PABPC1, PDE4DIP, PIM1, POU2AF1, POU2F2, PRDM1, PTEN, PTPN6, RAC2, RHOA, SESN3, SF3B1, SGK1, SMG7, SOCS1, SPEN, STAT3, TBL1XR1, TET2, TMEM30A, TMSB4X, TNFAIP3, TNFRSF14, TNIP1, TOX, TP53, TUBGCP5, UBE2A, VMP1, YY1, ZC3H12A, ZEB2, ZFP36L1, ZNF423, and ZNF608, by using a targeted sequencing panel to characterize classes of the variants in the sample based upon the characterization of alterations in the variants, wherein the alterations are selected from the group consisting of a mutation, a structural variant (SV), and a somatic copy number alteration (SCNA);
(b) assigning a classification-specific weighted value to each class of variant characterized, wherein each classification-specific weighted value reflects the magnitude of the characterized alteration in each class of variant;
(c) condensing the variant classification-specific weighted values into two or more metafeatures; and
(d) using the metafeatures as input variables for a computational analysis to assign the DLBCL to one of DLBCL subclasses C1 to C5, thereby characterizing the DLBCL.

2. The method of claim 1, further comprising characterizing the DLBCL as high-risk if the DLBCL is assigned to subclass C5 or C3.

3. A method for selecting a treatment for a subject having a diffuse large B-cell lymphoma (DLBCL), the method comprising:
(a) characterizing variants in a biological sample from the subject, wherein the variants are selected from the group consisting of 10q23.31, 11p, 11q, 11q23.3, 12p, 12p13.2, 12q, 13q, 13q14.2, 13q31.3, 13q34, 14q32.31, 15q15.3, 16q12.1, 17p, 17q24.3, 17q25.1, 18p, 18q, 18q21.32, 18q22.2, 18q23, 19p13.2, 19p13.3, 19q, 19q13.32.1, 19q13.42, 1p13.1, 1p31.1, 1p36.11, 1p36.32, 1q, 1932.1, 1942.12, 21q, 2p16.1, 2q22.2, 3p, 3p21.31, 3q28, 4q21.22, 5p, 6p, 6p21.1, 6p21.33, 6q14.1, 6q21, 7p, 7q, 7q22.1, 8q12.1, 8q24.22, 9p21.3, 9p24.1, ACTB, ARID1A, ATP2A2, B2M, BCL10, BCL2, BCL6, BCL7A, BRAF, BTG1, BTG2, CARD11, CCDC27, CD274, CD58, CD70, CD79B^{mut}, CD83, CREBBP, CRIP1, CXCR4, DTX1, DUSP2, EBF1, EEF1A1, EP300, ETV6, EZH2, FADD, FAS, GNA13, GNAI2, GRHPR, HIST1H1B, HIST1H1C, HIST1H1D, HIST1H1E, HIST1H2AC, HIST1H2AM, HIST1H2BC, HIST1H2BD, HLA-A, HLA-B, HLA-C, HVCN1, IGLL5, IKZF3, IRF2BP2, IRF8, KLHL6, KMT2D, KRAS, LTB, LYN, MAP2K1, MEF2B, MEF2C, MYC, MYD88, MYD88^{L265P}, MYD88^{OTHER}, NFKBIA, NFKBIE, NOTCH2, OSBPL10, PABPC1, PDE4DIP, PIM1, POU2AF1, POU2F2, PRDM1, PTEN, PTPN6, RAC2, RHOA, SESN3, SF3B1, SGK1, SMG7, SOCS1, SPEN, STAT3, TBL1XR1, TET2, TMEM30A, TMSB4X, TNFAIP3, TNFRSF14, TNIP1, TOX, TP53, TUBGCP5, UBE2A, VMP1, YY1, ZC3H12A, ZEB2, ZFP36L1, ZNF423, and ZNF608, by characterizing classes of the variants in the sample based upon the characterization of alterations in the variants, wherein the alterations are selected from the group consisting of a mutation, a structural variant (SV), and a somatic copy number alteration (SCNA);
(b) assigning a classification-specific weighted value to each class of variant characterized, wherein each classification-specific weighted value reflects the magnitude of the characterized alteration in each class of variant;
(c) condensing the variant classification-specific weighted values into two or more metafeatures;
(d) assigning the DLBCL as belonging to one of DLBCL subclasses C1 to C5 using a computational analysis, wherein the metafeatures are used as input variables for the computational analysis; and
(e) (i) if the DLBCL is assigned to class C1 or C5, administering to the subject a treatment comprising:
an agent selected from the group consisting of a NOTCH inhibitor, a BCL6 inhibitor and an activator of immune evasion, optionally an oligonucleotide inhibitor of NOTCH and/or BCL6, or
an agent selected from the group consisting of a BCR/TLR signaling inhibitor and a BCL2 inhibitor, optionally oblimersen, ABT-263, Venetoclax (ABT-199), an antibody or oligonucleotide inhibitor of BCR/TLR signaling and/or an oligonucleotide inhibitor of BCL2; or
(ii) if the DLBCL is assigned to DLBCL subclass C3 or C4 class, administering to the subject a treatment comprising:
an agent selected from the group consisting of a BCL2 inhibitor, a PI3K inhibitor and an epigenetic modifier, optionally oblimersen, ABT-263, Venetoclax (ABT-199), wortmannin, LY294002, an E2H2 inhibitor (optionally 3-deazaneplanocin A (DZNep), EPZ005687, ElIl, GSK126, and/or UNC1999), a CREBBP inhibitor, an oligonucleotide inhibitor of BCL2, an oligonucleotide inhibitor of PI3K and/or an oligonucleotide inhibitor of an epigenetic modifier; or
an agent selected from the group consisting of a JAK/STAT inhibitor and a BRAF/MEK1 inhibitor, optionally ruxolitinib, Vemurafenib, Cobimetinib, an oligonucleotide inhibitor of JAK/STAT and/or an oligonucleotide inhibitor of BRAF/MEK1; or
(iii) if the DLBCL is assigned to DLBCL subclass C2, selecting a treatment comprising a CDK inhibitor, thereby selecting a treatment for the subject having a DLBCL.

4. The method of claim 3, wherein the agent comprises an agent selected from the group consisting of rituximab, cyclophosphamide adriamycin, vincristine, prednisone, doxorubicin hydrochloride, and vincristine sulfate.

5. The method of claim 1, wherein less than 25 metafeatures are used as the input variables.

6. The method of claim 1, wherein, in step (a), characterization comprises (i) determining for the mutation classes of variants whether there is a mutation and, if there is a mutation, whether the mutation is a silent mutation or a non-synonymous mutation; (ii) determining for the SCNA mutation class of variants whether there is an SCNA and, if there is an SCNA, whether the SCNA is a low level copy number alteration or a high level copy number alteration; and/or (iii) determining for the SV mutation class of variants whether or not an SV is present.

7. The method of claim 1, wherein the computational method is an artificial neural network classification method.

8. The method of claim 1, wherein the biological sample comprises cell free DNA.

9. The method of claim 8, wherein the method comprises characterizing the variants in the cell free DNA.

10. A targeted sequencing panel comprising oligonucleotides suitable for use in targeted sequencing to characterize two or more classes of variants in a biological sample based upon the characterization of alterations in the variants, wherein the alterations are selected from the group consisting of a mutation, a structural variant (SV), and a somatic copy number alteration (SCNA), and wherein the variants are selected from the group consisting of 10q23.31, 11p, 11q, 11q23.3, 12p, 12p13.2, 12q, 13q, 13q14.2, 13q31.3, 13q34, 14q32.31, 15q15.3, 16q12.1, 17p, 17q24.3, 17q25.1, 18p, 18q, 18q21.32, 18q22.2, 18q23, 19p13.2, 19p13.3, 19q, 19q13.32.1, 19q13.42, 1p13.1, 1p31.1, 1p36.11, 1p36.32, 1q, 1932.1, 1942.12, 21q, 2p16.1, 2q22.2, 3p, 3p21.31, 3q28, 4q21.22, 5p, 6p, 6p21.1, 6p21.33, 6q14.1, 6q21, 7p, 7q, 722.1, 8q12.1, 8q24.22, 9p21.3, 9p24.1, ACTB, ARID1A, ATP2A2, B2M, BCL10, BCL2, BCL6, BCL7A, BRAF, BTG1, BTG2, CARD11, CCDC27, CD274, CD58, CD70, CD79B^{mut}, CD83, CREBBP, CRIP1, CXCR4, DTX1, DUSP2, EBF1, EEF1A1, EP300, ETV6, EZH2, FADD, FAS, GNA13, GNAI2, GRHPR, HIST1H1B, HIST1H1C, HIST1H1D, HIST1H1E, HIST1H2AC, HIST1H2AM, HIST1H2BC, HIST1H2BD, HLA-A, HLA-B, HLA-C, HVCN1, IGLL5, IKZF3, IRF2BP2, IRF8, KLHL6, KMT2D, KRAS, LTB, LYN, MAP2K1, MEF2B, MEF2C, MYC, MYD88, MYD88^{L265P}, MYD88^{OTHER}, NFKBIA, NFKBIE, NOTCH2, OSBPL10, PABPC1, PDE4DIP, PIM1, POU2AF1, POU2F2, PRDM1, PTEN, PTPN6, RAC2, RHOA, SESN3, SF3B1, SGK1, SMG7, SOCS1, SPEN, STAT3, TBL1XR1, TET2, TMEM30A, TMSB4X, TNFAIP3, TNFRSF14, TNIP1, TOX, TP53, TUBGCP5, UBE2A, VMP1, YY1, ZC3H12A, ZEB2, ZFP36L1, ZNF423, and ZNF608.

11. The targeted sequencing panel of claim 10, wherein the oligonucleotides are suitable for use in targeted sequencing to characterize all of the variant classes listed in Table 3.

12. The targeted sequencing panel of claim 10 further comprising oligonucleotide sequences suitable for use in targeted sequencing to measure microsatellite instability, tumor mutational burden, and/or to detect Epstein Barr virus.

13. The targeted sequencing panel of claim 10, wherein the targeted sequencing panel comprises polynucleotides sharing at least 85% sequence identity over a span of at least 80 nucleotides to at least one sequence of SEQ ID NOs: 1-9244 targeting microsatellite instability.

14. A method comprising:
instructing, by at least one processor, at least one computing device to render at least one diffuse large B-cell lymphoma (DLBCL) classification interface, the at least one DLBCL classification interface comprising at least one gene sample matrix (GSM) array input element configured to accept at least one GSM array input file storing at least one GSM array associated with at least one patient;
receiving, by the at least one processor, via the at least one GSM array input element the at least one GSM array input file, wherein the at least one GSM array input file represents at least one GSM array that characterizes classes of variants in the at least one sample of oligonucleotides, wherein the classes of variants are characterized using a targeted sequencing panel comprising the oligonucleotides suitable for use in targeted sequencing of the variant classes;
generating, by the at least one processor, at least one metafeature based at least in part on a weighted sum of the classes of the variants in the at least one GSM array;
utilizing, by the at least one processor, at least one DLBCL classification machine learning model to generate at least one cluster identification categorizing the at least one patient based at least in part on the at least one metafeature and at least one trained classification layer, wherein the at least one cluster identification characterizes DLBCL burden on a subject associated with the at least one patient; and
instructing, by the at least one processor, the at least one computing device to render at least one DLBCL classification results element in the at least one DLBCL classification interface, wherein the at least one DLBCL classification results element depicts a representation of the at least one cluster identification categorizing the at least one patient.

15. The method of claim 14, further comprising:
receiving, by the at least one processor, at least one DLBCL classification request from the at least one computing device associated with the at least one patient, wherein the at least one DLBCL classification request comprises at least one electronic request over a network; and
generating, by the at least one processor, at least one rendering instruction in response to the at least one DLBCL classification request, wherein the at least one rendering instruction is configured to instruct the at least one computing device to render the at least one DLBCL classification interface.

16. The method of claim 14, wherein the at least one trained classification layer comprises an artificial neural network having learned weights for each of a plurality of neural network nodes.

17. The method of claim 14, further comprising:
utilizing, by the at least one processor, at least one dimensionality reduction model to create a two-dimensional representation of the at least one metafeature; and
generating, by the at least one processor, the at least one DLBCL classification results element comprising a two-dimensional visualization of the two-dimensional representation of the at least one metafeature, wherein the two-dimensional visualization comprises at least one labelled data point representing the at least one metafeature and the at least one cluster identification associated with the at least one metafeature.

18. A system comprising:
at least one processor configured to execute software instructions which, upon execution, cause the at least one processor to perform steps to:
instruct at least one computing device to render at least one diffuse large B-cell lymphoma (DLBCL) classification interface, the at least one DLBCL classification interface comprising at least one gene sample matrix (GSM) array input element configured to accept at least one GSM array input file storing at least one GSM array associated with at least one patient;
receive via the at least one GSM array input element the at least one GSM array input file, wherein the at least one GSM array input file represents at least one GSM array that characterizes classes of variants in the at least one sample of oligonucleotides, wherein the classes of variants are characterized using a targeted sequencing panel comprising the oligonucleotides suitable for use in targeted sequencing of the variant classes;
generate at least one metafeature based at least in part on a weighted sum of the classes of the variants in the at least one GSM array;
utilize at least one DLBCL classification machine learning model to generate at least one cluster identification categorizing the at least one patient based at least in part on the at least one metafeature and at least one trained classification layer, wherein the at least one cluster identification characterizes DLBCL burden on a subject associated with the at least one patient; and
instruct the at least one computing device to render at least one DLBCL classification results element in the at least one DLBCL classification interface, wherein the at least one DLBCL classification results element depicts a representation of the at least one cluster identification categorizing the at least one patient.

19. The system of claim 18, wherein the at least one processor is further configured to execute software instructions which, upon execution, further cause the at least one processor to perform steps to:
receive at least one DLBCL classification request from the at least one computing device associated with the at least one patient, wherein the at least one DLBCL classification request comprises at least one electronic request over a network; and
generate at least one rendering instruction in response to the at least one DLBCL classification request, wherein the at least one rendering instruction is configured to instruct the at least one computing device to render the at least one DLBCL classification interface.

20. The system of claim 18, wherein the at least one trained classification layer comprises an artificial neural network having learned weights for each of a plurality of neural network nodes.

21. The system of claim 18, wherein the at least one processor is further configured to execute software instructions which, upon execution, further cause the at least one processor to perform steps to:
utilize at least one dimensionality reduction model to create a two-dimensional representation of the at least one metafeature; and
generate the at least one DLBCL classification results element comprising a two-dimensional visualization of the two-dimensional representation of the at least one metafeature, wherein the two-dimensional visualization comprises at least one labelled data point representing the at least one metafeature and the at least one cluster identification associated with the at least one metafeature.
